SEQUENCE LISTING (256 sequences)

SEQ ID NO: 1 - protein, 177 aa, Homo sapiens
  Ephrin type-A receptor 1
EVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLVSGSVKLNVERCSLGRLTRRGLYLAFHNPGACVALVSVRVFYQR

SEQ ID NO: 2 - protein, 173 aa, Homo sapiens
  Ephrin type-A receptor 2
EVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKK

SEQ ID NO: 3 - protein, 173 aa, Homo sapiens
  Ephrin type-A receptor 3
EVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKK

SEQ ID NO: 4 - protein, 174 aa, Homo sapiens
  Ephrin type-A receptor 4
EVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKK

SEQ ID NO: 5 - protein, 173 aa, Homo sapiens
  Ephrin type-A receptor 5
EVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKK

SEQ ID NO: 6 - protein, 173 aa, Homo sapiens
  Ephrin type-A receptor 6
QVVLLDTTTVLGELGWKTYPLNGWDAITEMDEHNRPIHTYQVCNVMEPNQNNWLRTNWISRDAAQKIYVEMKFTLRDCNSIPWVLGTCKETFNLFYMESDESHGIKFKPNQYTKIDTIAADESFTQMDLGDRILKLNTEIREVGPIERKGFYLAFQDIGACIALVSVRVFYKK

SEQ ID NO: 7 - protein, 175 aa, Homo sapiens
  Ephrin type-A receptor 7
AKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKK

SEQ ID NO: 8 - protein, 173 aa, Homo sapiens
  Ephrin type-A receptor 8
EVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKK

SEQ ID NO: 9 - protein, 176 aa, Homo sapiens
  Ephrin type-A receptor 10
EVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQ

SEQ ID NO: 10 - protein, 176 aa, Homo sapiens
  Ephrin type-B receptor 1
ETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKK

SEQ ID NO: 11 - protein, 178 aa, Homo sapiens
  Ephrin type-B receptor 2
VEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRK

SEQ ID NO: 12 - protein, 173 aa, Homo sapiens
  Ephrin type-B receptor 3
EETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKK

SEQ ID NO: 13 - protein, 180 aa, Homo sapiens
  Ephrin type-B receptor 4
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKK

SEQ ID NO: 14 - protein, 199 aa, Homo sapiens
  Ephrin type-B receptor 6
EEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYT

SEQ ID NO: 15 - protein, 174 aa, Artificial Sequence
  EphA4_mut F154A
EVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESATQVDIGDRIMKLNTEIRDVGPLSKKGFYLAFQDVGACIALVSVRVFYKK

SEQ ID NO: 16 - protein, 173 aa, Artificial Sequence
  EphA2_mut R103E
EVVLLDFAAAGGELGWLTHPYGKGWDLMQNIMNDMPIYMYSVCNVMSGDQDNWLRTNWVYRGEAERIFIELKFTVEDCNSFPGGASSCKETFNLYYAESDLDYGTNFQKRLFTKIDTIAPDEITVSSDFEARHVKLNVEERSVGPLTRKGFYLAFQDIGACVALLSVRVYYKK

SEQ ID NO: 17 - protein, 209 aa, Artificial Sequence
  EphA4_with Ephrin loop insertion
EVTLLDSRSVQGELGWIASPLGGSGGSGGSKFQLFTPFSLGFEFRPGRGGSGGSGGSGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESATQVDIGDRIMKLNTEIRDVGPLSKKGFYLAFQDVGACIALVSVRVFYKK

SEQ ID NO: 18 - protein, 139 aa, Homo sapiens
  Ephrin type-A receptor 1
CVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVAC

SEQ ID NO: 19 - protein, 138 aa, Homo sapiens
  Ephrin type-A receptor 2
CVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPC

SEQ ID NO: 20 - protein, 134 aa, Homo sapiens
  Ephrin type-A receptor 3
CVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMAC

SEQ ID NO: 21 - protein, 135 aa, Homo sapiens
  Ephrin type-A receptor 4
CIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPC

SEQ ID NO: 22 - protein, 135 aa, Homo sapiens
  Ephrin type-A receptor 5
CIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMAC

SEQ ID NO: 23 - protein, 135 aa, Homo sapiens
  Ephrin type-A receptor 6
CIALVSVRVFYKKCPFTVRNLAMFPDTIPRVDSSSLVEVRGSCVKSAEERDTPKLYCGADGDWLVPLGRCICSTGYEEIEGSCHACRPGFYKAFAGNTKCSKCPPHSLTYMEATSVCQCEKGYFRAEKDPPSMAC

SEQ ID NO: 24 - protein, 137 aa, Homo sapiens
  Ephrin type-A receptor 7
CIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVAC

SEQ ID NO: 25 - protein, 135 aa, Homo sapiens
  Ephrin type-A receptor 8
CLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSAC

SEQ ID NO: 26 - protein, 137 aa, Homo sapiens
  Ephrin type-A receptor 10
CVALVSVRVYYKQCRATVRGLATFPATAAESAFSTLVEVAGTCVAHSEGEPGSPPRMHCGADGEWLVPVGRCSCSAGFQERGDFCEACPPGFYKVSPRRPLCSPCPEHSRALENASTFCVCQDSYARSPTDPPSASC

SEQ ID NO: 27 - protein, 137 aa, Homo sapiens
  Ephrin type-B receptor 1
CMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVAC

SEQ ID NO: 28 - protein, 141 aa, Homo sapiens
  Ephrin type-B receptor 2
CMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTI

SEQ ID NO: 29 - protein, 138 aa, Homo sapiens
  Ephrin type-B receptor 3
CMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSAC

SEQ ID NO: 30 - protein, 137 aa, Homo sapiens
  Ephrin type-B receptor 4
CMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPC

SEQ ID NO: 31 - protein, 148 aa, Homo sapiens
  Ephrin type-B receptor 6
CLALVAVRLFSYTCPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPC

SEQ ID NO: 32 - protein, 114 aa, Homo sapiens
  Ephrin type-A receptor 1
PPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHA

SEQ ID NO: 33 - protein, 105 aa, Homo sapiens
  Ephrin type-A receptor 2
PPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASV

SEQ ID NO: 34 - protein, 111 aa, Homo sapiens
  Ephrin type-A receptor 3
PPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQA

SEQ ID NO: 35 - protein, 112 aa, Homo sapiens
  Ephrin type-A receptor 4
PPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQA

SEQ ID NO: 36 - protein, 111 aa, Homo sapiens
  Ephrin type-A receptor 5
PPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQA

SEQ ID NO: 37 - protein, 111 aa, Homo sapiens
  Ephrin type-A receptor 6
PPSAPRNVVFNINETALILEWSPPSDTGGRKDLTYSVICKKCGLDTSQCEDCGGGLRFIPRHTGLINNSVIVLDFVSHVNYTFEIEAMNGVSELSFSPKPFTAITVTTDQD

SEQ ID NO: 38 - protein, 111 aa, Homo sapiens
  Ephrin type-A receptor 7
PPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQA

SEQ ID NO: 39 - protein, 111 aa, Homo sapiens
  Ephrin type-A receptor 8
PPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQA

SEQ ID NO: 40 - protein, 113 aa, Homo sapiens
  Ephrin type-A receptor 10
APRDLQYSLSRSPLVLRLRWLPPADSGGRSDVTYSLLCLRCGREGPAGACEPCGPRVAFLPRQAGLRERAATLLHLRPGARYTVRVAALNGVSGPAAAAGTTYAQVTVSTGPG

SEQ ID NO: 41 - protein, 111 aa, Homo sapiens
  Ephrin type-B receptor 1
VPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQA

SEQ ID NO: 42 - protein, 111 aa, Homo sapiens
  Ephrin type-B receptor 2
IPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQA

SEQ ID NO: 43 - protein, 113 aa, Homo sapiens
  Ephrin type-B receptor 3
VPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQA

SEQ ID NO: 44 - protein, 110 aa, Homo sapiens
  Ephrin type-B receptor 4
PPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDRE

SEQ ID NO: 45 - protein, 118 aa, Homo sapiens
  Ephrin type-B receptor 6
PPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHE

SEQ ID NO: 46 - protein, 93 aa, Homo sapiens
  Ephrin type-A receptor 1
ESLSGLSLRLVKKEPRQLELTWAGSRPRSPGANLTYELHVLNQDEERYQMVLEPRVLLTELQPDTTYIVRVRMLTPLGPGPFSPDHEFRTSPP

SEQ ID NO: 47 - protein, 92 aa, Homo sapiens
  Ephrin type-A receptor 2
EPPKVRLEGRSTTSLSVSWSIPPPQQSRVWKYEVTYRKKGDSNSYNVRRTEGFSVTLDDLAPDTTYLVQVQALTQEGQGAGSKVHEFQTLSP

SEQ ID NO: 48 - protein, 96 aa, Homo sapiens
  Ephrin type-A receptor 3
APSPVLTIKKDRTSRNSISLSWQEPEHPNGIILDYEVKYYEKQEQETSYTILRARGTNVTISSLKPDTIYVFQIRARTAAGYGTNSRKFEFETSPD

SEQ ID NO: 49 - protein, 98 aa, Homo sapiens
  Ephrin type-A receptor 4
APSSIALVQAKEVTRYSVALAWLEPDRPNGVILEYEVKYYEKDQNERSYRIVRTAARNTDIKGLNPLTSYVFHVRARTAAGYGDFSEPLEVTTNTVPS

SEQ ID NO: 50 - protein, 95 aa, Homo sapiens
  Ephrin type-A receptor 5
APSPVTNVKKGKIAKNSISLSWQEPDRPNGIILEYEIKYFEKDQETSYTIIKSKETTITAEGLKPASVYVFQIRARTAAGYGVFSRRFEFETTPV

SEQ ID NO: 51 - protein, 96 aa, Homo sapiens
  Ephrin type-A receptor 6
APSLIGVVRKDWASQNSIALSWQAPAFSNGAILDYEIKYYEKEHEQLTYSSTRSKAPSVIITGLKPATKYVFHIRVRTATGYSGYSQKFEFETGDE

SEQ ID NO: 52 - protein, 96 aa, Homo sapiens
  Ephrin type-A receptor 7
APSQVSGVMKERVLQRSVELSWQEPEHPNGVITEYEIKYYEKDQRERTYSTVKTKSTSASINNLKPGTVYVFQIRAFTAAGYGNYSPRLDVATLEE

SEQ ID NO: 53 - protein, 96 aa, Homo sapiens
  Ephrin type-A receptor 8
APSQVVVIRQERAGQTSVSLLWQEPEQPNGIILEYEIKYYEKDKEMQSYSTLKAVTTRATVSGLKPGTRYVFQVRARTSAGCGRFSQAMEVETGKP

SEQ ID NO: 54 - protein, 102 aa, Homo sapiens
  Ephrin type-A receptor 10
APWEEDEIRRDRVEPQSVSLSWREPIPAGAPGANDTEYEIRYYEKGQSEQTYSMVKTGAPTVTVTNLKPATRYVFQIRAASPGPSWEAQSFNPSIEVQTLGE

SEQ ID NO: 55 - protein, 96 aa, Homo sapiens
  Ephrin type-B receptor 1
APSTVPIMHQVSATMRSITLSWPQPEQPNGIILDYEIRYYEKEHNEFNSSMARSQTNTARIDGLRPGMVYVVQVRARTVAGYGKFSGKMCFQTLTD

SEQ ID NO: 56 - protein, 96 aa, Homo sapiens
  Ephrin type-B receptor 2
APSAVSIMHQVSRTVDSITLSWSQPDQPNGVILDYELQYYEKELSEYNATAIKSPTNTVTVQGLKAGAIYVFQVRARTVAGYGRYSGKMYFQTMTE

SEQ ID NO: 57 - protein, 94 aa, Homo sapiens
  Ephrin type-B receptor 3
APSEVPTLRLHSSSGSSLTLSWAPPERPNGVILDYEMKYFEKSEGIASTVTSQMNSVQLDGLRPDARYVVQVRARTVAGYGQYSRPAEFETTSE

SEQ ID NO: 58 - protein, 97 aa, Homo sapiens
  Ephrin type-B receptor 4
VPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLD

SEQ ID NO: 59 - protein, 96 aa, Homo sapiens
  Ephrin type-B receptor 6
VPSAVPVVHQVSRASNSITVSWPQPDQTNGNILDYQLRYYDQAEDESHSFTLTSETNTATVTQLSPGHIYGFQVRARTAAGHGPYGGKVYFQTLPQ

SEQ ID NO: 60 - protein, 335 aa, Homo sapiens
  Ephrin type-A receptor 1
CPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPP

SEQ ID NO: 61 - protein, 329 aa, Homo sapiens
  Ephrin type-A receptor 2
CPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSP

SEQ ID NO: 62 - protein, 330 aa, Homo sapiens
  Ephrin type-A receptor 3
CPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPD

SEQ ID NO: 63 - protein, 334 aa, Homo sapiens
  Ephrin type-A receptor 4
CPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPS

SEQ ID NO: 64 - protein, 330 aa, Homo sapiens
  Ephrin type-A receptor 5
CPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPV

SEQ ID NO: 65 - protein, 331 aa, Homo sapiens
  Ephrin type-A receptor 6
CPFTVRNLAMFPDTIPRVDSSSLVEVRGSCVKSAEERDTPKLYCGADGDWLVPLGRCICSTGYEEIEGSCHACRPGFYKAFAGNTKCSKCPPHSLTYMEATSVCQCEKGYFRAEKDPPSMACTRPPSAPRNVVFNINETALILEWSPPSDTGGRKDLTYSVICKKCGLDTSQCEDCGGGLRFIPRHTGLINNSVIVLDFVSHVNYTFEIEAMNGVSELSFSPKPFTAITVTTDQDAPSLIGVVRKDWASQNSIALSWQAPAFSNGAILDYEIKYYEKEHEQLTYSSTRSKAPSVIITGLKPATKYVFHIRVRTATGYSGYSQKFEFETGDE

SEQ ID NO: 66 - protein, 333 aa, Homo sapiens
  Ephrin type-A receptor 7
CWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEE

SEQ ID NO: 67 - protein, 331 aa, Homo sapiens
  Ephrin type-A receptor 8
CPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKP

SEQ ID NO: 68 - protein, 344 aa, Homo sapiens
  Ephrin type-A receptor 10
CRATVRGLATFPATAAESAFSTLVEVAGTCVAHSEGEPGSPPRMHCGADGEWLVPVGRCSCSAGFQERGDFCEACPPGFYKVSPRRPLCSPCPEHSRALENASTFCVCQDSYARSPTDPPSASCTRPPSAPRDLQYSLSRSPLVLRLRWLPPADSGGRSDVTYSLLCLRCGREGPAGACEPCGPRVAFLPRQAGLRERAATLLHLRPGARYTVRVAALNGVSGPAAAAGTTYAQVTVSTGPGAPWEEDEIRRDRVEPQSVSLSWREPIPAGAPGANDTEYEIRYYEKGQSEQTYSMVKTGAPTVTVTNLKPATRYVFQIRAASPGPSWEAQSFNPSIEVQTLGE

SEQ ID NO: 69 - protein, 333 aa, Homo sapiens
  Ephrin type-B receptor 1
CPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTD

SEQ ID NO: 70 - protein, 334 aa, Homo sapiens
  Ephrin type-B receptor 2
CPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTE

SEQ ID NO: 71 - protein, 334 aa, Homo sapiens
  Ephrin type-B receptor 3
CASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSE

SEQ ID NO: 72 - protein, 333 aa, Homo sapiens
  Ephrin type-B receptor 4
CAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLD

SEQ ID NO: 73 - protein, 351 aa, Homo sapiens
  Ephrin type-B receptor 6
CPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPCTGPPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHEVPSAVPVVHQVSRASNSITVSWPQPDQTNGNILDYQLRYYDQAEDESHSFTLTSETNTATVTQLSPGHIYGFQVRARTAAGHGPYGGKVYFQTLPQ

SEQ ID NO: 74 - protein, 21 aa, Homo sapiens
  Ephrin type-A receptor 1
IVAVIFGLLLGAALLLGILVF

SEQ ID NO: 75 - protein, 21 aa, Homo sapiens
  Ephrin type-A receptor 2
IGGVAVGVVLLLVLAGVGFFI

SEQ ID NO: 76 - protein, 24 aa, Homo sapiens
  Ephrin type-A receptor 3
VVMIAISAAVAIILLTVVIYVLIG

SEQ ID NO: 77 - protein, 22 aa, Homo sapiens
  Ephrin type-A receptor 4
VLLVSVSGSVVLVVILIAAFVI

SEQ ID NO: 78 - protein, 21 aa, Homo sapiens
  Ephrin type-A receptor 5
VIAVSVTVGVILLAVVIGVLL

SEQ ID NO: 79 - protein, 21 aa, Homo sapiens
  Ephrin type-A receptor 6
IATAAVGGFTLLVILTLFFLI

SEQ ID NO: 80 - protein, 21 aa, Homo sapiens
  Ephrin type-A receptor 7
IIIAVVAVAGTIILVFMVFGF

SEQ ID NO: 81 - protein, 21 aa, Homo sapiens
  Ephrin type-A receptor 8
IVWICLTLITGLVVLLLLLIC

SEQ ID NO: 82 - protein, 21 aa, Homo sapiens
  Ephrin type-A receptor 10
IVVTVVTISALLVLGSVMSVL

SEQ ID NO: 83 - protein, 23 aa, Homo sapiens
  Ephrin type-B receptor 1
LIAGSAAAGVVFVVSLVAISIVC

SEQ ID NO: 84 - protein, 21 aa, Homo sapiens
  Ephrin type-B receptor 2
IIGSSAAGLVFLIAVVVIAIV

SEQ ID NO: 85 - protein, 21 aa, Homo sapiens
  Ephrin type-B receptor 3
IVGSATAGLVFVVAVVVIAIV

SEQ ID NO: 86 - protein, 21 aa, Homo sapiens
  Ephrin type-B receptor 4
LIAGTAVVGVVLVLVVIVVAV

SEQ ID NO: 87 - protein, 21 aa, Homo sapiens
  Ephrin type-B receptor 6
LVIGSILGALAFLLLAAITVL

SEQ ID NO: 88 - protein, 49 aa, Homo sapiens
  Ephrin type-A receptor 1
RRAQRQRQQRQRDRATDVDREDKLWLKPYVDLQAYEDPAQGALDFTREL

SEQ ID NO: 89 - protein, 50 aa, Homo sapiens
  Ephrin type-A receptor 2
HRRRKNQRARQSPEDVYFSKSEQLKPLKTYVDPHTYEDPNQAVLKFTTEI

SEQ ID NO: 90 - protein, 52 aa, Homo sapiens
  Ephrin type-A receptor 3
GRFCGYKSKHGADEKRLHFGNGHLKLPGLRTYVDPHTYEDPTQAVHEFAKEL

SEQ ID NO: 91 - protein, 47 aa, Homo sapiens
  Ephrin type-A receptor 4
SRRRSKYSKAKQEADEEKHLNQGVRTYVDPFTYEDPNQAVREFAKEI

SEQ ID NO: 92 - protein, 76 aa, Homo sapiens
  Ephrin type-A receptor 5
SGSCCECGCGRASSLCAVAHPSLIWRCGYSKAKQDPEEEKMHFHNGHIKLPGVRTYIDPHTYEDPNQAVHEFAKEI

SEQ ID NO: 93 - protein, 55 aa, Homo sapiens
  Ephrin type-A receptor 6
TGRCQWYIKAKMKSEEKRRNHLQNGHLRFPGIKTYIDPDTYEDPSLAVHEFAKEI

SEQ ID NO: 94 - protein, 50 aa, Homo sapiens
  Ephrin type-A receptor 7
GRRHCGYSKADQEGDEELYFHFKFPGTKTYIDPETYEDPNRAVHQFAKEL

SEQ ID NO: 95 - protein, 67 aa, Homo sapiens
  Ephrin type-A receptor 8
KKRHCGYSKAFQDSDEEKMHYQNGQAPPPVFLPLHHPPGKLPEPQFYAEPHTYEEPGRAGRSFTREI

SEQ ID NO: 96 - protein, 51 aa, Homo sapiens
  Ephrin type-A receptor 10
RRPCSYGKGGGDAHDEEELYFHFKVPTRRTFLDPQSCGDLLQAVHLFAKEL

SEQ ID NO: 97 - protein, 50 aa, Homo sapiens
  Ephrin type-B receptor 1
RKRAYSKEAVYSDKLQHYSTGRGSPGMKIYIDPFTYEDPNEAVREFAKEI

SEQ ID NO: 98 - protein, 50 aa, Homo sapiens
  Ephrin type-B receptor 2
RRGFERADSEYTDKLQHYTSGHMTPGMKIYIDPFTYEDPNEAVREFAKEI

SEQ ID NO: 99 - protein, 46 aa, Homo sapiens
  Ephrin type-B receptor 3
RKQRHGSDSEYTEKLQQYIAPGMKVYIDPFTYEDPNEAVREFAKEI

SEQ ID NO: 100 - protein, 47 aa, Homo sapiens
  Ephrin type-B receptor 4
RKQSNGREAEYSDKHGQYLIGHGTKVYIDPFTYEDPNEAVREFAKEI

SEQ ID NO: 101 - protein, 45 aa, Homo sapiens
  Ephrin type-B receptor 6
RKRRGTGYTEQLQQYSSPGLGVKYYIDPSTYEDPCQAIRELAREV

SEQ ID NO: 102 - protein, 261 aa, Homo sapiens
  Ephrin type-A receptor 1
LMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLL

SEQ ID NO: 103 - protein, 263 aa, Homo sapiens
  Ephrin type-A receptor 2
VTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLI

SEQ ID NO: 104 - protein, 262 aa, Homo sapiens
  Ephrin type-A receptor 3
ISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLI

SEQ ID NO: 105 - protein, 262 aa, Homo sapiens
  Ephrin type-A receptor 4
IKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLI

SEQ ID NO: 106 - protein, 262 aa, Homo sapiens
  Ephrin type-A receptor 5
ITIERVIGAGEFGEVCSGRLKLPGKRELPVAIKTLKVGYTEKQRRDFLGEASIMGQFDHPNIIHLEGVVTKSKPVMIVTEYMENGSLDTFLKKNDGQFTVIQLVGMLRGISAGMKYLSDMGYVHRDLAARNILINSNLVCKVSDFGLSRVLEDDPEAAYTTRGGKIPIRWTAPEAIAFRKFTSASDVWSYGIVMWEVVSYGERPYWEMTNQDVIKAVEEGYRLPSPMDCPAALYQLMLDCWQKERNSRPKFDEIVNMLDKLI

SEQ ID NO: 107 - protein, 304 aa, Homo sapiens
  Ephrin type-A receptor 6
IRIERVIGAGEFGEVCSGRLKTPGKREIPVAIKTLKGGHMDRQRRDFLREASIMGQFDHPNIIRLEGVVTKRSFPAIGVEAFCPSFLRAGFLNSIQAPHPVPGGGSLPPRIPAGRPVMIVVEYMENGSLDSFLRKHDGHFTVIQLVGMLRGIASGMKYLSDMGYVHRDLAARNILVNSNLVCKVSDFGLSRVLEDDPEAAYTTTGGKIPIRWTAPEAIAYRKFSSASDAWSYGIVMWEVMSYGERPYWEMSNQDVILSIEEGYRLPAPMGCPASLHQLMLHCWQKERNHRPKFTDIVSFLDKLI

SEQ ID NO: 108 - protein, 262 aa, Homo sapiens
  Ephrin type-A receptor 7
IKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMI

SEQ ID NO: 109 - protein, 262 aa, Homo sapiens
  Ephrin type-A receptor 8
IHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALI

SEQ ID NO: 110 - protein, 260 aa, Homo sapiens
  Ephrin type-A receptor 10
VTLERSLGGGRFGELCCGCLQLPGRQELLVAVHMLRDSASDSQRLGFLAEALTLGQFDHSHIVRLEGVVTRGSTLMIVTEYMSHGALDGFLRRHEGQLVAGQLMGLLPGLASAMKYLSEMGYVHRGLAARHVLVSSDLVCKISGFGRGPRDRSEAVYTTMSGRSPALWAAPETLQFGHFSSASDVWSFGIIMWEVMAFGERPYWDMSGQDVIKAVEDGFRLPPPRNCPNLLHRLMLDCWQKDPGERPRFSQIHSILSKMV

SEQ ID NO: 111 - protein, 264 aa, Homo sapiens
  Ephrin type-B receptor 1
VKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMI

SEQ ID NO: 112 - protein, 264 aa, Homo sapiens
  Ephrin type-B receptor 2
VKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMI

SEQ ID NO: 113 - protein, 264 aa, Homo sapiens
  Ephrin type-B receptor 3
VKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLI

SEQ ID NO: 114 - protein, 264 aa, Homo sapiens
  Ephrin type-B receptor 4
VKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMI

SEQ ID NO: 115 - protein, 250 aa, Homo sapiens
  Ephrin type-B receptor 6
IKIEEVIGTGSFGEVRQGRLQPRGRREQTVAIQALWAGGAESLQMTFLGRAAVLGQFQHPNILRLEGVVTKSRPLMVLTEFMELGPLDSFLRQREGQFSSLQLVAMQRGVAAAMQYLSSFAFVHRSLSAHSVLVNSHLVCKVARLGHSPQGPSCLLRWAAPEVIAHGKHTTSSDVWSFGILMWEVMSYGERPYWDMSEQEVLNAIEQEFRLPPPPGCPPGLHLLMLDTWQKDRARRPHFDQLVAAFDKMI

SEQ ID NO: 116 - protein, 28 aa, Homo sapiens
  Ephrin type-A receptor 1
TIANFDPRMTLRLPSLSGSDGIPYRTVS

SEQ ID NO: 117 - protein, 28 aa, Homo sapiens
  Ephrin type-A receptor 2
TLADFDPRVSIRLPSTSGSEGVPFRTVS

SEQ ID NO: 118 - protein, 25 aa, Homo sapiens
  Ephrin type-A receptor 3
TSAAARPSNLLLDQSNVDITTFRTT

SEQ ID NO: 119 - protein, 26 aa, Homo sapiens
  Ephrin type-A receptor 4
TGTESSRPNTALLDPSSPEFSAVVSV

SEQ ID NO: 120 - protein, 27 aa, Homo sapiens
  Ephrin type-A receptor 5
TLVNASCRVSNLLAEHSPLGSGAYRSV

SEQ ID NO: 121 - protein, 25 aa, Homo sapiens
  Ephrin type-A receptor 6
TLVEDILVMPESPGEVPEYPLFVTV

SEQ ID NO: 122 - protein, 27 aa, Homo sapiens
  Ephrin type-A receptor 7
TPLGTCSRPISPLLDQNTPDFTTFCSV

SEQ ID NO: 123 - protein, 31 aa, Homo sapiens
  Ephrin type-A receptor 8
TATVSRCPPPAFVRSCFDLRGGSGGGGGLTV

SEQ ID NO: 124 - protein, 24 aa, Homo sapiens
  Ephrin type-A receptor 10
TTCPRPPTPLADRAFSTFPSFGSV

SEQ ID NO: 125 - protein, 27 aa, Homo sapiens
  Ephrin type-A receptor 1
TVATITAVPSQPLLDRSIPDFTAFTTV

SEQ ID NO: 126 - protein, 27 aa, Homo sapiens
  Ephrin type-B receptor 2
AMAPLSSGINLPLLDRTIPDYTSFNTV

SEQ ID NO: 127 - protein, 24 aa, Homo sapiens
  Ephrin type-B receptor 3
ASAQSGMSQPLLDRTVPDYTTFTT

SEQ ID NO: 128 - protein, 25 aa, Homo sapiens
  Ephrin type-B receptor 4
ARENGGASHPLLDQRQPHYSAFGSV

SEQ ID NO: 129 - protein, 25 aa, Homo sapiens
  Ephrin type-B receptor 6
GDPGERPSQALLTPVALDFPCLDSP

SEQ ID NO: 130 - protein, 55 aa, Homo sapiens
  Ephrin type-A receptor 1
EWLESIRMKRYILHFHSAGLDTMECVLELTAEDLTQMGITLPGHQKRILCSIQGF

SEQ ID NO: 131 - protein, 61 aa, Homo sapiens
  Ephrin type-A receptor 2
EWLESIKMQQYTEHFMAAGYTAIEKVVQMTNDDIKRIGVRLPGHQKRIAYSLLGLKDQVNT

SEQ ID NO: 132 - protein, 58 aa, Homo sapiens
  Ephrin type-A receptor 3
GDWLNGVWTAHCKEIFTGVEYSSCDTIAKISTDDMKKVGVTVVGPQKKIISSIKALET

SEQ ID NO: 133 - protein, 65 aa, Homo sapiens
  Ephrin type-A receptor 4
GDWLQAIKMDRYKDNFTAAGYTTLEAVVHVNQEDLARIGITAITHQNKILSSVQAMRTQMQQMHG

SEQ ID NO: 134 - protein, 60 aa, Homo sapiens
  Ephrin type-A receptor 5
GEWLEAIKMGRYTEIFMENGYSSMDAVAQVTLEDLRRLGVTLVGHQKKIMNSLQEMKVQL

SEQ ID NO: 135 - protein, 58 aa, Homo sapiens
  Ephrin type-A receptor 6
GDWLDSIKMGQYKNNFVAAGFTTFDLISRMSIDDIRRIGVILIGHQRRIVSSIQTLRL

SEQ ID NO: 136 - protein, 60 aa, Homo sapiens
  Ephrin type-A receptor 7
GEWLQAIKMERYKDNFTAAGYNSLESVARMTIEDVMSLGITLVGHQKKIMSSIQTMRAQM

SEQ ID NO: 137 - protein, 60 aa, Homo sapiens
  Ephrin type-A receptor 8
GDWLDSIRMGRYRDHFAAGGYSSLGMVLRMNAQDVRALGITLMGHQKKILGSIQTMRAQL

SEQ ID NO: 138 - protein, 60 aa, Homo sapiens
  Ephrin type-A receptor 10
GAWLEALDLCRYKDSFAAAGYGSLEAVAEMTAQDLVSLGISLAEHREALLSGISALQARV

SEQ ID NO: 139 - protein, 59 aa, Homo sapiens
  Ephrin type-B receptor 1
DDWLSAIKMVQYRDSFLTAGFTSLQLVTQMTSEDLLRIGITLAGHQKKILNSIHSMRVQ

SEQ ID NO: 140 - protein, 62 aa, Homo sapiens
  Ephrin type-B receptor 2
DEWLEAIKMGQYKESFANAGFTSFDVVSQMMMEDILRVGVTLAGHQKKILNSIQVMRAQMNQ

SEQ ID NO: 141 - protein, 60 aa, Homo sapiens
  Ephrin type-B receptor 3
GDWLDAIKMGRYKESFVSAGFASFDLVAQMTAEDLLRIGVTLAGHQKKILSSIQDMRLQM

SEQ ID NO: 142 - protein, 61 aa, Homo sapiens
  Ephrin type-B receptor 4
GEWLRAIKMGRYEESFAAAGFGSFELVSQISAEDLLRIGVTLAGHQKKILASVQHMKSQAK

SEQ ID NO: 143 - protein, 60 aa, Homo sapiens
  Ephrin type-B receptor 6
QAWLSAIGLECYQDNFSKFGLCTFSDVAQLSLEDLPALGITLAGHQKKLLHHIQLLQQHL

SEQ ID NO: 144 - protein, 60 aa, Homo sapiens
  Poly [ADP-ribose] polymerase tankyrase-1
NISQFLKSLGLEHLRDIFETEQITLDVLADMGHEELKEIGINAYGHRHKLIKGVERLLGG

SEQ ID NO: 145 - protein, 64 aa, Homo sapiens
  Poly [ADP-ribose] polymerase tankyrase-2
GVDFSITQFVRNLGLEHLMDIFEREQITLDVLVEMGHKELKEIGINAYGHRHKLIKGVERLISG

SEQ ID NO: 146 - protein, 75 aa, Homo sapiens
  Transcription factor ETV6
LPAHLRLQPIYWSRDDVAQWLKWAENEFSLRPIDSNTFEMNGKALLLLTKEDFRYRSPHSGDVLYELLQHILKQR

SEQ ID NO: 147 - protein, 3 aa, Homo sapiens
  Ephrin type-A receptor 1
FKD

SEQ ID NO: 148 - protein, 3 aa, Homo sapiens
  Ephrin type-A receptor 2
IPI

SEQ ID NO: 149 - protein, 3 aa, Homo sapiens
  Ephrin type-A receptor 3
VPV

SEQ ID NO: 150 - protein, 3 aa, Homo sapiens
  Ephrin type-A receptor 4
VPV

SEQ ID NO: 151 - protein, 3 aa, Homo sapiens
  Ephrin type-A receptor 5
VPL

SEQ ID NO: 152 - protein, 3 aa, Homo sapiens
  Ephrin type-A receptor 6
FHV

SEQ ID NO: 153 - protein, 3 aa, Homo sapiens
  Ephrin type-A receptor 7
IQV

SEQ ID NO: 154 - protein, 3 aa, Homo sapiens
  Ephrin type-A receptor 8
RHL

SEQ ID NO: 155 - protein, 3 aa, Homo sapiens
  Ephrin type-B receptor 1
AMA

SEQ ID NO: 156 - protein, 3 aa, Homo sapiens
  Ephrin type-B receptor 2
VEG

SEQ ID NO: 157 - protein, 3 aa, Homo sapiens
  Ephrin type-B receptor 3
VQV

SEQ ID NO: 158 - protein, 3 aa, Homo sapiens
  Ephrin type-B receptor 4
PQY

SEQ ID NO: 159 - protein, 3 aa, Homo sapiens
  Ephrin type-B receptor 6
VEV

SEQ ID NO: 160 - protein, 305 aa, Homo sapiens
  Exemplary parental receptor of the cargo binding domain -EphA2
PPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTG

SEQ ID NO: 161 - protein, 95 aa, Homo sapiens
  Exemplary parental EphA4/EphB2 receptors of the cargo binding domain
WLQLQANAAAALHMLRTEPPGTFLVRKSNTRQCQALCMRLPEASGPSFVSSHYILESPGGVSLEGSELMFPDLVQLICAYCHTRDILLLPLQLPR

SEQ ID NO: 162 - protein, 95 aa, Homo sapiens
  Exemplary parental EphA2 receptor of the cargo binding domain
WYAGPMERAGAESILANRSDGTFLVRQRVKDAAEFAISIKYNVEVKHIKIMTAEGLYRITEKKAFRGLTELVEFYQQNSLKDCFKSLDTTLQFPF

SEQ ID NO: 163 - protein, 95 aa, Homo sapiens
  Exemplary parental EphA2/EphB2 receptors of the cargo binding domain
WFAGNMERQQTDNLLKSHASGTYLIRERPAEAERFAISIKFNDEVKHIKVVEKDNWIHITEAKKFDSLLELVEYYQCHSLKESFKQLDTTLKYPY

SEQ ID NO: 164 - protein, 95 aa, Homo sapiens
  Exemplary parental EphA2 receptor of the cargo binding domain
WYAGAMERLQAETELINRVNSTYLVRHRTKESGEYAISIKYNNEAKHIKILTRDGFFHIAENRKFKSLMELVEYYKHHSLKEGFRTLDTTLQFPY

SEQ ID NO: 165 - protein, 92 aa, Homo sapiens
  Exemplary parental EphA2 receptor of the cargo binding domain
WLFEGLGRDKAEELLQLPDTKVGSFMIRESETKKGFYSLSVRHRQVKHYRIFRLPNNWYYISPRLTFQCLEDLVNHYSEVADGLCCVLTTPC

SEQ ID NO: 166 - protein, 92 aa, Homo sapiens; identical to SEQ ID NO: 165
  Exemplary parental EphA2 receptor of the cargo binding domain

SEQ ID NO: 167 - protein, 98 aa, Homo sapiens
  Exemplary parental EphA/EphB receptors of the cargo binding domain
WYFGKITRRESERLLLNAENPRGTFLVRESETTKGAYCLSVSDFDNAKGLNVKHYKIRKLDSGGFYITSRTQFNSLQQLVAYYSKHADGLCHRLTTVC

SEQ ID NO: 168 - protein, 95 aa, Homo sapiens
  Exemplary parental EphB1/EphA4/EphA4 receptors of the cargo binding domain
WYYGKVTRHQAEMALNERGHEGDFLIRDSESSPNDFSVSLKAQGKNKHFKVQLKETVYCIGQRKFSTMEELVEHYKKAPIFTSEQGEKLYLVKHL

SEQ ID NO: 169 - protein, 96 aa, Homo sapiens
  Exemplary parental EphB2 receptor of the cargo binding domain
WYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 170 - protein, 103 aa, Homo sapiens
  Exemplary parental EphA1 receptor of the cargo binding domain
QPWPTLLKNWQLLAVNHPGYMAFLTYDEVQERLQACRDKPGSYIFRPSCTRLGQWAIGYVSSDGSILQTIPANKPLSQVLLEGQKDGFYLYPDGKTHNPDLTE

SEQ ID NO: 171 - protein, 103 aa, Homo sapiens; identical to SEQ ID NO: 170
  Exemplary parental EphA receptor of the cargo binding domain

SEQ ID NO: 172 - protein, 103 aa, Homo sapiens
  Exemplary parental EphA receptor of the cargo binding domain
QPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTG

SEQ ID NO: 173 - protein, 98 aa, Homo sapiens
  Exemplary parental EphA3/EphA4/EphB2 receptors of the cargo binding domain
WYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPC

SEQ ID NO: 174 - protein, 96 aa, Homo sapiens
  Exemplary parental EphA2/EphA4 receptors of the cargo binding domain
WYWGDISREEVNEKLRDTADGTFLVRDASTKMHGDYTLTLRKGGNNKLIKIFHRDGKYGFSDPLTFSSVVELINHYRNESLAQYNPKLDVKLLYPV

SEQ ID NO: 175 - protein, 95 aa, Homo sapiens
  Exemplary parental EphA2/EphA4 receptors of the cargo binding domain
WNVGSSNRNKAENLLRGKRDGTFLVRESSKQGCYACSVVVDGEVKHCVINKTATGYGFAEPYNLYSSLKELVLHYQHTSLVQHNDSLNVTLAYPV

SEQ ID NO: 176 - protein, 93 aa, Homo sapiens
  Exemplary parental EphB1/EphB2 receptors of the cargo binding domain
WFFGKIPRAKAEEMLSKQRHDGAFLIRESESAPGDFSLSVKFGNDVQHFKVLRDGAGKYFLWVVKFNSLNELVDYHRSTSVSRNQQIFLRDIE

SEQ ID NO: 177 - protein, 97 aa, Homo sapiens
  Exemplary parental EphA2/EphB1 receptors of the cargo binding domain
WFHGRISREESQRLIGQQGLVDGLFLVRESQRNPQGFVLSLCHLQKVKHYLILPSEEEGRLYFSMDDGQTRFTDLLQLVEFHQLNRGILPCLLRHCC

SEQ ID NO: 178 - protein, 91 aa, Homo sapiens
  Exemplary parental EphB2/EphB3 receptors of the cargo binding domain
WFHGKISKQEAYNLLMTVGQVCSFLVRPSDNTPGDYSLYFRTNENIQRFKICPTPNNQFMMGGRYYNSIGDIIDHYRKEQIVEGYYLKEPV

SEQ ID NO: 179 - protein, 91 aa, Homo sapiens; identical to SEQ ID NO: 178
  Exemplary parental EphB2/EphB3 receptors of the cargo binding domain

SEQ ID NO: 180 - protein, 106 aa, Homo sapiens
  Exemplary parental EphB2/EphB3 receptors of the cargo binding domain
WYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPV

SEQ ID NO: 181 - protein, 106 aa, Homo sapiens; identical to SEQ ID NO: 180
  Exemplary parental EphA3/EphA4/EphB2 receptors of the cargo binding domain

SEQ ID NO: 182 - protein, 91 aa, Homo sapiens
  Exemplary parental EphA2/EphB2 receptors of the cargo binding domain
WYHGPVSRNAAEYLLSSGINGSFLVRESESSPGQRSISLRYEGRVYHYRINTASDGKLYVSSESRFNTLAELVHHHSTVADGLITTLHYPA

SEQ ID NO: 183 - protein, 100 aa, Homo sapiens
  Exemplary parental EphB2 receptor of the cargo binding domain
WYHGRIPREVSETLVQRNGDFLIRDSLTSLGDYVLTCRWRNQALHFKINKVVVKAGESYTHIQYLFEQESFDHVPALVRYHVGSRKAVSEQSGAIIYCPV

SEQ ID NO: 184 - protein, 67 aa, Homo sapiens
  Exemplary parental EphA1/EphA2/EphA8 receptors of the cargo binding domain
TLEQSVGEWLESIGLQQYESKLLLNGFDDVHFLGSNVMEEQDLRDIGISDPQHRRKLLQAARSLPKV

SEQ ID NO: 185 - protein, 63 aa, Homo sapiens
  Exemplary parental EphA1/EphA2/EphA6 receptors of the cargo binding domain
LGEAGMSAWLRAIGLERYEEGLVHNGWDDLEFLSDITEEDLEEAGVQDPAHKRLLLDTLQLSK

SEQ ID NO: 186 - protein, 65 aa, Homo sapiens
  Exemplary parental EphA1/EphA2/EphA5/EphA6/EphA7/EphA8/ EphB1/EphB2/EphB3/EphB4 receptors of the cargo binding domain
MCTNIVYEWLKALQLPQYAESFVDNGYDDLEVCKQIGDPDLDAIGVLAPAHRRRILEAVRRLREQ

SEQ ID NO: 187 - protein, 87 aa, Homo sapiens
  Exemplary parental EphA3/EphA4/EphA6/EphA7EphB2/EphB3/EphB4/EphB6 receptors of the cargo binding domain
IITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 188 - protein, 83 aa, Homo sapiens
  Exemplary parental EphA7/EphB2 receptors of the cargo binding domain
TVELKRYGGPLGITISGTEEPFDPIIISSLTKGGLAERTGAIHIGDRILAINSSSLKGKPLSEAIHLLQMAGETVTLKIKKQT

SEQ ID NO: 189 - protein, 83 aa, Homo sapiens
  Exemplary parental EphB2 receptor of the cargo binding domain
TVELKRYGGPLGITISGTEEPFDPIVISGLTKRGLAERTGAIHVGDRILAINNVSLKGRPLSEAIHLLQVAGETVTLKIKKQL

SEQ ID NO: 190 - protein, 80 aa, Homo sapiens
  Exemplary parental EphA7 receptors of the cargo binding domain
EVILCKDQDGKIGLRLKSIDNGIFVQLVQANSPASLVGLRFGDQVLQINGENCAGWSSDKAHKVLKQAFGEKITMTIRDR

SEQ ID NO: 191 - protein, 76 aa, Homo sapiens
  Exemplary parental EphA7 receptors of the cargo binding domain
TITMHKDSTGHVGFIFKNGKITSIVKDSSAARNGLLTEHNICEINGQNVIGLKDSQIADILSTSGTVVTITIMPAF

SEQ ID NO: 192 - protein, 84 aa, Homo sapiens
  Exemplary parental EphA7/EphB1/EphB2 receptors of the cargo binding domain
KVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 193 - protein, 79 aa, Homo sapiens
  Exemplary parental EphA4/EphA6 receptors of the cargo binding domain
HVNYEGIVADVEPYGYAWQAGLRQGSRLVEICKVAVATLSHEQMIDLLRTSVTVKVVIIPPHDDCTPRRSCSETYRMPV

SEQ ID NO: 194 - protein, 81 aa, Homo sapiens
  Exemplary parental EphA4/ephA3/EphB2/EphB4 receptors of the cargo binding domain
LITLIKSEKGSLGFTVTKGNQRIGCYVHDVIQDPAKSDGRLKPGDRLIKVNDTDVTNMTHTDAVNLLRAASKTVRLVIGRV

SEQ ID NO: 195 - protein, 329 aa, Homo sapiens
  Exemplary parental EphA4 receptor of the cargo binding domain
KLQEAMFELVTSEASYYKSLNLLVSHFMENERIRKILHPSEAHILFSNVLDVLAVSERFLLELEHRMEENIVISDVCDIVYRYAADHFSVYITYVSNQTYQERTYKQLLQEKAAFRELIAQLELDPKCRGLPFSSFLILPFQRITRLKLLVQNILKRVEERSERECTALDAHKELEMVVKACNEGVRKMSRTEQMISIQKKMEFKIKSVPIISHSRWLLKQGELQQMSGPKTSRTLRTKKLFHEIYLFLFNDLLVICRQIPGDKYQVFDSAPRGLLRVEELEDQGQTLANVFILRLLENADDREATYMLKASSQSEMKRWMTSLAPNRR

SEQ ID NO: 196 - protein, 338 aa, Homo sapiens
  Exemplary parental EphA2 receptor of the cargo binding domain
RKRQEAMFEILTSEFSYQHSLSILVEEFLQSKELRATVTQMEHHHLFSNILDVLGASQRFFEDLEQRHKAQVLVEDISDILEEHAEKHFHPYIAYCSNEVYQQRTLQKLISSNAAFREALREIERRPACGGLPMLSFLILPMQRVTRLPLLMDTLCLKTQGHSERYKAASRALKAISKLVRQCNEGAHRMERMEQMYTLHTQLDFSKVKSLPLISASRWLLKRGELFLVEETGLFRKIASRPTCYLFLFNDVLVVTKKKSEESYMVQDYAQMNHIQVEKIEPSELPLPGGGNRSSSVPHPFQVTLLRNSEGRQEQLLLSSDSASDRARWIVALTHSER

SEQ ID NO: 197 - protein, 185 aa, Homo sapiens
  Exemplary parental EphB2 receptor of the cargo binding domain
RMQESLFEVVTSEASYLRSLRLLTDTFVLSQALRDTLTPRDHHTLFSNVQRVQGVSERFLATLLSRVRSSPHISDLCDVVHAHAVGPFSVYVDYVRNQQYQEETYSRLMDTNVRFSAELRRLQSLPKCERLPLPSFLLLPFQRITRLRMLLQNILRQTEEGSSRQENAQKALGAVSKIIERCSAE

SEQ ID NO: 198 - protein, 570 aa, Homo sapiens
  EphA1 (27-596)
EVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLVSGSVKLNVERCSLGRLTRRGLYLAFHNPGACVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPPVSRGLTGGEIVAVIFGLLLGAALLLGILVFRSRRAQRQRQQRQRDRATDVDREDKLWL

SEQ ID NO: 199 - protein, 558 aa, Homo sapiens
  EphA2 (28-585)
EVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPL

SEQ ID NO: 200 - protein, 562 aa, Homo sapiens
  EphA3 (29-590)
EVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKL

SEQ ID NO: 201 - protein, 561 aa, Homo sapiens
  EphA4 (30-590)
EVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLN

SEQ ID NO: 202 - protein, 560 aa, Homo sapiens
  EphA5 (60-619)
EVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPVFAASSDQSQIPVIAVSVTVGVILLAVVIGVLLSGSCCECGCGRASSLCAVAHPSLIW

SEQ ID NO: 203 - protein, 565 aa, Homo sapiens
  EphA6 (34-589)
QVVLLDTTTVLGELGWKTYPLNGWDAITEMDEHNRPIHTYQVCNVMEPNQNNWLRTNWISRDAAQKIYVEMKFTLRDCNSIPWVLGTCKETFNLFYMESDESHGIKFKPNQYTKIDTIAADESFTQMDLGDRILKLNTEIREVGPIERKGFYLAFQDIGACIALVSVRVFYKKCPFTVRNLAMFPDTIPRVDSSSLVEVRGSCVKSAEERDTPKLYCGADGDWLVPLGRCICSTGYEEIEGSCHACRPGFYKAFAGNTKCSKCPPHSLTYMEATSVCQCEKGYFRAEKDPPSMACTRPPSAPRNVVFNINETALILEWSPPSDTGGRKDLTYSVICKKCGLDTSQCEDCGGGLRFIPRHTGLINNSVIVLDFVSHVNYTFEIEAMNGVSELSFSPKPFTAITVTTDQDAPSLIGVVRKDWASQNSIALSWQAPAFSNGAILDYEIKYYEKEHEQLTYSSTRSKAPSVIITGLKPATKYVFHIRVRTATGYSGYSQKFEFETGDETSDMAAEQGQILVIATAAVGGFTLLVILTLFFLITGRCQWYIKAKMKSEEKRRNHLQNGHL

SEQ ID NO: 204 - protein, 578 aa, Homo sapiens
  EphA7 (30-607)
AKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKT

SEQ ID NO: 205 - protein, 559 aa, Homo sapiens
  EphA8 (31-589)
EVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQA

SEQ ID NO: 206 - protein, 570 aa, Homo sapiens
  EphA10  (35-604)
EVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQCRATVRGLATFPATAAESAFSTLVEVAGTCVAHSEGEPGSPPRMHCGADGEWLVPVGRCSCSAGFQERGDFCEACPPGFYKVSPRRPLCSPCPEHSRALENASTFCVCQDSYARSPTDPPSASCTRPPSAPRDLQYSLSRSPLVLRLRWLPPADSGGRSDVTYSLLCLRCGREGPAGACEPCGPRVAFLPRQAGLRERAATLLHLRPGARYTVRVAALNGVSGPAAAAGTTYAQVTVSTGPGAPWEEDEIRRDRVEPQSVSLSWREPIPAGAPGANDTEYEIRYYEKGQSEQTYSMVKTGAPTVTVTNLKPATRYVFQIRAASPGPSWEAQSFNPSIEVQTLGEAASGSRDQSPAIVVTVVTISALLVLGSVMSVLAIWRRPCSYGKGGGDAHD

SEQ ID NO: 207 - protein, 572 aa, Homo sapiens
  EphB1 (21-591)
ETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGM

SEQ ID NO: 208 - protein, 571 aa, Homo sapiens
  EphB2 (19-589)
VEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRGFERADSEYTDKLQHYTSGHM

SEQ ID NO: 209 - protein, 567 aa, Homo sapiens
  EphB3 (39-605)
EETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGM

SEQ ID NO: 210 - protein, 567 aa, Homo sapiens
  EphB4 (19-583)
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLI

SEQ ID NO: 211 - protein, 617 aa, Homo sapiens
  EphB6 (33-649)
EEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYTCPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPCTGPPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHEVPSAVPVVHQVSRASNSITVSWPQPDQTNGNILDYQLRYYDQAEDESHSFTLTSETNTATVTQLSPGHIYGFQVRARTAAGHGPYGGKVYFQTLPQGELSSQLPERLSLVIGSILGALAFLLLAAITVLAVVFQRKRRGTGYTEQLQQYSSPGLGVKYYIDPS

SEQ ID NO: 212 - protein, 976 aa, Homo sapiens
  Ephrin type-A receptor 1
MERRWPLGLGLVLLLCAPLPPGARAKEVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLVSGSVKLNVERCSLGRLTRRGLYLAFHNPGACVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPPVSRGLTGGEIVAVIFGLLLGAALLLGILVFRSRRAQRQRQQRQRDRATDVDREDKLWLKPYVDLQAYEDPAQGALDFTRELDPAWLMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLLANPHSLRTIANFDPRMTLRLPSLSGSDGIPYRTVSEWLESIRMKRYILHFHSAGLDTMECVLELTAEDLTQMGITLPGHQKRILCSIQGFKD

SEQ ID NO: 213 - protein, 976 aa, Homo sapiens
  Ephrin type-A receptor 2
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 214 - protein, 983 aa, Homo sapiens
  Ephrin type-A receptor 3
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 215 - protein, 986 aa, Homo sapiens
  Ephrin type-A receptor 4
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 216 - protein, 1037 aa, Homo sapiens
  Ephrin type-A receptor 5
MRGSGPRGAGRRRPPSGGGDTPITPASLAGCYSAPRRAPLWTCLLLCAALRTLLASPSNEVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPVFAASSDQSQIPVIAVSVTVGVILLAVVIGVLLSGSCCECGCGRASSLCAVAHPSLIWRCGYSKAKQDPEEEKMHFHNGHIKLPGVRTYIDPHTYEDPNQAVHEFAKEIEASCITIERVIGAGEFGEVCSGRLKLPGKRELPVAIKTLKVGYTEKQRRDFLGEASIMGQFDHPNIIHLEGVVTKSKPVMIVTEYMENGSLDTFLKKNDGQFTVIQLVGMLRGISAGMKYLSDMGYVHRDLAARNILINSNLVCKVSDFGLSRVLEDDPEAAYTTRGGKIPIRWTAPEAIAFRKFTSASDVWSYGIVMWEVVSYGERPYWEMTNQDVIKAVEEGYRLPSPMDCPAALYQLMLDCWQKERNSRPKFDEIVNMLDKLIRNPSSLKTLVNASCRVSNLLAEHSPLGSGAYRSVGEWLEAIKMGRYTEIFMENGYSSMDAVAQVTLEDLRRLGVTLVGHQKKIMNSLQEMKVQLVNGMVPL

SEQ ID NO: 217 - protein, 1036 aa, Homo sapiens
  Ephrin type-A receptor 6
MGGCEVREFLLQFGFFLPLLTAWPGDCSHVSNNQVVLLDTTTVLGELGWKTYPLNGWDAITEMDEHNRPIHTYQVCNVMEPNQNNWLRTNWISRDAAQKIYVEMKFTLRDCNSIPWVLGTCKETFNLFYMESDESHGIKFKPNQYTKIDTIAADESFTQMDLGDRILKLNTEIREVGPIERKGFYLAFQDIGACIALVSVRVFYKKCPFTVRNLAMFPDTIPRVDSSSLVEVRGSCVKSAEERDTPKLYCGADGDWLVPLGRCICSTGYEEIEGSCHACRPGFYKAFAGNTKCSKCPPHSLTYMEATSVCQCEKGYFRAEKDPPSMACTRPPSAPRNVVFNINETALILEWSPPSDTGGRKDLTYSVICKKCGLDTSQCEDCGGGLRFIPRHTGLINNSVIVLDFVSHVNYTFEIEAMNGVSELSFSPKPFTAITVTTDQDAPSLIGVVRKDWASQNSIALSWQAPAFSNGAILDYEIKYYEKEHEQLTYSSTRSKAPSVIITGLKPATKYVFHIRVRTATGYSGYSQKFEFETGDETSDMAAEQGQILVIATAAVGGFTLLVILTLFFLITGRCQWYIKAKMKSEEKRRNHLQNGHLRFPGIKTYIDPDTYEDPSLAVHEFAKEIDPSRIRIERVIGAGEFGEVCSGRLKTPGKREIPVAIKTLKGGHMDRQRRDFLREASIMGQFDHPNIIRLEGVVTKRSFPAIGVEAFCPSFLRAGFLNSIQAPHPVPGGGSLPPRIPAGRPVMIVVEYMENGSLDSFLRKHDGHFTVIQLVGMLRGIASGMKYLSDMGYVHRDLAARNILVNSNLVCKVSDFGLSRVLEDDPEAAYTTTGGKIPIRWTAPEAIAYRKFSSASDAWSYGIVMWEVMSYGERPYWEMSNQDVILSIEEGYRLPAPMGCPASLHQLMLHCWQKERNHRPKFTDIVSFLDKLIRNPSALHTLVEDILVMPESPGEVPEYPLFVTVGDWLDSIKMGQYKNNFVAAGFTTFDLISRMSIDDIRRIGVILIGHQRRIVSSIQTLRLHMMHIQEKGFHV

SEQ ID NO: 218 - protein, 998 aa, Homo sapiens
  Ephrin type-A receptor 7
MVFQTRYPSWIILCYIWLLRFAHTGEAQAAKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKTYIDPETYEDPNRAVHQFAKELDASCIKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMIRNPNSLKTPLGTCSRPISPLLDQNTPDFTTFCSVGEWLQAIKMERYKDNFTAAGYNSLESVARMTIEDVMSLGITLVGHQKKIMSSIQTMRAQMLHLHGTGIQV

SEQ ID NO: 219 - protein, 1005 aa, Homo sapiens
  Ephrin type-A receptor 8
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 220 - protein, 1008 aa, Homo sapiens
  Ephrin type-A receptor 10
METCAGPHPLRLFLCRMQLCLALLLGPWRPGTAEEVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQCRATVRGLATFPATAAESAFSTLVEVAGTCVAHSEGEPGSPPRMHCGADGEWLVPVGRCSCSAGFQERGDFCEACPPGFYKVSPRRPLCSPCPEHSRALENASTFCVCQDSYARSPTDPPSASCTRPPSAPRDLQYSLSRSPLVLRLRWLPPADSGGRSDVTYSLLCLRCGREGPAGACEPCGPRVAFLPRQAGLRERAATLLHLRPGARYTVRVAALNGVSGPAAAAGTTYAQVTVSTGPGAPWEEDEIRRDRVEPQSVSLSWREPIPAGAPGANDTEYEIRYYEKGQSEQTYSMVKTGAPTVTVTNLKPATRYVFQIRAASPGPSWEAQSFNPSIEVQTLGEAASGSRDQSPAIVVTVVTISALLVLGSVMSVLAIWRRPCSYGKGGGDAHDEEELYFHFKVPTRRTFLDPQSCGDLLQAVHLFAKELDAKSVTLERSLGGGRFGELCCGCLQLPGRQELLVAVHMLRDSASDSQRLGFLAEALTLGQFDHSHIVRLEGVVTRGSTLMIVTEYMSHGALDGFLRRHEGQLVAGQLMGLLPGLASAMKYLSEMGYVHRGLAARHVLVSSDLVCKISGFGRGPRDRSEAVYTTMSGRSPALWAAPETLQFGHFSSASDVWSFGIIMWEVMAFGERPYWDMSGQDVIKAVEDGFRLPPPRNCPNLLHRLMLDCWQKDPGERPRFSQIHSILSKMVQDPEPPKCALTTCPRPPTPLADRAFSTFPSFGSVGAWLEALDLCRYKDSFAAAGYGSLEAVAEMTAQDLVSLGISLAEHREALLSGISALQARVLQLQGQGVQV

SEQ ID NO: 221 - protein, 984 aa, Homo sapiens
  Ephrin type-B receptor 1
MALDYLLLLLLASAVAAMEETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 222 - protein, 1055 aa, Homo sapiens
  Ephrin type-B receptor 2
MALRRLGAALLLLPLLAAVEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEGQPLARRPRATGRTKRCQPRDVTKKTCNSNDGKKKGMGKKKTDPGRGREIQGIFFKEDSHKESNDCSCGG

SEQ ID NO: 223 - protein, 998 aa, Homo sapiens
  Ephrin type-B receptor 3
MARARPPPPPSPPPGLLPLLPPLLLLPLLLLPAGCRALEETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGMKVYIDPFTYEDPNEAVREFAKEIDVSCVKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLIRNAASLKVIASAQSGMSQPLLDRTVPDYTTFTTVGDWLDAIKMGRYKESFVSAGFASFDLVAQMTAEDLLRIGVTLAGHQKKILSSIQDMRLQMNQTLPVQV

SEQ ID NO: 224 - protein, 987 aa, Homo sapiens
  Ephrin type-B receptor 4
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 225 - protein, 1021 aa, Homo sapiens
  Ephrin type-B receptor 6
MATEGAAQLGNRVAGMVCSLWVLLLVSSVLALEEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYTCPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPCTGPPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHEVPSAVPVVHQVSRASNSITVSWPQPDQTNGNILDYQLRYYDQAEDESHSFTLTSETNTATVTQLSPGHIYGFQVRARTAAGHGPYGGKVYFQTLPQGELSSQLPERLSLVIGSILGALAFLLLAAITVLAVVFQRKRRGTGYTEQLQQYSSPGLGVKYYIDPSTYEDPCQAIRELAREVDPAYIKIEEVIGTGSFGEVRQGRLQPRGRREQTVAIQALWAGGAESLQMTFLGRAAVLGQFQHPNILRLEGVVTKSRPLMVLTEFMELGPLDSFLRQREGQFSSLQLVAMQRGVAAAMQYLSSFAFVHRSLSAHSVLVNSHLVCKVARLGHSPQGPSCLLRWAAPEVIAHGKHTTSSDVWSFGILMWEVMSYGERPYWDMSEQEVLNAIEQEFRLPPPPGCPPGLHLLMLDTWQKDRARRPHFDQLVAAFDKMIRKPDTLQAGGDPGERPSQALLTPVALDFPCLDSPQAWLSAIGLECYQDNFSKFGLCTFSDVAQLSLEDLPALGITLAGHQKKLLHHIQLLQQHLRQQGSVEV

SEQ ID NO: 226 - protein, 380 aa, Homo sapiens
  EphA4 fragment
LAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLN

SEQ ID NO: 227 - protein, 365 aa, Mouse
  Mouse FcRn
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 228 - protein, 365 aa, Homo sapiens
  Human FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 229 - protein, 1211 aa, Artificial Sequence
  Signal peptide-Monobody-Linker-modified Fc-Linker-EphB2-Turboluc
MALRRLGAALLLLPLLAAVSDVPRDLEVVAATPTSLLISWYYPFCAFYYRITYGETGGNSPVQEFTVPRSPDTATISGLKPGVDYTITVYAVTCLGSYSRPISINYRTGGGGSGGGGSGGAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGGGSGGGSGGGSGGGSRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDAVYNIICKSCGSGRGACTRCGDNVQYAPRQLGLTEPRIYASDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTEIQEKLPLIIGSSAAGLVFLIAVVVISIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSFGERPYWDMTNQDVINAIEQDYRLPPPMDCPSALHQLMLDCWQKDRNHRPKFGQIVNTLDKMIRNPNSLKAMAPLSSGINLPLGGGEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD

SEQ ID NO: 230 - protein, 20 aa, Artificial Sequence
  modified Fc domain
  misc_feature (8)..(8): Xaa can be any naturally occurring amino acid
  misc_feature (10)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (19)..(19): Xaa can be any naturally occurring amino acid
LNGEEFMXFXXXXGXWXGXW

SEQ ID NO: 231 - protein, 4 aa, Artificial Sequence
  peptide linker
  REPEAT (1)..(4): GGGS can be repeated 1-10 times
GGGS

SEQ ID NO: 232 - protein, 8 aa, Mouse
  peptide linker
GGGSGGGS

SEQ ID NO: 233 - protein, 12 aa, Artificial Sequence
  peptide linker
GGGSGGGSGGGS

SEQ ID NO: 234 - protein, 5 aa, Artificial Sequence
  peptide linker
  REPEAT (1)..(5): GGGGS can be repeated 1 to 10 times
GGGGS

SEQ ID NO: 235 - protein, 10 aa, Artificial Sequence
  peptide linker
GGGGSGGGGS

SEQ ID NO: 236 - protein, 15 aa, Artificial Sequence
  peptide linker
GGGGSGGGGSGGGGS

SEQ ID NO: 237 - protein, 14 aa, Artificial Sequence
  peptide linker
GGGGSGGGGSGGGG

SEQ ID NO: 238 - protein, 26 aa, Artificial Sequence
  peptide linker
GGGSGGGGSGGGGSGGGGSGGGGSGG

SEQ ID NO: 239 - protein, 8 aa, Artificial Sequence
  CRD homo-domain dimerization motif
  misc_feature (2)..(2): Xaa = Glu or Lys
  misc_feature (4)..(4): Xaa = Leu or Met
  misc_feature (6)..(6): Xaa = Pro or Ala
  misc_feature (7)..(7): Xaa = Val, Ile or Leu
GXWXVXXG

SEQ ID NO: 240 - protein, 10 aa, Artificial Sequence
  ephrin receptor JM domain motif
  misc_feature (2)..(2): Xaa = Ile or Val
  misc_feature (4)..(4): Xaa = Pro or Leu
  misc_feature (5)..(5): Xaa = Gln, His, Phe, Asp, Glu or Ser
  misc_feature (6)..(6): Xaa = Ala or Thr
YXDXXXYEDP

SEQ ID NO: 241 - protein, 10 aa, Artificial Sequence
  ephrin receptor JM domain motif
  misc_feature (2)..(2): Xaa = Ile or Val
  misc_feature (4)..(4): Xaa = Pro or Leu
  misc_feature (5)..(5): Xaa = Gln, His, Phe, Asp, Glu or Ser
  misc_feature (6)..(6): Xaa = Ala or Thr
FXDXXXFEDP

SEQ ID NO: 242 - protein, 5 aa, Artificial Sequence
  peptide linker
GGGGS

SEQ ID NO: 243 - protein, 66 aa, Homo sapiens
  LBD sequences of human EphA1 receptor
WSEQQQILNGTPLYMYQDCPMQGRRDTDHWLRSNWIYRGEEASRVHVELQFTVRDCKSFPGGAGPL

SEQ ID NO: 244 - protein, 63 aa, Homo sapiens
  LBD sequences of human EphA2 receptor
WDLMQNIMNDMPIYMYSVCNVMSGDQDNWLRTNWVYRGEAERIFIELKFTVRDCNSFPGGASS

SEQ ID NO: 245 - protein, 64 aa, Homo sapiens
  LBD sequences of human EphA3 receptor
WEEISGVDEHYTPIRTYQVCNVMDHSQNNWLRTNWVPRNSAQKIYVELKFTLRDCNSIPLVLGT

SEQ ID NO: 246 - protein, 64 aa, Homo sapiens
  LBD sequences of human EphA4 receptor
WEEVSIMDEKNTPIRTYQVCNVMEPSQNNWLRTDWITREGAQRVYIEIKFTLRDCNSLPGVMGT

SEQ ID NO: 247 - protein, 64 aa, Homo sapiens
  LBD sequences of human EphA5 receptor
WEEIGEVDENYAPIHTYQVCKVMEQNQNNWLLTSWISNEGASRIFIELKFTLRDCNSLPGGLGT

SEQ ID NO: 248 - protein, 64 aa, Homo sapiens
  LBD sequences of human EphA6 receptor
WDAITEMDEHNRPIHTYQVCNVMEPNQNNWLRTNWISRDAAQKIYVEMKFTLRDCNSIPWVLGT

SEQ ID NO: 249 - protein, 64 aa, Homo sapiens
  LBD sequences of human EphA7 receptor
WEEISGLDENYTPIRTYQVCQVMEPNQNNWLRTNWISKGNAQRIFVELKFTLRDCNSLPGVLGT

SEQ ID NO: 250 - protein, 64 aa, Homo sapiens
  LBD sequences of human EphA8 receptor
WDSINEVDESFQPIHTYQVCNVMSPNQNNWLRTSWVPRDGARRVYAEIKFTLRDCNSMPGVLGT

SEQ ID NO: 251 - protein, 64 aa, Homo sapiens
  LBD sequences of human EphA10 receptor
WEEISGVDEHDRPIRTYQVCNVLEPNQDNWLQTGWISRGRGQRIFVELQFTLRDCSSIPGAAGT

SEQ ID NO: 252 - protein, 64 aa, Homo sapiens
  LBD sequences of human EphB1 receptor
WEEVSGYDENLNTIRTYQVCNVFEPNQNNWLLTTFINRRGAHRIYTEMRFTVRDCSSLPNVPGS

SEQ ID NO: 253 - protein, 64 aa, Homo sapiens
  LBD sequences of human EphB2 receptor
WEEVSGYDENMNTIRTYQVCNVFESSQNNWLRTKFIRRRGAHRIHVEMKFSVRDCSSIPSVPGS

SEQ ID NO: 254 - protein, 64 aa, Homo sapiens
  LBD sequences of human EphB3 receptor
WEEVSGYDEAMNPIRTYQVCNVRESSQNNWLRTGFIWRRDVQRVYVELKFTVRDCNSIPNIPGS

SEQ ID NO: 255 - protein, 65 aa, Homo sapiens
  LBD sequences of human EphB4 receptor
WEELSGLDEEQHSVRTYEVCDVQRAPGQAHWLRTGWVPRRGAVHVYATLRFTMLECLSLPRAGRS

SEQ ID NO: 256 - protein, 3 aa, Artificial Sequence
  Ephrin type-A receptor 10
VQV

(19) United States
(12) Patent Application Publication
(10) Pub. No.: US 2024/0226321 A1
(43) Pub. Date: Jul. 11, 2024

(54) PEPTIDES, NANOVESICLES, AND USES THEREOF FOR DRUG DELIVERY

(71) Applicant: Anjarium Biosciences AG, Schlieren (CH)

(72) Inventors: Joel de Beer, Zollikon (CH); Monique Maurer, Zollikon (CH); Nicolas Meier, Thalwil (CH); Lavaniya Kunalingam, Zurich (CH); Marcello Clerici, Zurich (CH)

(21) Appl. No.: 18/555,446

(22) PCT Filed: Apr. 13, 2022
(86) PCT No.: PCT/EP2022/059940

§ 371 Date: Oct. 13, 2023

Related U.S. Application Data
(60) Provisional application No. 63/174,874, filed on Apr. 14, 2021.

(51) Int. Cl. A61K 47/69; A61K 47/68; C07K 14/71

(52) U.S. Cl. CPC A61K 47/6901; A61K 47/6851; A61K 47/6913; C07K 14/71; C07K 2319/01; C07K 2319/03

(57) ABSTRACT

The present disclosure pertains to polypeptides (in particular, polypeptides comprising Eph receptor domain(s), i.e., Eph receptor-derived polypeptides), nanovesicles (e.g., extracellular vesicles (EVs) and hybridosomes) comprising such polypeptides. Said polypeptides can act as membrane bound protein scaffolds to which molecules of interest can be attached. The polypeptides and nanovesicles can be used in targeting, therapeutic and/or diagnostic applications. Also provided are nucleic acids and expression vectors encoding such polypeptides as well as cells expressing said polypeptides. Further provided are methods for producing nanovesicles comprising such polypeptides. Compositions comprising such polypeptides or nanovesicles as well as their uses are also described.

### PRIORITY

This application claims the benefit of priority to U.S. Ser. No. 63/174,874 filed Apr. 14, 2021, which is incorporated herein by reference in its entirety.

### REFERENCE TO SEQUENCE LISTING SUBMITTED ELECTRONICALLY

This application incorporates by reference a Sequence Listing submitted with this application as a text file entitled “14497-007-228_Sequence_Listing.txt” created on Apr. 11, 2022 and having a size of 444,261 bytes.

### 1. FIELD

The present disclosure pertains to polypeptides (in particular, polypeptides comprising Eph receptor domain(s), i.e., Eph receptor-derived polypeptides), nanovesicles (e.g., extracellular vesicles (EVs) and hybridosomes) comprising such polypeptides. Said polypeptides can act as membrane bound protein scaffolds to which molecules of interest can be attached. The polypeptides and nanovesicles can be used in targeting, therapeutic and/or diagnostic applications. Also provided are nucleic acids and expression vectors encoding such polypeptides as well as cells expressing said polypeptides. Further provided are methods for producing nanovesicles comprising such polypeptides. Compositions comprising such polypeptides or nanovesicles as well as their uses are also described.

### 2. BACKGROUND

Despite major breakthroughs in the identification of new promising drug candidates, translating these findings into the clinic is often hampered by challenges in delivering an efficacious drug dosage to the site of the disease. A recently discovered cell-to-cell communication pathway may provide the missing puzzle piece for more precise drug delivery. It has emerged that almost all the cells within our body can establish links to neighboring as well as distant cells by the release of tiny “balloons”, termed extracellular vesicles (EVs). The discovery that these EVs, in particular exosomes, are functional shuttles of signaling molecules, led to the proposition that they could pose as ideal nanoscale candidates for drug delivery systems of modern-day pharmaceuticals. However, this notion is linked to several challenges. Accordingly, suitable methods and compositions for generating, isolating and purifying EVs are needed to improve therapeutic use and other applications of EV-based technologies.

Several strategies for customizing the EV surface and cargo are under development to enable the loading of EVs with pharmaceutical agents and/or the decoration of the EV surface with tissue targeting ligands. Bioengineering of EV producer cells to enable sorting of proteins of interest into EVs has been an area of interest. In particular, harnessing EV-associated proteins which are endogenously involved in EV biogenesis and vesicular protein sorting has been the focus of the art. A wide range of methods have been applied over the years for the identification of EV-associated proteins, and mass spectrometry (MS)-based proteomics has proven to be very useful. Classically, by combing proteomics data of purified EVs, highly enriched endogenous proteins (so called “EV-markers”) and proteins that associate with such markers have been identified and used as sorting scaffolds to load fusion proteins into EVs. Ubiquitously and highly expressed protein markers classically employed as sorting proteins include tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin, Prostaglandin F2 receptor negative regulator, Ubiquitin C, syntenin, syndecan and Alix (see review by Shi et al., 2020, Methods 177:95-102 (published online on Sep. 27, 2019)).

However, large differences in sorting between the endogenous and bioengineered proteins have been reported. A recent quantitative comparison for the most commonly used sorting proteins for bioengineering of EVs showed that among all EV-related target proteins screened, overexpressed fusion proteins of GFP and tetraspanins CD9, CD81, and CD63, as well as a myristoylation domain showed the highest abundance within EVs (Corso et al. 2019, 8(1):1663043). However, not all endogenous marker proteins enriched in EVs that were identified by western blotting or MS proteomics correlated with or led to efficient vesicular sorting of the corresponding GFP-fusion proteins. Furthermore, GFP-EV marker fusion proteins showed highly heterogeneous sorting efficiencies into EVs despite similar overexpression levels in the parental cells. A prominent example is the widely used EV marker Alix, which was hardly detectable in EVs when overexpressed as a fusion protein with a GFP. Additionally, despite having identified CD63-GFP fusion proteins as more abundant on EVs, substantial differences in loading efficiencies were observed, depending on whether GFP was attached to the N-terminus, C-terminus or second loop of CD63.

Accordingly, there is a need in the art for bioengineered proteins that are readily and efficiently incorporated into EVs and more generally into nanovesicles, preferably at high density.

Citation of a reference herein shall not be construed as an admission that such is prior art to the present disclosure.

### 3. SUMMARY OF THE DISCLOSURE

In one aspect, provided herein is an extracellular vesicle (EV) comprising a polypeptide, wherein the polypeptide comprises in N-terminus to C-terminus direction: a. an ephrin receptor cysteine-rich (CR) domain; b. a first ephrin receptor fibronectin type III (FN III) domain and a second ephrin receptor FN III domain; and c. a transmembrane (TM) domain; wherein the polypeptide lacks (i) ephrin binding activity, (ii) ephrin receptor kinase activity, or (iii) both ephrin binding activity and ephrin receptor kinase activity. In certain embodiments, the polypeptide lacks ephrin binding activity.

In another aspect, provided herein is a hybridosome comprising a polypeptide, wherein the polypeptide comprises in N-terminus to C-terminus direction: a. an ephrin receptor CR domain; b. a first ephrin receptor FN III domain and a second ephrin receptor FN III domain; and c. a TM domain; wherein the polypeptide lacks (i) ephrin binding activity, (ii) ephrin receptor kinase activity, or (iii) both ephrin binding activity and ephrin receptor kinase activity. In certain embodiments, the polypeptide lacks ephrin binding activity.

In various embodiments, the polypeptide further comprises a targeting domain N-terminal to the ephrin receptor CR domain. In certain embodiments, the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin. In certain embodiments, the targeting domain specifically binds to a marker. In specific embodiments, the marker is a tumor-associated antigen. In specific embodiments, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

In various embodiments, the polypeptide further comprises a cargo protein or a cargo binding domain C-terminal to the TM domain. In certain embodiments, the cargo protein or cargo binding domain is fused to the remaining portion of the polypeptide via a linker. In certain embodiments, the cargo protein or cargo binding domain is covalently fused to the remaining portion of the polypeptide via a linker. In specific embodiments, the linker is a peptide linker. In a specific embodiment, the peptide linker comprises an amino acid sequence of (GGGS)n (SEQ ID NO: 226), wherein n is an integer number from 1 to 10. In another specific embodiment, the peptide linker comprises an amino acid sequence of GGGS.

In various embodiments, the polypeptide comprises a cargo binding domain that is capable of binding to a cargo protein directly, or indirectly via a scaffold binding domain (SBD) linked to the cargo protein. In specific embodiments, the binding between the cargo binding domain and the cargo protein is a non-covalent binding. In specific embodiments, the binding between the cargo binding domain and the cargo protein is a reversible binding. In specific embodiments, the binding between the cargo binding domain and the cargo protein is capable of being controlled. In a specific embodiment, the binding between the cargo binding domain and the cargo protein is capable of being controlled by pH. In another specific embodiment, the binding between the cargo binding domain and the cargo protein is capable of being controlled by ionic strength. In some embodiments, the binding between the cargo binding domain and the cargo protein is capable of being controlled such that the cargo protein is bound to the cargo binding domain in vitro but is released from the cargo binding domain in vivo. In other embodiments, the binding between the cargo binding domain and the cargo protein is capable of being controlled such that the cargo protein is released from the cargo binding domain in a manner dependent on the subcellular compartment in which they are located. In specific embodiments, the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine. In a specific embodiment, the domain that is capable of binding to phosphotyrosine is a phosphotyrosine binding (PTB) domain. In another specific embodiment, the domain that is capable of binding to phosphotyrosine is a Src homology 2 (SH2) domain. In specific embodiments, the cargo binding domain comprises a first sterile α-motif (SAM) domain and the cargo protein or the SBD comprises a second SAM domain, and the binding between the cargo binding domain and the cargo protein is a binding between the first SAM domain and the second SAM domain. In specific embodiments, the cargo binding domain comprises a PDZ binding motif (PBM) domain and the cargo protein or the SBD comprises a PDZ domain, and the binding between the cargo binding domain and the cargo protein is a binding between the PBM domain and the PDZ domain. In specific embodiments, the cargo binding domain comprises a PDZ domain and the cargo protein or the SBD comprises a PBM domain, and the binding between the cargo binding domain and the cargo protein is a binding between the PDZ domain and the PBM domain.

In various embodiments, the polypeptide comprises a cargo protein.

In various embodiments, the polypeptide further comprises an ephrin receptor JM domain that is capable of binding to a cargo protein directly, or indirectly via a SBD linked to the cargo protein, and is C-terminal to the TM domain. In certain embodiments, the binding between the ephrin receptor JM domain and the cargo protein is a non-covalent binding. In certain embodiments, the binding between the ephrin receptor JM domain and the cargo protein is a reversible binding. In certain embodiments, the binding between the ephrin receptor JM domain and the cargo protein is capable of being controlled. In a specific embodiment, the binding between the ephrin receptor JM domain and the cargo protein is capable of being controlled by pH. In another specific embodiment, the binding between the ephrin receptor JM domain and the cargo protein is capable of being controlled by ionic strength. In some embodiments, the binding between the ephrin receptor JM domain and the cargo protein is capable of being controlled such that the cargo protein is bound to the ephrin receptor JM domain in vitro but is released from the ephrin receptor JM domain in vivo. In other embodiments, the binding between the ephrin receptor JM domain and the cargo protein is capable of being controlled such that the cargo protein is released from the ephrin receptor JM domain in a manner dependent on the subcellular compartment in which they are located. In certain embodiments, the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the ephrin receptor JM domain comprises: (i) a (X₁)-Ptyr-(X₂) motif, wherein Ptyr is a phosphotyrosine, X₁ is Y, P, V, I, T, or F, and X₂ is I, V, L, or A; (ii) a (X₃)-Ptyr-(X₄) motif, wherein Ptyr is a phosphotyrosine, X₃ is T, A, or S, and X₄ is E or G; or (iii) both (i) and (ii).

In various embodiments, the polypeptide further comprises an ephrin receptor KD that is capable of binding to a cargo protein directly, or indirectly via a SBD linked to the cargo protein, and is C-terminal to the TM domain. In certain embodiments, the binding between the ephrin receptor KD and the cargo protein is a non-covalent binding. In certain embodiments, the binding between the ephrin receptor KD and the cargo protein is a reversible binding. In certain embodiments, the binding between the ephrin receptor KD and the cargo protein is capable of being controlled. In a specific embodiment, the binding between the ephrin receptor KD and the cargo protein is capable of being controlled by pH. In another specific embodiment, the binding between the ephrin receptor KD and the cargo protein is capable of being controlled by ionic strength. In some embodiments, the binding between the ephrin receptor KD and the cargo protein is capable of being controlled such that the cargo protein is bound to the ephrin receptor KD in vitro but is released from the ephrin receptor KD in vivo. In other embodiments, the binding between the ephrin receptor KD and the cargo protein is capable of being controlled such that the cargo protein is released from the ephrin receptor KD in a manner dependent on the subcellular compartment in which they are located. In certain embodiments, the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the KD comprises an (X₇)-Ptyr-(X₈) motif in the activation loop, wherein Ptyr is a phosphotyrosine, X₇ is T, V, or A, and X₈ is E or T.

In various embodiments, the polypeptide further comprises a SAM linker domain that is capable of binding to a cargo protein directly, or indirectly via a SBD linked to the cargo protein, and is C-terminal to the TM domain. In certain embodiments, the binding between the SAM linker domain and the cargo protein is a non-covalent binding. In certain embodiments, the binding between the SAM linker domain and the cargo protein is a reversible binding. In certain embodiments, the binding between the SAM linker domain and the cargo protein is capable of being controlled. In a specific embodiment, the binding between the SAM linker domain and the cargo protein is capable of being controlled by pH. In another specific embodiment, the binding between the SAM linker domain and the cargo protein is capable of being controlled by ionic strength. In some embodiments, the binding between the SAM linker domain and the cargo protein is capable of being controlled such that the cargo protein is bound to the SAM linker domain in vitro but is released from the SAM linker domain in vivo. In other embodiments, the binding between the SAM linker domain and the cargo protein is capable of being controlled such that the cargo protein is released from the SAM linker domain in a manner dependent on the subcellular compartment in which they are located. In certain embodiments, the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid. In certain embodiments, the SAM linker domain is an ephrin receptor SAM linker domain.

In various embodiments, the polypeptide further comprises a SAM domain that is capable of binding to a cargo protein directly, or indirectly via a SBD linked to the cargo protein, and is C-terminal to the TM domain. In certain embodiments, the binding between the SAM domain and the cargo protein is a non-covalent binding. In certain embodiments, the binding between the SAM domain and the cargo protein is a reversible binding. In certain embodiments, the binding between the SAM domain and the cargo protein is capable of being controlled. In a specific embodiment, the binding between the SAM domain and the cargo protein is capable of being controlled by pH. In another specific embodiment, the binding between the SAM domain and the cargo protein is capable of being controlled by ionic strength. In some embodiments, the binding between the SAM domain and the cargo protein is capable of being controlled such that the cargo protein is bound to the SAM domain in vitro but is released from the SAM domain in vivo. In other embodiments, the binding between the SAM domain and the cargo protein is capable of being controlled such that the cargo protein is released from the SAM domain in a manner dependent on the subcellular compartment in which they are located. In certain embodiments, the cargo protein or the SBD comprises a second SAM domain, and the binding between the SAM domain and the cargo protein is a binding between the SAM domain and the second SAM domain. In certain embodiments, the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the SAM domain comprises a phosphotyrosine in the α2 helix. In a specific embodiment, the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T. In certain embodiments, the SAM domain is an ephrin receptor SAM domain.

In various embodiments, the polypeptide further comprises an ephrin receptor PDZ binding motif (PBM) domain that is capable of binding to a cargo protein directly, or indirectly via a SBD linked to the cargo protein, and is C-terminal to the TM domain. In certain embodiments, the binding between the ephrin receptor PBM domain and the cargo protein is a non-covalent binding. In certain embodiments, the binding between the ephrin receptor PBM domain and the cargo protein is a reversible binding. In certain embodiments, the binding between the ephrin receptor PBM domain and the cargo protein is capable of being controlled. In a specific embodiment, the binding between the ephrin receptor PBM domain and the cargo protein is capable of being controlled by pH. In another specific embodiment, the binding between the ephrin receptor PBM domain and the cargo protein is capable of being controlled by ionic strength. In some embodiments, the binding between the ephrin receptor PBM domain and the cargo protein is capable of being controlled such that the cargo protein is bound to the ephrin receptor PBM domain in vitro but is released from the ephrin receptor PBM domain in vivo. In other embodiments, the binding between the ephrin receptor PBM domain and the cargo protein is capable of being controlled such that the cargo protein is released from the ephrin receptor PBM domain in a manner dependent on the subcellular compartment in which they are located. In certain embodiments, the cargo protein or the SBD comprises a PDZ domain, and the binding between the ephrin receptor PBM domain and the cargo protein is a binding between the ephrin receptor PBM domain and the PDZ domain.

In various embodiments, the cargo protein is a therapeutic protein. In a specific embodiment, the therapeutic protein is a therapeutic antibody or an antigen binding fragment thereof. In another specific embodiment, the therapeutic protein is a gene editor or transposase. In various embodiments, the cargo protein is a diagnostic protein. In a specific embodiment, the diagnostic protein is a fluorescent protein.

In various embodiments, the polypeptide lacks an ephrin receptor ligand binding domain (LBD). In various embodiments, the polypeptide comprises a mutated ephrin receptor LBD.

In various embodiments, the polypeptide comprises two different domains that allow the polypeptide to undergo hetero-domain dimerization with another polypeptide identical to said polypeptide. In certain embodiments, the polypeptide comprises two different domains that allow the polypeptide to undergo hetero-domain dimerization with another polypeptide identical to said polypeptide, in a head-to-tail configuration.

In various embodiments, the TM domain is an ephrin receptor TM domain.

In various embodiments, any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof. In certain embodiments, any one or more of the ephrin receptor domains of the polypeptide are from or derived from EphA2, EphA4, EphB2, or a combination thereof.

In various embodiments, the polypeptide further comprises a modified Fc domain of an immunoglobulin. In certain embodiments, the modified Fc domain is N-terminal to the ephrin receptor CR domain. In specific embodiments, the modified Fc domain is fused to the remaining portion of the polypeptide by a linker sequence. In certain embodiments, the modified Fc domain a. is capable of specifically binding to the Fc binding site of a neonatal Fc receptor (FcRn); and b. lacks the ability to form homodimers. In certain embodiments, the dissociation constant of the modified Fc domain bound to the FcRn at a pH of 6.5 has a value of at most 10⁻⁴ M. In certain embodiments, the dissociation constant of the modified Fc domain bound to the FcRn at a pH of 7.4 has a value of at least 10⁻⁴ M. In specific embodiments, the modified Fc domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid. In specific embodiments, the modified Fc domain is capable of specifically binding to the amino acid sequence between position 135-158 of human FcRn (SEQ ID NO: 228) and/or mouse FcRn (SEQ ID NO: 227). In certain embodiments, the polypeptide does not substantially bind to C1q, FcγRI, FcγRII or FcγRIII. In certain embodiments, the complement dependent cytotoxicity (CDC) activity of the modified Fc domain, the antibody dependent cell mediated cytotoxicity (ADCC) activity of the modified Fe domain, the antibody dependent cell mediated phagocytosis (ADCP) activity of the modified Fc domain, and/or the antibody dependent intracellular neutralization (ADIN) activity of the modified Fc domain, is decreased by at least 10%, 20%, 30%, 40%, or 50% compared to an unmodified Fc domain. In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 1.5, 2, 3, 4, or 5-fold, compared to an unmodified Fc domain. In certain embodiments, the modified Fc domain comprises from N-terminus to C-terminus: a. a modified CH2 domain that is modified to decrease effector function relative to the unmodified CH2 domain; and b. a modified CH3 domain that is modified to lack the ability to form homodimers.

In various embodiments, the first ephrin receptor FN III domain and the second ephrin receptor FN III domain comprise different amino acid sequences.

In another aspect, provided herein is a method of delivering a therapeutic or diagnostic agent to a target cell or tissue, wherein the method comprises providing an extracellular vesicle or hybridosome described herein to said target cell or tissue.

In another aspect, provided herein is a polypeptide comprising in N-terminus to C-terminus direction: a. a targeting domain; b. an ephrin receptor CR domain; c. a first ephrin receptor FN III domain and a second ephrin receptor FN III domain; and d. a TM domain. In certain embodiments, the polypeptide lacks ephrin binding activity. In certain embodiments, the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin. In certain embodiments, the targeting domain specifically binds to a marker. In specific embodiments, the marker is a tumor-associated antigen. In specific embodiments, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

In another aspect, provided herein is a polypeptide comprising in N-terminus to C-terminus direction: a. an ephrin receptor CR domain; b. a first ephrin receptor FN III domain and a second ephrin receptor FN III domain; c. a TM domain; and d. a cargo protein or a cargo binding domain. In certain embodiments, the polypeptide lacks ephrin binding activity.

In another aspect, provided herein is a polypeptide comprising in N-terminus to C-terminus direction: a. a targeting domain; b. an ephrin receptor CR domain; c. a first ephrin receptor FN III domain and a second ephrin receptor FN III domain; d. a TM domain; and e. a cargo protein or a cargo binding domain. In certain embodiments, the polypeptide lacks ephrin binding activity. In certain embodiments, the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin. In certain embodiments, the targeting domain specifically binds to a marker. In specific embodiments, the marker is a tumor-associated antigen. In specific embodiments, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

In various embodiments, the cargo protein or cargo binding domain is fused to the remaining portion of the polypeptide via a linker. In certain embodiments, the cargo protein or cargo binding domain is covalently fused to the remaining portion of the polypeptide via a linker. In specific embodiments, the linker is a peptide linker. In a specific embodiment, the peptide linker comprises an amino acid sequence of (GGGS)n (SEQ ID NO: 226), wherein n is an integer number from 1 to 10. In another specific embodiment, the peptide linker comprises an amino acid sequence of GGGS.

In various embodiments, the polypeptide comprises a cargo binding domain that is capable of binding to a cargo protein directly, or indirectly via a SBD linked to the cargo protein. In specific embodiments, the binding between the cargo binding domain and the cargo protein is a non-covalent binding. In specific embodiments, the binding between the cargo binding domain and the cargo protein is a reversible binding. In specific embodiments, the binding between the cargo binding domain and the cargo protein is capable of being controlled. In a specific embodiment, the binding between the cargo binding domain and the cargo protein is capable of being controlled by pH. In another specific embodiment, the binding between the cargo binding domain and the cargo protein is capable of being controlled by ionic strength. In some embodiments, the binding between the cargo binding domain and the cargo protein is capable of being controlled such that the cargo protein is bound to the cargo binding domain in vitro but is released from the cargo binding domain in vivo. In other embodiments, the binding between the cargo binding domain and the cargo protein is capable of being controlled such that the cargo protein is released from the cargo binding domain in a manner dependent on the subcellular compartment in which they are located. In specific embodiments, the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine. In a specific embodiment, the domain that is capable of binding to phosphotyrosine is a phosphotyrosine binding (PTB) domain. In another specific embodiment, the domain that is capable of binding to phosphotyrosine is a Src homology 2 (SH2) domain. In specific embodiments, the cargo binding domain comprises a first sterile α-motif (SAM) domain and the cargo protein or the SBD comprises a second SAM domain, and the binding between the cargo binding domain and the cargo protein is a binding between the first SAM domain and the second SAM domain. In specific embodiments, the cargo binding domain comprises a PDZ binding motif (PBM) domain and the cargo protein or the SBD comprises a PDZ domain, and the binding between the cargo binding domain and the cargo protein is a binding between the PBM domain and the PDZ domain. In specific embodiments, the cargo binding domain comprises a PDZ domain and the cargo protein or the SBD comprises a PBM domain, and the binding between the cargo binding domain and the cargo protein is a binding between the PDZ domain and the PBM domain.

In various embodiments, the polypeptide comprises a cargo protein.

In various embodiments, the polypeptide further comprises an ephrin receptor JM domain that is capable of binding to a cargo protein directly, or indirectly via a SBD linked to the cargo protein, and is C-terminal to the TM domain. In certain embodiments, the binding between the ephrin receptor JM domain and the cargo protein is a non-covalent binding. In certain embodiments, the binding between the ephrin receptor JM domain and the cargo protein is a reversible binding. In certain embodiments, the binding between the ephrin receptor JM domain and the cargo protein is capable of being controlled. In a specific embodiment, the binding between the ephrin receptor JM domain and the cargo protein is capable of being controlled by pH. In another specific embodiment, the binding between the ephrin receptor JM domain and the cargo protein is capable of being controlled by ionic strength. In some embodiments, the binding between the ephrin receptor JM domain and the cargo protein is capable of being controlled such that the cargo protein is bound to the ephrin receptor JM domain in vitro but is released from the ephrin receptor JM domain in vivo. In other embodiments, the binding between the ephrin receptor JM domain and the cargo protein is capable of being controlled such that the cargo protein is released from the ephrin receptor JM domain in a manner dependent on the subcellular compartment in which they are located. In certain embodiments, the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the ephrin receptor JM domain comprises: (i) a (X₁)-Ptyr-(X₂) motif, wherein Ptyr is a phosphotyrosine, X₁ is Y, P, V, I, T, or F, and X₂ is I, V, L, or A; (ii) a (X₃)-Ptyr-(X₄) motif, wherein Ptyr is a phosphotyrosine, X₃ is T, A, or S, and X₄ is E or G; or (iii) both (i) and (ii).

In various embodiments, the polypeptide further comprises an ephrin receptor KD that is capable of binding to a cargo protein directly, or indirectly via a SBD linked to the cargo protein, and is C-terminal to the TM domain. In certain embodiments, the binding between the ephrin receptor KD and the cargo protein is a non-covalent binding. In certain embodiments, the binding between the ephrin receptor KD and the cargo protein is a reversible binding. In certain embodiments, the binding between the ephrin receptor KD and the cargo protein is capable of being controlled. In a specific embodiment, the binding between the ephrin receptor KD and the cargo protein is capable of being controlled by pH. In another specific embodiment, the binding between the ephrin receptor KD and the cargo protein is capable of being controlled by ionic strength. In some embodiments, the binding between the ephrin receptor KD and the cargo protein is capable of being controlled such that the cargo protein is bound to the ephrin receptor KD in vitro but is released from the ephrin receptor KD in vivo. In other embodiments, the binding between the ephrin receptor KD and the cargo protein is capable of being controlled such that the cargo protein is released from the ephrin receptor KD in a manner dependent on the subcellular compartment in which they are located. In certain embodiments, the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the KD comprises an (X₇)-Ptyr-(X₈) motif in the activation loop, wherein Ptyr is a phosphotyrosine, X₇ is T, V, or A, and X₈ is E or T.

In various embodiments, the polypeptide further comprises a SAM linker domain that is capable of binding to a cargo protein directly, or indirectly via a SBD linked to the cargo protein, and is C-terminal to the TM domain. In certain embodiments, the binding between the SAM linker domain and the cargo protein is a non-covalent binding. In certain embodiments, the binding between the SAM linker domain and the cargo protein is a reversible binding. In certain embodiments, the binding between the SAM linker domain and the cargo protein is capable of being controlled. In a specific embodiment, the binding between the SAM linker domain and the cargo protein is capable of being controlled by pH. In another specific embodiment, the binding between the SAM linker domain and the cargo protein is capable of being controlled by ionic strength. In some embodiments, the binding between the SAM linker domain and the cargo protein is capable of being controlled such that the cargo protein is bound to the SAM linker domain in vitro but is released from the SAM linker domain in vivo. In other embodiments, the binding between the SAM linker domain and the cargo protein is capable of being controlled such that the cargo protein is released from the SAM linker domain in a manner dependent on the subcellular compartment in which they are located. In certain embodiments, the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid. In certain embodiments, the SAM linker domain is an ephrin receptor SAM linker domain.

In various embodiments, the polypeptide further comprises a SAM domain that is capable of binding to a cargo protein directly, or indirectly via a SBD linked to the cargo protein, and is C-terminal to the TM domain. In certain embodiments, the binding between the SAM domain and the cargo protein is a non-covalent binding. In certain embodiments, the binding between the SAM domain and the cargo protein is a reversible binding. In certain embodiments, the binding between the SAM domain and the cargo protein is capable of being controlled. In a specific embodiment, the binding between the SAM domain and the cargo protein is capable of being controlled by pH. In another specific embodiment, the binding between the SAM domain and the cargo protein is capable of being controlled by ionic strength. In some embodiments, the binding between the SAM domain and the cargo protein is capable of being controlled such that the cargo protein is bound to the SAM domain in vitro but is released from the SAM domain in vivo. In other embodiments, the binding between the SAM domain and the cargo protein is capable of being controlled such that the cargo protein is released from the SAM domain in a manner dependent on the subcellular compartment in which they are located. In certain embodiments, the cargo protein or the SBD comprises a second SAM domain, and the binding between the SAM domain and the cargo protein is a binding between the SAM domain and the second SAM domain. In certain embodiments, the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine. In a specific embodiment, the domain that is capable of binding to phosphotyrosine is a PTB domain. In another specific embodiment, the domain that is capable of binding to phosphotyrosine is an SH2 domain. In specific embodiments, the SAM domain comprises a phosphotyrosine in the α2 helix. In a specific embodiment, the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T. In certain embodiments, the SAM domain is an ephrin receptor SAM domain.

In various embodiments, the polypeptide further comprises an ephrin receptor PDZ binding motif (PBM) domain that is capable of binding to a cargo protein directly, or indirectly via a SBD linked to the cargo protein, and is C-terminal to the TM domain. In certain embodiments, the binding between the ephrin receptor PBM domain and the cargo protein is a non-covalent binding. In certain embodiments, the binding between the ephrin receptor PBM domain and the cargo protein is a reversible binding. In certain embodiments, the binding between the ephrin receptor PBM domain and the cargo protein is capable of being controlled. In a specific embodiment, the binding between the ephrin receptor PBM domain and the cargo protein is capable of being controlled by pH. In another specific embodiment, the binding between the ephrin receptor PBM domain and the cargo protein is capable of being controlled by ionic strength. In some embodiments, the binding between the ephrin receptor PBM domain and the cargo protein is capable of being controlled such that the cargo protein is bound to the ephrin receptor PBM domain in vitro but is released from the ephrin receptor PBM domain in vivo. In other embodiments, the binding between the ephrin receptor PBM domain and the cargo protein is capable of being controlled such that the cargo protein is released from the ephrin receptor PBM domain in a manner dependent on the subcellular compartment in which they are located. In certain embodiments, the cargo protein or the SBD comprises a PDZ domain, and the binding between the ephrin receptor PBM domain and the cargo protein is a binding between the ephrin receptor PBM domain and the PDZ domain.

In various embodiments, the cargo protein is a therapeutic protein. In a specific embodiment, the therapeutic protein is a therapeutic antibody or an antigen binding fragment thereof. In another specific embodiment, the therapeutic protein is a gene editor or transposase. In various embodiments, the cargo protein is a diagnostic protein. In a specific embodiment, the diagnostic protein is a fluorescent protein.

In various embodiments, the polypeptide lacks an ephrin receptor ligand binding domain (LBD). In various embodiments, the polypeptide comprises a mutated ephrin receptor LBD.

In various embodiments, the polypeptide comprises two different domains that allow the polypeptide to undergo hetero-domain dimerization with another polypeptide identical to said polypeptide. In certain embodiments, the polypeptide comprises two different domains that allow the polypeptide to undergo hetero-domain dimerization with another polypeptide identical to said polypeptide, in a head-to-tail configuration.

In various embodiments, the TM domain is an ephrin receptor TM domain.

In various embodiments, any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof. In certain embodiments, any one or more of the ephrin receptor domains of the polypeptide are from or derived from EphA2, EphA4, EphB2, or a combination thereof.

In various embodiments, the polypeptide further comprises a modified Fc domain of an immunoglobulin. In certain embodiments, the modified Fc domain is N-terminal to the ephrin receptor CR domain. In specific embodiments, the modified Fc domain is fused to the remaining portion of the polypeptide by a linker sequence. In certain embodiments, the modified Fc domain a. is capable of specifically binding to the Fc binding site of a neonatal Fc receptor (FcRn); and b. lacks the ability to form homodimers. In certain embodiments, the dissociation constant of the modified Fc domain bound to the FcRn at a pH of 6.5 has a value of at most 10⁻⁴ M. In certain embodiments, the dissociation constant of the modified Fc domain bound to the FcRn at a pH of 7.4 has a value of at least 10⁻⁴ M. In specific embodiments, the modified Fc domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid. In specific embodiments, the modified Fc domain is capable of specifically binding to the amino acid sequence between position 135-158 of human FcRn (SEQ ID NO: 228) and/or mouse FcRn (SEQ ID NO: 227). In certain embodiments, the polypeptide does not substantially bind to C1q, FcγRI, FcγRII or FcγRIII. In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 10%, 20%, 30%, 40%, or 50% compared to an unmodified Fe domain. In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 1.5, 2, 3, 4, or 5-fold, compared to an unmodified Fc domain. In certain embodiments, the modified Fc domain comprises from N-terminus to C-terminus: a. a modified CH2 domain that is modified to decrease effector function relative to the unmodified CH2 domain; and b. a modified CH3 domain that is modified to lack the ability to form homodimers.

In various embodiments, the first ephrin receptor FN III domain and the second ephrin receptor FN III domain comprise different amino acid sequences.

In another aspect, provided herein is a nucleic acid encoding a polypeptide described herein.

In another aspect, provided herein is an expression vector comprising a nucleic acid described herein.

In another aspect, provided herein is a cell comprising a nucleic acid described herein or an expression vector described herein.

In another aspect, provided herein is a method of producing an EV, wherein the method comprises: a. transfecting cells with a nucleic acid described herein or an expression vector described herein; b. cultivating the cells under suitable conditions for the production of the EV; and c. collecting the EV secreted by the cells.

In another aspect, provided herein is a method of producing a hybridosome, wherein the method comprises contacting a first EV with a second EV, thereby uniting the first EV with the second EV and producing the hybridosome, wherein said first EV has been produced in vitro, and the first EV comprises (i) a membrane, and (ii) a fusogenic, ionizable, cationic lipid, and wherein said second EV has been produced by the method described above.

In another aspect, provided herein is a method of purifying an EV or a hybridosome, wherein the method comprises: a. providing the EV or hybridosome, wherein the EV or hybridosome comprises a first binding partner, wherein the first binding partner is capable of binding to the Fc binding site of an FcRn in a pH dependent manner; and b. contacting at a first pH the EV or hybridosome comprising the first binding partner with a second binding partner, wherein the second binding partner comprises the Fc binding site of the FcRn and is associated with a solid matrix; and c. eluting the EV or hybridosome comprising the first binding partner from the solid matrix at a second pH. In certain embodiments, the method further comprises a washing step at the first pH. In certain embodiments, the first pH is below 6.5. In certain embodiments, the second pH is above 7.4. In certain embodiments, the Fc binding site of the FcRn comprises the amino acid sequence of SEQ ID NO: 230.

In another aspect, provided herein is a method of purifying an EV or a hybridosome, wherein the method comprises: a. providing the EV or hybridosome, wherein the EV or hybridosome comprises a first binding partner, wherein the first binding partner is capable of binding to the Fc binding site of an FcRn in a pH dependent manner and comprises or consists of a polypeptide described herein; and b. contacting at a first pH the EV or hybridosome comprising the first binding partner with a second binding partner, wherein the second binding partner comprises the Fc binding site of the FcRn and is associated with a solid matrix; and c. eluting the EV or hybridosome comprising the first binding partner from the solid matrix at a second pH. In certain embodiments, the method further comprises a washing step at the first pH. In certain embodiments, the first pH is below 6.5. In certain embodiments, the second pH is above 7.4. In certain embodiments, the Fc binding site of the FcRn comprises the amino acid sequence of SEQ ID NO: 230.

In some embodiments, polypeptides provided herein are signal neutral in that there is reduced forward (i.e., luminal) signaling capacity either due to the lack of the endodomain or parts thereof (e.g., Sterile alpha motif (SAM) Domain and/or PDZ domain) or because the kinase activity of the parental Eph receptor has been inactivated, e.g., through mutation and/or deletion. Furthermore, the scaffolds are preferably extracellularly inert as the ligand binding domain is preferably modified or deleted such that binding to the natural ligand of the Eph receptor, ephrins, is diminished or abolished.

Importantly, such engineered Eph receptor variants of the disclosure have both the N- and the C-terminal accessible and free to which a molecule of interest (e.g., a cargo, a targeting domain, or a purification domain) can be fused. For example, the polypeptides can be fused in-frame with one or more targeting domains, e.g., allowing the nanovesicles (such as EVs and hybridosomes) comprising such polypeptide to be targeted to particular cell types upon administration to a subject. For a fusion moiety to be functional, it is beneficial to have a certain distance between the fusion moiety and the surface of the nanovesicle (e.g., EV or hybridosome). Fusing the targeting moiety to the N-terminal end of the ligand binding domain (LBD) of the Eph receptor derived polypeptides yields a structure which is flexible to bend and/or reconfigure but at the same time stable. Moreover, ectodomain of an Eph receptor provides a long protrusion for reach, as the ectodomain of the Eph receptor protrudes from the membrane.

In certain embodiments, the polypeptides disclosed herein as well as nanovesicles (e.g., EVs and hybridosomes) comprising these polypeptides are suitable for therapeutic applications.

In one aspect, the disclosure provides a polypeptide derived from an Eph receptor, said polypeptide
i. comprising an ephrin ligand binding domain exhibiting decreased or no binding to ephrins as compared to the parental Eph receptor; and
ii. comprising a transmembrane domain.

Preferably, said polypeptide is fused to one or more molecules of interest, preferably proteins.

In another aspect, a nucleic acid encoding a polypeptide described herein is provided.

In another aspect, an expression vector comprising a nucleic acid described herein is provided.

In still another aspect, a cell comprising such a nucleic acid or expression vector is provided. An exogenous nucleic acid or expression vector can be introduced transiently or stably into a cell. In preferred embodiments, such cell is a source cell capable of producing nanovesicles (e.g., EVs and hybridosomes) under suitable conditions.

In another aspect, the disclosure relates to a nanovesicle (e.g., an EV or hybridosome) comprising a polypeptide disclosed herein. In one embodiment, the nanovesicles are derived from a source cell (i.e., extracellular vesicles or “EVs”). In one embodiment, the nanovesicles are natural/synthetic hybrids (such as hybridosomes).

In another aspect, a method of producing a nanovesicle (e.g., an EV or hybridosome) being surface decorated with one or more heterologous polypeptides (e.g. targeting domains) is provided, comprising the steps of
(i) providing a nucleic acid or an expression vector encoding the polypeptide disclosed herein being fused to one or more protein(s) of interest;
(ii) transfecting cells with the nucleic acid or expression vector as described herein;
(iii) cultivating said cells under suitable conditions so that nanovesicles are produced; and
(iv) purifying the so produced nanovesicles from the cell culture.

Further provided are compositions comprising a nanovesicle as described herein, a nucleic acid as described herein, an expression vector as described herein and/or a cell as described herein. Such compositions may be used in the treatment of a disease or disorder.

Further provided is a method of treating a disease or disorder comprising administering to a subject a therapeutically effective amount of a composition described herein.

### 3.1 ILLUSTRATIVE EMBODIMENTS

1. An extracellular vesicle (EV) comprising a polypeptide, wherein the polypeptide comprises in N-terminus to C-terminus direction:
a. an ephrin receptor cysteine-rich (CR) domain;
b. a first ephrin receptor fibronectin type III (FN III) domain and a second ephrin receptor FN III domain; and
c. a transmembrane (TM) domain;
wherein the polypeptide lacks (i) ephrin binding activity, (ii) ephrin receptor kinase activity, or (iii) both ephrin binding activity and ephrin receptor kinase activity.

2. A hybridosome comprising a polypeptide, wherein the polypeptide comprises in N-terminus to C-terminus direction:
a. an ephrin receptor CR domain;
b. a first ephrin receptor FN III domain and a second ephrin receptor FN III domain; and
c. a TM domain;
wherein the polypeptide lacks (i) ephrin binding activity, (ii) ephrin receptor kinase activity, or (iii) both ephrin binding activity and ephrin receptor kinase activity.

3. The EV of paragraph 1 or the hybridosome of paragraph 2, wherein the polypeptide lacks ephrin binding activity.

4. The EV or hybridosome of any one of paragraphs 1-3, wherein the polypeptide further comprises a targeting domain N-terminal to the ephrin receptor CR domain.

5. The EV or hybridosome of paragraph 4, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

6. The EV or hybridosome of paragraph 4 or 5, wherein the targeting domain specifically binds to a marker.

7. The EV or hybridosome of paragraph 6, wherein the marker is a tumor-associated antigen.

8. The EV or hybridosome of paragraph 7, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

9. The EV or hybridosome of any one of paragraphs 1-8, wherein the polypeptide further comprises a cargo protein or a cargo binding domain C-terminal to the TM domain.

10. The EV or hybridosome of paragraph 9, wherein the cargo protein or cargo binding domain is fused to the remaining portion of the polypeptide via a linker.

11. The EV or hybridosome of paragraph 10, wherein the cargo protein or cargo binding domain is covalently fused to the remaining portion of the polypeptide via a linker.

12. The EV or hybridosome of paragraph 10 or 11, wherein the linker is a peptide linker.

13. The EV or hybridosome of paragraph 12, wherein the peptide linker comprises an amino acid sequence of (GGGS)n (SEQ ID NO: 226), wherein n is an integer number from 1 to 10.

14. The EV or hybridosome of paragraph 12, wherein the peptide linker comprises an amino acid sequence of GGGS.

15. The EV or hybridosome of any one of paragraphs 9-14, wherein the polypeptide comprises a cargo binding domain that is capable of binding to a cargo protein directly, or indirectly via a scaffold binding domain (SBD) linked to the cargo protein.

16. The EV or hybridosome of paragraph 15, wherein the binding between the cargo binding domain and the cargo protein is a non-covalent binding.

17. The EV or hybridosome of paragraph 15 or 16, wherein the binding between the cargo binding domain and the cargo protein is a reversible binding.

18. The EV or hybridosome of any one of paragraphs 15-17, wherein the binding between the cargo binding domain and the cargo protein is capable of being controlled.

19. The EV or hybridosome of paragraph 18, wherein the binding between the cargo binding domain and the cargo protein is capable of being controlled by pH.

20. The EV or hybridosome of paragraph 18, wherein the binding between the cargo binding domain and the cargo protein is capable of being controlled by ionic strength.

21. The EV or hybridosome of any one of paragraphs 15-20, wherein the binding between the cargo binding domain and the cargo protein is capable of being controlled such that the cargo protein is bound to the cargo binding domain in vitro but is released from the cargo binding domain in vivo.

22. The EV or hybridosome of any one of paragraphs 15-20, wherein the binding between the cargo binding domain and the cargo protein is capable of being controlled such that the cargo protein is released from the cargo binding domain in a manner dependent on the subcellular compartment in which they are located.

23. The EV or hybridosome of any one of paragraphs 15-22, wherein the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine.

24. The EV or hybridosome of paragraph 23, wherein the domain that is capable of binding to phosphotyrosine is a phosphotyrosine binding (PTB) domain.

25. The EV or hybridosome of paragraph 23, wherein the domain that is capable of binding to phosphotyrosine is a Src homology 2 (SH2) domain.

26. The EV or hybridosome of any one of paragraphs 15-22, wherein the cargo binding domain comprises a first sterile α-motif (SAM) domain and the cargo protein or the SBD comprises a second SAM domain, and the binding between the cargo binding domain and the cargo protein is a binding between the first SAM domain and the second SAM domain.

27. The EV or hybridosome of any one of paragraphs 15-22, wherein the cargo binding domain comprises a PDZ binding motif (PBM) domain and the cargo protein or the SBD comprises a PDZ domain, and the binding between the cargo binding domain and the cargo protein is a binding between the PBM domain and the PDZ domain.

28. The EV or hybridosome of any one of paragraphs 15-22, wherein the cargo binding domain comprises a PDZ domain and the cargo protein or the SBD comprises a PBM domain, and the binding between the cargo binding domain and the cargo protein is a binding between the PDZ domain and the PBM domain.

29. The EV or hybridosome of any one of paragraphs 9-14, wherein the polypeptide comprises a cargo protein.

30. The EV or hybridosome of any one of paragraphs 1-8, wherein the polypeptide further comprises an ephrin receptor JM domain that is capable of binding to a cargo protein directly, or indirectly via a SBD linked to the cargo protein, and is C-terminal to the TM domain.

31. The EV or hybridosome of paragraph 30, wherein the binding between the ephrin receptor JM domain and the cargo protein is a non-covalent binding.

32. The EV or hybridosome of paragraph 30 or 31, wherein the binding between the ephrin receptor JM domain and the cargo protein is a reversible binding.

33. The EV or hybridosome of any one of paragraphs 30-32, wherein the binding between the ephrin receptor JM domain and the cargo protein is capable of being controlled.

34. The EV or hybridosome of paragraph 33, wherein the binding between the ephrin receptor JM domain and the cargo protein is capable of being controlled by pH.

35. The EV or hybridosome of paragraph 33, wherein the binding between the ephrin receptor JM domain and the cargo protein is capable of being controlled by ionic strength.

36. The EV or hybridosome of any one of paragraphs 30-35, wherein the binding between the ephrin receptor JM domain and the cargo protein is capable of being controlled such that the cargo protein is bound to the ephrin receptor JM domain in vitro but is released from the ephrin receptor JM domain in vivo.

37. The EV or hybridosome of any one of paragraphs 30-35, wherein the binding between the ephrin receptor JM domain and the cargo protein is capable of being controlled such that the cargo protein is released from the ephrin receptor JM domain in a manner dependent on the subcellular compartment in which they are located.

38. The EV or hybridosome of any one of paragraphs 30-37, wherein the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine.

39. The EV or hybridosome of paragraph 38, wherein the domain that is capable of binding to phosphotyrosine is a PTB domain.

40. The EV or hybridosome of paragraph 38, wherein the domain that is capable of binding to phosphotyrosine is an SH2 domain.

41. The EV or hybridosome of any one of paragraphs 38-40, wherein the ephrin receptor JM domain comprises:
(i) a (X₁)-Ptyr-(X₂) motif, wherein Ptyr is a phosphotyrosine, X₁ is Y, P, V, I, T, or F, and X₂ is I, V, L, or A;
(ii) a (X₃)-Ptyr-(X₄) motif, wherein Ptyr is a phosphotyrosine, X₃ is T, A, or S, and X₄ is E or G; or
(iii) both (i) and (ii).

42. The EV or hybridosome of any one of paragraphs 1-8, wherein the polypeptide further comprises an ephrin receptor KD that is capable of binding to a cargo protein directly, or indirectly via a SBD linked to the cargo protein, and is C-terminal to the TM domain.

43. The EV or hybridosome of paragraph 42, wherein the binding between the ephrin receptor KD and the cargo protein is a non-covalent binding.

44. The EV or hybridosome of paragraph 42 or 43, wherein the binding between the ephrin receptor KD and the cargo protein is a reversible binding.

45. The EV or hybridosome of any one of paragraphs 42-44, wherein the binding between the ephrin receptor KD and the cargo protein is capable of being controlled.

46. The EV or hybridosome of paragraph 45, wherein the binding between the ephrin receptor KD and the cargo protein is capable of being controlled by pH.

47. The EV or hybridosome of paragraph 45, wherein the binding between the ephrin receptor KD and the cargo protein is capable of being controlled by ionic strength.

48. The EV or hybridosome of any one of paragraphs 42-47, wherein the binding between the ephrin receptor KD and the cargo protein is capable of being controlled such that the cargo protein is bound to the ephrin receptor KD in vitro but is released from the ephrin receptor KD in vivo.

49. The EV or hybridosome of any one of paragraphs 42-47, wherein the binding between the ephrin receptor KD and the cargo protein is capable of being controlled such that the cargo protein is released from the ephrin receptor KD in a manner dependent on the subcellular compartment in which they are located.

50. The EV or hybridosome of any one of paragraphs 42-49, wherein the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine.

51. The EV or hybridosome of paragraph 50, wherein the domain that is capable of binding to phosphotyrosine is a PTB domain.

52. The EV or hybridosome of paragraph 50, wherein the domain that is capable of binding to phosphotyrosine is an SH2 domain.

53. The EV or hybridosome of any one of paragraphs 50-52, wherein the KD comprises an (X₇)-Ptyr-(X₈) motif in the activation loop, wherein Ptyr is a phosphotyrosine, X₇ is T, V, or A, and X₈ is E or T.

54. The EV or hybridosome of any one of paragraphs 1-8, wherein the polypeptide further comprises a SAM linker domain that is capable of binding to a cargo protein directly, or indirectly via a SBD linked to the cargo protein, and is C-terminal to the TM domain.

55. The EV or hybridosome of paragraph 54, wherein the binding between the SAM linker domain and the cargo protein is a non-covalent binding.

56. The EV or hybridosome of paragraph 54 or 55, wherein the binding between the SAM linker domain and the cargo protein is a reversible binding.

57. The EV or hybridosome of any one of paragraphs 54-56, wherein the binding between the SAM linker domain and the cargo protein is capable of being controlled.

58. The EV or hybridosome of paragraph 57, wherein the binding between the SAM linker domain and the cargo protein is capable of being controlled by pH.

59. The EV or hybridosome of paragraph 57, wherein the binding between the SAM linker domain and the cargo protein is capable of being controlled by ionic strength.

60. The EV or hybridosome of any one of paragraphs 54-59, wherein the binding between the SAM linker domain and the cargo protein is capable of being controlled such that the cargo protein is bound to the SAM linker domain in vitro but is released from the SAM linker domain in vivo.

61. The EV or hybridosome of any one of paragraphs 54-59, wherein the binding between the SAM linker domain and the cargo protein is capable of being controlled such that the cargo protein is released from the SAM linker domain in a manner dependent on the subcellular compartment in which they are located.

62. The EV or hybridosome of any one of paragraphs 54-61, wherein the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid.

63. The EV or hybridosome of any one of paragraphs 54-62, wherein the SAM linker domain is an ephrin receptor SAM linker domain.

64. The EV or hybridosome of any one of paragraphs 1-8, wherein the polypeptide further comprises a SAM domain that is capable of binding to a cargo protein directly, or indirectly via a SBD linked to the cargo protein, and is C-terminal to the TM domain.

65. The EV or hybridosome of paragraph 64, wherein the binding between the SAM domain and the cargo protein is a non-covalent binding.

66. The EV or hybridosome of paragraph 64 or 65, wherein the binding between the SAM domain and the cargo protein is a reversible binding.

67. The EV or hybridosome of any one of paragraphs 64-66, wherein the binding between the SAM domain and the cargo protein is capable of being controlled.

68. The EV or hybridosome of paragraph 67, wherein the binding between the SAM domain and the cargo protein is capable of being controlled by pH.

69. The EV or hybridosome of paragraph 67, wherein the binding between the SAM domain and the cargo protein is capable of being controlled by ionic strength.

70. The EV or hybridosome of any one of paragraphs 64-69, wherein the binding between the SAM domain and the cargo protein is capable of being controlled such that the cargo protein is bound to the SAM domain in vitro but is released from the SAM domain in vivo.

71. The EV or hybridosome of any one of paragraphs 64-69, wherein the binding between the SAM domain and the cargo protein is capable of being controlled such that the cargo protein is released from the SAM domain in a manner dependent on the subcellular compartment in which they are located.

72. The EV or hybridosome of any one of paragraphs 64-71, wherein the cargo protein or the SBD comprises a second SAM domain, and the binding between the SAM domain and the cargo protein is a binding between the SAM domain and the second SAM domain.

73. The EV or hybridosome of any one of paragraphs 64-71, wherein the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine.

74. The EV or hybridosome of paragraph 73, wherein the domain that is capable of binding to phosphotyrosine is a PTB domain.

75. The EV or hybridosome of paragraph 73, wherein the domain that is capable of binding to phosphotyrosine is an SH2 domain.

76. The EV or hybridosome of any one of paragraphs 73-75, wherein the SAM domain comprises a phosphotyrosine in the α2 helix.

77. The EV or hybridosome of paragraph 76, wherein the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T.

78. The EV or hybridosome of any one of paragraphs 64-77, wherein the SAM domain is an ephrin receptor SAM domain.

79. The EV or hybridosome of any one of paragraphs 1-8, wherein the polypeptide further comprises an ephrin receptor PDZ binding motif (PBM) domain that is capable of binding to a cargo protein directly, or indirectly via a SBD linked to the cargo protein, and is C-terminal to the TM domain.

80. The EV or hybridosome of paragraph 79, wherein the binding between the ephrin receptor PBM domain and the cargo protein is a non-covalent binding.

81. The EV or hybridosome of paragraph 79 or 80, wherein the binding between the ephrin receptor PBM domain and the cargo protein is a reversible binding.

82. The EV or hybridosome of any one of paragraphs 79-81, wherein the binding between the ephrin receptor PBM domain and the cargo protein is capable of being controlled.

83. The EV or hybridosome of paragraph 82, wherein the binding between the ephrin receptor PBM domain and the cargo protein is capable of being controlled by pH.

84. The EV or hybridosome of paragraph 82, wherein the binding between the ephrin receptor PBM domain and the cargo protein is capable of being controlled by ionic strength.

85. The EV or hybridosome of any one of paragraphs 79-84, wherein the binding between the ephrin receptor PBM domain and the cargo protein is capable of being controlled such that the cargo protein is bound to the ephrin receptor PBM domain in vitro but is released from the ephrin receptor PBM domain in vivo.

86. The EV or hybridosome of any one of paragraphs 79-84, wherein the binding between the ephrin receptor PBM domain and the cargo protein is capable of being controlled such that the cargo protein is released from the ephrin receptor PBM domain in a manner dependent on the subcellular compartment in which they are located.

87. The EV or hybridosome of any one of paragraphs 79-86, wherein the cargo protein or the SBD comprises a PDZ domain, and the binding between the ephrin receptor PBM domain and the cargo protein is a binding between the ephrin receptor PBM domain and the PDZ domain.

88. The EV or hybridosome of any one of paragraphs 9-87, wherein the cargo protein is a therapeutic protein.

89. The EV or hybridosome of paragraph 88, wherein the therapeutic protein is a therapeutic antibody or an antigen binding fragment thereof.

90. The EV or hybridosome of paragraph 88, wherein the therapeutic protein is a gene editor or transposase.

91. The EV or hybridosome of any one of paragraphs 9-87, wherein the cargo protein is a diagnostic protein.

92. The EV or hybridosome of paragraph 91, wherein the diagnostic protein is a fluorescent protein.

93. The EV or hybridosome of any one of paragraphs 1-92, wherein the polypeptide lacks an ephrin receptor ligand binding domain (LBD).

94. The EV or hybridosome of any one of paragraphs 1-92, wherein the polypeptide comprises a mutated ephrin receptor LBD.

95. The EV or hybridosome of any one of paragraphs 1-94, wherein the polypeptide comprises two different domains that allow the polypeptide to undergo hetero-domain dimerization with another polypeptide identical to said polypeptide.

96. The EV or hybridosome of any one of paragraphs 1-94, wherein the polypeptide comprises two different domains that allow the polypeptide to undergo hetero-domain dimerization with another polypeptide identical to said polypeptide, in a head-to-tail configuration.

97. The EV or hybridosome of any one of paragraphs 1-96, wherein the TM domain is an ephrin receptor TM domain.

98. The EV or hybridosome of any one of paragraphs 1-97, wherein any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof.

99. The EV or hybridosome of any one of paragraphs 1-97, wherein any one or more of the ephrin receptor domains of the polypeptide are from or derived from EphA2, EphA4, EphB2, or a combination thereof.

100. The EV or hybridosome of any one of paragraphs 1-99, wherein the polypeptide further comprises a modified Fc domain of an immunoglobulin.

101. The EV or hybridosome of paragraph 100, wherein the modified Fc domain is N-terminal to the ephrin receptor CR domain.

102. The EV or hybridosome of paragraph 101, wherein the modified Fc domain is fused to the remaining portion of the polypeptide by a linker sequence.

103. The EV or hybridosome of any one of paragraphs 100-102, wherein the modified Fc domain
a. is capable of specifically binding to the Fc binding site of a neonatal Fc receptor (FcRn); and
b. lacks the ability to form homodimers.

104. The EV or hybridosome of any one of paragraph 100-103, wherein the dissociation constant of the modified Fc domain bound to the FcRn at a pH of 6.5 has a value of at most 10⁻⁴ M.

105. The EV or hybridosome of any one of paragraph 100-104, wherein the dissociation constant of the modified Fc domain bound to the FcRn at a pH of 7.4 has a value of at least 10⁻⁴ M.

106. The EV or hybridosome of any one of paragraph 100-105, wherein the modified Fe domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid.

107. The EV or hybridosome of any one of paragraph 100-106, wherein the modified Fc domain is capable of specifically binding to the amino acid sequence between position 135-158 of human FcRn (SEQ ID NO: 228) and/or mouse FcRn (SEQ ID NO: 227).

108. The EV or hybridosome of any one of paragraphs 100-107, wherein the polypeptide does not substantially bind to C1q, FcγRI, FcγRII or FcγRIII.

109. The EV or hybridosome of any one of paragraphs 100-108, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 10%, 20%, 30%, 40%, or 50% compared to an unmodified Fc domain.

110. The EV or hybridosome of any one of paragraphs 100-109, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 1.5, 2, 3, 4, or 5-fold, compared to an unmodified Fc domain.

111. The EV or hybridosome of any one of paragraphs 100-110, wherein the modified Fc domain comprises from N-terminus to C-terminus:
a. a modified CH2 domain that is modified to decrease effector function relative to the unmodified CH2 domain; and
b. a modified CH3 domain that is modified to lack the ability to form homodimers.

112. The EV or hybridosome of any one of paragraphs 1-111, wherein the first ephrin receptor FN III domain and the second ephrin receptor FN III domain comprise different amino acid sequences.

113. A method of delivering a therapeutic or diagnostic agent to a target cell or tissue, wherein the method comprises providing the extracellular vesicle or hybridosome of any one of paragraphs 1-112 to said target cell or tissue.

114. A polypeptide comprising in N-terminus to C-terminus direction:
a. a targeting domain;
b. an ephrin receptor CR domain;
c. a first ephrin receptor FN III domain and a second ephrin receptor FN III domain; and
d. a TM domain.

115. The polypeptide of paragraph 114, wherein the polypeptide lacks ephrin binding activity.

116. The polypeptide of paragraph 114 or 115, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

117. The polypeptide of any one of paragraphs 114-116, wherein the targeting domain specifically binds to a marker.

118. The polypeptide of paragraph 117, wherein the marker is a tumor-associated antigen.

119. The polypeptide of paragraph 118, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

120. A polypeptide comprising in N-terminus to C-terminus direction:
a. an ephrin receptor CR domain;
b. a first ephrin receptor FN III domain and a second ephrin receptor FN III domain;
c. a TM domain; and
d. a cargo protein or a cargo binding domain.

121. The polypeptide of paragraph 120, wherein the polypeptide lacks ephrin binding activity.

122. A polypeptide comprising in N-terminus to C-terminus direction:
a. a targeting domain;
b. an ephrin receptor CR domain;
c. a first ephrin receptor FN III domain and a second ephrin receptor FN III domain;
d. a TM domain; and
e. a cargo protein or a cargo binding domain.

123. The polypeptide of paragraph 122, wherein the polypeptide lacks ephrin binding activity.

124. The polypeptide of paragraph 122 or 123, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

125. The polypeptide of any one of paragraphs 122-124, wherein the targeting domain specifically binds to a marker.

126. The polypeptide of paragraph 125, wherein the marker is a tumor-associated antigen.

127. The polypeptide of paragraph 126, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

128. The polypeptide of any one of paragraphs 120-127, wherein the cargo protein or cargo binding domain is fused to the remaining portion of the polypeptide via a linker.

129. The polypeptide of paragraph 128, wherein the cargo protein or cargo binding domain is covalently fused to the remaining portion of the polypeptide via a linker.

130. The polypeptide of paragraph 128 or 129, wherein the linker is a peptide linker.

131. The polypeptide of paragraph 130, wherein the peptide linker comprises an amino acid sequence of (GGGS)n (SEQ ID NO: 226), wherein n is an integer number from 1 to 10.

132. The polypeptide of paragraph 130, wherein the peptide linker comprises an amino acid sequence of GGGS.

133. The polypeptide of any one of paragraphs 120-132, wherein the polypeptide comprises a cargo binding domain that is capable of binding to a cargo protein directly, or indirectly via a SBD linked to the cargo protein.

134. The polypeptide of paragraph 133, wherein the binding between the cargo binding domain and the cargo protein is a non-covalent binding.

135. The polypeptide of paragraph 133 or 134, wherein the binding between the cargo binding domain and the cargo protein is a reversible binding.

136. The polypeptide of any one of paragraphs 133-135, wherein the binding between the cargo binding domain and the cargo protein is capable of being controlled.

137. The polypeptide of paragraph 136, wherein the binding between the cargo binding domain and the cargo protein is capable of being controlled by pH.

138. The polypeptide of paragraph 136, wherein the binding between the cargo binding domain and the cargo protein is capable of being controlled by ionic strength.

139. The polypeptide of any one of paragraphs 133-138, wherein the binding between the cargo binding domain and the cargo protein is capable of being controlled such that the cargo protein is bound to the cargo binding domain in vitro but is released from the cargo binding domain in vivo.

140. The polypeptide of any one of paragraphs 133-138, wherein the binding between the cargo binding domain and the cargo protein is capable of being controlled such that the cargo protein is released from the cargo binding domain in a manner dependent on the subcellular compartment in which they are located.

141. The polypeptide of any one of paragraphs 133-140, wherein the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine.

142. The polypeptide of paragraph 141, wherein the domain that is capable of binding to phosphotyrosine is a PTB domain.

143. The polypeptide of paragraph 141, wherein the domain that is capable of binding to phosphotyrosine is an SH2 domain.

144. The polypeptide of any one of paragraphs 133-140, wherein the cargo binding domain comprises a first SAM domain and the cargo protein or the SBD comprises a second SAM domain, and the binding between the cargo binding domain and the cargo protein is a binding between the first SAM domain and the second SAM domain.

145. The polypeptide of any one of paragraphs 133-140, wherein the cargo binding domain comprises a PBM domain and the cargo protein or the SBD comprises a PDZ domain, and the binding between the cargo binding domain and the cargo protein is a binding between the PBM domain and the PDZ domain.

146. The polypeptide of any one of paragraphs 133-140, wherein the cargo binding domain comprises a PDZ domain and the cargo protein or the SBD comprises a PBM domain, and the binding between the cargo binding domain and the cargo protein is a binding between the PDZ domain and the PBM domain.

147. The polypeptide of any one of paragraphs 120-132, wherein the polypeptide comprises a cargo protein.

148. The polypeptide of any one of paragraphs 114-119, wherein the polypeptide further comprises an ephrin receptor JM domain that is capable of binding to a cargo protein directly, or indirectly via a SBD, and is C-terminal to the TM domain.

149. The polypeptide of paragraph 148, wherein the binding between the ephrin receptor JM domain and the cargo protein is a non-covalent binding.

150. The polypeptide of paragraph 148 or 149, wherein the binding between the ephrin receptor JM domain and the cargo protein is a reversible binding.

151. The polypeptide of any one of paragraphs 148-150, wherein the binding between the ephrin receptor JM domain and the cargo protein is capable of being controlled.

152. The polypeptide of paragraph 151, wherein the binding between the ephrin receptor JM domain and the cargo protein is capable of being controlled by pH.

153. The polypeptide of paragraph 151, wherein the binding between the ephrin receptor JM domain and the cargo protein is capable of being controlled by ionic strength.

154. The polypeptide of any one of paragraphs 148-153, wherein the binding between the ephrin receptor JM domain and the cargo protein is capable of being controlled such that the cargo protein is bound to the ephrin receptor JM domain in vitro but is released from the ephrin receptor JM domain in vivo.

155. The polypeptide of any one of paragraphs 148-153, wherein the binding between the ephrin receptor JM domain and the cargo protein is capable of being controlled such that the cargo protein is released from the ephrin receptor JM domain in a manner dependent on the subcellular compartment in which they are located.

156. The polypeptide of any one of paragraphs 148-155, wherein the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine.

157. The polypeptide of paragraph 156, wherein the domain that is capable of binding to phosphotyrosine is a PTB domain.

158. The polypeptide of paragraph 156, wherein the domain that is capable of binding to phosphotyrosine is an SH2 domain.

159. The polypeptide of any one of paragraphs 156-158, wherein the ephrin receptor JM domain comprises:
(i) a (X₁)-Ptyr-(X₂) motif, wherein Ptyr is a phosphotyrosine, X₁ is Y, P, V, I, T, or F, and X₂ is I, V, L, or A;
(ii) a (X₃)-Ptyr-(X₄) motif, wherein Ptyr is a phosphotyrosine, X₃ is T, A, or S, and X₄ is E or G; or
(iii) both (i) and (ii).

160. The polypeptide of any one of paragraphs 114-119, wherein the polypeptide further comprises an ephrin receptor KD that is capable of binding to a cargo protein directly, or indirectly via a SBD linked to the cargo protein, and is C-terminal to the TM domain.

161. The polypeptide of paragraph 160, wherein the binding between the ephrin receptor KD and the cargo protein is a non-covalent binding.

162. The polypeptide of paragraph 160 or 161, wherein the binding between the ephrin receptor KD and the cargo protein is a reversible binding.

163. The polypeptide of any one of paragraphs 160-162, wherein the binding between the ephrin receptor KD and the cargo protein is capable of being controlled.

164. The polypeptide of paragraph 163, wherein the binding between the ephrin receptor KD and the cargo protein is capable of being controlled by pH.

165. The polypeptide of paragraph 163, wherein the binding between the ephrin receptor KD and the cargo protein is capable of being controlled by ionic strength.

166. The polypeptide of any one of paragraphs 160-165, wherein the binding between the ephrin receptor KD and the cargo protein is capable of being controlled such that the cargo protein is bound to the ephrin receptor KD in vitro but is released from the ephrin receptor KD in vivo.

167. The polypeptide of any one of paragraphs 160-165, wherein the binding between the ephrin receptor KD and the cargo protein is capable of being controlled such that the cargo protein is released from the ephrin receptor KD in a manner dependent on the subcellular compartment in which they are located.

168. The polypeptide of any one of paragraphs 160-167, wherein the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine.

169. The polypeptide of paragraph 168, wherein the domain that is capable of binding to phosphotyrosine is a PTB domain.

170. The polypeptide of paragraph 168, wherein the domain that is capable of binding to phosphotyrosine is an SH2 domain.

171. The polypeptide of any one of paragraphs 168-170, wherein the KD comprises an (X₇)-Ptyr-(X₈) motif in the activation loop, wherein Ptyr is a phosphotyrosine, X₇ is T, V, or A, and X₈ is E or T.

172. The polypeptide of any one of paragraphs 114-119, wherein the polypeptide further comprises a SAM linker domain that is capable of binding to a cargo protein directly, or indirectly via a SBD linked to the cargo protein, and is C-terminal to the TM domain.

173. The polypeptide of paragraph 172, wherein the binding between the SAM linker domain and the cargo protein is a non-covalent binding.

174. The polypeptide of paragraph 172 or 173, wherein the binding between the SAM linker domain and the cargo protein is a reversible binding.

175. The polypeptide of any one of paragraphs 172-174, wherein the binding between the SAM linker domain and the cargo protein is capable of being controlled.

176. The polypeptide of paragraph 175, wherein the binding between the SAM linker domain and the cargo protein is capable of being controlled by pH.

177. The polypeptide of paragraph 175, wherein the binding between the SAM linker domain and the cargo protein is capable of being controlled by ionic strength.

178. The polypeptide of any one of paragraphs 172-177, wherein the binding between the SAM linker domain and the cargo protein is capable of being controlled such that the cargo protein is bound to the SAM linker domain in vitro but is released from the SAM linker domain in vivo.

179. The polypeptide of any one of paragraphs 172-177, wherein the binding between the SAM linker domain and the cargo protein is capable of being controlled such that the cargo protein is released from the SAM linker domain in a manner dependent on the subcellular compartment in which they are located.

180. The polypeptide of any one of paragraphs 172-179, wherein the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid.

181. The polypeptide of any one of paragraphs 172-180, wherein the SAM linker domain is an ephrin receptor SAM linker domain.

182. The polypeptide of any one of paragraphs 114-119, wherein the polypeptide further comprises a SAM domain that is capable of binding to a cargo protein directly, or indirectly via a SBD linked to the cargo protein, and is C-terminal to the TM domain.

183. The polypeptide of paragraph 182, wherein the binding between the SAM domain and the cargo protein is a non-covalent binding.

184. The polypeptide of paragraph 182 or 183, wherein the binding between the SAM domain and the cargo protein is a reversible binding.

185. The polypeptide of any one of paragraphs 182-184, wherein the binding between the SAM domain and the cargo protein is capable of being controlled.

186. The polypeptide of paragraph 185, wherein the binding between the SAM domain and the cargo protein is capable of being controlled by pH.

187. The polypeptide of paragraph 185, wherein the binding between the SAM domain and the cargo protein is capable of being controlled by ionic strength.

188. The polypeptide of any one of paragraphs 182-187, wherein the binding between the SAM domain and the cargo protein is capable of being controlled such that the cargo protein is bound to the SAM domain in vitro but is released from the SAM domain in vivo.

189. The polypeptide of any one of paragraphs 182-187, wherein the binding between the SAM domain and the cargo protein is capable of being controlled such that the cargo protein is released from the SAM domain in a manner dependent on the subcellular compartment in which they are located.

190. The polypeptide of any one of paragraphs 182-189, wherein the cargo protein or the SBD comprises a second SAM domain, and the binding between the SAM domain and the cargo protein is a binding between the SAM domain and the second SAM domain.

191. The polypeptide of any one of paragraphs 182-189, wherein the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine. 192. The polypeptide of paragraph 191, wherein the domain that is capable of binding to phosphotyrosine is a PTB domain.

193. The polypeptide of paragraph 191, wherein the domain that is capable of binding to phosphotyrosine is an SH2 domain.

194. The polypeptide of any one of paragraphs 191-193, wherein the SAM domain comprises a phosphotyrosine in the α2 helix.

195. The polypeptide of paragraph 194, wherein the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T.

196. The polypeptide of any one of paragraphs 182-195, wherein the SAM domain is an ephrin receptor SAM domain.

197. The polypeptide of any one of paragraphs 114-119, wherein the polypeptide further comprises an ephrin receptor PDZ binding motif (PBM) domain that is capable of binding to a cargo protein directly, or indirectly via a SBD linked to the cargo protein, and is C-terminal to the TM domain.

198. The polypeptide of paragraph 197, wherein the binding between the ephrin receptor PBM domain and the cargo protein is a non-covalent binding.

199. The polypeptide of paragraph 197 or 198, wherein the binding between the ephrin receptor PBM domain and the cargo protein is a reversible binding.

200. The polypeptide of any one of paragraphs 197-199, wherein the binding between the ephrin receptor PBM domain and the cargo protein is capable of being controlled.

201. The polypeptide of paragraph 200, wherein the binding between the ephrin receptor PBM domain and the cargo protein is capable of being controlled by pH.

202. The polypeptide of paragraph 200, wherein the binding between the ephrin receptor PBM domain and the cargo protein is capable of being controlled by ionic strength.

203. The polypeptide of any one of paragraphs 197-202, wherein the binding between the ephrin receptor PBM domain and the cargo protein is capable of being controlled such that the cargo protein is bound to the ephrin receptor PBM domain in vitro but is released from the ephrin receptor PBM domain in vivo.

204. The polypeptide of any one of paragraphs 197-202, wherein the binding between the ephrin receptor PBM domain and the cargo protein is capable of being controlled such that the cargo protein is released from the ephrin receptor PBM domain in a manner dependent on the subcellular compartment in which they are located.

205. The polypeptide of any one of paragraphs 197-204, wherein the cargo protein or the SBD comprises a PDZ domain, and the binding between the ephrin receptor PBM domain and the cargo protein is a binding between the ephrin receptor PBM domain and the PDZ domain.

206. The polypeptide of any one of paragraphs 120-205, wherein the cargo protein is a therapeutic protein.

207. The polypeptide of paragraph 206, wherein the therapeutic protein is a therapeutic antibody or an antigen binding fragment thereof.

208. The EV or hybridosome of paragraph 206, wherein the therapeutic protein is a gene editor or transposase.

209. The polypeptide of any one of paragraphs 120-205, wherein the cargo protein is a diagnostic protein.

210. The polypeptide of paragraph 209, wherein the diagnostic protein is a fluorescent protein.

211. The polypeptide of any one of paragraphs 114-210, wherein the polypeptide lacks an ephrin receptor ligand binding domain (LBD).

212. The polypeptide of any one of paragraphs 114-210, wherein the polypeptide comprises a mutated ephrin receptor LBD.

213. The polypeptide of any one of paragraphs 114-210, wherein the polypeptide comprises two different domains that allow the polypeptide to undergo hetero-domain dimerization with another polypeptide identical to said polypeptide.

214. The polypeptide of any one of paragraphs 114-210, wherein the polypeptide comprises two different domains that allow the polypeptide to undergo hetero-domain dimerization with another polypeptide identical to said polypeptide, in a head-to-tail configuration.

215. The polypeptide of any one of paragraphs 114-214, wherein the TM domain is an ephrin receptor TM domain.

216. The polypeptide of any one of paragraphs 114-215, wherein any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof.

217. The polypeptide of any one of paragraphs 114-215, wherein any one or more of the ephrin receptor domains of the polypeptide are from or derived from EphA2, EphA4, EphB2, or a combination thereof. 218. The polypeptide of any one of paragraphs 114-217, wherein the polypeptide further comprises a modified Fe domain of an immunoglobulin.

219. The polypeptide of paragraph 218, wherein the modified Fe domain is N-terminal to the ephrin receptor CR domain.

220. The polypeptide of paragraph 219, wherein the modified Fe domain is fused to the remaining portion of the polypeptide by a linker sequence.

221. The polypeptide of any one of paragraphs 218-220, wherein the modified Fe domain
a. is capable of specifically binding to the Fc binding site of a neonatal Fc receptor (FcRn); and
b. lacks the ability to form homodimers.

222. The polypeptide of any one of paragraph 218-221, wherein the dissociation constant of the modified Fe domain bound to the FcRn at a pH of 6.5 has a value of at most 10⁻⁴ M.

223. The polypeptide of any one of paragraph 218-222, wherein the dissociation constant of the modified Fe domain bound to the FcRn at a pH of 7.4 has a value of at least 10⁻⁴ M.

224. The polypeptide of any one of paragraph 218-223, wherein the modified Fe domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid.

225. The polypeptide of any one of paragraph 218-224, wherein the modified Fe domain is capable of specifically binding to the amino acid sequence between position 135-158 of human FcRn (SEQ ID NO: 228) and/or mouse FcRn (SEQ ID NO: 227).

226. The polypeptide of any one of paragraphs 218-225, wherein the polypeptide does not substantially bind to C1q, FcγRI, FcγRII or FcγRIII.

227. The polypeptide of any one of paragraphs 218-226, wherein:
a. the complement dependent cytotoxicity (CDC) activity of the modified Fe domain;
b. the antibody dependent cell mediated cytotoxicity (ADCC) activity of the modified Fe domain;
c. the antibody dependent cell mediated phagocytosis (ADCP) activity of the modified Fe domain; and/or
d. the antibody dependent intracellular neutralization (ADIN) activity of the modified Fc domain
is decreased by at least 10%, 20%, 30%, 40%, or 50% compared to an unmodified Fc domain.

228. The polypeptide of any one of paragraphs 218-227, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 1.5, 2, 3, 4, or 5-fold, compared to an unmodified Fc domain.

229. The polypeptide of any one of paragraphs 218-228, wherein the modified Fc domain comprises from N-terminus to C-terminus:
a. a modified CH2 domain that is modified to decrease effector function relative to the unmodified CH2 domain; and
b. a modified CH3 domain that is modified to lack the ability to form homodimers.

230. The polypeptide of any one of paragraphs 114-229, wherein the first ephrin receptor FN III domain and the second ephrin receptor FN III domain comprise different amino acid sequences.

231. A nucleic acid encoding the polypeptide of any one of paragraph 114-230.

232. An expression vector comprising the nucleic acid of paragraph 231.

233. A cell comprising the nucleic acid of paragraph 231 or the expression vector of paragraph 232.

234. A method of producing an EV, wherein the method comprises:
a. transfecting cells with the nucleic acid of paragraph 231 or the expression vector of paragraph 232;
b. cultivating the cells under suitable conditions for the production of the EV; and
c. collecting the EV secreted by the cells.

235. A method of producing a hybridosome, wherein the method comprises contacting a first EV with a second EV, thereby uniting the first EV with the second EV and producing the hybridosome,
wherein said first EV has been produced in vitro, and the first EV comprises (i) a membrane, and (ii) a fusogenic, ionizable, cationic lipid, and
wherein said second EV has been produced by the method of paragraph 234.

236. A method of purifying an EV or a hybridosome, wherein the method comprises:
a. providing the EV or hybridosome, wherein the EV or hybridosome comprises a first binding partner, wherein the first binding partner is capable of binding to the Fc binding site of an FcRn in a pH dependent manner; and
b. contacting at a first pH the EV or hybridosome comprising the first binding partner with a second binding partner, wherein the second binding partner comprises the Fc binding site of the FcRn and is associated with a solid matrix; and
c. eluting the EV or hybridosome comprising the first binding partner from the solid matrix at a second pH.

237. The method of paragraph 236, wherein the method further comprises a washing step at the first pH.

238. The method of paragraph 236 or 237, wherein the first pH is below 6.5.

239. The method of any one of paragraphs 236-238, wherein the second pH is above 7.4.

240. The method of any one of paragraphs 236-239, wherein the Fc binding site of the FcRn comprises the amino acid sequence of SEQ ID NO: 230.

241. A method of purifying an EV or a hybridosome, wherein the method comprises:
a. providing the EV or hybridosome, wherein the EV or hybridosome comprises a first binding partner, wherein the first binding partner is capable of binding to the Fc binding site of an FcRn in a pH dependent manner and comprises or consists of the polypeptide of any one of paragraphs 111-224; and
b. contacting at a first pH the EV or hybridosome comprising the first binding partner with a second binding partner, wherein the second binding partner comprises the Fc binding site of the FcRn and is associated with a solid matrix; and
c. eluting the EV or hybridosome comprising the first binding partner from the solid matrix at a second pH.

242. The method of paragraph 241, wherein the method further comprises a washing step at the first pH.

243. The method of paragraph 241 or 242, wherein the first pH is below 6.5.

244. The method of any one of paragraphs 241-243, wherein the second pH is above 7.4.

245. The method of any one of paragraphs 241-244, wherein the Fc binding site of the FcRn comprises the amino acid sequence of SEQ ID NO: 230.

### 4. BRIEF DESCRIPTION OF THE DRAWINGS

FIG. 1A is a drawing of the architecture of Eph receptors. FIG. 1B is a drawing of exemplary variations of scaffold proteins comprising the following domains: (i) a CRD-FNIII-FNIII-TM, (ii) CRD-FNIII-FNIII-TM-JM, (iii) CRD-FNIII-FNIII-TM-JM-KD, (iv) CRD-FNIII-FNIII-TM-JM-SAM-PBM, (v) LBD*-CRD-FNIII-FNIII-TM, (vi) LBD*-CRD-FNIII-FNIII-TM-JM, (vii) LBD*-CRD-FNIII-FNIII-TM-JM-KD, or (viii) LBD*-CRD-FNIII-FNIII-TM-JM-SAM-PBM. LBD* denotes a mutated LBD with decreased ephrin binding compared to wild type LBD

FIG. 2A depicts homo-domain dimerization interfaces and a dimer of polypeptides. FIG. 2B depicts hetero-domain dimerization domain interfaces and an oligomer of polypeptides in head-to-tail configuration.

FIG. 3A depicts interaction of the ephrin2 receptor binding domain residue E129 with EphA4 LBD residue R106. FIG. 3B depicts the interaction of the EphA4 FNIII residues N504 and N07 with EphA4 LBD residue R106 in a head-to-tail cluster. Mutagenesis of EphA4 LBD residue R106 to E impairs the EphA4-Ephrin2 interaction. R106 interacts (H-bonds) with N504 and T507. In order to retain the head to tail conformation with impaired ephrin binding, mutation T507N may be introduced, which will create a new H-bond with R106E, while N504 may be kept unchanged, maintaining its interaction with R106E.

FIG. 4. Drawing of the FNIII-LBD (head-to-tail) interaction between two EphA4 scaffolds and the N-terminus linked to a fusion moiety via a linker.

FIG. 5. Drawing of ephrin independent LBD-LBD interaction between two EphA4 LBDs and the N-terminus linked to a fusion moiety via a linker distal to the homo domain dimerization interface.

FIG. 6. Drawing of the interaction between two EphA4 scaffolds via the CRD and LBD homo-domain interface and the N-terminus linked to a fusion moiety via a linker.

FIG. 7. Drawing of the interaction between two LBD truncated EphA4 scaffolds via the CRD homo-domain interface and the N-terminus linked to a fusion moiety via a linker.

FIG. 8. Exemplary structures of new scaffolds derived from Eph receptors, with a mutated (including truncated) ligand binding domain (LBD) or no LBD, linked to targeting domains and/or modified Fc domains

FIG. 9. Exemplary schematic drawing of loading of cargo protein by covalent attachment to scaffold polypeptides during biogenesis of EVs

FIG. 10. Exemplary schematic drawing of phosphotyrosine-based reversible binding of cargo protein to scaffold polypeptides via a scaffold binding domain (SBD) during biogenesis of EVs

FIG. 11. Alignment of the LBD sequences of human Eph receptors showing of beta strands D-M and corresponding loops (SEQ ID NO: 243 from EphA1, SEQ ID NO: 244 from EphA2, SEQ ID NO: 245 from EphA3, SEQ ID NO: 246 from EphA4, SEQ ID NO: 247 from EphA5, SEQ ID NO: 248 from EphA6, SEQ ID NO: 249 from EphA7, SEQ ID NO: 250 from EphA8, SEQ ID NO: 251 from EphA10, SEQ ID NO: 252 from EphB1, SEQ ID NO: 253 from EphB2, SEQ ID NO: 254 from EphB3, and SEQ ID NO: 255 from EphB4).

FIG. 12. Exemplary schematic drawing of several scaffold proteins interacting with an adaptor protein.

FIG. 13. Western blot showing EVs purified from the conditioned media. Said EVs contained the full length scaffold protein with intraluminal turboluc

FIG. 14A and FIG. 14B depict binding curves from an FcRn binding immunoassay with EVs expressing the modified Fc domain (FIG. 14A), native EVs (FIG. 14A), human IgG1 (FIG. 14B) and mouse IgG1 (FIG. 14B).

FIG. 15. Anti-EphA4 western blot showing the detection of EphA4 fusion proteins expressed from constructs in concentrated conditioned media, which were loaded onto a scFcRn column. The first lane is the load, the second lane is a sample of the flow through and the third lane is a sample of the eluted fraction.

FIG. 16. Percentage of cells that were RFP+, as determined by flow cytometry of color switch HEK293T cells expressing EphA2 and treated with varying doses of Cre mRNA loaded hybridosomes derived from EVs comprising a scaffold protein targeting EphA2 or mouse CD64 as non-target control as well as LNPs.

FIG. 17A. Schematic illustration of a lentiviral polycistronic construct for non-covalent loading of cargo into the lumen of EVs. FIG. 17B. Anti-turboluc western blot showing presence of turboluc-SH2-SBX100 protein in harvested EV sample. FIG. 17C. Luminescence of harvested turboluc-SH2-SBX100 EVs treated with trypsin vs untreated.

FIG. 18. DNA vector copy number per ul of mouse plasma on days 3, 6, 21 and 24 after IV administration of EVs comprising a scaffold protein displaying a modified Fc domain vs a LNP formulation.

FIG. 19. Schematic of the following Eph receptor ectodomain chain crystal structures from the Protein Data Bank (PDB) and superimposed: EphB2_MOUSE_lbd (PDB:1kgy), EphB4_HUMAN_lbd (PDB:2bba), EphB2_HUMAN_lbd (PDB:2qbx), EphA4_HUMAN_lbd (PDB:2wol), EphA2_HUMAN_lbd_fn3_fn3 (PDB:3fl7), EphA2_HUMAN_lbd (PDB:3mbw), EphA2_HUMAN_lbd_fn3 (PDB:3mx0), EphA7_HUMAN_lbd (PDB:3nru), EphB3_HUMAN_lbd (PDB:3pli), EphA5_HUMAN_lbd (PDB:4et7), EphA3_HUMAN_lbd (PDB:410p), EphA4_HUMAN_lbd (PDB:4w50), according to Xu Q, Dunbrack R L Jr. Bioinformatics. 2012; 28(21):2763-2772).

### 5. DETAILED DESCRIPTION

The present disclosure is based, at least in part, on the discovery that ephrin (Eph) receptors can be sorted into nanovesicles. Ephrin receptors thus enable transporting, trafficking or shuttling of a cargo (e.g., a cargo protein) to a nanovesicle (e.g., an extracellular vesicle (EV) or a hybridosome). Ephrin receptors, in particular, variants of ephrin receptors engineered to have diminished or no reverse signaling as a result of decreased or no binding to ephrin, can therefore be used as neutral protein scaffolds amenable to load cargos (e.g., cargo proteins) onto or into nanovesicles. This makes the polypeptides described herein that comprise ephrin receptor domain(s) (also referred to herein as ephrin receptor derived polypeptides) attractive protein scaffolds to display cargos (e.g., cargo proteins) on the surface of or into nanovesicles (e.g., EVs and hybridosomes).

Provided herein are polypeptides comprising a transmembrane domain and further comprising an ectodomain and/or an endodomain that can be used to load a cargo (e.g., a cargo protein) on the surface of or into nanovesicles (e.g., EVs and hybridosomes). The cargo (e.g., a cargo protein) can be part of a polypeptide described herein. In other words, the cargo (e.g., a cargo protein) can be fused to the remaining portion of the polypeptide (e.g., via a linker). Alternatively, the cargo (e.g., a cargo protein) can be bound (preferably, reversibly bound) to the polypeptide through a domain that is capable of binding to the cargo (e.g., a cargo protein), i.e., a cargo binding domain. A cargo binding domain can bind to the cargo (e.g., cargo protein) directly, or indirectly via a scaffold binding domain (SBD) linked to the cargo (e.g., cargo protein). The singular forms “a”, “an”, and “the” as used herein include plural referents. As such, a polypeptide described herein can be used to deliver one or more (e.g., one, two, three, four, five or more) cargos, and a polypeptide described herein can comprise one or more (e.g., one, two, three, four, five or more) cargos or one or more (e.g., one, two, three, four, five or more) cargo binding domains.

A polypeptide described herein can further comprise one or more functional moieties, such as a targeting domain that is capable of targeting the nanovesicle (e.g., EV or hybridosome) to a specific organ, tissue, or cell type, and/or a purification domain that can facilitate purification of the nanovesicle (e.g., EV or hybridosome).

Preferably, a polypeptide described herein comprises one or more domains from or derived from one or more Eph receptors. Such a polypeptide is also referred to herein as an Eph receptor derived polypeptide or a polypeptide derived from an Eph receptor(s). An ephrin receptor derived polypeptide may or may not comprise one or more domains (e.g., a transmembrane domain) from or derived from a non-ephrin receptor protein. In various embodiments, a polypeptide described herein comprises an ectodomain or fragment thereof (e.g., a flexible domain) from or derived from one or more ephrin receptors, a transmembrane domain that is from or derived from an ephrin receptor or a non-ephrin receptor transmembrane protein, and optionally an endodomain or fragment thereof from or derived from one or more ephrin receptors.

In particular, provided herein are polypeptides comprising at least an ephrin receptor cysteine-rich (CR) domain, two ephrin receptor fibronectin type III (FN III) domains (i.e., a first ephrin receptor FN III domain (ephrin receptor FN1 domain), and a second ephrin receptor FN III domain (ephrin receptor FN2 domain)), and a transmembrane (TM) domain (e.g., an ephrin receptor TM domain), and optionally a cargo binding domain, an ephrin receptor juxtamembrane (JM) domain, an ephrin receptor kinase domain (KD), a sterile α-motif (SAM) linker domain (e.g., an ephrin receptor linker SAM domain), a SAM domain (e.g., an ephrin receptor SAM domain), an ephrin receptor PDZ binding motif (PBM) domain, a targeting domain, a purification domain, a modified Fc domain, and/or a ligand binding domain (LBD). Various aspects and embodiments of the polypeptides are described in Section 5.2. Ephrin receptor LBDs, ephrin receptor CR domains (CRDs), ephrin receptor FN III domains, and TM domains (e.g., ephrin receptor TM domains) are further described in Section 5.2.1. Ephrin receptor JM domains, ephrin receptor KDs, SAM linker domains (e.g., ephrin receptor SAM linker domains), SAM domains (e.g., ephrin receptor SAM domain), and ephrin receptor PBM domains are further described in Section 5.2.2. Cargo binding domains are further described in Section 5.2.3. Targeting domains and purification domains are further descried in Section 5.2.4. Modified Fc domains are further described in Section 5.2.5.

A polypeptide described herein can be used to deliver a cargo (e.g., a cargo protein), for example, by an extracellular vesicle (EV) or a hybridosome, e.g., for a therapeutic or diagnostic use. The cargo (e.g., a cargo protein) can be part of the polypeptide. In other words, the cargo (e.g., a cargo protein) can be fused to the remaining portion of the polypeptide (e.g., via a linker). Alternatively, the cargo (e.g., a cargo protein) can be bound (preferably, reversibly bound) to the polypeptide through a cargo binding domain. A cargo binding domain can bind to the cargo (e.g., cargo protein) directly, or indirectly via a SBD linked to the cargo (e.g., cargo protein). The cargo binding domain can be either an ephrin receptor domain (such as an ephrin receptor JM domain, ephrin receptor KD, ephrin receptor SAM linker domain, ephrin receptor SAM domain, or ephrin receptor PBM domain), or a domain capable of binding to a cargo but is not an ephrin receptor domain. A reversible binding between the cargo (e.g., a cargo protein) and the cargo binding domain can be, but is not limited to, a phosphotyrosine-based binding (such as a binding between a phosphotyrosine and a phosphotyrosine binding (PTB) domain, or a binding between a phosphotyrosine and a Src homology 2 (SH2) domain), a SAM domain-based binding, a PDZ domain-based binding, or a DH-PH motif-based binding). Cargos (e.g., cargo proteins) and cargo binding domains are further described in Section 5.2.3. Ephrin receptor domains are further described in Sections 5.2.1 and 5.2.2.

Any one or more of the ephrin receptor domains described herein can be from or derived from the same ephrin receptor or different ephrin receptors. In some embodiments, a polypeptide described herein comprises ephrin receptor domains from or derived from the same ephrin receptor. In other embodiments, a polypeptide described herein comprises ephrin receptor domains from or derived from two, three, or more than three ephrin receptors. In specific embodiments, any one or more of the ephrin receptor domains of a polypeptide described herein are from or derived from EphA1, EphA2, EphHA3, EphA4, EphA5, EphHA6, EphA7, EphA8, EphA10, EphB1, EphB2, EphB3, EphB4, EphB6, or a combination thereof. In specific embodiments, any one or more of the ephrin receptor domains of a polypeptide described herein are from or derived from EphA2, EphA4, EphB2, or a combination thereof.

Any one or more of the ephrin receptor domains described herein can be a wild-type or a mutant ephrin receptor domain(s). In certain embodiments, an ephrin receptor domain described herein has at least 70%, at least 80%, at least 85%, at least 90%, at least 95%, at least 96%, at least 97%, at least 98% or at least 99% sequence identity to the corresponding domain of a wild-type ephrin receptor (e.g., an ephrin receptor comprising an amino acid sequence selected from the group consisting of SEQ ID NOs: 212-225). In certain embodiments, an ephrin receptor domain described herein comprises the amino acid sequence of the corresponding domain of a wild-type ephrin receptor (e.g., an ephrin receptor comprising an amino acid sequence selected from the group consisting of SEQ ID NOs: 212-225) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In various embodiments, adaptor proteins can be used to bring polypeptides described herein into close proximity (e.g., cluster) of each other on a nanovesicle (e.g., an EV or hybridosome). Adaptor proteins are further described in Section 5.2.3(c).

Also provided are nucleic acids encoding a polypeptide described herein, expression vectors comprising a nucleic acid described herein, and cells comprising a nucleic acid or expression vector described herein, all of which are further described in Section 5.3.

Further provided are nanovesicles (e.g., EVs and hybridosomes) comprising a polypeptide described herein. Nanovesicles (e.g., EVs and hybridosomes) are further described in Section 5.4.

Methods of producing or purifying a nanovesicle (e.g., an EV or hybridosome) are also provided and are further described in Section 5.4.

Compositions and kits comprising a polypeptide, a nanovesicle (e.g., an EV or hybridosome), a nucleic acid, an expression vector, or a cell described herein are provided and further described in Section 5.5.

Therapeutic and diagnostic uses of a polypeptide, a nanovesicle (e.g., an EV or hybridosome), a composition, or a kit described herein are provided and further described in Section 5.6.

### 5.1 Definitions

As used herein, the singular forms “a”, “an”, and “the” include plural referents unless the content clearly dictates otherwise. Thus, for example, reference to “a polypeptide” may include two or more such molecules, and the like.

As used herein, the terms “about” and “approximately”, when used to modify an amount specified in a numeric value or range, indicate that the numeric value as well as reasonable deviations from the value known to the skilled person in the art, for example ±20%, ±10%, or +5%, are within the intended meaning of the recited value. It is furthermore understood that slight variations above and below a stated range can be used to achieve substantially the same results as a value within the range. Also, unless indicated otherwise, the disclosure of ranges is intended as a continuous range including every value between the minimum and maximum values.

The terms “comprising”, “having”, “including,” containing”, etc. shall be read expansively or open-ended and without limitation.

Unless otherwise indicated, the term “at least” preceding a series of elements is to be understood to refer to every element in the series. The terms “at least one” and “at least one of” include for example, one, two, three, four, or five or more elements.

The term “nanovesicles” refers to lipid nanovesicles derived from a source cell (i.e. extracellular vesicles), and synthetic lipid nanoparticles, and natural/synthetic hybrids (such as a hybridosome). A nanovesicle typically comprises lipids or fatty acids as well as polypeptides, and may further comprise a payload, a targeting moiety or other molecules. Furthermore, when teachings herein refer to a nanovesicle in singular it should be understood that all such teachings are equally relevant for and applicable to a plurality of nanovesicles and populations of nanovesicles. It will be clear to the skilled person that when describing medical and scientific uses and applications of the nanovesicles, the present disclosure normally relates to a plurality of nanovesicles, i.e. a population of nanovesicles which may comprise thousands, millions, billions or even trillions of nanovesicles. As can be seen from the experimental section below, nanovesicles may be present in concentrations such as 10⁵, 10⁸, 10¹⁰, 10¹¹, 10¹², 10¹³, 10¹⁴, 10¹⁵, 10¹⁸, 10²⁵, 10³⁰ particles per unit of volume (for instance per ml), or any other number larger, smaller or anywhere in between. Individual nanovesicles when present in a plurality constitute a nanovesicle population. Thus, naturally, the present disclosure pertains both to individual nanovesicles and populations comprising nanovesicles.

The terms “extracellular vesicle”, “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endosomal, lysosomal and/or endo-lysosomal pathway), an apoptotic body, an ARMM (arrestin domain containing protein 1 [ARRDC1]-mediated microvesicle), a fusosome, a microparticle and a cell derived vesicular structure. Generally, extracellular vesicles range in hydrodynamic diameter from 20 nm to 1000 nm and can comprise various macromolecular cargo (or “payload”) either within the internal space, displayed on the external surface of the extracellular vesicle, and/or spanning the membrane. Said cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion, sonication or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “hybridosome” refers to a hybrid biocompatible carrier which comprises structural and bioactive elements (e.g., lipids, carbohydrates, fatty acids, polynucleotides or polypeptides) originating from at least one extracellular vesicle (EV) and at least one engineered drug encapsulation module (EDEM) comprising a tunable fusogenic moiety. Said fusogenic moiety may be a fusogenic lipid or any other fusogenic component which enhances or enables the disruption of the membrane, or lipid mixing between a membrane and a lipid bilayer. The hybridosome results from uniting one EV with one EDEM, one EV with several EDEMs, several EVs with one EDEM, or several EVs with several EDEMs. The uniting event may be controlled via the size of the EVs and EDEMs, their respective charges, and the conditions applied during a uniting reaction such as the ratio EV/EDEM, the pH, the temperature and the reaction time. Hybridosomes as well as methods of producing these are described in detail in WO2015110957, which is hereby incorporated by reference in its entirety.

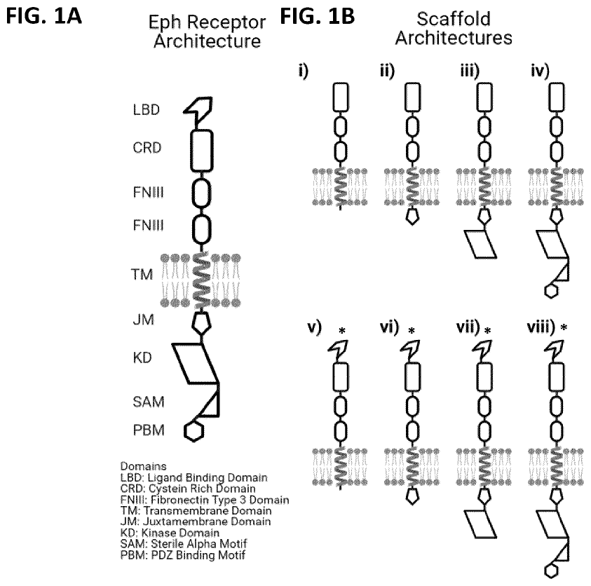

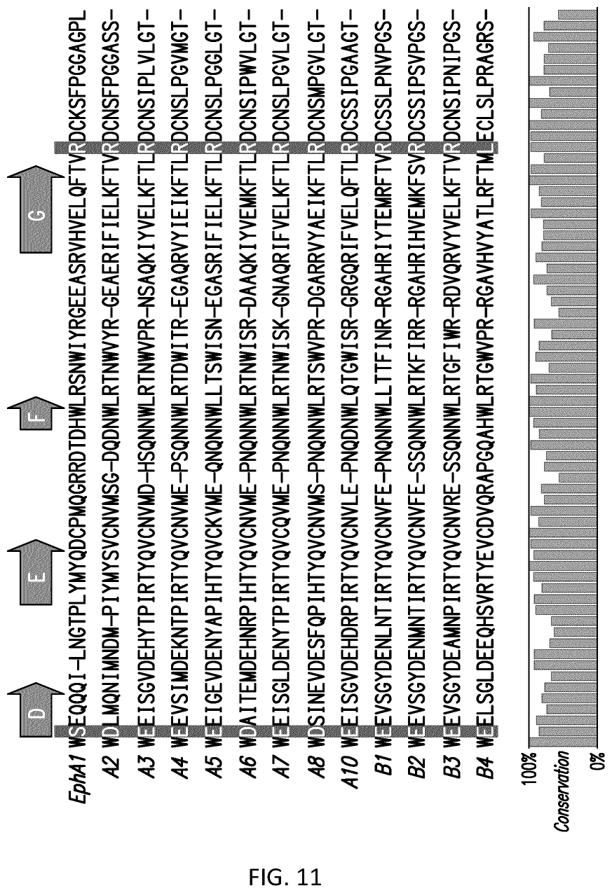

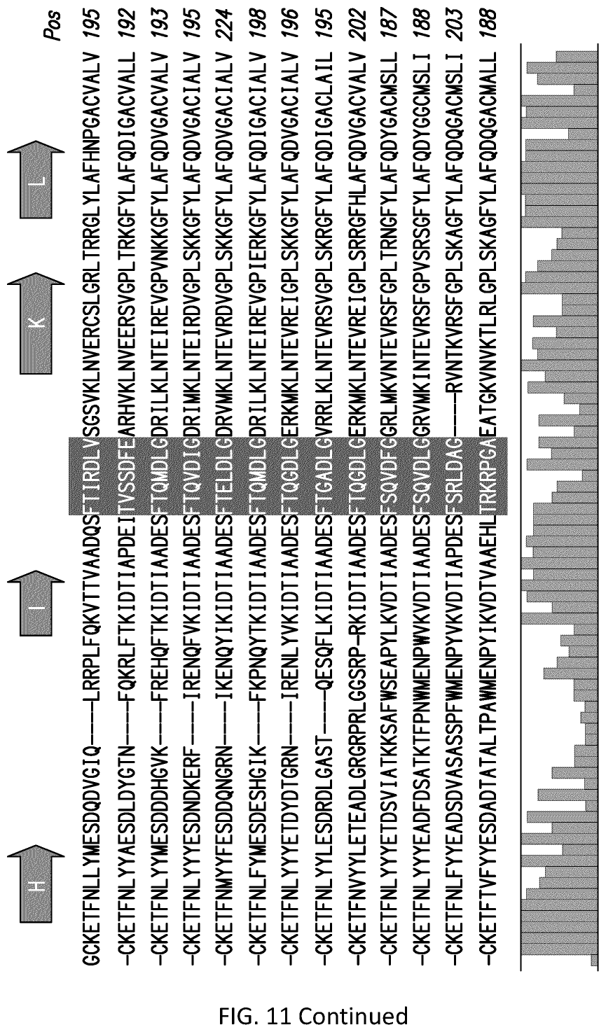

The term “Eph receptor” or “ephrin receptor” refers to a subfamily of receptor tyrosine kinases (RTKs), which bind a group of cell-membrane-associated ligands known as ephrins. Through ligand-induced activation of their kinase domain, Eph receptors transduce signals from the cell exterior to the interior. Eph receptors thus mediate contact-related cell-cell communication by interacting with ephrins on neighboring cells. Binding of the Eph receptor to ephrin leads to activation of the kinase domain of the Eph receptor. Eph receptor-ephrin binding events can lead to endocytosis of the receptor-ligand complex and the activated receptor continues to signal from intracellular compartments until it is inactivated by dephosphorylation and degradation or trafficked back to the cell surface. In humans, the family of Eph receptors have a highly conserved overall structure with the EphA and EphB receptors classes sharing the same structural features and domains. The domains of Eph receptors have been cataloged in the Conserved Domain Database at the National Center for Biotechnology Information (NCBI) including a listing of sequence/structure/function relationships. The classes consist of ten EphA members and five EphB members classified according to sequence homology. The ectodomain of Eph receptor region contains a conserved N-terminal ligand-binding domain (LBD, SMART accession number SM00615) which binds the receptors to their ephrin ligands. The LBD of Eph receptors consists of beta strands D-M and corresponding loops as depicted in FIG. 11. The formation of a complex between an Eph receptor and an ephrin is centered around the insertion of the ephrin G-H loop into the Eph receptor hydrophobic channel formed by the convex sheet of four β-strands together with the D-E, J-K, and G-H loops. The main sequence differences between EphA and EphB receptors reside in a region of the ligand binding domain determining ephrin subclass binding specificity. Adjacent to LBD is a cysteine-rich region comprising a Sushi domain and an epidermal growth factor (EGF)-like domain, followed by two fibronectin type III domains (FN1 and FN2). Transitioning from the transmembrane domain, the cytoplasmic Eph receptor region contains a kinase domain, a sterile alpha motif (SAM) domain, and a PDZ-binding motif (PBM). The LBD is unique to this family of RTKs and shares no significant amino-acid-sequence homology with other known proteins. See FIG. 1A for a schematic illustration of wild-type Eph receptors and FIG. 1B for schematic illustrations of exemplary scaffold proteins comprising domains from or derived from Eph receptors.

As used herein, the term “domain” refers to a unit (e.g., segment) of a polypeptide that can independently fold into a stable tertiary structure). Generally, domains are responsible for discrete functional properties of proteins, and in many cases may be added, removed, or transferred to other proteins without loss of function of the remainder of the protein and/or of the domain. Several distinct domains can be joined together in different combinations, forming multi-domain polypeptides. Traditionally, the length of polypeptides spanning domains have been elucidated by the use of atomic coordinates from experimentally determined three-dimensional structures of proteins. More recently, proteins lacking experimentally determined three-dimensional (3D) structures have been assigned domains by computational methods based on sequence homology. Since a large number of proteins do not have resolved structures, sequence-based approaches have been gaining much more attention. The sequence-based approaches include template-based, homologous-modeling-based and machine-learning-based techniques, depending on whether the prediction methods make use of 3D structure or homologous sequences as reviewed in Wang, Yan et al. Computational and structural biotechnology journal vol. 19 1145-1153. 2 Feb. 2021. Several computationally predicted domains are cataloged in publicly available databases (e.g., Pfam database as described in Pfam: The protein families database in 2021: J. Mistry, S. et al, Nucleic Acids Research (2020) or the NCBI Conserved Domain Database (CDD) https://www.ncbi.nlm.nih.gov/Structure/cdd/cdd.shtml).

The term “inter-domain linkers” refers to the segment of a polypeptide that ties two neighboring domains together. Inter-domain linkers provide flexibility to facilitate domain motions and to regulate the inter-domain geometry as described in Bhaskara R M, et al., J Biomol Struct Dyn. 2013 December; 31(12):1467-80. The inter-domain linkers modulate the interactions of adjacent domains by their lengths, conformations, intermolecular interactions, and local structure, thereby affecting the overall inter-domain geometry. Above mentioned databases based on predicted structural domains (Pfam database or NCBI Conserved Domain Database) provide generalizations of domains and may offer only an approximation of a domain boundary (e.g., to distinguish between residues that are within a domain or are inter-domain linkers). Hence, the domain sequences described herein (e.g., sequences in Tables 2-20) may include polypeptide sequences that comprise corresponding domain as well as inter-domain linkers. In some embodiments the 1, 2, 3, 4, 5, 6, 7, 8, 9 or 10 amino acid residues at the N- or C terminal of the cataloged domain sequences can be inter-domain linkers. Those skilled in the art may determine the segments of a polypeptide chain corresponding to domains and inter-domain linkers, and where a transition from a domain (i.e., at a domain boundary) to the inter-domain linker occurs.

The term “ectodomain” of a Ephrin receptor is well known in the art and refers to the extracellular part of the Eph receptor, i.e., the part of the RTK that is outside of the plasma membrane, and is devoid of a signal peptide.

A “ligand binding domain” or “LBD” is a peptide region that specifically binds one or more specific receptor ligands. If a plurality of ligands exists, those ligands share binding determinants sufficient to detectably bind to the binding domain. In some instances, the binding domain is a contiguous sequence of amino acids.

The term “surface decorated” as used herein refers to nanovesicles comprising a scaffold protein to which a molecule of interest (e.g., a protein), is attached. The scaffold protein can be changed by a chemical, a physical, or a biological method or by being produced from a cell being modified by a chemical, a physical, or a biological method. Specifically, the scaffold protein can be changed via genetic engineering so that a cell previously modified by genetic engineering produces such modified scaffold proteins.

As used herein, the term “biologically active molecule” refers to an agent that has activity in a biological system (e.g., a cell or a human subject), including, but not limited to: a protein, polypeptide or peptide, including, but not limited to, a structural protein, an enzyme, a cytokine (such as an interferon and/or an interleukin), an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g., a yeast artificial chromosome) or a part thereof, RNA, including mRNA, tRNA, rRNA or a ribozyme, or a peptide nucleic acid (PNA); a virus or virus-like particle; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In certain aspects, a biologically active molecule comprises a therapeutic molecule (e.g., an antigen), a targeting moiety (e.g., an antibody or an antigen-binding fragment thereof), an adjuvant, an immune modulator, or any combination thereof. In some aspects, the biologically active molecule comprises a macromolecule (e.g., a protein, an antibody, an enzyme, a peptide, DNA, RNA, or any combination thereof). In some aspects, the biologically active molecule comprises a small molecule (e.g., an antisense oligomer (ASO), a phosphorodiamidate morpholino oligomer (PMO), a peptide-conjugated phosphorodiamidate morpholino oligomer (PPMO), an siRNA, STING, a pharmaceutical drug, or any combination thereof). In some aspects, the biologically active molecules are exogenous to the EVs, i.e., not naturally found in the EVs.

As used herein, the term “scaffold protein” refers to a polypeptide that can be used to anchor a payload or any other compound of interest (e.g., a cargo protein) to the nanovesicle. In some aspects, the scaffold protein is a polypeptide that does not naturally exist in an EV. In some embodiments, the scaffold protein comprises a synthetic polypeptide. In some embodiments, the scaffold protein comprises a modified protein, wherein the corresponding unmodified protein naturally exists in the EV, e.g., the exosome. In some embodiments, the scaffold protein comprises a protein that naturally exists in the EV, or a fragment thereof, e.g., a fragment of an EV protein, where the protein is expressed at a higher level than the naturally occurring level. In some embodiments, the scaffold protein comprises a fusion protein, comprising (i) a naturally occurring EV protein or a fragment thereof and (ii) a heterologous peptide (e.g., an antigen binding domain, a cargo protein, a modified Fc, or any combination thereof). As used herein, the term “scaffold protein” of the present disclosure, or grammatical variants, can be:
(i) an ephrin receptor (naturally expressed, chemically or enzymatically synthesized, or produced recombinantly) that spans the membrane of nanovesicles, e.g., exosomes;
(ii) any functional fragment of (i);
(iii) any functional variant of (i) or (ii);
(iv) any derivative of any of (i)-(iii);
(v) any peptide corresponding to a domain or combination thereof derived from a protein in (i) that can span the membrane of nanovesicles, e.g., exosomes, or a molecule comprising such peptide;
(vi) an ephrin receptor-derived polypeptide described herein'
(vii) a molecule of any of (i) to (vi) comprising at least one non-natural amino acid; or
(viii) any combination of (i)-(vii);
which is suitable for use as a scaffold to target (attach) payloads, e.g., biologically active molecules to the surface (e.g., when the biologically active molecules comprise targeting domains) and/or lumen (e.g., when the biologically active molecules comprise cargo protein) of nanovesicles, e.g., exosomes.

The term “fragment” in reference to a polypeptide refers to any amino acid sequence present in a polypeptide, being shorter than the parental polypeptide as it has been N- and/or C-terminally deleted in comparison to the parental protein, but is still capable of performing the function of interest of the parental polypeptide.

The terms “source cell” or “EV source cell” or “cell source” or “EV-producing cell” or “producer cells” or any other similar terminology may be understood to relate to any type of cell that is capable of producing EVs under suitable conditions, for instance in suspension culture or in adherent culture or any in other type of culturing system.

The term “specifically binds” refers to a molecule (e.g., an antigen-binding molecule) that binds to an epitope or target with greater affinity, greater avidity, and/or greater duration to that epitope or target in a sample than it binds to another epitope or non-target compound (e.g., a structurally different antigen). In some embodiments, an molecule (e.g., an antigen-binding molecule) that specifically binds to an epitope or target binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding”, “specifically binds to,” or “is specific for” a particular epitope or target, as used herein, can be exhibited, for example, by a molecule having an equilibrium dissociation constant K_{d} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M or smaller. It will be recognized by one of skill that molecules (e.g., antigen-binding molecules) that specifically binds to a target from one species may also specifically bind to orthologs of that target.

The term “isolated” indicates that matter such as a polypeptide, a nucleic acid or a cell has been removed from its normal physiological environment, e.g. a natural source, or that a polypeptide or nucleic acid is synthesized. Use of the term “isolated” indicates that a naturally occurring sequence has been removed from its normal cellular (e.g., chromosomal) environment. Thus, the sequence may be in a cell-free solution or placed in a different cellular environment. “Isolated” in reference to a polypeptide or nucleic acid molecule means a polymer of two or more amino acids or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free of, e.g., non-amino acid material and/or non-nucleic acid material, respectively, naturally associated with it. An “isolated cell” refers to a cell that is separated from the molecular and/or cellular components that naturally accompany the cell.

In the context of nanovesicles, the terms “isolate”, “isolated”, and “isolating” or “purify”, “purified”, and “purifying” as well as “extracted” and “extracting” are used interchangeably and refer to the state of a preparation (e.g., a plurality of known or unknown amount and/or concentration) of desired nanovesicles, that have undergone one or more processes of purification, e.g., a selection or an enrichment of the desired nanovesicle preparation. In some embodiments, isolating or purifying as used herein is the process of removing, partially removing (e.g., a fraction) of the nanovesicles from a sample containing source cells.

The terms “polynucleotide” and “nucleic acid” interchangeably refer to chains of nucleotides of any length and encompasses DNA, RNA, heteroduplexes, and synthetic molecules capable of encoding a polypeptide. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The term “amino acid sequence” is interchangeably used with the terms “polypeptide”, “protein”, and “peptide”. The conventional one-letter or three-letter codes for amino acid residues are used, with amino acid sequences being presented in the standard amino-to-carboxy terminal orientation (i.e., N→C).

As used herein, a “kinase dead domain” refers to an Eph receptor which is defective for intracellular signal transmission. The kinase domain of the corresponding wildtype Eph receptor may either be absent (partially or in its entirety) or rendered unfunctional through one or more mutations.

The term “parental” or “reference” with respect to a polypeptide or polynucleotide sequence means a polypeptide or polynucleotide sequence that serves as the template sequence used for generating altered (or variant) forms of the polypeptide or polynucleotide.

The terms “wild-type”, “native”, and “naturally occurring” with respect to an Eph receptor are used herein to refer to a domain that has a sequence that occurs in nature. The wild-type polypeptide is understood to include the mature form of the polypeptide. A “mature” polypeptide (or variant thereof) is one in which a signal sequence is absent, for example, cleaved from an immature form of the polypeptide during or following expression of the polypeptide.

As used herein, the term “mutant” with respect to a mutant polypeptide or mutant polynucleotide is used interchangeably with “variant.” A variant with respect to a given wild-type Eph receptor reference sequence can include naturally occurring allelic variants. A “non-naturally” occurring Eph receptor domain refers to a variant or mutant domain that is not present in a cell in nature and that is produced by genetic modification, e.g., using genetic engineering technology or mutagenesis techniques, of a parental Eph receptor polynucleotide introducing appropriate modifications into the nucleic acid sequence encoding the polypeptide, or by protein/peptide synthesis. A “variant” includes any sequence comprising at least one amino acid mutation with respect to wild-type. Mutations may include substitutions, insertions, and deletions (e.g., truncation) of one or more amino acids as well as frameshift or rearrangement in another protein. Similarly, the term “variant,” with respect to a polynucleotide, refers to a polynucleotide that differs in nucleotide sequence from a specified parental polynucleotide. The identity of the parental polypeptide or polynucleotide will be apparent from context. A variant can include one or more specific substitutions, insertions, and/or deletions as well as having a % sequence identity to the parental sequence.

The term “amino acid substitution” denotes the replacement of at least one existing amino acid residue with another different amino acid residue (replacing amino acid residue). The replacing amino acid residue may be a “naturally occurring amino acid residues” and selected from the group consisting of alanine (three letter code: ala, one letter code: A), arginine (arg, R), asparagine (asn, N), aspartic acid (asp, D), cysteine (cys, C), glutamine (gin, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “amino acid insertion” denotes the incorporation of at least one amino acid residue at a predetermined position in an amino acid sequence. In one embodiment the insertion will be the insertion of one or two amino acid residues. The inserted amino acid residue(s) can be any naturally occurring or non-naturally occurring amino acid residue. The term “amino acid deletion” denotes the removal of at least one amino acid residue at a predetermined position in an amino acid sequence.

The term “non-naturally occurring amino acid residue” denotes an amino acid residue, other than the naturally occurring amino acid residues as listed above, which can be covalently bound to the adjacent amino acid residues in a polypeptide chain. Examples of non-naturally occurring amino acid residues are norleucine, ornithine, norvaline, homoserine. Further examples are listed in Ellman, et al., Meth. Enzym. 202 (1991) 301-336. Exemplary method for the synthesis of non-naturally occurring amino acid residues are reported in, e. g., Noren, et al., Science 244 (1989) 182 and Ellman et al., supra.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. The same principle applies to nucleic acid sequences. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. Two molecules having the same primary amino acid or nucleic acid sequence are identical irrespective of any chemical and/or biological modification. A sequence being longer than any of the sequences provided herein, for example because it comprises additional domains, shall nevertheless be identical to the reference sequence disclosed herein if sequence identity over a comparison window is given, such as a comparison window covering the entire sequence as claimed.

The term “heterologous” or “exogenous” refers to such molecules that are not normally found in a given context, e.g., in a cell or in a polypeptide. For example, an exogenous or heterologous molecule can be introduced into a cell and is only present after manipulation of the cell, e.g., by transfection or other forms of genetic engineering. As another example, a heterologous amino acid sequence can be present in a protein in which it is not naturally found.

The term “endogenous” with reference to a polynucleotide or polypeptide refers to a polynucleotide or polypeptide that occurs naturally in the host cell.

“Fused” polypeptide sequences are connected via a peptide bond between two subject polypeptide sequences.

The terms “associated with”, “bound to”, “linked to”, “conjugated to” and their grammatical variants are used interchangeably herein to refer to a direct or indirect interaction between two or more elements. Two elements can be associated with each other by a covalent bond or a non-covalent bond and/or interaction. In some embodiments, a first element, e.g., a targeting domain, is associated with a second element, e.g., a scaffold protein, by a peptide bond. In some embodiments, a first element (e.g., a cargo protein linked to a scaffold binding domain) is associated with a second element, e.g., a scaffold protein, by a non-covalent interaction, e.g., phosphotyrosine-based binding (such as a binding between a phosphotyrosine and a phosphotyrosine binding (PTB) domain a binding between a phosphotyrosine and a Src homology 2 (SH2) domain), an electrostatic interaction, a hydrogen bond, a van der Waals interaction, a hydrophobic interaction, an ion induced dipole, a dipole induced dipole, an ionic bond, a coordination bond, a chelation, or any combination thereof. The first element and the second element can be associated directly, e.g., wherein a scaffold protein is linked to a cargo protein by a peptide bond; or the first element can be associated with the second element through an indirect association, e.g., wherein the cargo protein is associated with the scaffold protein through the interaction of an intermediary scaffold binding domain and the scaffold protein, wherein the scaffold protein binds the scaffold binding domain covalently linked to the cargo protein.

The phrase “binding partner” refers to a molecule that is a member of a specific binding pair, which is one of two different molecules that specifically binds to and is thereby defined as complementary with the other molecule in the pair. For example, one member of the specific binding pair may have an area on the surface or in a cavity that specifically binds to a particular spatial and polar organization of the other member of the specific binding pair.

As used herein, the term “dimerizing agent” or “dimerization agent” refers to one member of at least two elements that interact with each other to form a multimer (e.g., a dimer). In some embodiments, the dimerization agent is a first binding partner that interacts with a second binding partner. In some embodiments, the dimerization agent is a first binding partner that interacts with a second binding partner and/or a third binding partner. Any dimerizing agents can be used in the compositions and methods disclosed herein. In some embodiments, the dimerization agent can be a polypeptide, a polynucleotide, a fatty acid, a small molecule, or any combination thereof.

As used herein, the term “adaptor protein” refers to a polypeptide dimerization agent and said polypeptide can comprise, two or more scaffold binding domains that simultaneously interact with two scaffold proteins and/or a third scaffold protein. Said adaptor proteins can interact with more than one scaffold protein simultaneously and, through interaction with the scaffold protein, confine the scaffold proteins spatially at the membrane of a cell or nanovesicle, and thus serve as dimerization or oligomerization agent.

As used herein, the term “chemically induced dimerization agent” refers to dimerizing agent (e.g., the first binding partner and/or the second binding partner) that forms a dimer in the presence of a small molecule. In some embodiments, the chemically induced dimerization agent is selected from a first and a second binding partners of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737).

As used herein, the term “scaffold binding domain” refers to a first member of at least two binding partners that interact with each other to form a multimer (e.g., a dimer), where at least a second member is the scaffold protein, a cargo binding domain present on the scaffold protein, or a cargo binding domain that is covalently linked to the scaffold protein.

The term “pharmaceutically acceptable” refers to those active compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

In the medical/physiological context, i.e. in the context of a physiological state, the term “preventing” refers to decreasing the probability that an organism contracts or develops an abnormal condition.

The terms “subject” and “individual” are used interchangeably herein and refer to a human or non-human animal, generally a mammal. A subject may be a mammalian species such as a rabbit, a mouse, a rat, a guinea pig, a dog, a cat, a pig, a cow, a horse, a monkey, or a human. Thus, the methods, uses and compositions described in this document are applicable to both human and veterinary use. Where the subject is a human who is receiving medical care for a disease or condition, it is also addressed as a “patient”.

It is understood that wherever aspects or embodiments are described herein with the language “comprising,” otherwise analogous aspects described in terms of “consisting of” and/or “consisting essentially of” are also provided.

The scope and meaning of any use of a term will be apparent from the specific context in which the term is used. Certain further definitions for selected terms used throughout this document are given in the appropriate context of the detailed description, as applicable. Further, depending of the specific embodiment, selected definitions, embodiments or ranges may not apply.

Any embodiments specifically and explicitly recited herein may form the basis of a disclaimer either alone or in combination with one or more further embodiments.

Unless defined otherwise, all technical and scientific terms used herein have the same meaning as commonly understood to one of ordinary skill in the art to which the disclosures described herein belong. All publications and patents mentioned herein are incorporated herein by reference in their entirety for the purpose of describing and disclosing, for example, the constructs and methodologies that are described in the publications, which might be used in connection with the presently described disclosures.

Various aspects of the disclosure are described in further detail in the following subsections. It is understood that the various embodiments, preferences and ranges may be combined at will.

### 5.2 Polypeptides of the Disclosure

An aspect of the present invention relates to identification, use and modification of transmembrane polypeptides which are suitable for use as a scaffold to target (tether) payloads, e.g., biologically active molecules (e.g., cargo protein) to the surface and/or into the lumen of nanovesicles (e.g., EVs and hybridosomes). Another aspect of the present invention relates to generation and use of nanovesicles comprising scaffold polypeptides. One or more transmembrane polypeptides identified herein can be selectively used depending on a producer cell, production condition, purification methods, or intended application of the nanovesicles e.g., EVs and hybridosomes).

Provided herein are polypeptides comprising a transmembrane domain and further comprising an ectodomain and/or an endodomain that can be used to load a cargo (e.g., a cargo protein) on the surface of or into nanovesicles (e.g., EVs and hybridosomes). The cargo (e.g., a cargo protein) can be part of a polypeptide described herein. In other words, the cargo (e.g., a cargo protein) can be fused to the remaining portion of the polypeptide (e.g., via a linker). Alternatively, the cargo (e.g., a cargo protein) can be bound (preferably, reversibly bound) to the polypeptide through a cargo binding domain. A cargo binding domain can bind to the cargo (e.g., cargo protein) directly, or indirectly via a scaffold binding domain (SBD) linked to the cargo (e.g., cargo protein). The singular forms “a”, “an”, and “the” as used herein include plural referents. As such, a polypeptide described herein can be used to deliver one or more (e.g., one, two, three, four, five or more) cargos, and a polypeptide described herein can comprise one or more (e.g., one, two, three, four, five or more) cargo binding domains.

A polypeptide described herein can further comprise one or more functional moieties, such as a targeting domain that is capable of targeting the nanovesicle (e.g., EV or hybridosome) to a specific organ, tissue, or cell type, and/or a purification domain that can facilitate purification of the nanovesicle (e.g., EV or hybridosome).

In certain aspects, a polypeptide described herein comprises one or more domains from or derived from one or more ephrin receptors and locates to the membrane of a nanovesicle. Such a polypeptide is also referred to herein as an ephrin receptor derived polypeptide or a polypeptide derived from an ephrin receptor(s). An ephrin receptor derived polypeptide may or may not comprise one or more domains (e.g., a transmembrane domain) from or derived from a non-ephrin receptor protein. In various embodiments, a polypeptide described herein comprises an ectodomain or fragment thereof (e.g., a flexible domain) from or derived from one or more ephrin receptors, a transmembrane domain that is from or derived from an ephrin receptor or a non-ephrin receptor transmembrane protein, and optionally an endodomain or fragment thereof from or derived from one or more ephrin receptors.

A wild-type Eph receptor is typically composed of an ectodomain, a transmembrane domain, and an endodomain. The ectodomain comprises, in N→C order, a ligand binding domain (LBD), a cysteine-rich region comprising a Sushi domain and an epidermal growth factor (EGF)-like domain, followed by two fibronectin type III domains (FN1 domain and FN2 domain). The ectodomain is further described in section 5.2.1. The cysteine-rich region and the two fibronectin type III domains will hereinafter be referred to as “flexible domain”. The endodomain comprises a, juxtamembrane domain, a kinase domain, a sterile alpha motif (SAM) domain, and a PDZ-binding motif (the endodomain is further described in section 5.2.2).

In particular, provided herein are certain scaffold polypeptides comprising at least an ephrin receptor cysteine-rich (CR) domain, two ephrin receptor fibronectin type III (FN III) domains (i.e., a first ephrin receptor FN III domain (ephrin receptor FN1 domain), and a second ephrin receptor FN III domain (ephrin receptor FN2 domain)), and a transmembrane (TM) domain (e.g., an ephrin receptor TM domain), and optionally a cargo binding domain, an ephrin receptor juxtamembrane (JM) domain, an ephrin receptor kinase domain (KD), a sterile α-motif (SAM) linker domain (e.g., an ephrin receptor SAM linker domain), a SAM domain (e.g., an ephrin receptor SAM domain), an ephrin receptor PDZ binding motif (PBM) domain, a targeting domain, a purification domain, a modified Fc domain, and/or a ligand binding domain (LBD).

In one aspect, provided herein is a polypeptide comprising in N-terminus to C-terminus direction: a. an ephrin receptor CR domain; b. a first ephrin receptor FN III domain; and a second ephrin receptor FN III domain; and c. a TM domain (e.g., an ephrin receptor TM domain). In certain embodiments, the polypeptide is fused to a cargo (e.g., a cargo protein). In certain embodiments, the polypeptide associates with (i.e., binds to) a cargo (e.g., a cargo protein).

In one aspect, provided herein is a polypeptide comprising in N-terminus to C-terminus direction: a. a targeting domain; b. an ephrin receptor CR domain; c. a first ephrin receptor FN III domain and a second ephrin receptor FN III domain; and d. a TM domain (e.g., an ephrin receptor TM domain). In certain embodiments, the polypeptide is fused to a cargo (e.g., a cargo protein). In certain embodiments, the polypeptide associates with (i.e., binds to) a cargo (e.g., a cargo protein).

In one aspect, provided herein is a polypeptide comprising in N-terminus to C-terminus direction: a. an ephrin receptor CR domain; b. a first ephrin receptor FN III domain and a second ephrin receptor FN III domain; c. a TM domain (e.g., an ephrin receptor TM domain); and d. a cargo protein or a cargo binding domain.

In one aspect, provide herein is a polypeptide comprising in N-terminus to C-terminus direction: a. a targeting domain; b. an ephrin receptor CR domain; c. a first ephrin receptor FN III domain and a second ephrin receptor FN III domain; d. a TM domain (e.g., an ephrin receptor TM domain); and e. a cargo protein or a cargo binding domain.

In specific embodiments, the polypeptide lacks ephrin binding activity. In a specific embodiment, the polypeptide lacks an ephrin receptor LBD. In a specific embodiment, the polypeptide comprises an inactivated ephrin receptor LBD, for example, a modified (e.g., mutated) ephrin receptor LBD that lacks ephrin binding activity. In a specific embodiment, an ephrin receptor LBD may become inactivated due to one or more mutations in one or more domains outside the ephrin receptor LBD.

In specific embodiments, the polypeptide lacks ephrin receptor kinase activity. In a specific embodiment, the polypeptide lacks an ephrin receptor KD. In a specific embodiment, the polypeptide comprises an inactivated ephrin receptor KD, for example, a modified (e.g., mutated) ephrin receptor KD that lacks ephrin receptor kinase activity. In a specific embodiment, an ephrin receptor KD may become inactivated due to one or more mutations in one or more domains outside the ephrin receptor KD.

In specific embodiments, the polypeptide lacks both ephrin binding activity and ephrin receptor kinase activity. In a specific embodiment, the polypeptide lacks both an ephrin receptor LBD and an ephrin receptor KD. In a specific embodiment, the polypeptide comprises an inactivated ephrin receptor LBD, for example, a modified (e.g., mutated) ephrin receptor LBD that lacks ephrin binding activity, and lacks an ephrin receptor KD. In a specific embodiment, the polypeptide lacks an ephrin receptor LBD and comprises an inactivated ephrin receptor KD, for example, a modified (e.g., mutated) ephrin receptor KD that lacks ephrin receptor kinase activity. In a specific embodiment, the polypeptide comprises an inactivated ephrin receptor LBD, for example, a modified (e.g., mutated) ephrin receptor LBD that lacks ephrin binding activity, and comprises an inactivated ephrin receptor KD, for example, a modified (e.g., mutated) ephrin receptor KD that lacks ephrin receptor kinase activity. In a specific embodiment, an ephrin receptor LBD may become inactivated due to one or more mutations in one or more domains outside the ephrin receptor LBD. In a specific embodiment, an ephrin receptor KD may become inactivated due to one or more mutations in one or more domains outside the ephrin receptor KD.

In certain aspects, provided herein are Eph receptor derived polypeptides which may serve as signal neutral protein scaffold in nanovesicles (e.g., extracellular vesicles (EVs) and hybridosomes) for attaching molecules of interest. In certain embodiments, the polypeptides are membrane bound and (i) have reduced or no ability for cytoplasmic kinase activation in a cell and (ii) have diminished or no ligand binding capacity to ephrins expressed on other cells.

The polypeptides provided herein have several advantages over protein scaffolds described so far. They are endocytic recycling proteins and thus may be sorted by source cells into nanovesicles (e.g., EVs and hybridosomes). The ectodomain (in particular, the ectodomain of an Eph receptor) or a fragment thereof can be fused to molecules of interest, thereby allowing for engineering of the nanovesicles (e.g., EVs and hybridosomes) for additional functionalities such as cell type-specific targeting, receptor decoys, or purification. The protein scaffold protrudes from membrane, thereby allowing access to fused moieties. Also, because of the long protrusion the polypeptides described herein are flexible to bend and/or reconfigure while maintaining stability. A stable membrane anchoring can streamline the configuration of the resulting fusion protein, in that the molecule of interest may be directed to the outer surface or inside the lumen of a nanovesicle (e.g., an EV or hybridosome) or cell. Both the N- and C-termini of the polypeptide are accessible and free to attach a biologically active molecule (e.g., fusion moiety).

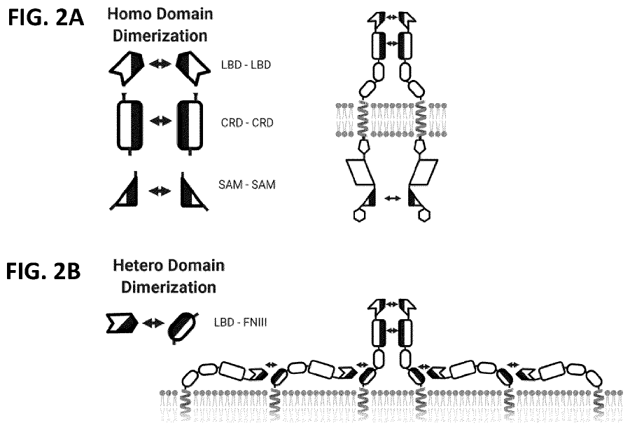

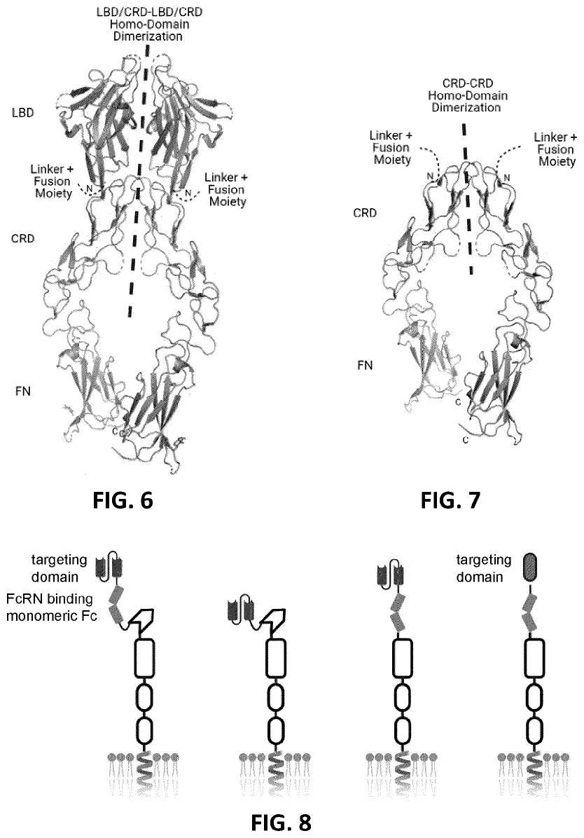

In addition, the polypeptides described herein have superior characteristics over protein scaffolds described in the art so far, as they comprise homo-dimer interaction interfaces that confer a propensity to oligomerize, e.g., cluster, thereby allowing for high density surface decoration on nanovesicles (e.g., EVs and hybridosomes) (see, e.g., FIG. 2A, FIG. 5, FIG. 6, and FIG. 7). The major residues involved in clustering of the Eph LBD-LBD interface (e.g., D104, K116, E117 and T144 of EphA2) and homo-domain dimerization of the CRD-CRD interface (e.g., CRD homo-domain dimerization motif: GX₁WX₂VX₃X₄G where X₁=E or K, X₂=L or M, X₃=P or A, X₄=V, I or L (SEQ ID NO: 239)) are highly conserved across EphA and EphB receptors. In certain embodiments, the polypeptide described herein comprises domains that can undergo hetero-domain dimerization, which can lead to the oligomerization (e.g., the hetero-domain dimerization between LBD-FN1) (see, e.g., FIG. 2B and FIG. 4). In certain embodiments, the polypeptide undergoes hetero-domain dimerization, in a head to tail configuration (e.g., dimerization between LBD-FN1 in EphA4 and EphA2 or dimerization between LBD-FN2 in EphB6).

In certain embodiments, the clustering propensity can further be enhanced or disrupted by modifications of the amino sequence of the domains. Further examples of clustering modifications are described in the sections below.

There are a number of assays to detect clustering of polypeptides of the disclosure, these include microscopy techniques for visualizing polypeptide clustering at the membrane, which include, but are not limited to, confocal microscopy and lateral membrane diffusion by fluorescent correlation spectroscopy. For example, polypeptide and/or polypeptide-specific antibodies can be labeled, and these labels can be detected to visualize clustering of polypeptide elements. In one example of this type of assay, a cell comprising the polylpeptide of interest is contacted with a polypeptide-specific antibody, and a fluorescently labeled secondary antibody that binds to the polypeptide-specific antibody. Examples for such techniques are described in Mély, Y., et al, 2013. Fluorescent methods to study biological membranes. Berlin: Springer Berlin Heidelberg, He L, Hristova K (2014). Quantification of the effects of mutations on receptor tyrosine kinase (RTK) activation in mammalian cells. Receptor Tyrosine Kinases: Methods and Protocols. 81-87 Christopher King, et a al, Fully quantified spectral imaging reveals in vivo membrane protein interactions, Integrative Biology, Volume 8, Issue 2, February 2016, Pages 216-229.

In one aspect, a polypeptide derived from an Eph receptor is provided, wherein said polypeptide
i. comprises an ephrin ligand binding domain exhibiting decreased or no binding to ephrins as compared to the parental Eph receptor; and
ii. comprises a transmembrane domain.

Although the parental Eph receptor may stem from any mammalian species, including human, mouse or rat, the parental Eph receptor is preferably a human Eph receptor (hEph). In some embodiments, the parental Eph receptor comprises an amino acid sequence that is selected from the group consisting of SEQ ID NOs: 212-225 and fragments thereof. However, in some embodiments, the sources of the individual domains of the polypeptide may be mixed. By way of example, the polypeptide may comprise the LBD of one receptor (e.g. mutated LBD of EphA2, see SEQ ID NO:16), the flexible domain of another receptor (e.g. flexible domain of EphA4, see SEQ ID NO:63) and the transmembrane domain of a third receptor (e.g. TM domain of EphA1, see SEQ ID NO: 74).

Any combination(s) of deletions, substitutions, additions, modifications and insertions can be made to the Eph receptor derived polypeptides, provided that the generated variant possesses the desired characteristics for which it can be screened using appropriate methods. Of particular interest are substitutions, preferably conservative substitutions. The polypeptide described herein may comprise one or more, such as two, three, four, five, six, seven, eight, nine, ten, eleven, twelve or more of such substitutions.

### 5.2.1 Ectodomain and Transmembrane Domain

As explained above, the ephrin receptor ectodomain comprises, in N→C order, a ligand binding domain (LBD), a cysteine-rich region (CR domain) comprising a Sushi domain and an epidermal growth factor (EGF)-like domain, followed by two fibronectin type III domains (FN III domains-FN1 domain and FN2 domain). The transmembrane domain is C-terminal to the two FN III domains.

In certain embodiments, a polypeptide described herein comprises an ephrin receptor ectodomain and an ephrin receptor transmembrane domain (TM domain) from or derived from the same ephrin receptor. In certain embodiments, a polypeptide described herein comprises an ephrin receptor ectodomain and an ephrin receptor TM domain from or derived from two different ephrin receptors. In certain embodiments, a polypeptide described herein comprises a wild-type ephrin receptor ectodomain and a wild-type ephrin receptor TM domain. In certain embodiments, a polypeptide described herein comprises a mutant ephrin receptor ectodomain and a mutant ephrin receptor TM domain. In certain embodiments, a polypeptide described herein comprises a wild-type ephrin receptor ectodomain and a mutant ephrin receptor TM domain. In certain embodiments, a polypeptide described herein comprises a mutant ephrin receptor ectodomain and a wild-type ephrin receptor TM domain. In various embodiments, a mutant ephrin receptor ectodomain has at least 70%, at least 80%, at least 85%, at least 90%, at least 95%, at least 96%, at least 97%, at least 98% or at least 99% sequence identity to a wild-type ephrin receptor ectodomain. In various embodiments, a mutant ephrin receptor TM domain has at least 70%, at least 80%, at least 85%, at least 90%, at least 95%, at least 96%, at least 97%, at least 98% or at least 99% sequence identity to a wild-type ephrin receptor TM domain.

In certain embodiments, a polypeptide described herein comprises an ephrin receptor CR domain (a wild-type or a mutant ephrin receptor CR domain), a first ephrin receptor FN III domain (a wild-type or a mutant first ephrin receptor FN III domain), a second ephrin receptor FN III domain (a wild-type or a mutant second ephrin receptor FN III domain), and an ephrin receptor TM domain (a wild-type or a mutant ephrin receptor TM domain), wherein all of the four ephrin receptor domains are from or derived from the same ephrin receptor. In certain embodiments, a polypeptide described herein comprises an ephrin receptor CR domain (a wild-type or a mutant ephrin receptor CR domain), a first ephrin receptor FN III domain (a wild-type or a mutant first ephrin receptor FN III domain), a second ephrin receptor FN III domain (a wild-type or a mutant second ephrin receptor FN III domain), and an ephrin receptor TM domain (a wild-type or a mutant ephrin receptor TM domain), wherein the four ephrin receptor domains are from or derived from two, three, or four ephrin receptors. In various embodiments, a mutant ephrin receptor domain has at least 70%, at least 80%, at least 85%, at least 90%, at least 95%, at least 96%, at least 97%, at least 98% or at least 99% sequence identity to a wild-type ephrin receptor domain.

Both the ecto- and endodomains of the natural Eph receptor may comprise protease cleavage sites. In some embodiments, it may be beneficial to remove one or more protease cleavage sites so that the polypeptide remains intact when in a cellular environment. In some embodiments, the one or more cleavage sites are specific for metalloproteases, such as a ADAMs (A Disintegrin And Metalloprotein, members of the zinc protease superfamily). In some embodiments, the one or more cleavage sites are specific for γ-secretases.

In some embodiments, one cleavage site may be between the FN2 domain and the transmembrane domain. For example, one or more modifications in the amino acid stretch 533-547 of the EphA4 fragment (SEQ ID NO:226) can be made to remove protease cleavage sites. In specific embodiments, such modification is a mutation. Thus, the polypeptide may comprise a sequence wherein one or more mutations are present when compared to the parental Eph receptor at amino acid position 533, 534, 535, 536, 537, 538, 539, 540, 541, 542, 543, 544, 545, 546 and/or 547 of SEQ ID NO:226. A further example for the removal of a cleavage site between the FN2 domain and the transmembrane domain is one or more modifications at amino acid position 536 of the EphB2 ectodomain (SEQ ID NO: 208), such as a S536E modification.

In a further embodiment, an Eph receptor can be made more resistant to protease cleavage by one or more modifications of the amino acid in the FN1 domain. For example, EphB2 may be made resistant to protease cleavage by one or more modifications in FN1 of amino acids L356 and 1395 (e.g., L356A, I395A) of SEQ ID NO: 208.

In a further embodiment, an Eph receptor can be made more resistant to protease cleavage by one or more modifications of the amino acid in the transmembrane domain. For example, EphB2 may be made resistant to protease cleavage by one or more modifications in the transmembrane domain of amino acid A562 (e.g., A562S) of SEQ ID NO: 208.

Thus, the polypeptide may comprise a sequence wherein one or more mutations are present in the FN1, FN2 and/or transmembrane domain when compared to the parental Eph receptor. In some embodiments, the polypeptide is more resistant to cleavage and has one, two, three, four, five, six, seven, eight, nine or ten mutations in the ectodomain and/or transmembrane domain when compared to the parental Eph receptor.

In certain embodiments, the polypeptides of the disclosure comprise no protease cleavage site. In some embodiments, the polypeptides comprise one, two or three protease cleavage sites.

### (a) Ligand Binding Domain (LBD)

In various embodiments, a polypeptide described herein exhibits reduced ephrin binding activity or lacks ephrin binding activity. In certain embodiments, a polypeptide described herein lack an ephrin receptor ligand binding domain (LBD). See FIG. 1B for schematic illustrations of exemplary Eph receptor-derived polypeptides, with a mutated (including truncated) ligand binding domain (LBD) or no LBD. Decreasing or abolishing ephrin ligand binding may be useful in that the Eph receptor derived polypeptides of the disclosure do not elicit reverse signaling in a cell they enter in close contact with. This may be beneficial when the polypeptides described herein are present on nanovesicles (e.g., EVs and hybridosomes), in that the respective signaling pathways of the target cell are not triggered upon contact of the nanovesicle with its target cell.

In certain embodiments, an Eph receptor-derived polypeptide described herein comprises an ephrin ligand binding domain which is modified such that it exhibits decreased or no binding to ephrins. The parental Eph receptor may serve as a reference for determining the affinity of ephrin binding. In some embodiments, the ephrin ligand binding domain is modified through mutation (e.g., substitution, insertion, and/or deletion), preferably through substitution of one or more amino acids. Receptor-ligand binding activity may be measured using methods known in the art, see, for example, Elliott S., et al., (1997) Blood, 89:493-502.

In some embodiments, ephrin ligand binding is decreased by at least 10%, 20%, 25%, 30%, 35%, 40%, 45%, 50%, 60%, 65%, 70%, 75%, 80%, 85%, 90%, 95%, or 99% as compared to the parental Eph receptor.

In some embodiments, the binding affinity of the polypeptide of the disclosure to ephrins is at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 100-fold lower, at least 150-fold lower, or 10-50-fold lower, 50-100-fold lower, 100-150-fold lower, 150-200-fold lower, or more than 200-fold lower relative to that of the parental Eph receptor.

In various embodiments, the Eph receptor derived polypeptide of the disclosure comprises one or more mutations that cause said polypeptide to have substantially reduced or ablated affinity or activity, e.g. binding affinity (e.g. K_{D}) and/or activation activity (for instance, when the ligand is an agonist to the ephrin receptor, measurable as, for example, K_{A} and/or EC₅₀) and/or inhibition activity (for instance, when the ligand is an antagonist to the ephrin receptor, measurable as, for example, K_{I} and/or IC₅₀), relative to parental Eph receptors. In such embodiments, the polypeptide has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, or about 80%-100% of the affinity for Ephrin relative to the parental Eph receptor. In some embodiments, the binding affinity is at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 100-fold lower, at least 150-fold lower, or 10-50-fold lower, 50-100-fold lower, 100-150-fold lower, 150-200-fold lower, or more than 200-fold lower relative to the parental Eph receptor.

Methods for analyzing binding affinity and binding kinetics between ephrins and LBDs are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet® (ForteBio, Inc., Menlo Park, CA)), and Western blot analysis. In some embodiments, ELISA is used to determine binding affinity. In some embodiments, surface plasmon resonance (SPR) is used to determine binding affinity and/or binding kinetics. In some embodiments, kinetic exclusion assays are used to determine binding affinity and/or binding kinetics. In some embodiments, BioLayer interferometry assays are used to determine binding affinity and/or binding kinetics.

Specific positions within the LBD constitute the ephrin binding site and interact with ephrins. A non-exhaustive list of LBD amino acid positions that interact with ephrins and make up the ephrin binding site have been cataloged in the NCBI Conserved Domain Database (CDD), (e.g., see positions in Table 1). Thus, in certain embodiments, the parental Eph receptor comprises a LBD wherein an arginine (R) is replaced by glutamic acid (E) in the loop between the G and H beta-strands (e.g., the position R104 for EphA1/3, R103 for EphA2, R106 for EphA4/8, R107 for EphA7, R109 for EphA6, R110 for EphA10, R135 for EphA5, R94 for EphB1, R95 for EphB2, R115 for EphB3, or R112 for EphB6). In some embodiments, the leucine L95 of EphB4 can be replaced by arginine (R). Additionally or alternatively, the amino acid at position 154 of the parental EphA4 LBD is replaced by alanine (A) (see SEQ ID NO:15). The skilled person is well capable of identifying further positions which will decrease or abolish ephrin binding. In some embodiments, the LBD of the polypeptide has at least 70%, at least 80%, at least 85%, at least 90%, at least 95%, at least 96%, at least 97%, at least 98% or at least 99% sequence identity to the LBD of a wild-type ephrin receptor (e.g., an ephrin receptor LBD comprising an amino acid sequence selected from the group consisting of SEQ ID NOs:1-14).

In some embodiments, the LBD of the polypeptide is the LBD of a wild-type ephrin receptor (e.g., an ephrin receptor LBD comprising an amino acid sequence selected from the group consisting of SEQ ID NOs: 1-14).

TABLE 1
Positions of the LBD ephrin binding site (CDD: cl02704).

| Protein | | |
|---|---|---|
| (UniProt | NCBI | |
| ID No.) | CDD # | Positions |
| EPHA1 | 198447 | S52, Q54, Q55, Q56, I57, L58, Y64, M65, Q67, C69, T102, R104, |
| (P21709) | | F109, P110, F154, D158, L159, G162, S163, V164, C191, V192, |
| | | A193, V195 |
| EPHA2 | 198448 | D53, M55, Q56, N57, I58, M59, D61, P63, Y65, M66, S68, V69, |
| (P29317) | | C70, M73, T101, R103, F108, T151, D155, F156, R159, H160, |
| | | V161, C188, V189, A190, L192 |
| EPHA3 | 198449 | E53, I55, S56, G57, V58, D59, R66, T67, Q69, C71, T102, R104, |
| (P29320) | | I109, P110, F152, D156, L157, R160, I161, L162, C189, V190, |
| | | A191, V193 |
| EPHA4 | 198450 | Q40, E42, E51, E55, E56, V57, S58, I59, M60, E62, N64, R68, |
| (P54764) | | Q71, C73, T104, L105, R106, L111, P112, F154, V157, D158, I163, |
| | | M164, L166, C191, I192, A193, V195 |
| EPHA5 | 198451 | E84, I86, G87, E88, V89, D90, H97, T98, Q100, C102, T133, R135, |
| (P54756) | | L140, P141, F183, D187, L188, R191, V192, M193, C220, I221, |
| | | A222, V224 |
| EPHA6 | 198452 | D58, I60, T61, E62, M63, D64, H71, T72, Q74, C76, T107, R109, |
| (Q9UF33) | | I114, P115, F157, D161, L162, R165, I166, L167, C194, I195, |
| | | A196, V198 |
| EPHA7 | 198453 | E56, I58, S59, G60, L61, D62, R69, T70, Q72, C74, T105, R107, |
| (Q15375) | | L112, P113, F155, D159, L160, R163, K164, M165, C192, I193, |
| | | A194, V196 |
| EPHA8 | 198439 | D43, D55, S56, I57, N58, V60, D61, P66, H68, Q71, T104, R106, |
| (P29322) | | F154, R162, R163, L164, C191, L192, A193, L195 |
| EPHA10 | 198439 | E47, E59, E60, I61, S62, V64, D65, P70, R72, Q75, T108, R110, |
| (Q5JZY3) | | F161, R169, K170, M171, C198, V199, A200, V202 |
| EPHB1 | 198444 | T27, A28, T29, E31, E43, E44, V46, S48, Y52, L54, T56, R59, |
| (P54762) | | Q92, T93, V94, R98, S100, P146, F148, Q154, R155, L157, K158, |
| | | V159, C183, S185, L187 |
| EPHB2 | 198445 | T28, A29, T30, E32, E44, E45, V46, S47, Y49, M53, T55, R57, |
| (P29323) | | Q60, S93, V94, R95, S99, P101, F147, Q149, V150, D151, L152, |
| | | G153, G154, R155, V156, K158, I159, C184 |
| EPHB3 | 198446 | W47, V48, T49, E51, E63, E64, V65, S66, Y68, M72, P74, R76, |
| (P54753) | | Q79, T112, V113, R114, S118, P120, F166, R168, D170, A171, |
| | | R173, V174, C199, S201, I203 |
| EPHB4 | 198442 | T27, D29, E43, E44, L45, S46, L48, D49, E50, Q52, S54, R56, |
| (P54760) | | E59, V60, T93, L95, T147, K149, A155, T156, G157, V159, |
| | | C184, M185, A186, L188 |
| EPHB6 | 198443 | E45, D57, E58, V59, S60, L62, D63, L68, R70, E73, S110, R112, |
| (O15197) | | F164, A190, G191, L192, C219, L220, A221, V223 |

In one aspect, the polypeptide described herein may comprises a LBD and said LBD exhibits a three dimensional structure that can be superimposed with the LBD structure of a wild type ephrin receptor. In certain embodiments, the polypeptide described herein comprises a LBD and said LBD exhibits a three dimensional structure, whose portion between equivalent Cα positions can be superimposed with a wild type Eph receptor LBD with root-mean-square deviations (RMSDs) of at most 1, 2, 4, 4, 5, 6, 7, 8, 9 or 10 Å. For example, the structures of unbound EphA2 LBD and EphB2 LBD can be superimposed with a RMSD of 1.7 Å between corresponding Ca positions as described in Himanen, J. P et al. (2009). EMBO reports, 10: 722-728. As a further example, the structures of unbound EphB4 LBD and EphB2 LBD can be superimposed with an overall RMSD of 1.08 Å between equivalent Ca positions as described in Chrencik et al., Structure, 14, 2, (2006), 321-330). Methods for comparing two biological structures by calculating the RMSD of superimposed structures are well known in the art (as described in Xu, Y., Xu, D. and Liang, J., 2007. Computational methods for protein structure prediction and modeling. Springer.) Two identical structures will display a zero RMSD, whereas two distinct ones will display values proportional to their dissimilarity. Further examples of superimposed structures are illustrated in FIG. 19.

In some embodiments, a scaffold polypeptide described herein comprises a LBD domain and said LBD has reduced ephrin binding activity described above and has at least 70%, at least 80%, at least 85%, at least 90%, at least 95%, at least 96%, at least 97%, at least 98% or at least 99% sequence identity to the LBD domain of a wild-type ephrin receptor (e.g., an ephrin receptor LBD comprising an amino acid sequence selected from the group consisting of SEQ ID NOs:1-14 as shown in Table 2).

TABLE 2
LBD (CDD Superfamily: cl02704, ProRule:
PRU00883).

| SEQ | Protein | | | |
|---|---|---|---|---|
| ID | (UniProt | NCBI | | |
| NO: | ID No.) | CDD # | Region | Sequence |
| 1 | EPHA1 | cd10479 | 27 . . . 203 | EVTLMDTSKAQGELGW |
| | (P21709) | | | LLDPPKDGWSEQQQIL |
| | | | | NGTPLYMYQDCPMQGR |
| | | | | RDTDHWLRSNWIYRGE |
| | | | | EASRVHVELQFTVRDC |
| | | | | KSFPGGAGPLGCKETF |
| | | | | NLLYMESDQDVGIQLR |
| | | | | RPLFQKVTTVAADQSF |
| | | | | TIRDLVSGSVKLNVER |
| | | | | CSLGRLTRRGLYLAFH |
| | | | | NPGACVALVSVRVFYQ |
| | | | | R |
| 2 | EPHA2 | cd10480 | 28 . . . 200 | EVVLLDFAAAGGELGW |
| | (P29317) | | | LTHPYGKGWDLMQNIM |
| | | | | NDMPIYMYSVCNVMSG |
| | | | | DQDNWLRTNWVYRGEA |
| | | | | ERIFIELKFTVRDCNS |
| | | | | FPGGASSCKETFNLYY |
| | | | | AESDLDYGTNFQKRLF |
| | | | | TKIDTIAPDEITVSSD |
| | | | | FEARHVKLNVEERSVG |
| | | | | PLTRKGFYLAFQDIGA |
| | | | | CVALLSVRVYYKK |
| 3 | EPHA3 | cd10481 | 29 . . . 201 | EVNLLDSKTIQGELGW |
| | (P29320) | | | ISYPSHGWEEISGVDE |
| | | | | HYTPIRTYQVCNVMDH |
| | | | | SQNNWLRTNWVPRNSA |
| | | | | QKIYVELKFTLRDCNS |
| | | | | IPLVLGTCKETFNLYY |
| | | | | MESDDDHGVKFREHQF |
| | | | | TKIDTIAADESFTQMD |
| | | | | LGDRILKLNTEIREVG |
| | | | | PVNKKGFYLAFQDVGA |
| | | | | CVALVSVRVYFKK |
| 4 | EPHA4 | cd10482 | 30 . . . 203 | EVTLLDSRSVQGELGW |
| | (P54764) | | | IASPLEGGWEEVSIMD |
| | | | | EKNTPIRTYQVCNVME |
| | | | | PSQNNWLRTDWITREG |
| | | | | AQRVYIEIKFTLRDCN |
| | | | | SLPGVMGTCKETFNLY |
| | | | | YYESDNDKERFIRENQ |
| | | | | FVKIDTIAADESFTQV |
| | | | | DIGDRIMKLNTEIRDV |
| | | | | GPLSKKGFYLAFQDVG |
| | | | | ACIALVSVRVFYKK |
| 5 | EPHA5 | cd10483 | 60 . . . 232 | EVNLLDSRTVMGDLGW |
| | (P54756) | | | IAFPKNGWEEIGEVDE |
| | | | | NYAPIHTYQVCKVMEQ |
| | | | | NQNNWLLTSWISNEGA |
| | | | | SRIFIELKFTLRDCNS |
| | | | | LPGGLGTCKETFNMYY |
| | | | | FESDDQNGRNIKENQY |
| | | | | IKIDTIAADESFTELD |
| | | | | LGDRVMKLNTEVRDVG |
| | | | | PLSKKGFYLAFQDVGA |
| | | | | CIALVSVRVYYKK |
| 6 | EPHA6 | cd10484 | 34 . . . 206 | QVVLLDTTTVLGELGW |
| | (Q9UF33) | | | KTYPLNGWDAITEMDE |
| | | | | HNRPIHTYQVCNVMEP |
| | | | | NQNNWLRTNWISRDAA |
| | | | | QKIYVEMKFTLRDCNS |
| | | | | IPWVLGTCKETFNLFY |
| | | | | MESDESHGIKFKPNQY |
| | | | | TKIDTIAADESFTQMD |
| | | | | LGDRILKLNTEIREVG |
| | | | | PIERKGFYLAFQDIGA |
| | | | | CIALVSVRVFYKK |
| 7 | EPHA7 | cd10485 | 30 . . . 204 | AKEVLLLDSKAQQTEL |
| | (Q15375) | | | EWISSPPNGWEEISGL |
| | | | | DENYTPIRTYQVCQVM |
| | | | | EPNQNNWLRTNWISKG |
| | | | | NAQRIFVELKFTLRDC |
| | | | | NSLPGVLGTCKETFNL |
| | | | | YYYETDYDTGRNIREN |
| | | | | LYVKIDTIAADESFTQ |
| | | | | GDLGERKMKLNTEVRE |
| | | | | IGPLSKKGFYLAFQDV |
| | | | | GACIALVSVKVYYKK |
| 8 | EPHA8 | c102704 | 31 . . . 203 | EVNLLDTSTIHGDWGW |
| | (P29322) | | | LTYPAHGWDSINEVDE |
| | | | | SFQPIHTYQVCNVMSP |
| | | | | NQNNWLRTSWVPRDGA |
| | | | | RRVYAEIKFTLRDCNS |
| | | | | MPGVLGTCKETFNLYY |
| | | | | LESDRDLGASTQESQF |
| | | | | LKIDTIAADESFTGAD |
| | | | | LGVRRLKLNTEVRSVG |
| | | | | PLSKRGFYLAFQDIGA |
| | | | | CLAILSLRIYYKK |
| 9 | EPHA10 | c102704 | 35 . . . 210 | EVILLDSKASQAELGW |
| | (Q5JZY3) | | | TALPSNGWEEISGVDE |
| | | | | HDRPIRTYQVCNVLEP |
| | | | | NQDNWLQTGWISRGRG |
| | | | | QRIFVELQFTLRDCSS |
| | | | | IPGAAGTCKETFNVYY |
| | | | | LETEADLGRGRPRLGG |
| | | | | SRPRKIDTIAADESFT |
| | | | | QGDLGERKMKLNTEVR |
| | | | | EIGPLSRRGFHLAFQD |
| | | | | VGACVALVSVRVYYKQ |
| 10 | EPHB1 | cd10476 | 20 . . . 195 | ETLMDTRTATAELGWT |
| | (P54762) | | | ANPASGWEEVSGYDEN |
| | | | | LNTIRTYQVCNVFEPN |
| | | | | QNNWLLTTFINRRGAH |
| | | | | RIYTEMRFTVRDCSSL |
| | | | | PNVPGSCKETFNLYYY |
| | | | | ETDSVIATKKSAFWSE |
| | | | | APYLKVDTIAADESFS |
| | | | | QVDFGGRLMKVNTEVR |
| | | | | SFGPLTRNGFYLAFQD |
| | | | | YGACMSLLSVRVFFKK |
| 11 | EPHB2 | cd10477 | 19 . . . 196 | VEETLMDSTTATAELG |
| | (P29323) | | | WMVHPPSGWEEVSGYD |
| | | | | ENMNTIRTYQVCNVFE |
| | | | | SSQNNWLRTKFIRRRG |
| | | | | AHRIHVEMKFSVRDCS |
| | | | | SIPSVPGSCKETFNLY |
| | | | | YYEADFDSATKTFPNW |
| | | | | MENPWVKVDTIAADES |
| | | | | FSQVDLGGRVMKINTE |
| | | | | VRSFGPVSRSGFYLAF |
| | | | | QDYGGCMSLIAVRVFY |
| | | | | RK |
| 12 | EPHB3 | cd10478 | 39 . . . 211 | EETLMDTKWVTSELAW |
| | (P54753) | | | TSHPESGWEEVSGYDE |
| | | | | AMNPIRTYQVCNVRES |
| | | | | SQNNWLRTGFIWRRDV |
| | | | | QRVYVELKFTVRDCNS |
| | | | | IPNIPGSCKETFNLFY |
| | | | | YEADSDVASASSPFWM |
| | | | | ENPYVKVDTIAPDESF |
| | | | | SRLDAGRVNTKVRSFG |
| | | | | PLSKAGFYLAFQDQGA |
| | | | | CMSLISVRAFYKK |
| 13 | EPHB4 | cd10474 | 17 . . . 196 | EETLLNTKLETADLKW |
| | (P54760) | | | VTFPQVDGQWEELSGL |
| | | | | DEEQHSVRTYEVCDVQ |
| | | | | RAPGQAHWLRTGWVPR |
| | | | | RGAVHVYATLRFTMLE |
| | | | | CLSLPRAGRSCKETFT |
| | | | | VFYYESDADTATALTP |
| | | | | AWMENPYIKVDTVAAE |
| | | | | HLTRKRPGAEATGKVN |
| | | | | VKTLRLGPLSKAGFYL |
| | | | | AFQDQGACMALLSLHL |
| | | | | FYKK |
| 14 | EPHB6 | cd10475 | 33 . . . 231 | EEVLLDTTGETSEIGW |
| | (O15197) | | | LTYPPGGWDEVSVLDD |
| | | | | QRRLTRTFEACHVAGA |
| | | | | PPGTGQDNWLQTHFVE |
| | | | | RRGAQRAHIRLHFSVR |
| | | | | ACSSLGVSGGTCRETF |
| | | | | TLYYRQAEEPDSPDSV |
| | | | | SSWHLKRWTKVDTIAA |
| | | | | DESFPSSSSSSSSSSS |
| | | | | AAWAVGPHGAGQRAGL |
| | | | | QLNVKERSFGPLTQRG |
| | | | | FYVAFQDTGACLALVA |
| | | | | VRLFSYT |

In some embodiments, the ligand binding domain comprises the amino acid sequence of the LBD of a wild-type ephrin receptor (e.g., an ephrin receptor LBD comprising an amino acid sequence selected from the group consisting of SEQ ID NOs: 1-14) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the ligand binding domain comprises an amino acid sequence shown in Table 2.

In some embodiments, the ligand binding domain comprises an amino acid sequence shown in Table 2.

TABLE 3
Exemplary LBDs with ephrin binding site
mutations.

| SEQ | | |
|---|---|---|
| ID | | |
| No: | Name | Sequence |
| 15 | EphA4_mut | EVTLLDSRSVQGELGWIASPLEGGWEE |
| | F154A | VSIMDEKNTPIRTYQVCNVMEPSQNNW |
| | | LRTDWITREGAQRVYIEIKFTLRDCNS |
| | | LPGVMGTCKETFNLYYYESDNDKERFI |
| | | RENQFVKIDTIAADESATQVDIGDRIM |
| | | KLNTEIRDVGPLSKKGFYLAFQDVGAC |
| | | IALVSVRVFYKK |
| 16 | EphA2_mut | EVVLLDFAAAGGELGWLTHPYGKGWDL |
| | R103E | MQNIMNDMPIYMYSVCNVMSGDQDNWL |
| | | RTNWVYRGEAERIFIELKFTVEDCNSF |
| | | PGGASSCKETFNLYYAESDLDYGTNFQ |
| | | KRLFTKIDTIAPDEITVSSDFEARHVK |
| | | LNVEERSVGPLTRKGFYLAFQDIGACK |
| | | VALLSVRVYYK |
| 17 | EphA4 | EVTLLDSRSVQGELGWIASPLGGSGGS |
| | with | GGSKFQLFTPFSLGFEFRPGRGGSGGS |
| | Ephrin | GGSGGWEEVSIMDEKNTPIRTYQVCNV |
| | loop | MEPSQNNWLRTDWITREGAQRVYIEIK |
| | insertion | FTLRDCNSLPGVMGTCKETFNLYYYES |
| | (the | DNDKERFIRENQFVKIDTIAADESATQ |
| | underlined | VDIGDRIMKLNTEIRDVGPLSKKGFYL |
| | sequence | AFQDVGACIALVSVRVFYKK |
| | is a | |
| | linker- | |
| | loop- | |
| | linker | |
| | sequence) | |

In some embodiments, the ligand binding domain comprises the amino acid sequence of a wild-type ephrin receptor (e.g., an ephrin receptor LBD comprising an amino acid sequence selected from the group consisting of SEQ ID NOs: 1-14) and its length is one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of the amino acid sequence of the ligand binding domain of the wild-type ephrin receptor (e.g., SEQ ID NOs: 1-14).

In some embodiments, the LBD comprises a chimera of amino acid sequences encoding for beta strands and joining loops that are from different Eph receptors (e.g., EphA2 parental sequence with the H-J loop and J beta strand and J-K loop derived from the EphA4 LBD as described in Li Peng et al, Journal of Molecular Biology, 2011)

In some embodiments, a fragment of an ephrin receptor binding domain is inserted into the BC loop of the LBD via a linker, and thereby mimicking the ligand bound state of the LBD and promoting clustering on the LBD clustering interfaces. For example, the GH loop of the receptor binding domain of Ephrin A2 can be inserted into the BC loop of an EphA4 LBD as depicted in SEQ ID: 17 in In some embodiments, the ligand binding domain comprises an amino acid sequence shown in Table 2.

### Table 3.

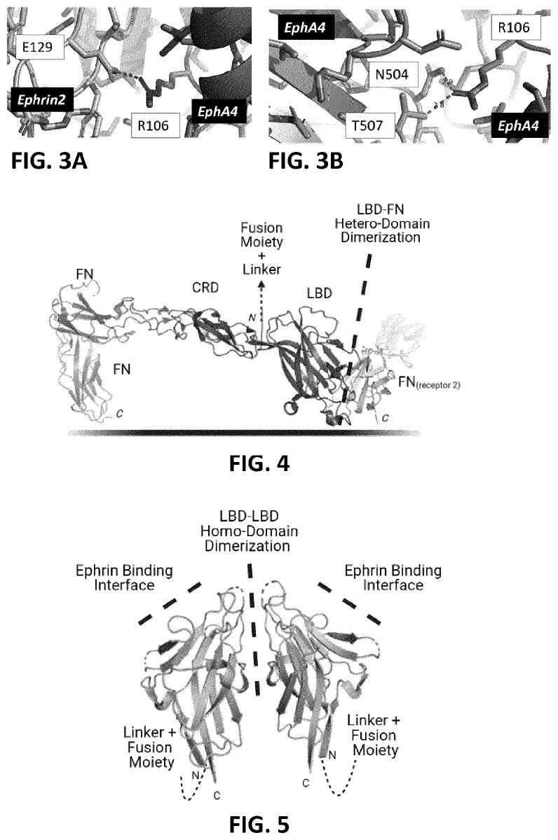

In some embodiments, a modified LBD may have the propensity to elicit head-to-tail hetero-domain dimer formation, i.e., the modified LBD may bind to the FN2 domain of another Eph receptor derived polypeptide, thereby increasing oligomerization of the polypeptides on cellular surfaces or nanovesicles (e.g., EVs and hybridosomes). In one embodiment, the Eph receptor derived polypeptide comprises a FN2 domain which has been modified to increase oligomerization. In certain embodiments, the LBD is modified to decrease Ephrin binding and the FN2 is modified to improve head-to-tail binding, for example, by replacing, in EphA4, an arginine at position 106 with a glutamic acid (R106E) and the threonine at positions 507 with an asparagine (T507N) in the FN2 domain 2. The proximity of these residues is shown in FIG. 3B. The interaction of the ephrin2 receptor binding domain residue E129 with EphA4 LBD residue R106 is shown in FIG. 3A.

### (b) Cysteine-Rich (CR) Domain and Fibronectin Type III (FN III) Domains

The cysteine-rich region and the two fibronectin type III domains (i.e., FN1 and FN2) are referred to as “flexible domain”. In some embodiments the flexible domain of a polypeptide described herein is chosen from the sequences in Table 7.

The polypeptides of the disclosure may comprise the flexible domains of the parental Eph receptors or not. In some embodiments, said flexible domains are lacking partially or in their entirety. For example, the flexible domains may be partially or entirely replaced by other polypeptides, such as a linker or a functional protein. Additionally or alternatively, protein sequences of interest (e.g., targeting domains and/or purification domains) may be inserted or attached to the flexible domain (e.g., the CR domain, the FN1 domain, or the FN2 domain) of the polypeptide, thereby adding an additional functionality. In certain embodiments, the polypeptides of the disclosure comprise the flexible domains of Eph receptors.

In some embodiments, the polypeptide comprises the cysteine-rich region, the FN1 domain and/or the FN2 domain of a parental Eph receptor, wherein the cysteine-rich region, the FN1 domain and/or the FN2 domain comprises one or more modifications to increase interaction between one or more polypeptides. In certain embodiments, said one or more modifications are mutation(s), which can be substitution(s), insertion(s), deletion(s), or any combination thereof.

In specific embodiments, the parental Eph receptor comprises SEQ ID NO:202 and amino acid at position 504 is replaced by aspartic acid (D) and/or the amino acid at position 507 is replaced by aspartic acid (D). In a further specific embodiment, amino acid position 154 of the parental LBD is replaced by alanine (A). It is within routine experimentation to identify further mutations within the flexible domains of this or other parental Eph receptors to increase oligomerization of the resulting polypeptides.

In some embodiments, the polypeptide comprises a CRD homo-domain dimerization motif which increases interaction between two or more of the polypeptides. In certain embodiments the CRD homo-domain dimerization motif is GX₁WX₂VX₃X₄G, where X₁=E or K, X₂=L or M, X₃=P or A, X₄=V, I or L (SEQ ID NO: 239).

In some embodiments, the CR domain of the polypeptide has at least 70%, at least 80%, at least 85%, at least 90%, at least 95%, at least 96%, at least 97%, at least 98% or at least 99% sequence identity to the CR domain of a wild-type ephrin receptor (e.g., an ephrin receptor CR domain comprising an amino acid sequence selected from the group consisting of SEQ ID NOs:18-31). In some embodiments, the CR domain of the polypeptide is the CR domain of a wild-type ephrin receptor (e.g., an ephrin receptor CR domain comprising an amino acid sequence selected from the group consisting of SEQ ID NOs: 18-31).

In one aspect, the polypeptide described herein may comprise a CRD and said CRD exhibits a three dimensional structure that can be superimposed with the CRD structure of a wild type ephrin receptor. In certain embodiments, the polypeptide described herein may comprise a CRD and said CRD exhibits a three dimensional structure, whose portion between equivalent Ca positions can be superimposed with a wild type Eph receptor CRD with root-mean-square deviations (RMSDs) of at most 1, 2, 4, 4, 5, 6, 7, 8, 9 or 10 Å.

TABLE 4
CRD Sequences.

| | Protein | | |
|---|---|---|---|
| SEQ | (UniProt | Re- | |
| ID: | ID No.) | gion | Sequence |
| 18 | EPHA1 | 191- | CVALVSVRVFYQRCPETLNGLAQFPDTL |
| | (P21709) | 329 | PGPAGLVEVAGTCLPHARASPRPSGAPR |
| | | | MHCSPDGEWLVPVGRCHCEPGYEEGGSG |
| | | | EACVACPSGSYRMDMDTPHCLTCPQQST |
| | | | AESEGATICTCESGHYRAPGEGPQVAC |
| 19 | EPHA2 | 188- | CVALLSVRVYYKKCPELLQGLAHFPETI |
| | (P29317) | 325 | AGSDAPSLATVAGTCVDHAVVPPGGEEP |
| | | | RMHCAVDGEWLVPIGQCLCQAGYEKVED |
| | | | ACQACSPGFFKFEASESPCLECPEHTLP |
| | | | SPEGATSCECEEGFFRAPQDPASMPC |
| 20 | EPHA3 | 189- | CVALVSVRVYFKKCPFTVKNLAMFPDTV |
| | (P29320) | 322 | PMDSQSLVEVRGSCVNNSKEEDPPRMYC |
| | | | STEGEWLVPIGKCSCNAGYEERGFMCQA |
| | | | CRPGFYKALDGNMKCAKCPPHSSTQEDG |
| | | | SMNCRCENNYFRADKDPPSMAC |
| 21 | EPHA4 | 191- | CIALVSVRVFYKKCPLTVRNLAQFPDTI |
| | (P54764) | 325 | TGADTSSLVEVRGSCVNNSEEKDVPKMY |
| | | | CGADGEWLVPIGNCLCNAGHEERSGECQ |
| | | | ACKIGYYKALSTDATCAKCPPHSYSVWE |
| | | | GATSCTCDRGFFRADNDAASMPC |
| 22 | EPHA5 | 220- | CIALVSVRVYYKKCPSVVRHLAVFPDTI |
| | (P54756) | 354 | TGADSSQLLEVSGSCVNHSVTDEPPKMH |
| | | | CSAEGEWLVPIGKCMCKAGYEEKNGTCQ |
| | | | VCRPGFFKASPHIQSCGKCPPHSYTHEE |
| | | | ASTSCVCEKDYFRRESDPPTMAC |
| 23 | EPHA6 | 194- | CIALVSVRVFYKKCPFTVRNLAMFPDTI |
| | (Q9UF33) | 328 | PRVDSSSLVEVRGSCVKSAEERDTPKLY |
| | | | CGADGDWLVPLGRCICSTGYEEIEGSCH |
| | | | ACRPGFYKAFAGNTKCSKCPPHSLTYME |
| | | | ATSVCQCEKGYFRAEKDPPSMAC |
| 24 | EPHA7 | 192- | CIALVSVKVYYKKCWSIIENLAIFPDTV |
| | (Q15375) | 328 | TGSEFSSLVEVRGTCVSSAEEEAENAPR |
| | | | MHCSAEGEWLVPIGKCICKAGYQQKGDT |
| | | | CEPCGRGFYKSSSQDLQCSRCPTHSFSD |
| | | | KEGSSRCECEDGYYRAPSDPPYVAC |
| 25 | EPHA8 | 191- | CLAILSLRIYYKKCPAMVRNLAAFSEAV |
| | (P29322) | 325 | TGADSSSLVEVRGQCVRHSEERDTPKMY |
| | | | CSAEGEWLVPIGKCVCSAGYEERRDACV |
| | | | ACELGFYKSAPGDQLCARCPPHSHSAAP |
| | | | AAQACHCDLSYYRAALDPPSSAC |
| 26 | EPHA10 | 198- | CVALVSVRVYYKQCRATVRGLATFPATA |
| | (Q5JZY3) | 334 | AESAFSTLVEVAGTCVAHSEGEPGSPPR |
| | | | MHCGADGEWLVPVGRCSCSAGFQERGDF |
| | | | CEACPPGFYKVSPRRPLCSPCPEHSRAL |
| | | | ENASTFCVCQDSYARSPTDPPSASC |
| 27 | EPHB1 | 183- | CMSLLSVRVFFKKCPSIVQNFAVFPETM |
| | (P54762) | 319 | TGAESTSLVIARGTCIPNAEEVDVPIKL |
| | | | YCNGDGEWMVPIGRCTCKPGYEPENSVA |
| | | | CKACPAGTFKASQEAEGCSHCPSNSRSP |
| | | | AEASPICTCRTGYYRADFDPPEVAC |
| 28 | EPHB2 | 184- | CMSLIAVRVFYRKCPRIIQNGAIFQETL |
| | (P29323) | 324 | SGAESTSLVAARGSCIANAEEVDVPIKL |
| | | | YCNGDGEWLVPIGRCMCKAGFEAVENGT |
| | | | VCRGCPSGTFKANQGDEACTHCPINSRT |
| | | | TSEGATNCVCRNGYYRADLDPLDMPCTT |
| | | | I |
| 29 | EPHB3 | 199- | CMSLISVRAFYKKCASTTAGFALFPETL |
| | (P54753) | 336 | TGAEPTSLVIAPGTCIPNAVEVSVPLKL |
| | | | YCNGDGEWMVPVGACTCATGHEPAAKES |
| | | | QCRPCPPGSYKAKQGEGPCLPCPPNSRT |
| | | | TSPAASICTCHNNFYRADSDSADSAC |
| 30 | EPHB4 | 184- | CMALLSLHLFYKKCAQLTVNLTRFPETV |
| | (P54760) | 320 | PRELVVPVAGSCVVDAVPAPGPSPSLYC |
| | | | REDGQWAEQPVTGCSCAPGFEAAEGNTK |
| | | | CRACAQGTFKPLSGEGSCQPCPANSHSN |
| | | | TIGSAVCQCRVGYFRARTDPRGAPC |
| 31 | EPHB6 | 219- | CLALVAVRLFSYTCPAVLRSFASFPETQ |
| | (O15197) | 366 | ASGAGGASLVAAVGTCVAHAEPEEDGVG |
| | | | GQAGGSPPRLHCNGEGKWMVAVGGCRCQ |
| | | | PGYQPARGDKACQACPRGLYKSSAGNAP |
| | | | CSPCPARSHAPNPAAPVCPCLEGFYRAS |
| | | | SDPPEAPC |

In some embodiments, the CR domain comprises the amino acid sequence of the CR domain of a wild-type ephrin receptor (e.g., an ephrin receptor CR domain comprising an amino acid sequence selected from the group consisting of SEQ ID NOs: 18-31) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the FN1 domain of the polypeptide has at least 70%, at least 80%, at least 85%, at least 90%, at least 95%, at least 96%, at least 97%, at least 98% or at least 99% sequence identity to the FN1 domain of a wild-type ephrin receptor (e.g., an ephrin receptor FN1 domain comprising an amino acid sequence selected from the group consisting of SEQ ID NOs:32-45). In some embodiments, the FN1 domain of the polypeptide is the FN1 domain of a wild-type ephrin receptor (e.g., an ephrin receptor FN1 domain comprising an amino acid sequence selected from the group consisting of SEQ TD NOs: 32-45).

In one aspect, the polypeptide described herein may comprise a FN1 and said FN1 exhibits a three dimensional structure that can be superimposed with the FN1 structure of a wild type ephrin receptor. In certain embodiments, the polypeptide described herein may comprise a FN1 and said FN1 exhibits a three dimensional structure, whose portion between equivalent Cα positions can be superimposed with a wild type Eph receptor FN1 with root-mean-square deviations (RMSDs) of at most 1, 2, 4, 4, 5, 6, 7, 8, 9 or 10 Å.

TABLE 5
FNIII-1 (CDD: 365830 ProRule: PRU00316).

| | Protein | | | |
|---|---|---|---|---|
| SEQ | (UniProt | NCBI | Re- | |
| ID: | ID No.) | CDD # | gion | Sequence |
| 32 | EPHA1 | 365830 | 332- | PPSAPRNLSFSASGTQLSLRWEP |
| | (P21709) | | 445 | PADTGGRQDVRYSVRCSQCQGTA |
| | | | | QDGGPCQPCGVGVHFSPGARGLT |
| | | | | TPAVHVNGLEPYANYTENVEAQN |
| | | | | GVSGLGSSGHASTSVSISMGHA |
| 33 | EPHA2 | 365830 | 328- | PPSAPHYLTAVGMGAKVELRWTP |
| | (P29317) | | 432 | PQDSGGREDIVYSVTCEQCWPES |
| | | | | GECGPCEASVRYSEPPHGLTRTS |
| | | | | VTVSDLEPHMNYTFTVEARNGVS |
| | | | | GLVTSRSFRTASV |
| 34 | EPHA3 | 365830 | 325- | PPSSPRNVISNINETSVILDWSW |
| | (P29320) | | 435 | PLDTGGRKDVTFNIICKKCGWNI |
| | | | | KQCEPCSPNVRFLPRQFGLTNTT |
| | | | | VTVTDLLAHTNYTFEIDAVNGVS |
| | | | | ELSSPPRQFAAVSITTNQA |
| 35 | EPHA4 | 365830 | 328- | PPSAPLNLISNVNETSVNLEWSS |
| | (P54764) | | 439 | PQNTGGRQDISYNVVCKKCGAGD |
| | | | | PSKCRPCGSGVHYTPQQNGLKTT |
| | | | | KVSITDLLAHTNYTFEIWAVNGV |
| | | | | SKYNPNPDQSVSVTVTTNQA |
| 36 | EPHA5 | 365830 | 357- | PPSAPRNAISNVNETSVFLEWIP |
| | (P54756) | | 467 | PADTGGRKDVSYYIACKKCNSHA |
| | | | | GVCEECGGHVRYLPRQSGLKNTS |
| | | | | VMMVDLLAHTNYTFEIEAVNGVS |
| | | | | DLSPGARQYVSVNVTTNQA |
| 37 | EPHA6 | 365830 | 331- | PPSAPRNVVFNINETALILEWSP |
| | (Q9UF33) | | 441 | PSDTGGRKDLTYSVICKKCGLDT |
| | | | | SQCEDCGGGLRFIPRHTGLINNS |
| | | | | VIVLDFVSHVNYTFEIEAMNGVS |
| | | | | ELSFSPKPFTAITVTTDQD |
| 38 | EPHA7 | 365830 | 331- | PPSAPQNLIFNINQTTVSLEWSP |
| | (Q15375) | | 441 | PADNGGRNDVTYRILCKRCSWEQ |
| | | | | GECVPCGSNIGYMPQQTGLEDNY |
| | | | | VTVMDLLAHANYTFEVEAVNGVS |
| | | | | DLSRSQRLFAAVSITTGQA |
| 39 | EPHA8 | 365830 | 328- | PPSAPVNLISSVNGTSVTLEWAP |
| | (P29322) | | 438 | PLDPGGRSDITYNAVCRRCPWAL |
| | | | | SRCEACGSGTRFVPQQTSLVQAS |
| | | | | LLVANLLAHMNYSFWIEAVNGVS |
| | | | | DLSPEPRRAAVVNITTNQA |
| 40 | EPHA10 | 365830 | 340- | APRDLQYSLSRSPLVLRLRWLPP |
| | (Q5JZY3) | | 452 | ADSGGRSDVTYSLLCLRCGREGP |
| | | | | AGACEPCGPRVAFLPRQAGLRER |
| | | | | AATLLHLRPGARYTVRVAALNGV |
| | | | | SGPAAAAGTTYAQVTVSTGPG |
| 41 | EPHB1 | 365830 | 322- | VPSGPRNVISIVNETSIILEWHP |
| | (P54762) | | 432 | PRETGGRDDVTYNIICKKCRADR |
| | | | | RSCSRCDDNVEFVPRQLGLTECR |
| | | | | VSISSLWAHTPYTFDIQAINGVS |
| | | | | SKSPFPPQHVSVNITTNQA |
| 42 | EPHB2 | 365830 | 324- | IPSAPQAVISSVNETSLMLEWTP |
| | (P29323) | | 434 | PRDSGGREDLVYNIICKSCGSGR |
| | | | | GACTRCGDNVQYAPRQLGLTEPR |
| | | | | IYISDLLAHTQYTFEIQAVNGVT |
| | | | | DQSPFSPQFASVNITTNQA |
| 43 | EPHB3 | 365830 | 339- | VPSPPRGVISNVNETSLILEWSE |
| | (P54753) | | 451 | PRDLGGRDDLLYNVICKKCHGAG |
| | | | | GASACSRCDDNVEFVPRQLGLTE |
| | | | | RRVHISHLLAHTRYTFEVQAVNG |
| | | | | VSGKSPLPPRYAAVNITTNQA |
| 44 | EPHB4 | 365830 | 323- | PPSAPRSVVSRLNGSSLHLEWSA |
| | (P54760) | | 432 | PLESGGREDLTYALRCRECRPGG |
| | | | | SCAPCGGDLTFDPGPRDLVEPWV |
| | | | | VVRGLRPDFTYTFEVTALNGVSS |
| | | | | LATGPVPFEPVNVTTDRE |
| 45 | EPHB6 | 365830 | 369- | PPSAPQELWFEVQGSALMLHWRL |
| | (O15197) | | 486 | PRELGGRGDLLFNVVCKECEGRQ |
| | | | | EPASGGGGTCHRCRDEVHFDPRQ |
| | | | | RGLTESRVLVGGLRAHVPYILEV |
| | | | | QAVNGVSELSPDPPQAAAINVST |
| | | | | SHE |

In some embodiments, the FN1 domain comprises the amino acid sequence of the FN1 domain of a wild-type ephrin receptor (e.g., an ephrin receptor FN1 domain comprising an amino acid sequence selected from the group consisting of SEQ ID NOs: 32-45) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the FN2 domain of the polypeptide has at least 70%, at least 80%, at least 85%, at least 90%, at least 95%, at least 96%, at least 97%, at least 98% or at least 99% sequence identity to the FN2 domain of a wild-type ephrin receptor (e.g., an ephrin receptor FN2 domain comprising an amino acid sequence selected from the group consisting of SEQ ID NOs: 46-59). In some embodiments, the FN2 domain of the polypeptide is the FN2 domain of a wild-type ephrin receptor (e.g., an ephrin receptor FN2 domain comprising an amino acid sequence selected from the group consisting of SEQ ID NOs: 46-59).

In one aspect, the polypeptide described herein may comprise a FN2 and said FN2 exhibits a three dimensional structure that can be superimposed with the FN2 structure of a wild type ephrin receptor. In certain embodiments, the polypeptide described herein may comprise a FN2 and said FN2 exhibits a three dimensional structure, whose portion between equivalent Cα positions can be superimposed with a wild type Eph receptor FN2 with root-mean-square deviations (RMSDs) of at most 1, 2, 4, 4, 5, 6, 7, 8, 9 or 10 Å.

TABLE 6
FNIII-2 (CDD: 365830 ProRule: PRU00316).

| | Protein | | | |
|---|---|---|---|---|
| SEQ | (UniProt | NCBI | Re- | |
| ID: | ID No.) | CDD # | gion | Sequence |
| 46 | EPHA1 | 365830 | 446- | ESLSGLSLRLVKKEPRQLELTWAGS |
| | (P21709) | | 538 | RPRSPGANLTYELHVLNQDEERYQM |
| | | | | VLEPRVLLTELQPDTTYIVRVRMLT |
| | | | | PLGPGPFSPDHEFRTSPP |
| 47 | EPHA2 | 365830 | 438- | EPPKVRLEGRSTTSLSVSWSIPPPQ |
| | (P29317) | | 529 | QSRVWKYEVTYRKKGDSNSYNVRRT |
| | | | | EGFSVTLDDLAPDTTYLVQVQALTQ |
| | | | | EGQGAGSKVHEFQTLSP |
| 48 | EPHA3 | 365830 | 436- | APSPVLTIKKDRTSRNSISLSWQEP |
| | (P29320) | | 531 | EHPNGIILDYEVKYYEKQEQETSYT |
| | | | | ILRARGTNVTISSLKPDTIYVFQIR |
| | | | | ARTAAGYGTNSRKFEFETSPD |
| 49 | EPHA4 | 365830 | 440- | APSSIALVQAKEVTRYSVALAWLEP |
| | (P54764) | | 537 | DRPNGVILEYEVKYYEKDQNERSYR |
| | | | | IVRTAARNTDIKGLNPLTSYVFHVR |
| | | | | ARTAAGYGDFSEPLEVTTNTVPS |
| 50 | EPHA5 | 365830 | 468- | APSPVTNVKKGKIAKNSISLSWQEP |
| | (P54756) | | 562 | DRPNGIILEYEIKYFEKDQETSYTI |
| | | | | IKSKETTITAEGLKPASVYVFQIRA |
| | | | | RTAAGYGVFSRRFEFETTPV |
| 51 | EPHA6 | 365830 | 442- | APSLIGVVRKDWASQNSIALSWQAP |
| | (Q9UF33) | | 537 | AFSNGAILDYEIKYYEKEHEQLTYS |
| | | | | STRSKAPSVIITGLKPATKYVFHIR |
| | | | | VRTATGYSGYSQKFEFETGDE |
| 52 | EPHA7 | 365830 | 442- | APSQVSGVMKERVLQRSVELSWQEP |
| | (Q15375) | | 537 | EHPNGVITEYEIKYYEKDQRERTYS |
| | | | | TVKTKSTSASINNLKPGTVYVFQIR |
| | | | | AFTAAGYGNYSPRLDVATLEE |
| 53 | EPHA8 | 365830 | 439- | APSQVVVIRQERAGQTSVSLLWQEP |
| | (P29322) | | 534 | EQPNGIILEYEIKYYEKDKEMQSYS |
| | | | | TLKAVTTRATVSGLKPGTRYVFQVR |
| | | | | ARTSAGCGRFSQAMEVETGKP |
| 54 | EPHA10 | 365830 | 453- | APWEEDEIRRDRVEPQSVSLSWREP |
| | (Q5JZY3) | | 554 | IPAGAPGANDTEYEIRYYEKGQSEQ |
| | | | | TYSMVKTGAPTVTVTNLKPATRYVF |
| | | | | QIRAASPGPSWEAQSFNPSIEVQTL |
| | | | | GE |
| 55 | EPHB1 | 365830 | 433- | APSTVPIMHQVSATMRSITLSWPQP |
| | (P54762) | | 528 | EQPNGIILDYEIRYYEKEHNEFNSS |
| | | | | MARSQTNTARIDGLRPGMVYVVQVR |
| | | | | ARTVAGYGKFSGKMCFQTLTD |
| 56 | EPHB2 | 365830 | 435- | APSAVSIMHQVSRTVDSITLSWSQP |
| | (P29323) | | 530 | DQPNGVILDYELQYYEKELSEYNAT |
| | | | | AIKSPTNTVTVQGLKAGAIYVFQVR |
| | | | | ARTVAGYGRYSGKMYFQTMTE |
| 57 | EPHB3 | 365830 | 452- | APSEVPTLRLHSSSGSSLTLSWAPP |
| | (P54753) | | 545 | ERPNGVILDYEMKYFEKSEGIASTV |
| | | | | TSQMNSVQLDGLRPDARYVVQVRAR |
| | | | | TVAGYGQYSRPAEFETTSE |
| 58 | EPHB4 | 365830 | 433- | VPPAVSDIRVTRSSPSSLSLAWAVP |
| | (P54760) | | 529 | RAPSGAVLDYEVKYHEKGAEGPSSV |
| | | | | RFLKTSENRAELRGLKRGASYLVQV |
| | | | | RARSEAGYGPFGQEHHSQTQLD |
| 59 | EPHB6 | 365830 | 487- | VPSAVPVVHQVSRASNSITVSWPQP |
| | (O15197) | | 582 | DQTNGNILDYQLRYYDQAEDESHSF |
| | | | | TLTSETNTATVTQLSPGHIYGFQVR |
| | | | | ARTAAGHGPYGGKVYFQTLPQ |

In some embodiments, the FN2 domain comprises the amino acid sequence o the FN2 domain of a wild-type ephrin receptor (e.g., an ephrin receptor FN2 domain comprising an amino acid sequence selected from the group consisting of SEQ ID NOs: 46-59) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In certain embodiments, the first ephrin receptor FN III domain (i.e., FN1) and the second ephrin receptor FN III domain (i.e., FN2) comprise different amino acid sequences. In certain embodiments, the first ephrin receptor FN III domain (i.e., FN1) and the second ephrin receptor FN III domain (i.e., FN2) comprise the same amino acid sequence.

In one aspect, the polypeptide described herein may comprise a flexible domain and said flexible domain exhibits a three dimensional structure that can be superimposed with the flexible domain structure of a wild type ephrin receptor. In certain embodiments, the polypeptide described herein may comprise a flexible domain and said flexible domain exhibits a three dimensional structure, whose portion between equivalent Ca positions can be superimposed with a wild type Eph receptor flexible with root-mean-square deviations (RMSDs) of at most 1, 2, 4, 4, 5, 6, 7, 8, 9 or 10 Å.

In some embodiments, the flexible domain of the polypeptide has at least 70%, at least 80%, at least 85%, at least 90%, at least 95%, at least 96%, at least 97%, at least 98% or at least 99% sequence identity to the flexible domain of a wild-type ephrin receptor (e.g., an ephrin receptor flexible domain comprising an amino acid sequence selected from the group consisting of SEQ ID NOs:60-73). In some embodiments, the flexible domain of the polypeptide is the flexible domain of a wild-type ephrin receptor (e.g., an ephrin receptor flexible domain comprising an amino acid sequence selected from the group consisting of SEQ ID NOs: 60-73).

TABLE 7
Exemplary Flexible Domains (CRD + FN1 + FN2).

| | Protein | |
|---|---|---|
| SEQ | (UniProt | |
| ID: | ID No.) | Sequence |
| 60 | EPHA1 | CPETLNGLAQFPDTLPGPAGLVEVAGTCLP |
| | (P21709) | HARASPRPSGAPRMHCSPDGEWLVPVGRCH |
| | | CEPGYEEGGSGEACVACPSGSYRMDMDTPH |
| | | CLTCPQQSTAESEGATICTCESGHYRAPGE |
| | | GPQVACTGPPSAPRNLSFSASGTQLSLRWE |
| | | PPADTGGRQDVRYSVRCSQCQGTAQDGGPC |
| | | QPCGVGVHFSPGARGLTTPAVHVNGLEPYA |
| | | NYTFNVEAQNGVSGLGSSGHASTSVSISMG |
| | | HAESLSGLSLRLVKKEPRQLELTWAGSRPR |
| | | SPGANLTYELHVLNQDEERYQMVLEPRVLL |
| | | TELQPDTTYIVRVRMLTPLGPGPFSPDHEF |
| | | RTSPP |
| 61 | EPHA2 | CPELLQGLAHFPETIAGSDAPSLATVAGTC |
| | (P29317) | VDHAVVPPGGEEPRMHCAVDGEWLVPIGQC |
| | | LCQAGYEKVEDACQACSPGFFKFEASESPC |
| | | LECPEHTLPSPEGATSCECEEGFFRAPQDP |
| | | ASMPCTRPPSAPHYLTAVGMGAKVELRWTP |
| | | PQDSGGREDIVYSVTCEQCWPESGECGPCE |
| | | ASVRYSEPPHGLTRTSVTVSDLEPHMNYTF |
| | | TVEARNGVSGLVTSRSFRTASVSINQTEPP |
| | | KVRLEGRSTTSLSVSWSIPPPQQSRVWKYE |
| | | VTYRKKGDSNSYNVRRTEGFSVTLDDLAPD |
| | | TTYLVQVQALTQEGQGAGSKVHEFQTLSP |
| 62 | EPHA3 | CPFTVKNLAMFPDTVPMDSQSLVEVRGSCV |
| | (P29320) | NNSKEEDPPRMYCSTEGEWLVPIGKCSCNA |
| | | GYEERGFMCQACRPGFYKALDGNMKCAKCP |
| | | PHSSTQEDGSMNCRCENNYFRADKDPPSMA |
| | | CTRPPSSPRNVISNINETSVILDWSWPLDT |
| | | GGRKDVTFNIICKKCGWNIKQCEPCSPNVR |
| | | FLPRQFGLTNTTVTVTDLLAHTNYTFEIDA |
| | | VNGVSELSSPPRQFAAVSITTNQAAPSPVL |
| | | TIKKDRTSRNSISLSWQEPEHPNGIILDYE |
| | | VKYYEKQEQETSYTILRARGTNVTISSLKP |
| | | DTIYVFQIRARTAAGYGTNSRKFEFETSPD |
| 63 | EPHA4 | CPLTVRNLAQFPDTITGADTSSLVEVRGSC |
| | (P54764) | VNNSEEKDVPKMYCGADGEWLVPIGNCLCN |
| | | AGHEERSGECQACKIGYYKALSTDATCAKC |
| | | PPHSYSVWEGATSCTCDRGFFRADNDAASM |
| | | PCTRPPSAPLNLISNVNETSVNLEWSSPQN |
| | | TGGRQDISYNVVCKKCGAGDPSKCRPCGSG |
| | | VHYTPQQNGLKTTKVSITDLLAHTNYTFEI |
| | | WAVNGVSKYNPNPDQSVSVTVTTNQAAPSS |
| | | IALVQAKEVTRYSVALAWLEPDRPNGVILE |
| | | YEVKYYEKDQNERSYRIVRTAARNTDIKGL |
| | | NPLTSYVFHVRARTAAGYGDFSEPLEVTTN |
| | | TVPS |
| 64 | EPHA5 | CPSVVRHLAVFPDTITGADSSQLLEVSGSC |
| | (P54756) | VNHSVTDEPPKMHCSAEGEWLVPIGKCMCK |
| | | AGYEEKNGTCQVCRPGFFKASPHIQSCGKC |
| | | PPHSYTHEEASTSCVCEKDYFRRESDPPTM |
| | | ACTRPPSAPRNAISNVNETSVFLEWIPPAD |
| | | TGGRKDVSYYIACKKCNSHAGVCEECGGHV |
| | | RYLPRQSGLKNTSVMMVDLLAHTNYTFEIE |
| | | AVNGVSDLSPGARQYVSVNVTTNQAAPSPV |
| | | TNVKKGKIAKNSISLSWQEPDRPNGIILEY |
| | | EIKYFEKDQETSYTIIKSKETTITAEGLKP |
| | | ASVYVFQIRARTAAGYGVFSRRFEFETTPV |
| 65 | EPHA6 | CPFTVRNLAMFPDTIPRVDSSSLVEVRGSC |
| | (Q9UF33) | VKSAEERDTPKLYCGADGDWLVPLGRCICS |
| | | TGYEEIEGSCHACRPGFYKAFAGNTKCSKC |
| | | PPHSLTYMEATSVCQCEKGYFRAEKDPPSM |
| | | ACTRPPSAPRNVVFNINETALILEWSPPSD |
| | | TGGRKDLTYSVICKKCGLDTSQCEDCGGGL |
| | | RFIPRHTGLINNSVIVLDFVSHVNYTFEIE |
| | | AMNGVSELSFSPKPFTAITVTTDQDAPSLI |
| | | GVVRKDWASQNSIALSWQAPAFSNGAILDY |
| | | EIKYYEKEHEQLTYSSTRSKAPSVIITGLK |
| | | PATKYVFHIRVRTATGYSGYSQKFEFETGD |
| | | E |
| 66 | EPHA7 | CWSIIENLAIFPDTVTGSEFSSLVEVRGTC |
| | (Q15375) | VSSAEEEAENAPRMHCSAEGEWLVPIGKCI |
| | | CKAGYQQKGDTCEPCGRGFYKSSSQDLQCS |
| | | RCPTHSFSDKEGSSRCECEDGYYRAPSDPP |
| | | YVACTRPPSAPQNLIFNINQTTVSLEWSPP |
| | | ADNGGRNDVTYRILCKRCSWEQGECVPCGS |
| | | NIGYMPQQTGLEDNYVTVMDLLAHANYTFE |
| | | VEAVNGVSDLSRSQRLFAAVSITTGQAAPS |
| | | QVSGVMKERVLQRSVELSWQEPEHPNGVIT |
| | | EYEIKYYEKDQRERTYSTVKTKSTSASINN |
| | | LKPGTVYVFQIRAFTAAGYGNYSPRLDVAT |
| | | LEE |
| 67 | EPHA8 | CPAMVRNLAAFSEAVTGADSSSLVEVRGQC |
| | (P29322) | VRHSEERDTPKMYCSAEGEWLVPIGKCVCS |
| | | AGYEERRDACVACELGFYKSAPGDQLCARC |
| | | PPHSHSAAPAAQACHCDLSYYRAALDPPSS |
| | | ACTRPPSAPVNLISSVNGTSVTLEWAPPLD |
| | | PGGRSDITYNAVCRRCPWALSRCEACGSG |
| | | TRFVPQQTSLVQASLLVANLLAHMNYSFWI |
| | | EAVNGVSDLSPEPRRAAVVNITTNQAAPSQ |
| | | VVVIRQERAGQTSVSLLWQEPEQPNGIILE |
| | | YEIKYYEKDKEMQSYSTLKAVTTRATVSGL |
| | | KPGTRYVFQVRARTSAGCGRFSQAMEVETG |
| | | KP |
| 68 | EPHA10 | CRATVRGLATFPATAAESAFSTLVEVAGTC |
| | (Q5JZY3) | VAHSEGEPGSPPRMHCGADGEWLVPVGRCS |
| | | CSAGFQERGDFCEACPPGFYKVSPRRPLCS |
| | | PCPEHSRALENASTFCVCQDSYARSPTDPP |
| | | SASCTRPPSAPRDLQYSLSRSPLVLRLRWL |
| | | PPADSGGRSDVTYSLLCLRCGREGPAGACE |
| | | PCGPRVAFLPRQAGLRERAATLLHLRPGAR |
| | | YTVRVAALNGVSGPAAAAGTTYAQVTVSTG |
| | | PGAPWEEDEIRRDRVEPQSVSLSWREPIPA |
| | | GAPGANDTEYEIRYYEKGQSEQTYSMVKTG |
| | | APTVTVTNLKPATRYVFQIRAASPGPSWEA |
| | | QSFNPSIEVQTLGE |
| 69 | EPHB1 | CPSIVQNFAVFPETMTGAESTSLVIARGTC |
| | (P54762) | IPNAEEVDVPIKLYCNGDGEWMVPIGRCTC |
| | | KPGYEPENSVACKACPAGTFKASQEAEGCS |
| | | HCPSNSRSPAEASPICTCRTGYYRADFDPP |
| | | EVACTSVPSGPRNVISIVNETSIILEWHPP |
| | | RETGGRDDVTYNIICKKCRADRRSCSRCDD |
| | | NVEFVPRQLGLTECRVSISSLWAHTPYTFD |
| | | IQAINGVSSKSPFPPQHVSVNITTNQAAPS |
| | | TVPIMHQVSATMRSITLSWPQPEQPNGIIL |
| | | DYEIRYYEKEHNEFNSSMARSQTNTARIDG |
| | | LRPGMVYVVQVRARTVAGYGKFSGKMCFQT |
| | | LTD |
| 70 | EPHB2 | CPRIIQNGAIFQETLSGAESTSLVAARGSC |
| | (P29323) | IANAEEVDVPIKLYCNGDGEWLVPIGRCMC |
| | | KAGFEAVENGTVCRGCPSGTFKANQGDEAC |
| | | THCPINSRTTSEGATNCVCRNGYYRADLDP |
| | | LDMPCTTIPSAPQAVISSVNETSLMLEWTP |
| | | PRDSGGREDLVYNIICKSCGSGRGACTRCG |
| | | DNVQYAPRQLGLTEPRIYISDLLAHTQYTF |
| | | EIQAVNGVTDQSPFSPQFASVNITTNQAAP |
| | | SAVSIMHQVSRTVDSITLSWSQPDQPNGVI |
| | | LDYELQYYEKELSEYNATAIKSPTNTVTVQ |
| | | GLKAGAIYVFQVRARTVAGYGRYSGKMYFQ |
| | | TMTE |
| 71 | EPHB3 | CASTTAGFALFPETLTGAEPTSLVIAPGTC |
| | (P54753) | IPNAVEVSVPLKLYCNGDGEWMVPVGACTC |
| | | ATGHEPAAKESQCRPCPPGSYKAKQGEGPC |
| | | LPCPPNSRTTSPAASICTCHNNFYRADSDS |
| | | ADSACTTVPSPPRGVISNVNETSLILEWSE |
| | | PRDLGGRDDLLYNVICKKCHGAGGASACSR |
| | | CDDNVEFVPRQLGLTERRVHISHLLAHTRY |
| | | TFEVQAVNGVSGKSPLPPRYAAVNITTNQA |
| | | APSEVPTLRLHSSSGSSLTLSWAPPERPNG |
| | | VILDYEMKYFEKSEGIASTVTSQMNSVQLD |
| | | GLRPDARYVVQVRARTVAGYGQYSRPAEFE |
| | | TTSE |
| 72 | EPHB4 | CAQLTVNLTRFPETVPRELVVPVAGSCVVD |
| | (P54760) | AVPAPGPSPSLYCREDGQWAEQPVTGCSCA |
| | | PGFEAAEGNTKCRACAQGTFKPLSGEGSCQ |
| | | PCPANSHSNTIGSAVCQCRVGYFRARTDPR |
| | | GAPCTTPPSAPRSVVSRLNGSSLHLEWSAP |
| | | LESGGREDLTYALRCRECRPGGSCAPCGGD |
| | | LTFDPGPRDLVEPWVVVRGLRPDFTYTFEV |
| | | TALNGVSSLATGPVPFEPVNVTTDREVPPA |
| | | VSDIRVTRSSPSSLSLAWAVPRAPSGAVLD |
| | | YEVKYHEKGAEGPSSVRFLKTSENRAELRG |
| | | LKRGASYLVQVRARSEAGYGPFGQEHHSQT |
| | | QLD |
| 73 | EPHB6 | CPAVLRSFASFPETQASGAGGASLVAAVGT |
| | (O15197) | CVAHAEPEEDGVGGQAGGSPPRLHCNGEGK |
| | | WMVAVGGCRCQPGYQPARGDKACQACPRGL |
| | | YKSSAGNAPCSPCPARSHAPNPAAPVCPCL |
| | | EGFYRASSDPPEAPCTGPPSAPQELWFEVQ |
| | | GSALMLHWRLPRELGGRGDLLFNVVCKECE |
| | | GRQEPASGGGGTCHRCRDEVHFDPRQRGLT |
| | | ESRVLVGGLRAHVPYILEVQAVNGVSELSP |
| | | DPPQAAAINVSTSHEVPSAVPVVHQVSRAS |
| | | NSITVSWPQPDQTNGNILDYQLRYYDQAED |
| | | ESHSFTLTSETNTATVTQLSPGHIYGFQVR |
| | | ARTAAGHGPYGGKVYFQTLPQ |

(c) Transmembrane (TM) Domain

Transmembrane domains of the disclosure are polypeptide domains of membrane-bound proteins or transmembrane proteins that comprise one or more transmembrane regions that are embedded in and traverse at least once a cellular membrane. Such a transmembrane region or a functional fragment thereof may be used as membrane anchors of a polypeptide of the disclosure (in particular, an Eph receptor derived polypeptide). A transmembrane domain useful in such polypeptide of the disclosure may originate from a transmembrane protein that is associated with any of a variety of membranes of a cell including, but not limited to, a plasma membrane, an endoplasmic reticulum membrane, a Golgi complex membrane, a lysosomal membrane, a nuclear membrane, and a mitochondrial membrane. In a certain embodiment, the transmembrane domain is derived from a mammal protein, preferably a human protein.

A transmembrane domain in a polypeptide of the disclosure (in particular, an Eph receptor derived polypeptide) comprises all or part of a transmembrane region of a transmembrane protein that normally traverses the membrane of a cell with which the transmembrane protein is normally associated. A transmembrane domain may comprise not only a membrane-spanning region of a transmembrane protein but also additional amino acids of the transmembrane protein that are located in flanking regions, either upstream (N-terminal) and/or downstream (C-terminal) to the membrane-spanning or membrane-embedded region of the transmembrane protein. For example, in particular embodiments, the entire transmembrane region of a transmembrane protein will be used. In additional embodiments, the entire transmembrane region and all or part of any upstream or downstream region of the membrane-embedded portion of a transmembrane protein may be used. Additional amino acids located either upstream (N-terminal) and/or downstream (C-terminal) from the membrane-embedded portion of a transmembrane protein that may be part of a transmembrane domain of a polypeptide of the disclosure (in particular, an Eph receptor derived polypeptide) may have a range of sizes including, but not limited to, 1 to 10 amino acids, 1 to 20 amino acids, 1 to 30 amino acids, 1 to 40 amino acids, 1 to 50 amino acids, 1 to 60 amino acids, 1 to 70 amino acids, 1 to 80 amino acids, 1 to 90 amino acids, 1 to 100 amino acids, 1 to 200 amino acids, 1 to 300 amino acids, 1 to 400 amino acids, 1 to 500 amino acids, 1 to 600 amino acids, 1 to 700 amino acids, 1 to 800 amino acids, and 1 to 900 amino acids. In some embodiments, a transmembrane domain lacks at least 5, 10, 50, 100, 200, 300, 400, 500, 600, 700, or 800 amino acids from the N-terminus of the native transmembrane protein. In some embodiments, a transmembrane domain lacks at least 5, 10, 50, 100, 200, 300, 400, 500, 600, 700, or 800 amino acids from the C-terminus of the native transmembrane protein. In some embodiments, a transmembrane domain lacks at least 5, 10, 50, 100, 200, 300, 400, 500, 600, 700, or 800 amino acids from both the N-terminus and C-terminus of the native transmembrane protein. In some embodiments, a transmembrane domain lacks one or more functional or structural domains of the native transmembrane protein.

The transmembrane domain is, in some embodiments, the transmembrane domain of a parental Eph receptor (e.g., an Eph receptor TM domain, such as an Eph receptor TM domain having an amino acid sequence selected from the group consisting of SEQ ID NOs:74-87). A transmembrane domain of a polypeptide described herein may also comprise the entire cytoplasmic region attached to a transmembrane region of a transmembrane protein or a truncation of the cytoplasmic region by one or more amino acids, for example, to eliminate an undesired signaling function of the cytoplasmic tail. As described above, if the membrane-embedded (transmembrane) region and all or part of the adjacent cytoplasmic C-terminal region of an kinase transmembrane protein is to be used as a transmembrane domain of a fusion protein of the disclosure, any known functional kinase signal should be eliminated or disrupted so that a fusion protein comprising the transmembrane region and any adjacent cytoplasmic region does not activate the host cell.

In some embodiments, the transmembrane domain comprises an amino acid sequence that is at least 70%, at least 75%, at least 80%, at least 85%, at least 90%, at least 95%, at least 96%, at least 97%, at least 98%, at least 99%, or 100% identical to the transmembrane domain of a wild-type ephrin receptor (e.g., an ephrin receptor TM domain comprising an amino acid sequence selected from the group consisting of SEQ ID NOs: 74-87). In some embodiments, the transmembrane domain of the polypeptide is the transmembrane domain of a wild-type ephrin receptor (e.g., an ephrin receptor TM domain comprising an amino acid sequence selected from the group consisting of SEQ ID NOs: 74-87).

TABLE 8
TM domains.

| | Protein | | |
|---|---|---|---|
| SEQ | (UniProt | | |
| ID: | ID No.) | Region | Sequence |
| 74 | EPHA1 | 548-568 | IVAVIFGLLLGAALLLGILVF |
| | (P21709) | | |
| 75 | EPHA2 | 538-558 | IGGVAVGVVLLLVLAGVGFFI |
| | (P29317) | | |
| 76 | EPHA3 | 542-565 | VVMIAISAAVAIILLTVVIYV |
| | (P29320) | | LIG |
| 77 | EPHA4 | 548-569 | VLLVSVSGSVVLVVILIAAFV |
| | (P54764) | | I |
| 78 | EPHA5 | 574-594 | VIAVSVTVGVILLAVVIGVLL |
| | (P54756) | | |
| 79 | EPHA6 | 551-571 | IATAAVGGFTLLVILTLFFLI |
| | (Q9UF33) | | |
| 80 | EPHA7 | 556-576 | IIIAVVAVAGTIILVFMVFGF |
| | (Q15375) | | |
| 81 | EPHA8 | 543-563 | IVWICLTLITGLVVLLLLLIC |
| | (P29322) | | |
| 82 | EPHA10 | 566-586 | IVVTVVTISALLVLGSVMSVL |
| | (Q5JZY3) | | |
| 83 | EPHB1 | 541-563 | LIAGSAAAGVVFVVSLVAISI |
| | (P54762) | | VC |
| 84 | EPHB2 | 544-564 | IIGSSAAGLVFLIAVVVIAIV |
| | (P29323) | | |
| 85 | EPHB3 | 560-580 | IVGSATAGLVFVVAVVVIAIV |
| | (P54753) | | |
| 86 | EPHB4 | 540-560 | LIAGTAVVGVVLVLVVIVVAV |
| | (P54760) | | |
| 87 | EPHB6 | 595-615 | LVIGSILGALAFLLLAAITVL |
| | (015197) | | |

In some embodiments, the transmembrane domain comprises the amino acid sequence of the transmembrane domain of a wild-type ephrin receptor (e.g., an ephrin receptor TM domain comprising an amino acid sequence selected from the group consisting of SEQ ID NOs: 74-87) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the polypeptide comprises a transmembrane domain homo-domain dimerization motif which increases interaction between two or more of the polypeptides at the transmembrane domain. In certain embodiments, the transmembrane domain homo-domain dimer motif is a transmembrane leucine zipper motif. In certain embodiments, the transmembrane domain homo-dimer motif is a transmembrane glycine zipper motif. Methods to modify and assay transmembrane domain dimerization are known in the art, see, e.g., Bocharov et al. J Biol Chem. 2008 Oct. 24; 283(43):29385-95.

In some embodiments, the transmembrane domain comprises the amino acid sequence of the transmembrane domain of a wild-type ephrin receptor (e.g., an ephrin receptor TM domain comprising an amino acid sequence selected from the group consisting of SEQ ID NOs: 74-87) and its length is 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of the amino acid sequence of the transmembrane domain of the wild-type ephrin receptor (e.g., SEQ ID NOs: 74-87).

Knowing that a transmembrane region is derived from a particular type of transmembrane protein suggests a preferred orientation and location for the transmembrane domain relative to the polypeptide of the disclosure (in particular, an Eph receptor derived polypeptide). This is particularly important with respect to Type I and Type II transmembrane proteins, which have fixed orientations and locations for their N-and C-termini with respect to the cytoplasm and nanovesicle lumen on either side of the transmembrane region. For example, when a transmembrane region from a Type I transmembrane protein is used as the transmembrane domain of a polypeptide of the disclosure, the polypeptide is oriented at the distal position from the membrane. Thus, the most common configurations of a polypeptide of the present disclosure that have a Type I transmembrane protein-derived transmembrane domain will comprise an N-terminal to C-terminal linear structure illustrated as follows:
(1) (ectodomain)-L-(transmembrane domain),
where L in the formula represents a direct peptide bond linking two domains or a linker of one or more amino acid residues.

In a specific embodiment, a polypeptide described herein comprising a Type I-derived transmembrane domain preferably comprises an N-terminal signal sequence, which can direct the N-terminus of the polypeptide through the ER membrane and into the ER lumen.

As a non-limiting example, Eph receptor derived polypeptides described herein are constructed by inserting an ectodomain after the N-terminal signal peptide of a Type I derived transmembrane domain such as the one of EphA4 (SEQ ID NO:77) or a transmembrane domain that shares at least 70%, at least 80%, at least 85%, at least 90%, at least 95%, at least 96%, at least 97%, at least 98%, or at least 99% sequence identity with the transmembrane domain of EphA4 (SEQ ID NO: 77).

### 5.2.2 Endodomain (Juxtamembrane (JM) Domain, Kinase Domain (KD), Sterile α-Motif (SAM) Linker Domain, SAM Domain, and PDZ Binding Motif (PBM) Domain)

For a wild-type ephrin receptor, the ectodomain is connected by the transmembrane domain, which is extended intracellularly to a juxtamembrane (JM) domain that tethers the kinase domain, which is part of the endodomain.

As outline before, the endodomain of a wild-type ephrin receptor comprises a juxtamembrane (JM) domain, a kinase domain (KD), a sterile alpha motif (SAM) linker domain, a SAM domain, and a PDZ-binding motif (PBM) domain. Of the Eph receptors, EphB6 and EphA10 have alterations in essential motifs that contribute to their catalytic tyrosine kinase activity, leaving them catalytically defective. In some cases, Eph receptors can be naturally expressed splicing isoforms that do not comprise the KD, SAM linker domain, SAM domain and/or PBM domain, for example, isoforms of mouse EphA4 and EphA7.

The polypeptides of the disclosure may optionally comprise a JM domain, a KD, a SAM linker domain, a SAM domain, and/or a PBM domain or fragment(s) thereof. In certain embodiments, a polypeptide described herein comprises, in N-terminus to C-terminus direction, an ephrin receptor CR domain, two ephrin receptor FN III domains, a TM domain (e.g., an ephrin receptor TM domain), and further comprises one, two, three, four, or all of the following domains C-terminal to the TM domain (e.g., the ephrin receptor TM domain): ephrin receptor JM domain, ephrin receptor KD, SAM linker domain (e.g., ephrin receptor SAM linker domain), SAM domain (e.g., ephrin receptor SAM domain), and ephrin receptor PBM domain.

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., the ephrin receptor TM domain).

In some embodiments the polypeptides of the disclosure (in particular, Eph receptor derived polypeptides) are forward signaling incompetent. Accordingly, in some embodiments, the polypeptide lacks ephrin receptor kinase activity. In some embodiments, the polypeptide lacks the endodomain of the parental Eph receptor in its entirety. In some embodiments, the polypeptide lacks parts the endodomain of the parental Eph receptor. In some embodiments thereof, the polypeptide lacks the kinase domain of the parental Eph receptor or fragments thereof. In some embodiments thereof, the polypeptide lacks the tyrosine amino acids in the kinase domain of the parental Eph receptor. In some embodiments thereof, the polypeptide lacks the SAM linker domain of the parental Eph receptor. In some embodiments thereof, the polypeptide lacks the SAM domain of the parental Eph receptor. In some embodiments thereof, the polypeptide lacks the PBM domain of the parental Eph receptor.

Therefore, in specific embodiments, the polypeptides provided herein are signal neutral with regards to forward (i.e., luminal) signaling capacity of the receptor expressing cell. The capacity for forward signaling can be tested through methods known in the art, see, e.g., Germano, S., 2015. Receptor tyrosine kinases. Totowa, N.J.: Humana Press.

In certain embodiments, the cellular localization, ubiquitination and trafficking of the polypeptide can be redirected by modification of critical residues in the KD and JM domain. Methods to modify these processes are known in the art, e.g., as described in Sabet, O. et al. Ubiquitination switches EphA2 vesicular traffic from a continuous safeguard to a finite signalling mode. Nat. Commun. 6:804.

In certain embodiments, the polypeptides of the disclosure (in particular, Eph receptor derived polypeptides) lack both ephrin receptor kinase activity and ephrin binding activity. In specific embodiments, the polypeptides of the disclosure (in particular, Eph receptor derived polypeptides) lack both an ephrin receptor kinase domain and an ephrin receptor ligand binding domain. In specific embodiments, the polypeptides of the disclosure (in particular, Eph receptor derived polypeptides) lack an ephrin receptor kinase domain and comprise a modified ephrin receptor ligand binding domain as described in Section 5.2.1. In specific embodiments, the polypeptides of the disclosure (in particular, Eph receptor derived polypeptides) comprise a modified ephrin receptor kinase domain as described herein and lack an ephrin receptor ligand binding domain. In specific embodiments, the polypeptides of the disclosure (in particular, Eph receptor derived polypeptides) comprise a modified ephrin receptor kinase domain as described herein and comprise a modified ephrin receptor ligand binding domain as described in Section 5.2.1.

### (a) Juxtamembrane Domain

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., the ephrin receptor TM domain).

In some embodiments, the JM domain of the polypeptide has at least 70%, at least 80%, at least 85%, at least 90%, at least 95%, at least 96%, at least 97%, at least 98% or at least 99% sequence identity to the JM domain of a wild-type ephrin receptor (e.g., an ephrin receptor JM domain comprising an amino acid sequence selected from the group consisting of SEQ ID NOs: 88-101 in Table 9). In some embodiments, the JM domain of the polypeptide is the JM domain of a wild-type ephrin receptor (e.g., an ephrin receptor JM domain comprising an amino acid sequence selected from the group consisting of SEQ ID NOs: 88-101).

TABLE 9
JM (CDD Superfamily: cl25995,
ProRule: PRU00159).

| SEQ | Protein | | | |
|---|---|---|---|---|
| ID | (UniProt | NCBI | | |
| NO: | ID No.) | CDD # | Region | Sequence |
| 88 | EPHA1 | cl25995 | 571- | RRAQRQRQQRQRDRA |
| | (P21709) | | 619 | TDVDREDKLWLKPYV |
| | | | | DLQAYEDPAQGALDF |
| | | | | TREL |
| 89 | EPHA2 | 291255 | 559- | HRRRKNQRARQSPED |
| | (P29317) | | 608 | VYFSKSEQLKPLKTY |
| | | | | VDPHTYEDPNQAVL |
| | | | | KFTTEI |
| 90 | EPHA3 | 291255 | 564- | GRFCGYKSKHGADEK |
| | (P29320) | | 616 | RLHFGNGHLKLPGLR |
| | | | | TYVDPHTYEDPTQAV |
| | | | | HEFAKEL |
| 91 | EPHA4 | 317033 | 570- | SRRRSKYSKAKQEAD |
| | P54764 | | 616 | EEKHLNQGVRTYVDP |
| | | | | FTYEDPNQAVREFAK |
| | | | | EI |
| 92 | EPHA5 | 317033 | 595- | SGSCCECGCGRASSL |
| | (P54756) | | 670 | CAVAHPSLIWRCGYS |
| | | | | KAKQDPEEEKMHFHN |
| | | | | GHIKLPGVRTYIDPH |
| | | | | TYEDPNQAVHEFAKE |
| | | | | I |
| 93 | EPHA6 | 291255 | 572- | TGRCQWYIKAKMKSE |
| | (Q9UF33) | | 626 | EKRRNHLQNGHLRFP |
| | | | | GIKTYIDPDTYEDPS |
| | | | | LAVHEFAKEI |
| 94 | EPHA7 | 317033 | 579- | GRRHCGYSKADQEGD |
| | (Q15375) | | 628 | EELYFHFKFPGTKTY |
| | | | | IDPETYEDPNRAVHQ |
| | | | | FAKEL |
| 95 | EPHA8 | 291255 | 563- | KKRHCGYSKAFQDSD |
| | (P29322) | | 630 | EEKMHYQNGQAPPPV |
| | | | | FLPLHHPPGKLPEPQ |
| | | | | FYAEPHTYEEPGRAG |
| | | | | RSFTREI |
| 96 | EPHA10 | 291255 | 589- | RRPCSYGKGGGDAHD |
| | (Q5JZY3) | | 640 | EEELYFHFKVPTRRT |
| | | | | FLDPQSCGDLLQAVH |
| | | | | LFAKEL |
| 97 | EPHB1 | 291255 | 565- | RKRAYSKEAVYSDKL |
| | (P54762) | | 614 | QHYSTGRGSPGMKIY |
| | | | | IDPFTYEDPNEAVRE |
| | | | | FAKEI |
| 98 | EPHB2 | 291255 | 566- | RRGFERADSEYTDKL |
| | (P29323) | | 616 | QHYTSGHMTPGMKIY |
| | | | | IDPFTYEDPNEAVR |
| | | | | EFAKEI |
| 99 | EPHB3 | 317033 | 582- | RKQRHGSDSEYTEKL |
| | P54753) | | 628 | QQYIAPGMKVYIDPF |
| | | | | TYEDPNEAVREFAKE |
| | | | | I |
| 100 | EPHB4 | 317033 | 564- | RKQSNGREAEYSDKH |
| | (P54760) | | 610 | GQYLIGHGTKVYIDP |
| | | | | FTYEDPNEAVREFAK |
| | | | | EI |
| 101 | EPHB6 | 291255 | 620- | RKRRGTGYTEQLQQY |
| | (015197) | | 665 | SSPGLGVKYYIDPST |
| | | | | YEDPCQAIRELAREV |

In some embodiments, the JM domain comprises the amino acid sequence o t e JM domain of a wild-type ephrin receptor (e.g., an ephrin receptor JM domain comprising an amino acid sequence selected from the group consisting of SEQ ID NOs: 88-101) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In specific embodiments, the ephrin receptor JM domain comprises: (i) a (X₁)-Ptyr-(X₂) motif, wherein Ptyr is a phosphotyrosine, X₁ is Y, P, V, I, T, or F, and X₂ is I, V, L, or A; (ii) a (X₃)-Ptyr-(X₄) motif, wherein Ptyr is a phosphotyrosine, X₃ is T, A, or S, and X₄ is E or G; or (iii) both (i) and (ii).

In specific embodiments, the ephrin receptor JM domain comprises: (i) a YX₁DX₂X₃X₄YEDP motif, wherein X₁ is I or V, X₂ is P or L, X₃ is Q, H, F, D, E, or S, X₄ is A or T (SEQ ID NO:240); or (ii) a FX₁DX₂X₃X₄FEDP motif, wherein X₁ is I or V, X₂ is P or L, X₃ is Q, H, F, D, E, or S, X₄ is A or T (SEQ ID NO:241).

### (b) Kinase Domain

In one aspect, the polypeptides described herein can comprise a kinase domain, which is a conserved protein domain family (NCBI CDD accession number cd05066 for EphA and cd05033 for EphB).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., ephrin receptor TM domain), and an ephrin receptor KD C-terminal to (e.g., fused to the C-terminus of (either with a linker such as a peptide linker described herein, or without a linker)) the ephrin receptor JM domain.

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor KD C-terminal to (e.g., fused to the C-terminus of (either with a linker such as a peptide linker described herein, or without a linker)) the TM domain (e.g., the ephrin receptor TM domain).

In some embodiments, the endodomain of the parental Eph receptor is modified such that it is rendered signaling incompetent. For example, the polypeptide may comprise a kinase domain having one or more amino acid mutations which inactivate kinase activity. Specific embodiments thereof are kinase-dead Eph receptor variants wherein the conserved lysine in the ATP binding site has been mutated by substitution of lysine (L) for arginine (R) (e.g., at amino acid positions L656, L646, L653, L707, L663, L665, L667, L651, L654, L665, and L647 of parental EphA1-8 and EphB1-4, respectively), thereby inactivating the enzymatic activity of the kinase domain. In a further non-limiting example, a kinase dead Eph receptor variant can be generated by introducing one or more point mutations to affect a residue essential to the kinase activity, such as by ablating the conserved tyrosine residue (e.g., at positions 781, 772, 779, 779, 883, 831, 791, 793, 793, 778, 780, 792, and 774 of parental EphA1-8 and EphB1-4, respectively) in the tyrosine kinase domain, resulting in its inability to phosphorylate its substrates. For example, a kinase dead Eph receptor has been described in Truitt L, Freywald A, Dancing with the dead: Eph receptors and their kinase-null partners, Biochem Cell Biol. 2011 April, 89(2):115-129. For example, a mutant kinase dead Eph receptor has been described in Peter W. Janes, et. al., Eph receptor function is modulated by heterooligomerization of A and B type Eph receptors, J Cell Biol, 2011 December, 195 (6): 1033-1045.

In some embodiments, the KD of the polypeptide has at least 70%, at least 80%, at least 85%, at least 90%, at least 95%, at least 96%, at least 97%, at least 98% or at least 99% sequence identity to the KD of a wild-type ephrin receptor (e.g., an ephrin receptor KD comprising an amino acid sequence selected from the group consisting of SEQ ID NOs 102-115 of Table 10). In some embodiments, the KD of the polypeptide is the KD of a wild-type ephrin receptor (e.g., an ephrin receptor KD comprising an amino acid sequence selected from the group consisting of SEQ ID NOs: 102-115).

In some embodiments, the polypeptide described herein may comprise a KD and said KD exhibits a three dimensional structure that can be superimposed with the KD structure of a wild type ephrin receptor. In certain embodiments, the polypeptide described herein may comprise a KD and said KD exhibits a three dimensional structure, whose portion between equivalent Ca positions can be superimposed with a wild type Eph receptor KD with root-mean-square deviations (RMSDs) of at most 1, 2, 4, 4, 5, 6, 7, 8, 9 or 10 Å.

TABLE 10
KD (CDD Superfamily: cl21453,
ProRule: PRU00159).

| SEQ | Protein | | | |
|---|---|---|---|---|
| ID | (UniProt | NCBI | | |
| NO: | ID No.) | CDD # | Region | Sequence |
| 102 | EPHA1 | cl21453 | 624- | LMVDTVIGEGEFGEVYRGTL |
| | (P21709) | | 884 | RLPSQDCKTVAIKTLKDTSP |
| | | | | GGQWWNFLREATIMGQFSHP |
| | | | | HILHLEGVVTKRKPIMIITE |
| | | | | FMENGALDAFLREREDQLVP |
| | | | | GQLVAMLQGIASGMNYLSNH |
| | | | | NYVHRDLAARNILVNQNLCC |
| | | | | KVSDFGLTRLLDDFDGTYET |
| | | | | QGGKIPIRWTAPEAIAHRIF |
| | | | | TTASDVWSFGIVMWEVLSFG |
| | | | | DKPYGEMSNQEVMKSIEDGY |
| | | | | RLPPPVDCPAPLYELMKNCW |
| | | | | AYDRARRPHFQKLQAHLEQL |
| | | | | L |
| 103 | EPHA2 | cd05063 | 613- | VTRQKVIGAGEFGEVYKGML |
| | (P29317) | | 875 | KTSSGKKEVPVAIKTLKAGY |
| | | | | TEKQRVDFLGEAGIMGQFSH |
| | | | | HNIIRLEGVISKYKPMMIIT |
| | | | | EYMENGALDKFLREKDGEFS |
| | | | | VLQLVGMLRGIAAGMKYLAN |
| | | | | MNYVHRDLAARNILVNSNLV |
| | | | | CKVSDFGLSRVLEDDPEATY |
| | | | | TTSGGKIPIRWTAPEAISYR |
| | | | | KFTSASDVWSFGIVMWEVMT |
| | | | | YGERPYWELSNHEVMKAIND |
| | | | | GFRLPTPMDCPSAIYQLMMQ |
| | | | | CWQQERARRPKFADIVSILD |
| | | | | KLI |
| 104 | EPHA3 | cd05066 | 621- | ISIDKVVGAGEFGEVCSGRL |
| | (P29320) | | 882 | KLPSKKEISVAIKTLKVGYT |
| | | | | EKQRRDFLGEASIMGQFDHP |
| | | | | NIIRLEGVVTKSKPVMIVTE |
| | | | | YMENGSLDSFLRKHDAQFTV |
| | | | | IQLVGMLRGIASGMKYLSDM |
| | | | | GYVHRDLAARNILINSNLVC |
| | | | | KVSDFGLSRVLEDDPEAAYT |
| | | | | TRGGKIPIRWTSPEAIAYRK |
| | | | | FTSASDVWSYGIVLWEVMSY |
| | | | | GERPYWEMSNQDVIKAVDEG |
| | | | | YRLPPPMDCPAALYQLMLDC |
| | | | | WQKDRNNRPKFEQIVSILDK |
| | | | | LI |
| 105 | EPHA4 | cd05066 | 621- | IKIEKVIGVGEFGEVCSGRL |
| | (P54764) | | 882 | KVPGKREICVAIKTLKAGYT |
| | | | | DKQRRDFLSEASIMGQFDHP |
| | | | | NIIHLEGVVTKCKPVMIITE |
| | | | | YMENGSLDAFLRKNDGRFTV |
| | | | | IQLVGMLRGIGSGMKYLSDM |
| | | | | SYVHRDLAARNILVNSNLVC |
| | | | | KVSDFGMSRVLEDDPEAAYT |
| | | | | TRGGKIPIRWTAPEAIAYRK |
| | | | | FTSASDVWSYGIVMWEVMSY |
| | | | | GERPYWDMSNQDVIKAIEEG |
| | | | | YRLPPPMDCPIALHQLMLDC |
| | | | | WQKERSDRPKFGQIVNMLDK |
| | | | | LI |
| 106 | EPHA5 | cd05066 | 675- | ITIERVIGAGEFGEVCSGRL |
| | (P54756) | | 936 | KLPGKRELPVAIKTLKVGYT |
| | | | | EKQRRDFLGEASIMGQFDHP |
| | | | | NIIHLEGVVTKSKPVMIVTE |
| | | | | YMENGSLDTFLKKNDGQFTV |
| | | | | IQLVGMLRGISAGMKYLSDM |
| | | | | GYVHRDLAARNILINSNLVC |
| | | | | KVSDFGLSRVLEDDPEAAYT |
| | | | | TRGGKIPIRWTAPEAIAFRK |
| | | | | FTSASDVWSYGIVMWEVVSY |
| | | | | GERPYWEMTNQDVIKAVEEG |
| | | | | YRLPSPMDCPAALYQLMLDC |
| | | | | WQKERNSRPKFDEIVNMLDK |
| | | | | LI |
| 107 | EPHA6 | cd05066 | 631- | IRIERVIGAGEFGEVCSGRL |
| | (Q9UF33) | | 934 | KTPGKREIPVAIKTLKGGHM |
| | | | | DRQRRDFLREASIMGQFDHP |
| | | | | NIIRLEGVVTKRSFPAIGVE |
| | | | | AFCPSFLRAGFLNSIQAPHP |
| | | | | VPGGGSLPPRIPAGRPVMIV |
| | | | | VEYMENGSLDSFLRKHDGHF |
| | | | | TVIQLVGMLRGIASGMKYLS |
| | | | | DMGYVHRDLAARNILVNSNL |
| | | | | VCKVSDFGLSRVLEDDPEAA |
| | | | | YTTTGGKIPIRWTAPEAIAY |
| | | | | RKFSSASDAWSYGIVMWEVM |
| | | | | SYGERPYWEMSNQDVILSIE |
| | | | | EGYRLPAPMGCPASLHQLML |
| | | | | HCWQKERNHRPKFTDIVSFL |
| | | | | DKLI |
| 108 | EPHA7 | cd05066 | 633- | IKIERVIGAGEFGEVCSGRL |
| | (Q15375) | | 894 | KLPGKRDVAVAIKTLKVGYT |
| | | | | EKQRRDFLCEASIMGQFDHP |
| | | | | NVVHLEGVVTRGKPVMIVIE |
| | | | | FMENGALDAFLRKHDGQFTV |
| | | | | IQLVGMLRGIAAGMRYLADM |
| | | | | GYVHRDLAARNILVNSNLVC |
| | | | | KVSDFGLSRVIEDDPEAVYT |
| | | | | TTGGKIPVRWTAPEAIQYRK |
| | | | | FTSASDVWSYGIVMWEVMSY |
| | | | | GERPYWDMSNQDVIKAIEEG |
| | | | | YRLPAPMDCPAGLHQLMLDC |
| | | | | WQKERAERPKFEQIVGILDK |
| | | | | MI |
| 109 | EPHA8 | cd05066 | 635- | IHIEKIIGSGDSGEVCYGRL |
| | (P29322) | | 896 | RVPGQRDVPVAIKALKAGYT |
| | | | | ERQRRDFLSEASIMGQFDHP |
| | | | | NIIRLEGVVTRGRLAMIVTE |
| | | | | YMENGSLDTFLRTHDGQFTI |
| | | | | MQLVGMLRGVGAGMRYLSDL |
| | | | | GYVHRDLAARNVLVDSNLVC |
| | | | | KVSDFGLSRVLEDDPDAAYT |
| | | | | TTGGKIPIRWTAPEAIAFRT |
| | | | | FSSASDVWSFGVVMWEVLAY |
| | | | | GERPYWNMTNRDVISSVEEG |
| | | | | YRLPAPMGCPHALHQLMLDC |
| | | | | WHKDRAQRPRFSQIVSVLDA |
| | | | | LI |
| 110 | EPHA10 | cd05064 | 645- | VTLERSLGGGRFGELCCGCL |
| | (Q5JZY3) | | 904 | QLPGRQELLVAVHMLRDSAS |
| | | | | DSQRLGFLAEALTLGQFDHS |
| | | | | HIVRLEGVVTRGSTLMIVTE |
| | | | | YMSHGALDGFLRRHEGQLVA |
| | | | | GQLMGLLPGLASAMKYLSEM |
| | | | | GYVHRGLAARHVLVSSDLVC |
| | | | | KISGFGRGPRDRSEAVYTTM |
| | | | | SGRSPALWAAPETLQFGHFS |
| | | | | SASDVWSFGIIMWEVMAFGE |
| | | | | RPYWDMSGQDVIKAVEDGFR |
| | | | | LPPPRNCPNLLHRLMLDCWQ |
| | | | | KDPGERPRFSQIHSILSKMV |
| 111 | EPHB 1 | cd05065 | 619- | VKIEEVIGAGEFGEVYKGRL |
| | (P54762) | | 882 | KLPGKREIYVAIKTLKAGYS |
| | | | | EKQRRDFLSEASIMGQFDHP |
| | | | | NIIRLEGVVTKSRPVMIITE |
| | | | | FMENGALDSFLRQNDGQFTV |
| | | | | IQLVGMLRGIAAGMKYLAEM |
| | | | | NYVHRDLAARNILVNSNLVC |
| | | | | KVSDFGLSRYLQDDTSDPTY |
| | | | | TSSLGGKIPVRWTAPEAIAY |
| | | | | RKFTSASDVWSYGIVMWEVM |
| | | | | SFGERPYWDMSNQDVINAIE |
| | | | | QDYRLPPPMDCPAALHQLML |
| | | | | DCWQKDRNSRPRFAEIVNTL |
| | | | | DKMI |
| 112 | EPHB2 | cd05065 | 621- | VKIEQVIGAGEFGEVCSGHL |
| | (P29323) | | 884 | KLPGKREIFVAIKTLKSGYT |
| | | | | EKQRRDFLSEASIMGQFDHP |
| | | | | NVIHLEGVVTKSTPVMIITE |
| | | | | FMENGSLDSFLRQNDGQFTV |
| | | | | IQLVGMLRGIAAGMKYLADM |
| | | | | NYVHRDLAARNILVNSNLVC |
| | | | | KVSDFGLSRFLEDDTSDPTY |
| | | | | TSALGGKIPIRWTAPEAIQY |
| | | | | RKFTSASDVWSYGIVMWEVM |
| | | | | SYGERPYWDMTNQDVINAIE |
| | | | | QDYRLPPPMDCPSALHQLML |
| | | | | DCWQKDRNHRPKFGQIVNTL |
| | | | | DKMI |
| 113 | EPHB3 | cd05065 | 633- | VKIEEVIGAGEFGEVCRGRL |
| | (P54753) | | 896 | KQPGRREVFVAIKTLKVGYT |
| | | | | ERQRRDFLSEASIMGQFDHP |
| | | | | NIIRLEGVVTKSRPVMILTE |
| | | | | FMENCALDSFLRLNDGQFTV |
| | | | | IQLVGMLRGIAAGMKYLSEM |
| | | | | NYVHRDLAARNILVNSNLVC |
| | | | | KVSDFGLSRFLEDDPSDPTY |
| | | | | TSSLGGKIPIRWTAPEAIAY |
| | | | | RKFTSASDVWSYGIVMWEVM |
| | | | | SYGERPYWDMSNQDVINAVE |
| | | | | QDYRLPPPMDCPTALHQLML |
| | | | | DCWVRDRNLRPKFSQIVNTL |
| | | | | DKLI |
| 114 | EPHB4 | cd05065 | 615- | VKIEEVIGAGEFGEVCRGRL |
| | (P54760) | | 878 | KAPGKKESCVAIKTLKGGYT |
| | | | | ERQRREFLSEASIMGQFEHP |
| | | | | NIIRLEGVVTNSMPVMILTE |
| | | | | FMENGALDSFLRLNDGQFTV |
| | | | | IQLVGMLRGIASGMRYLAEM |
| | | | | SYVHRDLAARNILVNSNLVC |
| | | | | KVSDFGLSRFLEENSSDPTY |
| | | | | TSSLGGKIPIRWTAPEAIAF |
| | | | | RKFTSASDAWSYGIVMWEVM |
| | | | | SFGERPYWDMSNQDVINAIE |
| | | | | QDYRLPPPPDCPTSLHQLML |
| | | | | DCWQKDRNARPRFPQVVSAL |
| | | | | DKMI |
| 115 | EPHB6 | c121453 | 670- | IKIEEVIGTGSFGEVRQGRL |
| | (015197) | | 919 | QPRGRREQTVAIQALWAGGA |
| | | | | ESLQMTFLGRAAVLGQFQHP |
| | | | | NILRLEGVVTKSRPLMVLTE |
| | | | | FMELGPLDSFLRQREGQFSS |
| | | | | LQLVAMQRGVAAAMQYLSSF |
| | | | | AFVHRSLSAHSVLVNSHLVC |
| | | | | KVARLGHSPQGPSCLLRWAA |
| | | | | PEVIAHGKHTTSSDVWSFGI |
| | | | | LMWEVMSYGERPYWDMSEQE |
| | | | | VLNAIEQEFRLPPPPGCPPG |
| | | | | LHLLMLDTWQKDRARRPHFD |
| | | | | QLVAAFDKMI |

In some embodiments, the KD comprises the amino acid sequence of the KD of a wild-type ephrin receptor (e.g., an ephrin receptor KD comprising an amino acid sequence selected from the group consisting of SEQ ID NOs: 102-115) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In specific embodiments, the ephrin receptor KD comprises an (X₇)-Ptyr-(X₈) motif in the activation loop, wherein Ptyr is a phosphotyrosine, X₇ is T, V, or A, and X₈ is E or T.

### (c) SAM Linker Domain

In certain embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., the ephrin receptor TM domain), an ephrin receptor KD C-terminal to (e.g., fused to the C-terminus of (either with a linker such as a peptide linker described herein, or without a linker)) the ephrin receptor JM domain, and a SAM linker domain (e.g., an ephrin receptor SAM linker domain) C-terminal to (e.g., fused to the C-terminus of (either with a linker such as a peptide linker described herein, or without a linker)) the ephrin receptor KD.

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor KD C-terminal to (e.g., fused to the C-terminus of (either with a linker such as a peptide linker described herein, or without a linker)) the TM domain (e.g., the ephrin receptor TM domain), and a SAM linker domain (e.g., an ephrin receptor SAM linker domain) C-terminal to (e.g., fused to the C-terminus of (either with a linker such as a peptide linker described herein, or without a linker)) the ephrin receptor KD.

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises a SAM linker domain (e.g., an ephrin receptor SAM linker domain) C-terminal to (e.g., fused to the C-terminus of (either with a linker such as a peptide linker described herein, or without a linker)) the TM domain (e.g., the ephrin receptor TM domain).

In some embodiments, the SAM linker domain of the polypeptide has at least 70%, at least 80%, at least 85%, at least 90%, at least 95%, at least 96%, at least 97%, at least 98% or at least 99% sequence identity to the SAM linker domain of a wild-type ephrin receptor (e.g., an ephrin receptor SAM linker domain comprising an amino acid sequence selected from the group consisting of SEQ ID NOs:116-129 of Table 12). In some embodiments, the SAM domain of the polypeptide is the SAM domain of a wild-type ephrin receptor (e.g., an ephrin receptor SAM linker domain comprising an amino acid sequence selected from the group consisting of SEQ ID NOs: 116-129).

In some embodiments, the SAM linker domain contains multiple amino acids that serve as phosphorylation sites. In some embodiments, the SAM linker domain is a variant that comprises at least one, two, three, four, five, six, seven or eight phosphorylation sites (e.g. tyrosine (Y), serine (S) and threonine (T) sites) that are replaced by phosphomimetic amino acids (e.g., glutamic acid or aspartic acid, to mimic the negative charge of the phosphate group). In certain embodiments, the SAM linker domain comprising phosphomimetic amino acids leads to a conformational change (extension of the C-terminal away from the kinase domain). In some embodiments, the SAM linker domain comprises at least one, two, three, four, five, six, seven or eight phosphorylation sites (e.g., tyrosine, serine and threonine residues) that are replaced by non-phosphorylatable amino acids (e.g., alanine).

In a specific embodiment, the SAM linker domain described herein is an ephrin receptor SAM linker domain.

TABLE 11
SAM linker domain.

| SEQ | Pro- | | |
|---|---|---|---|
| ID NO: | tein | Region | Sequence |
| 116 | EPHA1 | 892-917 | TIANFDPRMT |
| | | | LRLPSLSGSD |
| | | | GIPYRTVS |
| 117 | EPHA2 | 883-909 | TLADFDPRVS |
| | | | IRLPSTSGSE |
| | | | GVPFRTVS |
| 118 | EPHA3 | 892-916 | TSAAARPSNL |
| | | | LLDQSNVDIT |
| | | | TFRTT |
| 119 | EPHA4 | 891-916 | TGTESSRPNT |
| | | | ALLDPSSPEF |
| | | | SAVVSV |
| 120 | EPHA5 | 944-970 | TLVNASCRVS |
| | | | NLLAEHSPLG |
| | | | SGAYRSV |
| 121 | EPHA6 | 942-966 | TLVEDILVMP |
| | | | ESPGEVPEYP |
| | | | LFVTV |
| 122 | EPHA7 | 902-928 | TPLGTCSRPI |
| | | | SPLLDQNTPD |
| | | | FTTFCSV |
| 123 | EPHA8 | 905-935 | TATVSRCPPP |
| | | | AFVRSCFDLR |
| | | | GGSGGGGGLT |
| | | | V |
| 124 | EPHA10 | 915-938 | TTCPRPPTPL |
| | | | ADRAFSTFPS |
| | | | FGSV |
| 125 | EPHB1 | 893-916 | TVATITAVPS |
| | | | QPLLDRSIPD |
| | | | FTAFTTV |
| 126 | EPHB2 | 897-918 | AMAPLSSGIN |
| | | | LPLLDRTIPD |
| | | | YTSFNTV |
| 127 | EPHB3 | 907-930 | ASAQSGMSQP |
| | | | LLDRTVPDYT |
| | | | TFTT |
| 128 | EPHB4 | 888-912 | ARENGGASHP |
| | | | LLDQRQPHYS |
| | | | AFGSV |
| 129 | EPHB6 | 929-955 | GDPGERPSQA |
| | | | LLTPVALDFP |
| | | | CLDSP |

(d) SAM Domain

In one aspect, the polypeptides described herein can comprise a SAM domain which is a conserved protein domain family (NCBI CDD accession number cl26516 or Simple Modular Architecture Research Tool (SMART) accession number smart00454). In certain embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., an ephrin receptor TM domain), an ephrin receptor KD C-terminal to (e.g., fused to the C-terminus of (either with a linker such as a peptide linker described herein, or without a linker)) the ephrin receptor JM domain, a SAM linker domain (e.g., an ephrin receptor SAM linker domain) C-terminal to (e.g., fused to the C-terminus of (either with a linker such as a peptide linker described herein, or without a linker)) the ephrin receptor KD, and a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor KD C-terminal to (e.g., fused to the C-terminus of (either with a linker such as a peptide linker described herein, or without a linker)) the TM domain (e.g., the ephrin receptor TM domain), a SAM linker domain (e.g., an ephrin receptor SAM linker domain) C-terminal to (e.g., fused to the C-terminus of (either with a linker such as a peptide linker described herein, or without a linker)) the ephrin receptor KD, and a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises a SAM linker domain (e.g., an ephrin receptor SAM linker domain) C-terminal to (e.g., fused to the C-terminus of (either with a linker such as a peptide linker described herein, or without a linker)) the TM domain (e.g., the ephrin receptor TM domain), and a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises a SAM domain (e.g., an ephrin receptor SAM domain) C-terminal to (e.g., fused to the C-terminus of (either with a linker such as a peptide linker described herein, or without a linker)) the TM domain (e.g., the ephrin receptor TM domain).

In some embodiments, the SAM domain of the polypeptide has at least 70%, at least 80%, at least 85%, at least 90%, at least 95%, at least 96%, at least 97%, at least 98% or at least 99% sequence identity to the SAM domain of a wild-type ephrin receptor (e.g., an ephrin receptor SAM domain comprising an amino acid sequence selected from the group consisting of SEQ ID NOs:130-143 of Table 12). In some embodiments, the SAM domain of the polypeptide is the SAM domain of a wild-type ephrin receptor (e.g., an ephrin receptor SAM domain comprising an amino acid sequence selected from the group consisting of SEQ ID NOs: 130-143).

TABLE 12
SAM (CDD: cd09488, ProRule: RU00184).

| SEQ | | | | |
|---|---|---|---|---|
| ID | Pro- | NCBI | | |
| NO: | tein | CDD# | Region | Sequence |
| 130 | EPHA1 | cd09542 | 919- | EWLESIRMKRYILHFHSAGL |
| | | | 974 | DTMECVLELTAEDLTQMGIT |
| | | | | LPGHQKRILCSIQGF |
| 131 | EPHA2 | cd09543 | 911- | EWLESIKMQQYTEHFMAAGY |
| | | | 971 | TAIEKVVQMTNDDIKRIGVR |
| | | | | LPGHQKRIAYSLLGLKDQVN |
| | | | | T |
| 132 | EPHA3 | c115755 | 917- | GDWLNGVWTAHCKEIFTGVE |
| | | | 974 | YSSCDTIAKISTDDMKKVGV |
| | | | | TVVGPQKKIISSIKALET |
| 133 | EPHA4 | cd09545 | 917- | GDWLQAIKMDRYKDNFTAAG |
| | | | 981 | YTTLEAVVHVNQEDLARIGI |
| | | | | TAITHQNKILSSVQAMRTQM |
| | | | | QQMHG |
| 134 | EPHA5 | cd09546 | 971- | GEWLEAIKMGRYTEIFMENG |
| | | | 1030 | YSSMDAVAQVTLEDLRRLGV |
| | | | | TLVGHQKKIMNSLQEMKVQL |
| 135 | EPHA6 | cd09547 | 967- | GDWLDSIKMGQYKNNFVAAG |
| | | | 1024 | FTTFDLISRMSIDDIRRIGV |
| | | | | ILIGHQRRIVSSIQTLRL |
| 136 | EPHA7 | cd09548 | 929- | GEWLQAIKMERYKDNFTAAG |
| | | | 988 | YNSLESVARMTIEDVMSLGI |
| | | | | TLVGHQKKIMSSIQTMRAQM |
| 137 | EPHA8 | cd09550 | 936- | GDWLDSIRMGRYRDHFAAGG |
| | | | 995 | YSSLGMVLRMNAQDVRALGI |
| | | | | TLMGHQKKILGSIQTMRAQL |
| 138 | EPHA10 | c115755 | 939- | GAWLEALDLCRYKDSFAAAG |
| | | | 998 | YGSLEAVAEMTAQDLVSLGI |
| | | | | SLAEHREALLSGISALQARV |
| 139 | EPHB1 | cd09551 | 917- | DDWLSAIKMVQYRDSFLTAG |
| | | | 975 | FTSLQLVTQMTSEDLLRIGI |
| | | | | TLAGHQKKILNSIHSMRVQ |
| 140 | EPHB2 | cd09552 | 919- | DEWLEAIKMGQYKESFANAG |
| | | | 980 | FTSFDVVSQMMMEDILRVGV |
| | | | | TLAGHQKKILNSIQVMRAQM |
| | | | | NQ |
| 141 | EPHB3 | c115755 | 931- | GDWLDAIKMGRYKESFVSAG |
| | | | 990 | FASFDLVAQMTAEDLLRIGV |
| | | | | TLAGHQKKILSSIQDMRLQM |
| 142 | EPHB4 | cd09554 | 913- | GEWLRAIKMGRYEESFAAA |
| | | | 973 | GFGSFELVSQISAEDLLRI |
| | | | | GVTLAGHQKKILASVQHMKS |
| | | | | QAK |
| 143 | EPHB6 | cd09555 | 954- | QAWLSAIGLECYQDNFSKFG |
| | | | 1013 | LCTFSDVAQLSLEDLPALGI |
| | | | | TLAGHQKKLLHHIQLLQQHL |

In some embodiments, the SAM domain comprises the amino acid sequence o the SAM domain of a wild-type ephrin receptor (e.g., an ephrin receptor SAM domain comprising an amino acid sequence selected from the group consisting of SEQ ID NOs: 130-143) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the SAM domain is not derived from an Ephrin receptor. In specific embodiments, the SAM domain comprises an amino acid sequence selected from the group consisting of SEQ ID NOs: 144-146 of Table 13. In certain embodiments the non-ephrin receptor SAM domain have a higher propensity to oligomerize than ephrin receptor SAM domains. In specific embodiments, the non-ephrin receptor SAM domains that have a higher propensity to oligomerize adapt a head-to-tail configuration, non-limiting examples include e.g. SAM domains of TEL, TNKS1 or TNKS2. Several human SAM domains can oligomerize, with different self-affinities, ranging from high micromolar to nanomolar and ways to identify and assay such domains are known in the art (e.g. Knight, et al (2011). Protein Science, 20: 1697-1706)

In some embodiments, the polypeptide described herein may comprises a SAM domain and said SAM domain exhibits a three dimensional structure that can be superimposed with the structure of the SAM domain structure of a wild type ephrin receptor. In certain embodiments, the polypeptide described herein may comprises a SAM domain and said SAM domain exhibits a three dimensional structure, whose between equivalent Ca positions can be superimposed with a wild type Eph receptor SAM domain with root-mean-square deviations (rmds) of at most 1, 2, 4, 4, 5, 6, 7, 8, 9 or 10 Å.

TABLE 13
Non-Ephrin SAM domains.

| | Corresponding | | |
|---|---|---|---|
| | Protein | | |
| SEQ | (UniProt | | |
| ID | ID No.) | Region | Sequence |
| 144 | Poly | 1030-1089 | NISQFLKSLGLEHLRDIFET |
| | [ADP-ribose] | | EQITLDVLADMGHEELKEIG |
| | polymerase | | INAYGHRHKLIKGVERLLGG |
| | tankyrase-1 | | |
| | (TNKS1 | | |
| | (O9527)) | | |
| 145 | Poly | 873-936 | GVDFSITQFVRNLGLEHLMD |
| | [ADP-ribose] | | IFEREQITLDVLVEMGHKEL |
| | polymerase | | KEIGINAYGHRHKLIKGVER |
| | tankyrase-2 | | LISG |
| | (TNKS2 | | |
| | (Q9H2K2) | | |
| 146 | Transcription | 50-124 | LPAHLRLQPIYWSRDDVAQW |
| | factor ETV6 | | LKWAENEFSLRPIDSNTFEM |
| | (TEL | | NGKALLLLTKEDFRYRSPHS |
| | (P41212)) | | GDVLYELLQHILKQR |

In specific embodiments, the SAM domain comprises a phosphotyrosine in t e α2 helix. In a particular embodiment, the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T.

In a specific embodiment, the SAM domain described herein is an ephrin receptor SAM domain.

### (e) PBM Domain

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor JM domain C-terminal to (e.g., fused to the C-terminus of (either with a linker such as a peptide linker described herein, or without a linker)) the TM domain (e.g., the ephrin receptor TM domain), an ephrin receptor KD C-terminal to (e.g., fused to the C-terminus of (either with a linker such as a peptide linker described herein, or without a linker)) the ephrin receptor JM domain, a SAM linker domain (e.g., an ephrin receptor SAM linker domain) C-terminal to (e.g., fused to the C-terminus of (either with a linker such as a peptide linker described herein, or without a linker)) the ephrin receptor KD, a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain), and an ephrin receptor PBM domain C-terminal to (e.g., fused to the C-terminus of (either with a linker such as a peptide linker described herein, or without a linker)) the SAM domain (e.g., the ephrin receptor SAM domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor KD C-terminal to (e.g., fused to the C-terminus of (either with a linker such as a peptide linker described herein, or without a linker)) the TM domain (e.g., the ephrin receptor TM domain), a SAM linker domain (e.g., an ephrin receptor SAM linker domain) C-terminal to (e.g., fused to the C-terminus of (either with a linker such as a peptide linker described herein, or without a linker)) the ephrin receptor KD, a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain), and an ephrin receptor PBM domain C-terminal to (e.g., fused to the C-terminus of (either with a linker such as a peptide linker described herein, or without a linker)) the SAM domain (e.g., the ephrin receptor SAM domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises a SAM linker domain (e.g., an ephrin receptor SAM linker domain) C-terminal to (e.g., fused to the C-terminus of (either with a linker such as a peptide linker described herein, or without a linker)) the TM domain (e.g., the ephrin receptor TM domain), a SAM domain (e.g., an ephrin receptor SAM domain) C-terminal to (e.g., fused to the C-terminus of (either with a linker such as a peptide linker described herein, or without a linker)) the SAM linker domain (e.g., the ephrin receptor SAM linker domain), and an ephrin receptor PBM domain C-terminal to (e.g., fused to the C-terminus of (either with a linker such as a peptide linker described herein, or without a linker)) the SAM domain (e.g., the ephrin receptor SAM domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises a SAM domain (e.g., an ephrin receptor SAM domain) C-terminal to (e.g., fused to the C-terminus of (either with a linker such as a peptide linker described herein, or without a linker)) the TM domain (e.g., the ephrin receptor TM domain), and an ephrin receptor PBM domain C-terminal to (e.g., fused to the C-terminus of (either with a linker such as a peptide linker described herein, or without a linker)) the SAM domain (e.g., the ephrin receptor SAM domain).

In specific embodiments, a polypeptide described herein comprises, in N-terminus to C-terminus direction, an ephrin receptor CR domain, two ephrin receptor FN III domains, a TM domain (e.g., an ephrin receptor TM domain), and further comprises an ephrin receptor PBM domain C-terminal to (e.g., fused to the C-terminus of (either with a linker such as a peptide linker described herein, or without a linker)) the TM domain (e.g., the ephrin receptor TM domain).

In some embodiments, the PBM domain of the polypeptide has at least 33%, at least 66%, or at least 99% sequence identity to the PBM domain of a wild-type ephrin receptor (e.g., an ephrin receptor PBM domain comprising an amino acid sequence selected from the group consisting of SEQ TD NOs: 147-159 and 256 of Table 14). In some embodiments, the PBM domain of the polypeptide is the PBM domain of a wild-type ephrin receptor (e.g., an ephrin receptor PBM domain comprising an amino acid sequence selected from the group consisting of SEQ TD NOs: 147-159 and 256).

TABLE 14
PDZ-Binding Motif.

| SEQ | | | |
|---|---|---|---|
| ID NO: | Protein | Region | Sequence |
| 147 | EPHA1 | 974-976 | FKD |
| | HUMAN | | |
| 148 | EPHA2 | 974-976 | IPI |
| | HUMAN | | |
| 149 | EPHA3 | 981-983 | VPV |
| | HUMAN | | |
| 150 | EPHA4 | 984-986 | VPV |
| | HUMAN | | |
| 151 | EPHA5 | 1035-1037 | VPL |
| | HUMAN | | |
| 152 | EPHA6 | 1034-1036 | FHV |
| | HUMAN | | |
| 153 | EPHA7 | 996-998 | IQV |
| | HUMAN | | |
| 154 | EPHA8 | 1003-1005 | RHL |
| | HUMAN | | |
| 256 | EphA10 | 1005-1008 | VQV |
| | HUMAN | | |
| 155 | EPHB 1 | 982-984 | AMA |
| | HUMAN | | |
| 156 | EPHB2 | 984-986 | VEG |
| | HUMAN | | |
| 157 | EPHB3 | 996-998 | VQV |
| | HUMAN | | |
| 158 | EPHB4 | 985-987 | PQY |
| | HUMAN | | |
| 159 | EPHB6 | 1019-1021 | VEV |
| | HUMAN | | |

In some embodiments, the PBM domain comprises the amino acid sequence of the PBM domain of a wild-type ephrin receptor (e.g., an ephrin receptor PBM domain comprising an amino acid sequence selected from the group consisting of SEQ ID NOs: 147-159 and 256) except one amino acid mutation or two amino acid mutations. The mutation(s) can be substitution(s), insertion(s), deletion(s), or any combination thereof.

### 5.2.3 Cargo and Cargo Binding Domain

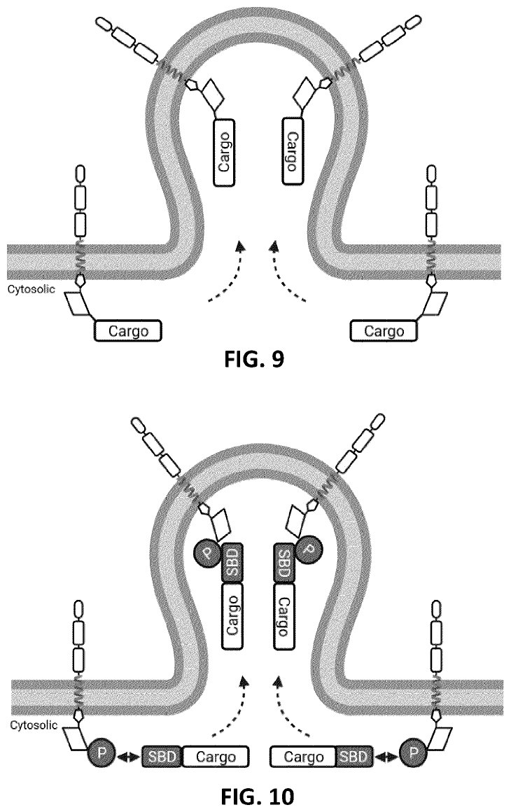

A polypeptide described herein can be used to deliver a cargo (e.g., a cargo protein), for example, by an extracellular vesicle (EV) or a hybridosome, e.g., for a therapeutic or diagnostic use. The cargo (e.g., a cargo protein) can be part of the polypeptide. In other words, the cargo (e.g., a cargo protein) can be fused to the remaining portion of the polypeptide (e.g., via a linker) (see, e.g., FIG. 9). Alternatively, the cargo (e.g., a cargo protein) can be bound (preferably, reversibly bound) to the polypeptide through a cargo binding domain. A cargo binding domain can bind to the cargo (e.g., cargo protein) directly, or indirectly via a scaffold binding domain (SBD) linked to the cargo (e.g., cargo protein). The cargo binding domain can be either an ephrin receptor domain (such as an ephrin receptor JM domain, ephrin receptor KD, ephrin receptor SAM linker domain, ephrin receptor SAM domain, or ephrin receptor PBM domain, see, e.g., FIG. 10), or a domain capable of binding to a cargo but is not an ephrin receptor domain. Accordingly, in specific embodiments, a polypeptide described herein comprises a cargo (e.g., a cargo protein) or a cargo binding domain. The singular forms “a”, “an”, and “the” as used herein include plural referents. As such, a polypeptide described herein can be used to deliver one or more (e.g., one, two, three, four, five or more) cargos, and a polypeptide described herein can comprise one or more (e.g., one, two, three, four, five or more) cargo binding domains.

Exemplary cargos (such as cargo proteins) include, without being limited to, therapeutic molecules (e.g., therapeutic proteins), adjuvants, diagnostic proteins, and/or reporter proteins. The cargo may be a large polypeptide or a peptide, such as RGD or an antimicrobial peptide.

A therapeutic molecule refers to any molecule that can have a therapeutic use. The therapeutic molecule may be any inorganic or organic compound. A therapeutic molecule may decrease, suppress, attenuate, diminish, arrest, or stabilize the development or progression of disease, disorder, or cell growth in an animal such as a mammal or human. Examples of therapeutic molecule that can be introduced into a nanovesicle comprising Eph receptor derived polypeptides include therapeutic agents such as, nucleic acids (e.g., DNA or mRNA molecules that encode a polypeptide such as an enzyme, mRNA molecules that encode a polypeptide such as an antigen or RNA molecules that have regulatory function such as miRNA, dsDNA, and lncRNA), amino acids (e.g., amino acids comprising a detectable moiety or a toxin or that disrupt translation), polypeptides (e.g., enzymes, enzymes for gene editing, nucleic acid binding proteins, antibodies, intrabodies, single chain variable fragments (scFv), affibodies, bi- and multispecific antibodies or binders, affibodies, darpins, receptors, ligands, or fragments thereof), lipids, carbohydrates, and small molecules (e.g., small molecule drugs and toxins). In certain embodiments, the therapeutic molecules may be a substance used in the diagnosis, treatment, or prevention of a disease or as a component of a medication. In some embodiments, a “payload” may refer to a compound that facilitates obtaining diagnostic information about a targeted site in a body of a living organism, such as a mammal or in particular a human. For example, imaging agents may be classified as active agents in the present disclosure as they are substances that provide imaging information required for diagnosis.

Further non-limiting examples of therapeutic nucleic acids intended to be used in the present disclosure are siRNA, small or short hairpin RNA (shRNA), guide RNA (gRNA), single guide RNA (sgRNA), clustered regularly interspaced short palindromic repeat RNA (crRNA), trans-activating clustered regularly interspaced short palindromic repeat RNA (tracrRNA) immune-stimulating oligonucleotides, plasmids, antisense nucleic acids and ribozymes. In certain embodiments the therapeutic nucleic acid may be linear DNA, circular DNA, or an artificial chromosome. In some embodiments the therapeutic DNA is maintained episomally. In some embodiments the therapeutic DNA is integrated into the genome. The therapeutic RNA may be chemically modified RNA, e.g., the therapeutic RNA may comprise one or more backbone modification, sugar modifications, noncanonical bases, or caps. Backbone modifications include, e.g., phosphorothioate, N3′ phosphoramidite, boranophosphate, phosphonoacetate, thio-PACE, morpholino phosphoramidites, or PNA. Sugar modifications include, e.g., 2′-O-Me, LNA, UNA, and 2′-O-MOE. Noncanonical bases include, e.g., 5-bromo-U, and 5-iodo-U, 2,6-diaminopurine, C-5 propynyl pyrimidine, difluorotoluene, difluorobenzene, dichlorobenzene, 2-thiouridine, pseudouridine, and dihydrouridine. Caps include, e.g., ARCA. Additional modifications are discussed, e.g., in Deleavey et al., “Designing Chemically Modified Oligonucleotides for Targeted Gene Silencing” Chemistry & Biology Volume 19, Issue 8, 24 Aug. 2012, Pages 937-954.

Non-limiting examples of other suitable therapeutic molecules include pharmacologically active drugs and genetically active molecules, including antineoplastic agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Suitable payloads further include toxins, and biological and chemical warfare agents, for example see Somani, S. M. (ed.), Chemical Warfare Agents, Academic Press, New York (1992)).

Additional non-limiting examples of therapeutic molecules include: antigen-binding molecules (e.g., therapeutic antibodies or antigen binding fragments thereof), gene editors, transposases, enzymes or fragments thereof; ligands or fragments thereof, receptors or fragments thereof, antimicrobial peptides or fragments thereof, amino acids, and any combination thereof. In some embodiments, the therapeutic molecule is non-proteic and attached via a linker to the Eph receptor derived polypeptide.

Antigen binding molecules serving as therapeutic molecules, may be monospecific, bispecific or multispecific, i.e., they may target one or more epitopes of the same target or different targets. The more specificities are displayed on the nanovesicle, the more specific its targeting is. In some embodiments, the antigen binding molecule is selected from the group consisting of:
i) a full-length antibody molecule (such as an IgG, an IgM, an IgA, an IgM or an IgE);
ii) an antibody fragment such as a CDR, a Dab, a Fab, a Fab′, a F(ab)′2, a Fd fragment, a Fv fragment, a disulfide linked Fv, a scFab, a nanobody, a minimal recognition unit, a VHH or a V-NAR domain;
iii) a non-antibody scaffold such as an affibody, an affilin molecule, an affitin, an AdNectin, an anticalin, an avimer, a centyrin, a lipocalin mutein, a DARPin, a fynomer, a Knottin, a Kunitz-type domain, a nanofitin, a tetranectin or a trans-body; iv) a fusion polypeptide comprising one or more antibody domains, such as a bi-scFv, aBITE, a diabody, di-scFv, probody, tascFv (tandem scFv), triabody, tribody, tetrabody, IgGACH2, DVD-Ig, MATCH, a minibody, a scFv, a scFv-Fc, bispecific F(ab′)2, F(ab′)3, monovalent IgG;
v) a soluble T-cell receptor (sTCR);
vi) a peptide, such as natural peptide, a recombinant peptide, a synthetic peptide; and/or
vii) a viral protein such as the receptor binding domain of a viral spike protein (such as of coronavirus) or hemagglutinin (HA) of influenza, or fragments thereof, respectively.

Several reporter proteins are known in the art, such as green fluorescent protein (GFP) or luciferase. Reporter proteins are useful for observing intracellular trafficking and/or uptake of nanovesicles in recipient cells.

In certain embodiments, diagnostic proteins can be fluorescent proteins. In specific embodiments, diagnostic proteins can be fusion proteins comprising a moiety that can bind to a biomarker of interest and a fluorescent protein (e.g., GFP).

### (a) Cargo Fusion Protein

As described above, a polypeptide provided herein can comprise one or more cargos (such as cargo proteins), preferably biologically active molecules. Thereby, when sorted into nanovesicles, the polypeptide serves as a scaffold protein for such cargos (such as cargo proteins). The polypeptides provide a protein scaffold amenable to load any molecule of interest onto nanovesicles in a predefined manner, e.g., by genetic fusion. In certain embodiments, the cargo is a cargo protein (e.g., a cargo peptide or cargo polypeptide) which is fused in-frame to the remaining portion of the polypeptide. In certain embodiments, the cargo protein is fused to the remaining portion of the polypeptide via a linker. In some embodiments, the cargo protein is covalently fused to the remaining portion of the polypeptide via a linker. In specific embodiments, the linker is a peptide linker. In a specific embodiment, the peptide linker comprises an amino acid sequence of (GGGS)n (SEQ ID NO:231), wherein n is an integer number from 1 to 10. In a specific embodiment, the peptide linker comprises an amino acid sequence of GGGS. In a specific embodiment, the peptide linker comprises an amino acid sequence of (GGGS)2 (SEQ ID NO:232). In a specific embodiment, the peptide linker comprises an amino acid sequence of (GGGS)3 (SEQ ID NO: 233)

Such one or more cargos (such as cargo proteins) can be N-terminal and/or C-terminal to (e.g., N-terminally and/or C-terminally fused to) the remaining portion of the polypeptide or placed between the different domains of the remaining portion of the polypeptide. In certain embodiments, the one or more cargos (such as cargo proteins) are presented towards the lumen of a nanovesicle. In some embodiments, the one or more cargos (such as cargo proteins) are C-terminal to (e.g., C-terminally fused to) the TM domain (e.g., the ephrin receptor TM domain). In some embodiments, the one or more cargos (such as cargo proteins) are C-terminal to (e.g., C-terminally fused to) the ephrin receptor JM domain. In some embodiments, the one or more cargos (such as cargo proteins) are C-terminal to (e.g., C-terminally fused to) the ephrin receptor KD. In some embodiments, the one or more cargos (such as cargo proteins) are C-terminal to (e.g., C-terminally fused to) the SAM linker domain (e.g., the ephrin receptor SAM linker domain). In some embodiments, the one or more cargos (such as cargo proteins) are C-terminal to (e.g., C-terminally fused to) the SAM domain (e.g., the ephrin receptor SAM domain). In some embodiments, the one or more cargos (such as cargo proteins) are C-terminal to (e.g., C-terminally fused to) the ephrin receptor PBM domain. In certain embodiments, the one or more cargos (such as cargo proteins) are presented towards the external space of a nanovesicle. In some embodiments, the one or more cargos (such as cargo proteins) are N-terminal to (e.g., N-terminally fused to) the ephrin ligand binding domain of the polypeptide. In some embodiments, the one or more cargos (such as cargo proteins) are N-terminal to (e.g., N-terminally fused to) the ephrin receptor cysteine rich domain of the polypeptide. In some embodiments, the one or more cargos (such as cargo proteins) are N-terminal to (e.g., N-terminally fused to) the ephrin receptor FN1 domain. In some embodiments, the one or more cargos (such as cargo proteins) are N-terminal to (e.g., N-terminally fused to) the ephrin receptor FN2 domain. In some embodiments, the one or more cargos (such as cargo proteins) are N-terminal to (e.g., N-terminally fused to) the TM domain (e.g., the ephrin receptor TM domain).

In certain embodiments, the one or more cargos (such as cargo proteins) are N-terminal to (e.g., N-terminally fused to) a targeting domain described in this disclosure. In certain embodiments, the one or more cargos (such as cargo proteins) are C-terminal to (e.g., C-terminally fused to) a targeting domain described in this disclosure. In certain embodiments, the one or more cargos (such as cargo proteins) are N-terminal to (e.g., N-terminally fused to) a purification domain described in this disclosure. In certain embodiments, the one or more cargos (such as cargo proteins) are C-terminal to (e.g., C-terminally fused to) a purification domain described in this disclosure. In certain embodiments, the one or more cargos (such as cargo proteins) are N-terminal to (e.g., N-terminally fused to) a modified Fc domain described in this disclosure. In certain embodiments, the one or more cargos (such as cargo proteins) are C-terminal to (e.g., C-terminally fused to) a modified Fc domain described in this disclosure.

### (b) Cargo Binding Domain

A polypeptide described herein can be used to deliver a cargo (e.g., a cargo protein) associated (preferably, non-covalently bound) with the polypeptide through a cargo binding domain. The cargo binding domain can be either an ephrin receptor domain (such as an ephrin receptor JM domain, ephrin receptor KD, ephrin receptor SAM linker domain, ephrin receptor SAM domain, or ephrin receptor PBM domain, see, e.g., FIG. 10), or a domain capable of binding to a cargo but is not an ephrin receptor domain (e.g., a SAM domain or SAM linker domain not derived from an ephrin receptor). Accordingly, in specific embodiments, a polypeptide described herein comprises a cargo binding domain. A cargo binding domain can bind to the cargo (e.g., cargo protein) directly, or indirectly via a scaffold binding domain (SBD) linked to the cargo (e.g., cargo protein). In a specific embodiment, a cargo binding domain is capable of specifically binding to the cargo (e.g., cargo protein). The singular forms “a”, “an”, and “the” as used herein include plural referents. As such, a polypeptide described herein can be used to deliver one or more (e.g., one, two, three, four, five or more) cargos, and a polypeptide described herein can comprise one or more (e.g., one, two, three, four, five or more) cargo binding domains.

In various embodiments, a cargo binding domain can be fused in-frame to the remaining portion of the polypeptide. In certain embodiments, the cargo binding domain is fused to the remaining portion of the polypeptide via a linker. In some embodiments, the cargo binding domain is covalently fused to the remaining portion of the polypeptide via a linker. In specific embodiments, the linker is a peptide linker. In a specific embodiment, the peptide linker comprises an amino acid sequence of (GGGS)n (SEQ ID NO: 231), wherein n is an integer number from 1 to 10. In a specific embodiment, the peptide linker comprises an amino acid sequence of GGGS. In a specific embodiment, the peptide linker comprises an amino acid sequence of (GGGS)2 (SEQ ID NO: 232). In a specific embodiment, the peptide linker comprises an amino acid sequence of (GGGS)3 (SEQ ID NO: 233).

Such a cargo binding domain can be N- or C-terminal to (e.g., N-terminally and/or C-terminally fused to) the remaining portion of the polypeptide or placed between the different domains of the remaining portion of the polypeptide. In certain embodiments, the cargo binding domain is presented towards the lumen of a nanovesicle. In some embodiments, the cargo binding domain is C-terminal to (e.g., C-terminally fused to) the TM domain (e.g., the ephrin receptor TM domain). In some embodiments, the cargo binding domain is C-terminal to (e.g., C-terminally fused to) the ephrin receptor JM domain. In some embodiments, the cargo binding domain is C-terminal to (e.g., C-terminally fused to) the ephrin receptor KD. In some embodiments, the cargo binding domain is C-terminal to (e.g., C-terminally fused to) the SAM linker domain (e.g., the ephrin receptor SAM linker domain). In some embodiments, the cargo binding domain is C-terminal to (e.g., C-terminally fused to) the SAM domain (e.g., the ephrin receptor SAM domain). In some embodiments, the cargo binding domain is C-terminal to (e.g., C-terminally fused to) the ephrin receptor PBM domain. In certain embodiments, the cargo binding domain is presented towards the external space of a nanovesicle. In some embodiments, the cargo binding domain is N-terminal to (e.g., N-terminally fused to) the ephrin ligand binding domain of the polypeptide. In some embodiments, the cargo binding domain is N-terminal to (e.g., N-terminally fused to) the ephrin receptor cysteine rich domain of the polypeptide. In some embodiments, the cargo binding domain is N-terminal to (e.g., N-terminally fused to) the ephrin receptor FN1 domain. In some embodiments, the cargo binding domain is N-terminal to (e.g., N-terminally fused to) the ephrin receptor FN2 domain. In some embodiments, the cargo binding domain is N-terminal to (e.g., N-terminally fused to) the TM domain (e.g., the ephrin receptor TM domain).

In certain embodiments, the cargo binding domain is N-terminal to (e.g., N-terminally fused to) a targeting domain described in this disclosure. In certain embodiments, the cargo binding domain is C-terminal to (e.g., C-terminally fused to) a targeting domain described in this disclosure. In certain embodiments, the cargo binding domain is N-terminal to (e.g., N-terminally fused to) a purification domain described in this disclosure. In certain embodiments, the cargo binding domain is C-terminal to (e.g., C-terminally fused to) a purification domain described in this disclosure. In certain embodiments, the cargo binding domain is N-terminal to (e.g., N-terminally fused to) a modified Fc domain described in this disclosure. In certain embodiments, the cargo binding domain is C-terminal to (e.g., C-terminally fused to) a modified Fc domain described in this disclosure.

In some embodiments, the binding between the cargo binding domain and the cargo (e.g., cargo protein) is a covalent binding. In further embodiments, the association between the cargo binding domain and the cargo (e.g., cargo protein) is a non-covalent binding (e.g., as depicted in FIG. 10). Preferably, the association between the cargo binding domain and the cargo (e.g., cargo protein) is a reversible association.

In specific embodiments, the binding between the cargo binding domain and the cargo (e.g., cargo protein) is capable of being controlled. In specific embodiments, the binding between the cargo binding domain and the cargo (e.g., cargo protein) is capable of being controlled by a parameter whose value depends on the location of the polypeptide, for example, whether the polypeptide is located in vitro or in vivo, or which organ, tissue, cell, or subcellular compartment the polypeptide is located. In a specific embodiment, the binding between the cargo binding domain and the cargo (e.g., cargo protein) is capable of being controlled by pH. In a specific embodiment, the binding between the cargo binding domain and the cargo (e.g., cargo protein) is capable of being controlled by ionic strength. In a specific embodiment, the binding between the cargo binding domain and the cargo (e.g., cargo protein) is capable of being controlled by the presence or absence of a phosphatase. In specific embodiments, the binding between the cargo binding domain and the cargo (e.g., cargo protein) is capable of being controlled such that the cargo (e.g., cargo protein) is bound to the cargo binding domain in vitro but is released from the cargo binding domain in vivo. For example, the binding between the cargo binding domain and the cargo (e.g., cargo protein) may have a higher (e.g., at least 2-fold higher, at least 5-fold higher, at least 10-folder higher, at least 20-fold higher, at least 50-fold higher, at least 100-fold higher, at least 200-fold higher, at least 500-fold higher, or at least 1000-fold higher) binding affinity in an in vitro environment relative to an in vivo environment. In specific embodiments, the binding between the cargo binding domain and the cargo (e.g., cargo protein) is capable of being controlled such that the cargo (e.g., cargo protein) is released from the cargo binding domain in a manner dependent on the subcellular compartment in which they are located. For example, in a specific embodiment, the binding between the cargo binding domain and the cargo (e.g., cargo protein) is capable of being controlled such that the cargo (e.g., cargo protein) is released from the cargo binding domain when they into the cytosol. In a further non-limiting example, the binding between the cargo binding domain and the cargo (e.g., cargo protein) is capable of being controlled such that the cargo (e.g., cargo protein) is released from the cargo binding domain when the nanovesicle comprising the cargo protein in its lumen fuses with an endosomal membrane and is in contact with the cytosol.

In some embodiments, the cargo binding domain of the polypeptide and the cargo, e.g., cargo protein, is associated through an intermediary. In some embodiments, the cargo, e.g., a cargo protein, is linked to scaffold binding domain, and the scaffold protein comprises a cargo binding domain, wherein the cargo binding domain of the scaffold protein associates with the scaffold binding domain linked to the cargo (e.g., a cargo protein). In some embodiments the cargo protein has at least one scaffold binding domain. The cargo protein can comprise or be linked to a scaffold binding domain at any position of the protein, be it at the C- or N-terminal or somewhere in between. In some embodiments, the binding between the cargo binding domain and a scaffold binding domain linked to a cargo protein is capable of being controlled by taking into account the binding affinity, binding kinetics (e.g., intrinsic equilibrium dissociation constant) and the concentration of the binding pair. Methods for analyzing binding affinity and binding kinetics between scaffold binding domain and cargo binding domains are known in the art (e.g., SPR, BLI, ELISA).

In certain embodiments, the binding between the cargo binding domain and the cargo (e.g., cargo protein) is a phosphotyrosine-based binding (such as a binding between a phosphotyrosine and a phosphotyrosine binding (PTB) domain, a binding between a phosphotyrosine and a Src homology 2 (SH2) domain, or a binding between a phosphotyrosine and a HYB domain, a GEP100 PH domain, a PKCδ domain, a PKCθ C2 domain, a catalytically inactive PTP domain, or a Raf-1 kinase inhibitory protein (RKIP) domain). In specific embodiments, the cargo binding domain comprises a phosphotyrosine and the cargo (e.g., cargo protein) or the scaffold binding domain (SBD) comprises a domain that is capable of binding to phosphotyrosine, and the binding between the cargo binding domain and the cargo (e.g., cargo protein) is a binding between the phosphotyrosine and the domain that is capable of binding to phosphotyrosine.

In a specific embodiment, the domain that is capable of binding to phosphotyrosine is a PTB domain. In a specific embodiment, the PTB domain is derived from CBL (UniProt ID No. P22681) or comprises an amino acid sequence identical or similar to that of SEQ ID NO:160 as listed in Table 15 below, and the cargo binding domain comprises a phosphotyrosine. In a specific embodiment, the PTB domain is derived from CBL (UniProt ID No. P22681) or comprises an amino acid sequence of SEQ ID NO:160 as listed in Table 15 below, and the cargo binding domain comprises a phosphotyrosine and is from or derived from EphA2. In a specific embodiment, the domain that is capable of binding to phosphotyrosine is a functional variant of a SH2 domain (NCBI CDD accension number cl15255). In a specific embodiment, the domain that is capable of binding to phosphotyrosine is an SH2 domain derived from a protein listed in Table 16 below or comprises an amino acid sequence identical or similar to an amino acid sequence listed in Table 16, and the cargo binding domain comprises a phosphotyrosine. In a specific embodiment, the domain that is capable of binding to phosphotyrosine is an SH2 domain derived from a protein listed in Table 16 below or comprises an amino acid sequence identical or similar to an amino acid sequence listed in Table 16, and the cargo binding domain comprises a phosphotyrosine and is from or derived from a corresponding parental Eph receptor listed in Table 16. In a specific embodiment, the domain that is capable of binding to phosphotyrosine is a HYB domain, a GEP100 PH domain, a PKCδ domain, a PKCθ C2 domain, a catalytically inactive PTP domain, or a Raf-1 kinase inhibitory protein (RKIP) domain.

TABLE 15
Exemplary phosphotyrosine and PTB
domain-based binding.

| | Protein | | |
|---|---|---|---|
| | from | Exemplary | |
| | which | parental | |
| | scaffold | Eph | |
| | binding | receptor | |
| | domain is | (s) of | |
| | derived | the | |
| SEQ | (UniProt | cargo | |
| ID | ID | binding | PTB domain |
| NO: | No.) | domain | sequence |
| 160 | CBL | EphA2 | PPGTVDKKMVEKCWKLMDKV |
| | (P22681) | | VRLCQNPKLALKNSPPYILD |
| | | | LLPDTYQHLRTILSRYEGKM |
| | | | ETLGENEYFRVFMENLMKKT |
| | | | KQTISLFKEGKERMYEENSQ |
| | | | PRRNLTKLSLIFSHMLAELK |
| | | | GIFPSGLFQGDTFRITKADA |
| | | | AEFWRKAFGEKTIVPWKSFR |
| | | | QALHEVHPISSGLEAMALKS |
| | | | TIDLTCNDYISVFEFDIFTR |
| | | | LFQPWSSLLRNWNSLAVTHP |
| | | | GYMAFLTYDEVKARLQKFIH |
| | | | KPGSYIFRLSCTRLGQWAIG |
| | | | YVTADGNILQTIPHNKPLFQ |
| | | | ALIDGFREGFYLFPDGRNQN |
| | | | PDLTG |

TABLE 16
Exemplary phosphotyrosine and
SH2 domain-based binding.

| | Protein | Exemplary | |
|---|---|---|---|
| | from which | parental | |
| | scaffold | Eph | |
| | binding | receptor (s) | |
| | domain is | of the | |
| SEQ | derived | cargo | |
| ID | (UniProt ID | binding | |
| NO: | No.) | domain | SH2 domain sequence |
| 161 | RIN1 | EphA4/ | WLQLQANAAAALHMLRTEPP |
| | (Q13671) | EphB2 | GTFLVRKSNTRQCQALCMRL |
| | | | PEASGPSFVSSHYILESPGG |
| | | | VSLEGSELMFPDLVQLICAY |
| | | | CHTRDILLLPLQLPR |
| 162 | VAV1 | EphA2 | WYAGPMERAGAESILANRSD |
| | (P15498) | | GTFLVRQRVKDAAEFAISIK |
| | | | YNVEVKHIKIMTAEGLYRIT |
| | | | EKKAFRGLTELVEFYQQNSL |
| | | | KDCFKSLDTTLQFPF |
| 163 | VAV2 | EphA2/ | WFAGNMERQQTDNLLKSHAS |
| | (P52735) | EphB2 | GTYLIRERPAEAERFAISIK |
| | | | FNDEVKHIKVVEKDNWIHIT |
| | | | EAKKFDSLLELVEYYQCHSL |
| | | | KESFKQLDTTLKYPY |
| 164 | VAV3 | EphA2 | WYAGAMERLQAETELINRVN |
| | (Q9UKW4) | | STYLVRHRTKESGEYAISIK |
| | | | YNNEAKHIKILTRDGFFHIA |
| | | | ENRKFKSLMELVEYYKHHSL |
| | | | KEGFRTLDTTLQFPY |
| 165 | SOCS2 | EphA2 | WLFEGLGRDKAEELLQLPDT |
| | (O14508) | | KVGSFMIRESETKKGFYSLS |
| | | | VRHRQVKHYRIFRLPNNWYY |
| | | | ISPRLTFQCLEDLVNHYSEV |
| | | | ADGLCCVLTTPC |
| 166 | SLAP2 | EphA2 | WLFEGLGRDKAEELLQLPDT |
| | (Q9H6Q3) | | KVGSFMIRESETKKGFYSLS |
| | | | VRHRQVKHYRIFRLPNNWYY |
| | | | ISPRLTFQCLEDLVNHYSEV |
| | | | ADGLCCVLTTPC |
| 167 | SRC | EphA/ | WYFGKITRRESERLLLNAEN |
| | (P12931) | EphB | PRGTFLVRESETTKGAYCLS |
| | | | VSDFDNAKGLNVKHYKIRKL |
| | | | DSGGFYITSRTQFNSLQQLV |
| | | | AYYSKHADGLCHRLTTVC |
| 168 | NCK1 | EphB1, | WYYGKVTRHQAEMALNERGH |
| | (P16333) | EphA4, | EGDFLIRDSESSPNDFSVSL |
| | | EphA3 | KAQGKNKHFKVQLKETVYCI |
| | | | GQRKFSTMEELVEHYKKAPI |
| | | | FTSEQGEKLYLVKHL |
| 169 | NCK2 | EphB2 | WYYGNVTRHQAECALNERGV |
| | (043639) | | EGDFLIRDSESSPSDFSVSL |
| | | | KASGKNKHFKVQLVDNVYCI |
| | | | GQRRFHTMDELVEHYKKAPI |
| | | | FTSEHGEKLYLVRALQ |
| 170 | CBL | EphA | QPWPTLLKNWQLLAVNHPGY |
| | (P22681) | | MAFLTYDEVQERLQACRDKP |
| | | | GSYIFRPSCTRLGQWAIGYV |
| | | | SSDGSILQTIPANKPLSQVL |
| | | | LEGQKDGFYLYPDGKTHNPD |
| | | | LTE |
| 171 | CBL-C | EphA | QPWPTLLKNWQLLAVNHPGY |
| | (Q9ULV8) | | MAFLTYDEVQERLQACRDKP |
| | | | GSYIFRPSCTRLGQWAIGYV |
| | | | SSDGSILQTIPANKPLSQVL |
| | | | LEGQKDGFYLYPDGKTHNPD |
| | | | LTE |
| 172 | CBL-B | EphA | QPWGSILRNWNFLAVTHPGY |
| | (Q13191) | | MAFLTYDEVKARLQKYSTKP |
| | | | GSYIFRLSCTRLGQWAIGYV |
| | | | TGDGNILQTIPHNKPLFQAL |
| | | | IDGSREGFYLYPDGRSYNPD |
| | | | LTG |
| 173 | FYN | EphA3/Ep | WYFGKLGRKDAERQLLSFGN |
| | (P06241) | hA4/ | PRGTFLIRESETTKGAYSLS |
| | | EphB2 | IRDWDDMKGDHVKHYKIRKL |
| | | | DNGGYYITTRAQFETLQQLV |
| | | | QHYSERAAGLCCRLVVPC |
| 174) | PIK3R1 | EphA2/ | WYWGDISREEVNEKLRDTAD |
| | (P27986) | EphA4 | GTFLVRDASTKMHGDYTLTL |
| | | | RKGGNNKLIKIFHRDGKYGF |
| | | | SDPLTFSSVVELINHYRNES |
| | | | LAQYNPKLDVKLLYPV |
| 175 | PIK3R1 | EphA2/ | WNVGSSNRNKAENLLRGKRD |
| | (P27986) | EphA4 | GTFLVRESSKQGCYACSVVV |
| | | | DGEVKHCVINKTATGYGFAE |
| | | | PYNLYSSLKELVLHYQHTSL |
| | | | VQHNDSLNVTLAYPV |
| 176 | GRB2 | EphB1/ | WFFGKIPRAKAEEMLSKQRH |
| | (P62993) | EphB2 | DGAFLIRESESAPGDFSLSV |
| | | | KFGNDVQHFKVLRDGAGKYF |
| | | | LWVVKFNSLNELVDYHRSTS |
| | | | VSRNQQIFLRDIE |
| 177 | GRB7 | EphA2/ | WFHGRISREESQRLIGQQGL |
| | (Q14451) | EphB1 | VDGLFLVRESQRNPQGFVLS |
| | | | LCHLQKVKHYLILPSEEEGR |
| | | | LYFSMDDGQTRFTDLLQLVE |
| | | | FHQLNRGILPCLLRHCC |
| 178 | RASGAP | EphB2/ | WFHGKISKQEAYNLLMTVGQ |
| | (P20936) | EphB3 | VCSFLVRPSDNTPGDYSLYF |
| | | | RTNENIQRFKICPTPNNQFM |
| | | | MGGRYYNSIGDIIDHYRKEQ |
| | | | IVEGYYLKEPV |
| 179 | RASGAP | EphB2/Eph | WFHGKISKQEAYNLLMTVGQ |
| | (P20936) | B3 | VCSFLVRPSDNTPGDYSLYF |
| | | | RTNENIQRFKICPTPNNQFM |
| | | | MGGRYYNSIGDIIDHYRKEQ |
| | | | IVEGYYLKEPV |
| 180 | CRK | EphB2/ | WYWGRLSRQEAVALLQGQRH |
| | (P46108) | EphB3 | GVFLVRDSSTSPGDYVLSVS |
| | | | ENSRVSHYIINSSGPRPPVP |
| | | | PSPAQPPPGVSPSRLRIGDQ |
| | | | EFDSLPALLEFYKIHYLDTT |
| | | | TLIEPV |
| 181 | YES1 | EphA3/ | WYWGRLSRQEAVALLQGQRH |
| | (P07947) | EphA4 | GVFLVRDSSTSPGDYVLSVS |
| | | /EphB2 | ENSRVSHYIINSSGPRPPVP |
| | | | PSPAQPPPGVSPSRLRIGDQ |
| | | | EFDSLPALLEFYKIHYLDTT |
| | | | TLIEPV |
| 182 | ABL | EphA2/ | WYHGPVSRNAAEYLLSSGIN |
| | (P00519) | EphB2 | GSFLVRESESSPGQRSISLR |
| | | | YEGRVYHYRINTASDGKLYV |
| | | | SSESRENTLAELVHHHSTVA |
| | | | DGLITTLHYPA |
| 183 | SHEP1 | EphB2 | WYHGRIPREVSETLVQRNGD |
| | (Q8N5H7) | | FLIRDSLTSLGDYVLTCRWR |
| | | | NQALHFKINKVVVKAGESYT |
| | | | HIQYLFEQESFDHVPALVRY |
| | | | HVGSRKAVSEQSGAIIYCPV |

In certain embodiments, the binding between the cargo binding domain and the cargo (e.g., cargo protein) is a SAM domain-based binding. In specific embodiments, the cargo binding domain comprises a first SAM domain and the cargo (e.g., cargo protein) or the SBD comprises a domain capable of binding to the first SAM domain (e.g., a second SAM domain), and the binding between the cargo binding domain and the cargo (e.g., cargo protein) is a binding between the first SAM domain and the domain capable of binding to the first SAM domain (e.g., the second SAM domain). In some embodiments, the second SAM domain belongs to CDD ascension number cl15755. The first and the second SAM domains can be identical or different SAM domains. In a specific embodiment, the second SAM domain is derived from a protein listed in Table 17 below or comprises an amino acid sequence identical or similar to an amino acid sequence listed in Table 17, and the cargo binding domain comprises a first SAM domain. In a specific embodiment, the second SAM domain is derived from a protein listed in Table 17 below or comprises an amino acid sequence identical or similar to an amino acid sequence listed in Table 17, and the cargo binding domain comprises a first SAM domain and is from or derived from a corresponding parental Eph receptor listed in Table 17.

TABLE 17
Exemplary SAM domain-based binding.

| | Protein | | |
|---|---|---|---|
| | from | | |
| | which | Exemplary | |
| | scaffold | parental | |
| | binding | Eph | |
| | domain | receptor (s) | |
| | is derived | of | |
| SEQ | (UniProt | the cargo | |
| ID | ID | binding | SAM domain |
| NO: | No.) | domain | sequence |
| 184 | Ankyrin | EphAl/EphA2/ | TLEQSVGEWLESIGLQQYES |
| | repeat | EphA8 | KLLLNGFDDVHFLGSNVMEE |
| | and SAM | | QDLRDIGISDPQHRRKLLQA |
| | domain- | | ARSLPKV |
| | containing | | |
| | protein 1A | | |
| | (Q92625) | | |
| 185 | SHIP2 | EphA1/EphA2/ | LGEAGMSAWLRAIGLERYEE |
| | (O15357) | EphA6 | GLVHNGWDDLEFLSDITEED |
| | | | LEEAGVQDPAHKRLLLDTLQ |
| | | | LSK |
| 186 | SAMD5 | EphA1/EphA2/ | MCTNIVYEWLKALQLPQYAE |
| | (Q5TGI4) | EphA5/EphA6/ | SFVDNGYDDLEVCKQIGDPD |
| | | EphA7/EphA8/ | LDAIGVLAPAHRRRILEAVR |
| | | EphB1/EphB2/ | RLREQ |
| | | EphB3/EphB4 | |

In certain embodiments, the binding between the cargo binding domain and the cargo (e.g., cargo protein) is a PDZ or PBM domain-based binding. In specific embodiments, the cargo binding domain comprises a PBM domain and the cargo or the SBD comprises a domain capable of binding to the PBM domain (e.g., a PDZ domain), wherein the binding between the cargo binding domain and the cargo (e.g., cargo protein) is a binding between the PBM domain and the domain capable of binding to the PBM domain (e.g., the PDZ domain). In a specific embodiment, the PDZ domain is derived from a protein listed in Table 18 below or comprises an amino acid sequence identical or similar to an amino acid sequence listed in Table 18, and the cargo binding domain comprises a PBM domain. In a specific embodiment, the PDZ domain is derived from a protein listed in Table 18 below or comprises an amino acid sequence identical or similar to an amino acid sequence listed in Table 18, and the cargo binding domain comprises a PBM domain and is from or derived from a corresponding parental Eph receptor listed in Table 18. In some embodiments, the cargo binding domain comprises a PDZ domain (CDD accension number cl00117) and the cargo (e.g., cargo protein) or the SBD comprises a domain capable of binding to the PDZ domain (e.g., a PBM domain), and the binding between the cargo binding domain and the cargo (e.g., cargo protein) is a binding between the PDZ domain and the domain capable of binding to the PDZ domain (e.g., the PBM domain).

TABLE 18
Exemplary PDZ domain-based binding.

| | Protein | | |
|---|---|---|---|
| | from | Exemplary | |
| | which | parental | |
| | scaffold | Eph | |
| | binding | receptor (s) | |
| | domain | of | |
| SEQ | is derived | the cargo | |
| ID | (UniProt | binding | PDZ domain |
| NO: | ID No.) | domain | sequence |
| 187 | AF6 | EphA3/EphA4/ | IITVTLKKQNGMGLSIVAAK |
| | (P55196) | EphA6/EphA7 | GAGQDKLGIYVKSVVKGGAA |
| | | EphB2/EphB3/ | DVDGRLAAGDQLLSVDGRSL |
| | | EphB4/EphB6/ | VGLSQERAAELMTRTSSVVT |
| | | | LEVAKQG |
| 188 | GRIP1 | EphA7/EphB2 | TVELKRYGGPLGITISGTEE |
| | (Q9Y3R0) | | PFDPIIISSLTKGGLAERTG |
| | | | AIHIGDRILAINSSSLKGKP |
| | | | LSEAIHLLQMAGETVTLKIK |
| | | | KQT |
| 189 | GRIP2 | EphB2 | TVELKRYGGPLGITISGTEE |
| | (Q9C0E4) | | PFDPIVISGLTKRGLAERTG |
| | | | AIHVGDRILAINNVSLKGRP |
| | | | LSEAIHLLQVAGETVTLKIK |
| | | | KQL |
| 190 | Syntenin | EphA7 | EVILCKDQDGKIGLRLKSID |
| | Domain 1 | | NGIFVQLVQANSPASLVGLR |
| | (O00560) | | FGDQVLQINGENCAGWSSDK |
| | | | AHKVLKQAFGEKITMTIRDR |
| 191 | Syntenin | EphA7 | TITMHKDSTGHVGFIFKNGK |
| | Domain 2 | | ITSIVKDSSAARNGLLTEHN |
| | (O00560) | | ICEINGQNVIGLKDSQIADI |
| | | | LSTSGTVVTITIMPAF |
| 192 | Pick1 | EphA7/ | KVTLQKDAQNLIGISIGGGA |
| | (Q9NRD5) | EphB1/ | QYCPCLYIVQVFDNTPAALD |
| | | EphB2 | GTVAAGDEITGVNGRSIKGK |
| | | | TKVEVAKMIQEVKGEVTIHY |
| | | | NKLQ |
| 193 | SIPA1L1 | EphA4/ | HVNYEGIVADVEPYGYAWQA |
| | (043166) | EphA6 | GLRQGSRLVEICKVAVATLS |
| | | | HEQMIDLLRTSVTVKVVIIP |
| | | | PHDDCTPRRSCSETYRMPV |
| 194 | PTPN13 | EphA4/ | LITLIKSEKGSLGFTVTKGN |
| | (Q12923) | ephA3/ | QRIGCYVHDVIQDPAKSDGR |
| | | EphB2/ | LKPGDRLIKVNDTDVTNMTH |
| | | EphB4 | TDAVNLLRAASKTVRLVIGR |
| | | | V |

The PDZ-binding motifs at the C-terminal end of Eph receptors may serve as phosphorylation independent scaffold binding domains sites for PDZ domain-containing proteins (e.g. cargo proteins).

In certain embodiments, the binding between the cargo binding domain and the cargo (e.g., cargo protein) is a Dbl-homology-pleckstrin homology (DH-PH) motif-based binding. The pleckstrin homology domain is characterized by NCBI CDD accession number cl17171. In specific embodiments, the cargo binding domain comprises a DH-PH motif and the cargo or the SBD comprises a domain that is capable of binding to a DH-PH motif, and the binding between the cargo binding domain and the cargo (e.g., cargo protein) is a binding between the DH-PH motif and the domain that is capable of binding to a DH-PH motif. In specific embodiments, the cargo binding domain comprises a domain that is capable of binding to a DH-PH motif and the cargo (e.g., cargo protein) or the SBD comprises a DH-PH motif, and the binding between the cargo binding domain and the cargo (e.g., cargo protein) is a binding between the domain that is capable of binding to a DH-PH motif and the DH-PH motif. In a specific embodiment, the cargo (e.g., cargo protein) or the SBD comprises a DH-PH motif derived from a protein listed in Table 19 below or comprises an amino acid sequence identical or similar to an amino acid sequence listed in Table 19, and the cargo binding domain comprises a domain that is capable of binding to a DH-PH motif. In a specific embodiment, the cargo (e.g., cargo protein) or the SBD comprises a DH-PH motif derived from a protein listed in Table 19 below or comprises an amino acid sequence identical or similar to an amino acid sequence listed in Table 19, and the cargo binding domain comprises a domain that is capable of binding to a DH-PH motif and is from or derived from a corresponding parental Eph receptor listed in Table 19.

TABLE 19
Exemplary DH-PH motif-based binding.

| | Protein | | |
|---|---|---|---|
| | from | | |
| | which | Exemplary | |
| | scaffold | parental | |
| | binding | Eph | |
| | domain | receptor | |
| | is | (s) | |
| | derived | of the | |
| SEQ | (UniProt | cargo | |
| ID | ID | binding | |
| NO: | No.) | domain | DH-PH motif sequence |
| 195 | NGEF | EphA4 | KLQEAMFELVTSEASYYKSL |
| | (Q8N5V2) | | NLLVSHFMENERIRKILHPS |
| | | | EAHILFSNVLDVLAVSERFL |
| | | | LELEHRMEENIVISDVCDIV |
| | | | YRYAADHFSVYITYVSNQTY |
| | | | QERTYKQLLQEKAAFRELIA |
| | | | QLELDPKCRGLPFSSFLILP |
| | | | FQRITRLKLLVQNILKRVEE |
| | | | RSERECTALDAHKELEMVVK |
| | | | ACNEGVRKMSRTEQMISIQK |
| | | | KMEFKIKSVPIISHSRWLLK |
| | | | QGELQQMSGPKTSRTLRTKK |
| | | | LFHEIYLFLFNDLLVICRQI |
| | | | PGDKYQVFDSAPRGLLRVEE |
| | | | LEDQGQTLANVFILRLLENA |
| | | | DDREATYMLKASSQSEMKRW |
| | | | MTSLAPNRR |
| 196 | Ephexin-4 | EphA2 | RKRQEAMFEILTSEFSYQHS |
| | (Q5VV41) | | LSILVEEFLQSKELRATVTQ |
| | | | MEHHHLFSNILDVLGASQRF |
| | | | FEDLEQRHKAQVLVEDISDI |
| | | | LEEHAEKHFHPYIAYCSNEV |
| | | | YQQRTLQKLISSNAAFREAL |
| | | | REIERRPACGGLPMLSFLIL |
| | | | PMQRVTRLPLLMDTLCLKTQ |
| | | | GHSERYKAASRALKAISKLV |
| | | | RQCNEGAHRMERMEQMYTLH |
| | | | TQLDFSKVKSLPLISASRWL |
| | | | LKRGELFLVEETGLFRKIAS |
| | | | RPTCYLFLFNDVLVVTKKKS |
| | | | EESYMVQDYAQMNHIQVEKI |
| | | | EPSELPLPGGGNRSSSVPHP |
| | | | FQVTLLRNSEGRQEQLLLSS |
| | | | DSASDRARWIVALTHSER |

In certain embodiments, the binding between the cargo binding domain and the cargo (e.g., cargo protein) is a Dbl-homology (DH) motif-based binding. In specific embodiments, the cargo binding domain comprises a DH motif and the cargo (e.g., cargo protein) or the SBD comprises a domain that is capable of binding to a DH motif, and the binding between the cargo binding domain and the cargo (e.g., cargo protein) is a binding between the DH motif and the domain that is capable of binding to a DH motif. In specific embodiments, the cargo binding domain comprises a domain that is capable of binding to a DH motif and the cargo (e.g., cargo protein) or the SBD comprises a DH motif, and the binding between the cargo binding domain and the cargo (e.g., cargo protein) is a binding between the domain that is capable of binding to a DH motif and the DH motif.

In certain embodiments, the binding between the cargo binding domain and the cargo (e.g., cargo protein) is a pleckstrin homology (PH) motif-based binding. In specific embodiments, the cargo binding domain comprises a PH motif and the cargo (e.g., cargo protein) or the SBD comprises a domain that is capable of binding to a PH motif, and the binding between the cargo binding domain and the cargo (e.g., cargo protein) is a binding between the PH motif and the domain that is capable of binding to a PH motif. In specific embodiments, the cargo binding domain comprises a domain that is capable of binding to a PH motif and the cargo (e.g., cargo protein) or the SBD comprises a PH motif, and the binding between the cargo binding domain and the cargo (e.g., cargo protein) is a binding between the domain that is capable of binding to a PH motif and the PH motif.

In certain embodiments, a cargo binding domain described herein is from or derived from an ephrin receptor. In certain embodiments, a cargo binding domain described herein is not from and not derived from an ephrin receptor. In certain embodiments, a polypeptide described herein comprises a cargo binding domain that is from or derived from an ephrin receptor, and a cargo binding domain not from and not derived from an ephrin receptor. The polypeptide described herein can comprise one or more additional domains from or derived from one or more ephrin receptors that do not serve as cargo binding domain(s), for example, the ephrin receptor CR domain, two ephrin receptor FN III domains, an ephrin receptor TM domain, and optionally an ephrin receptor JM domain, an ephrin receptor KD, an ephrin receptor SAM linker domain, an ephrin receptor SAM domain, an ephrin receptor PBM domain, and/or a preferably inactivated ephrin receptor LBD. The polypeptide described herein can further comprise a targeting domain, a purification domain, and/or a modified Fc domain.

In certain embodiments, a cargo binding domain described herein is an ephrin receptor JM domain that is capable of binding to a cargo (e.g., a cargo protein) directly, or indirectly via a SBD linked to the cargo (e.g., cargo protein). In a specific embodiment, the ephrin receptor JM domain is C-terminal to the TM domain (e.g., the ephrin receptor TM domain). In a specific embodiment, the ephrin receptor JM domain is fused to the C-terminus of the TM domain (e.g., the ephrin receptor TM domain) (either with a linker such as a peptide linker described herein, or without a linker). In specific embodiments, the ephrin receptor JM domain comprises a phosphotyrosine and the cargo (e.g., cargo protein) or the SBD comprises a domain that is capable of binding to phosphotyrosine, and the binding between the ephrin receptor JM domain and the cargo (e.g., cargo protein) is a binding between the phosphotyrosine and the domain that is capable of binding to phosphotyrosine. In a specific embodiment, the domain that is capable of binding to phosphotyrosine is a PTB domain. In a specific embodiment, the domain that is capable of binding to phosphotyrosine is an SH2 domain. In a specific embodiment, the domain that is capable of binding to phosphotyrosine is a HYB domain, a GEP100 PH domain, a PKCδ domain, a PKCθ C2 domain, a catalytically inactive PTP domain, or a Raf-1 kinase inhibitory protein (RKIP) domain. In specific embodiments, the ephrin receptor JM domain comprises: (i) a (X₁)-Ptyr-(X₂) motif, wherein Ptyr is a phosphotyrosine, X₁ is Y, P, V, I, T, or F, and X₂ is I, V, L, or A; (ii) a (X₃)-Ptyr-(X₄) motif, wherein Ptyr is a phosphotyrosine, X₃ is T, A, or S, and X₄ is E or G; or (iii) both (i) and (ii). When a polypeptide described herein comprises a cargo binding domain that is an ephrin receptor JM domain, in specific embodiments, the polypeptide further comprises one, two, three, or four of the following: (1) an ephrin receptor KD, preferably C-terminal to the ephrin receptor JM domain; (2) a SAM linker domain (e.g., an ephrin receptor SAM linker domain), preferably C-terminal to the ephrin receptor JM domain; (3) a SAM domain (e.g., an ephrin receptor SAM domain), preferably C-terminal to the ephrin receptor JM domain; and (4) an ephrin receptor PBM domain, preferably C-terminal to the ephrin receptor JM domain.

In certain embodiments, a cargo binding domain described herein is an ephrin receptor KD that is capable of binding to a cargo (e.g., a cargo protein) directly, or indirectly via a SBD linked to the cargo (e.g., cargo protein). In a specific embodiment, the ephrin receptor KD is C-terminal to the TM domain (e.g., the ephrin receptor TM domain). In a specific embodiment, the ephrin receptor KD is fused to the C-terminus of the TM domain (e.g., the ephrin receptor TM domain) (either with a linker such as a peptide linker described herein, or without a linker). In a specific embodiment, the ephrin receptor KD is C-terminal to the ephrin receptor JM domain. In a specific embodiment, the ephrin receptor KD is fused to the C-terminus of the ephrin receptor JM domain (either with a linker such as a peptide linker described herein, or without a linker). In specific embodiments, the ephrin receptor KD comprises a phosphotyrosine and the cargo (e.g., cargo protein) or the SBD comprises a domain that is capable of binding to phosphotyrosine, and the binding between the ephrin receptor KD and the cargo (e.g., cargo protein) is a binding between the phosphotyrosine and the domain that is capable of binding to phosphotyrosine. In a specific embodiment, the domain that is capable of binding to phosphotyrosine is a PTB domain. In a specific embodiment, the domain that is capable of binding to phosphotyrosine is an SH2 domain. In a specific embodiment, the domain that is capable of binding to phosphotyrosine is a HYB domain, a GEP100 PH domain, a PKCδ domain, a PKCθ C2 domain, a catalytically inactive PTP domain, or a Raf-1 kinase inhibitory protein (RKIP) domain. In specific embodiments, the ephrin receptor KD comprises an (X₇)-Ptyr-(X₈) motif in the activation loop, wherein Ptyr is a phosphotyrosine, X₇ is T, V, or A, and X₈ is E or T. When a polypeptide described herein comprises a cargo binding domain that is an ephrin receptor KD, in specific embodiments, the polypeptide further comprises one, two, three, or four of the following: (1) an ephrin receptor JM domain, preferably N-terminal to the ephrin receptor KD; (2) a SAM linker domain (e.g., an ephrin receptor SAM linker domain), preferably C-terminal to the ephrin receptor KD; (3) a SAM domain (e.g., an ephrin receptor SAM domain), preferably C-terminal to the ephrin receptor KD; and (4) an ephrin receptor PBM domain, preferably C-terminal to the ephrin receptor KD. In a specific embodiment, a polypeptide provided herein comprise an EphB2 CR domain, a first EphB2 FN III domain, and a second EphB2 FN III domain, and further comprises an EphA2 KD serving as the cargo binding domain. In a specific embodiment, a polypeptide provided herein comprise an EphB2 CR domain, a first EphB2 FN III domain, a second EphB2 FN III domain, and an EphB2 TM domain, and further comprises an EphA2 KD serving as the cargo binding domain. In a specific embodiment, a polypeptide provided herein comprise an EphB2 CR domain, a first EphB2 FN III domain, a second EphB2 FN III domain, an EphB2 TM domain, and an EphA2 JM domain, and further comprises an EphA2 KD serving as the cargo binding domain.

In certain embodiments, a cargo binding domain described herein is an SAM linker domain (e.g., an ephrin receptor SAM linker domain) that is capable of binding to a cargo (e.g., a cargo protein) directly, or indirectly via a SBD linked to the cargo (e.g., cargo protein). In a specific embodiment, the SAM linker domain (e.g., the ephrin receptor SAM linker domain) is C-terminal to the TM domain (e.g., the ephrin receptor TM domain). In a specific embodiment, the SAM linker domain (e.g., the ephrin receptor SAM linker domain) is fused to the C-terminus of the TM domain (e.g., the ephrin receptor TM domain) (either with a linker such as a peptide linker described herein, or without a linker). In a specific embodiment, the SAM linker domain (e.g., the ephrin receptor SAM linker domain) is C-terminal to the ephrin receptor JM domain. In a specific embodiment, the SAM linker domain (e.g., the ephrin receptor SAM linker domain) is fused to the C-terminus of the ephrin receptor JM domain (either with a linker such as a peptide linker described herein, or without a linker). In a specific embodiment, the SAM linker domain (e.g., the ephrin receptor SAM linker domain) is C-terminal to the ephrin receptor KD. In a specific embodiment, the SAM linker domain (e.g., the ephrin receptor SAM linker domain) is fused to the C-terminus of the ephrin receptor KD (either with a linker such as a peptide linker described herein, or without a linker). In specific embodiments, the cargo (e.g., cargo protein) or the SBD comprises a domain capable of binding to the SAM linker domain (e.g., the ephrin receptor SAM linker domain), and the binding between the SAM linker domain (e.g., the ephrin receptor SAM linker domain) and the cargo (e.g., cargo protein) is a binding between the SAM linker domain (e.g., the ephrin receptor SAM linker domain) and the domain capable of binding to the SAM linker domain (e.g., the ephrin receptor SAM linker domain). In specific embodiments, the SAM linker domain (e.g., the ephrin receptor SAM linker domain) comprises a phosphorylated amino acid or a phosphomimetic amino acid and the cargo (e.g., cargo protein) or the SBD comprises a domain that is capable of binding to the phosphorylated amino acid or phosphomimetic amino acid, and the binding between the SAM linker domain (e.g., the ephrin receptor SAM linker domain) and the cargo (e.g., cargo protein) is a binding between the phosphorylated amino acid or phosphomimetic amino acid and the domain that is capable of binding to the phosphorylated amino acid or phosphomimetic amino acid. When a polypeptide described herein comprises a cargo binding domain that is a SAM linker domain (e.g., an ephrin receptor SAM linker domain), in specific embodiments, the polypeptide further comprises one, two, three, or four of the following: (1) an ephrin receptor JM domain, preferably N-terminal to the SAM linker domain (e.g., the ephrin receptor SAM linker domain); (2) an ephrin receptor KD, preferably N-terminal to the SAM linker domain (e.g., the ephrin receptor SAM linker domain); (3) a SAM domain (e.g., an ephrin receptor SAM domain), preferably C-terminal to the SAM linker domain (e.g., the ephrin receptor SAM linker domain); and (4) an ephrin receptor PBM domain, preferably C-terminal to the SAM linker domain (e.g., the ephrin receptor SAM linker domain).

In certain embodiments, a cargo binding domain described herein is a SAM domain (e.g., an ephrin receptor SAM domain) that is capable of binding to a cargo (e.g., a cargo protein) directly, or indirectly via a SBD linked to the cargo (e.g., cargo protein). In a specific embodiment, the SAM domain (e.g., the ephrin receptor SAM domain) is C-terminal to the TM domain (e.g., the ephrin receptor TM domain). In a specific embodiment, the SAM domain (e.g., the ephrin receptor SAM domain) is fused to the C-terminus of the TM domain (e.g., the ephrin receptor TM domain) (either with a linker such as a peptide linker described herein, or without a linker). In a specific embodiment, the SAM domain (e.g., the ephrin receptor SAM domain) is C-terminal to the ephrin receptor JM domain. In a specific embodiment, the SAM domain (e.g., the ephrin receptor SAM domain) is fused to the C-terminus of the ephrin receptor JM domain (either with a linker such as a peptide linker described herein, or without a linker). In a specific embodiment, the SAM domain (e.g., the ephrin receptor SAM domain) is C-terminal to the ephrin receptor KD. In a specific embodiment, the SAM domain (e.g., the ephrin receptor SAM domain) is fused to the C-terminus of the ephrin receptor KD (either with a linker such as a peptide linker described herein, or without a linker). In a specific embodiment, the SAM domain (e.g., the ephrin receptor SAM domain) is C-terminal to the SAM linker domain (e.g., the ephrin receptor SAM linker domain). In a specific embodiment, the SAM domain (e.g., the ephrin receptor SAM domain) is fused to the C-terminus of the SAM linker domain (e.g., the ephrin receptor SAM linker domain) (either with a linker such as a peptide linker described herein, or without a linker). In specific embodiments, the cargo protein or the SBD comprises a second SAM domain, and the binding between the SAM domain (e.g., the ephrin receptor SAM domain) and the cargo protein is a binding between the SAM domain (e.g., the ephrin receptor SAM domain) and the second SAM domain. The SAM domain (e.g., the ephrin receptor SAM domain) and the second SAM domain can be identical or different SAM domains. In specific embodiments, the SAM domain (e.g., the ephrin receptor SAM domain) comprises a phosphotyrosine and the cargo (e.g., cargo protein) or the SBD comprises a domain that is capable of binding to phosphotyrosine, and the binding between the SAM domain (e.g., the ephrin receptor SAM domain) and the cargo (e.g., cargo protein) is a binding between the phosphotyrosine and the domain that is capable of binding to phosphotyrosine. In a specific embodiment, the domain that is capable of binding to phosphotyrosine is a PTB domain. In a specific embodiment, the domain that is capable of binding to phosphotyrosine is an SH2 domain (e.g., the SH2 domain of Grb2 or the SH2 domain of Grb7). In a specific embodiment, the domain that is capable of binding to phosphotyrosine is a HYB domain, a GEP100 PH domain, a PKCδ domain, a PKCθ C2 domain, a catalytically inactive PTP domain, or a Raf-1 kinase inhibitory protein (RKIP) domain. In specific embodiments, the SAM domain (e.g., the ephrin receptor SAM domain) comprises a phosphotyrosine in the α2 helix. In a particular embodiment, the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T. When a polypeptide described herein comprises a cargo binding domain that is a SAM domain (e.g., an ephrin receptor SAM domain), in specific embodiments, the polypeptide further comprises one, two, three, or four of the following: (1) an ephrin receptor JM domain, preferably N-terminal to the SAM domain (e.g., the ephrin receptor SAM domain); (2) an ephrin receptor KD, preferably N-terminal to the SAM domain (e.g., the ephrin receptor SAM domain); (3) a SAM linker domain (e.g., an ephrin receptor SAM linker domain), preferably N-terminal to the SAM domain (e.g., the ephrin receptor SAM domain); and (4) an ephrin receptor PBM domain, preferably C-terminal to the SAM domain (e.g., the ephrin receptor SAM domain).

In certain embodiments, a cargo binding domain described herein is an ephrin receptor PBM domain that is capable of binding to a cargo (e.g., a cargo protein) directly, or indirectly via a SBD linked to the cargo (e.g., cargo protein). In a specific embodiment, the ephrin receptor PBM domain is C-terminal to the TM domain (e.g., the ephrin receptor TM domain). In a specific embodiment, the ephrin receptor PBM domain is fused to the C-terminus of the TM domain (e.g., the ephrin receptor TM domain) (either with a linker such as a peptide linker described herein, or without a linker). In a specific embodiment, the ephrin receptor PBM domain is C-terminal to the ephrin receptor JM domain. In a specific embodiment, the ephrin receptor PBM domain is fused to the C-terminus of the ephrin receptor JM domain (either with a linker such as a peptide linker described herein, or without a linker). In a specific embodiment, the ephrin receptor PBM domain is C-terminal to the ephrin receptor KD. In a specific embodiment, the ephrin receptor PBM domain is fused to the C-terminus of the ephrin receptor KD (either with a linker such as a peptide linker described herein, or without a linker). In a specific embodiment, the ephrin receptor PBM domain is C-terminal to the SAM linker domain (e.g., the ephrin receptor SAM linker domain). In a specific embodiment, the ephrin receptor PBM domain is fused to the C-terminus of the SAM linker domain (e.g., the ephrin receptor SAM linker domain) (either with a linker such as a peptide linker described herein, or without a linker). In a specific embodiment, the ephrin receptor PBM domain is C-terminal to the SAM domain (e.g., the ephrin receptor SAM domain). In a specific embodiment, the ephrin receptor PBM domain is fused to the C-terminus of the SAM domain (e.g., the ephrin receptor SAM domain) (either with a linker such as a peptide linker described herein, or without a linker). In specific embodiments, the cargo (e.g., cargo protein) or the SBD comprises a PDZ domain, and the binding between the ephrin receptor PBM domain and the cargo (e.g., cargo protein) is a binding between the ephrin receptor PBM domain and the PDZ domain. When a polypeptide described herein comprises a cargo binding domain that is an ephrin receptor PBM domain, in specific embodiments, the polypeptide further comprises one, two, three, or four of the following: (1) an ephrin receptor JM domain, preferably N-terminal to the ephrin receptor PBM domain; (2) an ephrin receptor KD, preferably N-terminal to the ephrin receptor PBM domain; (3) a SAM linker domain (e.g., an ephrin receptor SAM linker domain), preferably N-terminal to the ephrin receptor PBM domain; and (4) a SAM domain (e.g., an ephrin receptor SAM domain), preferably N-terminal to the ephrin receptor PBM domain.

### (c) Adaptor Proteins and Dimerization Agents

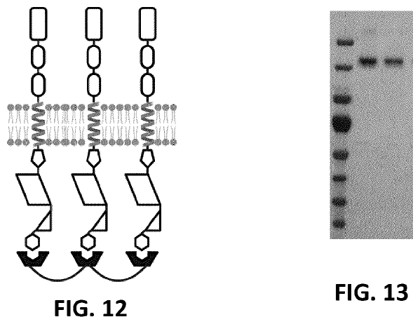

In one aspect, the polypeptides described herein can further be controlled spatially and temporally in relation to each other and brought into close proximity (e.g., cluster) by adaptor proteins (see, e.g., FIG. 12). The propensity of the transmembrane scaffold proteins to form multimers and the size of the resulting cluster depend on the total number of scaffold proteins in the membrane (e.g., a cell membrane or a nanovesicle membrane); but typically a large cluster is not easily formed, as new scaffold proteins need time to be synthesized and incorporated into the membrane and there is an equilibrium of synthesis versus degradation of scaffold proteins. In some embodiments, the use of adaptor proteins that bind to a cytosolic domain of the scaffold proteins can modify (e.g., increase) scaffold protein-scaffold protein interactions. In some embodiments, the binding of adaptor protein to scaffold proteins can be controlled spatially and temporally through processes including synthesis, buffering or enzymatic modifications like phosphorylation, methylation or cleavage of the adaptor protein.

In some embodiments, functional fragments of an adaptor protein are synthesized (e.g., by the source cell) and said fragments comprise a scaffold binding domain linked to an inducible dimerization agent (e.g., a chemically inducible dimerization agent), which fragments, upon addition of the dimerization chemical (e.g., rapamycin), form an adaptor protein, thereby bringing scaffold proteins into close proximity of each other.

In certain embodiments, the adaptor protein comprises a number of covalently linked scaffold binding domains as described in Section 5.2.3(b). In certain embodiments, the adaptor protein comprises a number of identical (e.g., repeated) covalently linker scaffold binding domains as described in Section 5.2.3(b). In certain embodiments, the adaptor protein comprises a number of heterologous covalently linked scaffold binding domains as described in Section 5.2.3(b).

In a certain embodiment, the adaptor protein comprises one or more scaffold binding domains that are capable of binding to phosphotyrosine, such as SBDs comprising PTB domains. In a specific embodiment, the adaptor protein comprises a PTB domain which is derived from CBL (UniProt ID No. P22681) or comprises an amino acid sequence of SEQ ID NO:160 as listed in Table 15, and the scaffold protein comprises a phosphotyrosine.

In a certain embodiment, the adaptor protein comprises one or more scaffold binding domains comprising domains that are capable of binding to phosphotyrosine, such as SH domains or variants thereof. In a specific embodiment, the adaptor protein comprises one or more scaffold binding domains comprising functional variants of a SH2 domain (NCBI CDD accension number cl15255). In a specific embodiment, adaptor protein comprises SH2 domains derived from one or more proteins listed in Table 16 or comprise one or more amino acid sequences identical or similar to those listed in Table 16.

In a certain embodiment, the adaptor protein comprises one or more scaffold binding domains comprising domains that are capable of binding to SAM domains. In a specific embodiment, the adaptor protein comprises one or more SAM domains derived from one or more proteins listed in Table 17 or comprises one or more amino acid sequences identical or similar to those listed in Table 17, and the scaffold protein also comprises a SAM domain.

In certain embodiments, the binding between the adaptor protein and the scaffold protein is a PDZ domain-based binding. In specific embodiments, the adaptor protein comprises a scaffold binding domain comprising a PDZ domain and the scaffold protein comprises a PBM domain, and the binding between the adaptor protein and the scaffold protein is a binding between the PDZ domain and the PBM domain. In a specific embodiment, the adaptor protein comprises a PDZ domain derived from a protein listed in Table 18 or comprises an amino acid sequence identical or similar to an amino acid sequence listed in Table 18, and the scaffold protein comprises a PBM domain.

In some embodiments, the adaptor protein comprises two, three, four or five scaffold binding domains as described above. In certain embodiments, the adaptor protein comprises two or more linked hetero-domain scaffold binding domains. In some embodiments, the adaptor protein comprises one, two, three, four and/or five scaffold binding domains and each scaffold domain interacts with one scaffold protein.

### 5.2.4 Targeting Domain and Purification Domain

As described above, a polypeptide provided herein can also comprise one or more functional moieties (e.g., fusion moieties, see, e.g., FIGS. 4-7), preferably a targeting domain that is capable of targeting a nanovesicle (e.g., EV or hybridosome) comprising the polypeptide to a specific organ, tissue, or cell type, and/or a purification domain that can facilitate purification of such a nanovesicle (e.g., EV or hybridosome). See FIG. 8 for schematic illustrations of exemplary Eph receptor derived polypeptides, with a targeting domain. In a preferred embodiment, the one or more functional moieties are proteins (e.g., peptides or polypeptides). In a preferred embodiment, the one or more functional moieties are fused in-frame to the remaining portion of the polypeptide. In certain embodiments, the one or more functional moieties are covalently fused to the remaining portion of the polypeptide via a linker. In specific embodiments, the linker is a peptide linker. In a specific embodiment, the peptide linker comprises an amino acid sequence of (GGGS)n (SEQ ID NO:231), wherein n is an integer number from 1 to 10. In a specific embodiment, the peptide linker comprises an amino acid sequence of GGGS. In a specific embodiment, the peptide linker comprises an amino acid sequence of (GGGS)2 (SEQ ID NO:232). In a specific embodiment, the peptide linker comprises an amino acid sequence of (GGGS)3 (SEQ ID NO:233).

Such one or more functional moieties can be N- or C-terminal to (e.g., N-terminally and/or C-terminally fused to) the remaining portion of the polypeptide or placed between the different domains of the remaining portion of the polypeptide. In certain embodiments, the one or more functional moieties are presented towards the external space of a nanovesicle. In some embodiments, the one or more functional moieties are N-terminal to (e.g., N-terminally fused to) the ephrin ligand binding domain of the polypeptide. In some embodiments, the one or more functional moieties are N-terminal to (e.g., N-terminally fused to) the ephrin receptor cysteine rich domain of the polypeptide. In some embodiments, the one or more functional moieties are N-terminal to (e.g., N-terminally fused to) the ephrin receptor FN1 domain. In some embodiments, the one or more functional moieties are N-terminal to (e.g., N-terminally fused to) the ephrin receptor FN2 domain. In some embodiments, the one or more functional moieties are N-terminal to (e.g., N-terminally fused to) the TM domain (e.g., the ephrin receptor TM domain). In certain embodiments, the one or more functional moieties are presented towards the lumen of a nanovesicle. In some embodiments, the one or more functional moieties are C-terminal to (e.g., C-terminally fused to) the TM domain (e.g., the ephrin receptor TM domain). In some embodiments, the one or more functional moieties are C-terminal to (e.g., C-terminally fused to) the ephrin receptor JM domain. In some embodiments, the one or more functional moieties are C-terminal to (e.g., C-terminally fused to) the ephrin receptor KD. In some embodiments, the one or more functional moieties are C-terminal to (e.g., C-terminally fused to) the SAM linker domain (e.g., the ephrin receptor SAM linker domain). In some embodiments, the one or more functional moieties are C-terminal to (e.g., C-terminally fused to) the SAM domain (e.g., the ephrin receptor SAM domain). In some embodiments, the one or more functional moieties are C-terminal to (e.g., C-terminally fused to) the ephrin receptor PBM domain.

In certain embodiments, the one or more functional moieties are N-terminal to (e.g., N-terminally fused to) a cargo (e.g., a cargo protein) described in this disclosure. In certain embodiments, the one or more functional moieties are C-terminal to (e.g., C-terminally fused to) a cargo (e.g., a cargo protein) described in this disclosure. In certain embodiments, the one or more functional moieties are N-terminal to (e.g., N-terminally fused to) a cargo binding domain described in this disclosure. In certain embodiments, the one or more functional moieties are C-terminal to (e.g., C-terminally fused to) a cargo binding domain described in this disclosure. In certain embodiments, the one or more functional moieties are N-terminal to (e.g., N-terminally fused to) a modified Fc domain described in this disclosure. In certain embodiments, the one or more functional moieties are C-terminal to (e.g., C-terminally fused to) a modified Fc domain described in this disclosure.

Exemplary functional moieties include, without being limited to, targeting domains and purification domains such as affinity tags. The functional moieties may be a large polypeptide or a peptide.

In certain embodiments, a targeting domain described herein is N-terminal to (e.g., N-terminally fused to) a purification domain described herein. In certain embodiments, a targeting domain described herein is C-terminal to (e.g., C-terminally fused to) a purification domain described herein.

In some embodiments targeting domains are preferably located on the surface of a nanovesicle. Thus, in some embodiments, a targeting domain is fused to the N-terminal of the scaffold. A targeting domain aids directing the nanovesicle towards a specific organ, tissue, or cell and is preferably specific to an organ, a tissue, or a cell. One or more targeting domains may be fused to the remaining portion of the polypeptide. The presence of more than one targeting domain may increase specificity for the targeted organ, tissue, or cell. In some embodiments, the targeting domain is or comprises one or more antigen binding molecules. In some embodiments, the targeting domain specifically targets an antigen expressed on cancer, metastatic, dendritic, stem or immunological cell. Exemplary antigens expressed on tumor cells include, without being limited to, BAGE, BCMA, CEA, CD19, CD20, CD33, CD123, CEA, FAP, HER2, LMP1, LMP2, MAGE, Mart1/MelanA, NY-ESO, PSA, PSMA, RAGE and survivin.

In some embodiments targeting domains are located in the lumen of a nanovesicle. Thus, in some embodiments, a targeting domain is fused to the C-terminal of the scaffold. A targeting domain aids attaching cytoplasmic components (e.g. proteins, protein-complex, viruses) to the scaffold prior to invagination and vesicle formation. One or more targeting domains may be fused to C-terminus of the polypeptide. The presence of more than one targeting domain may increase loading efficiency of cytoplasmic components into the lumen of the nanovesicle during biogenesis. In some embodiments, the targeting domain is or comprises one or more antigen binding molecules. In some embodiments, the targeting domain specifically targets an antigen expressed on adeno-associated viruses.

In certain embodiments, the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

In certain embodiments, the targeting domain specifically binds to a marker. In specific embodiments, the marker is a tumor-associated antigen. In a specific embodiment, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

As outlined above, in certain embodiments, a polypeptide described herein is engineered such that the polypeptide has reduced affinity for an ephrin. In some embodiments, the affinity of the polypeptide for an ephrin is lower than the binding affinity of the targeting domain for its target. In some embodiments, this binding affinity differential is between the polypeptide and the targeting domain and its target on the same recipient cell. In some embodiments, this binding affinity differential allows for the polypeptide scaffold to have localized, on-target effects and to minimize off-target effects that underlie side effects that are observed with the wildtype Eph receptor. In some embodiments, this binding affinity of the polypeptide for ephrin is at least 2-fold, or at least 5-fold, or at least 10-fold, or at least 15-fold lower, or at least 25-fold, or at least 50-fold lower, or at least 100-fold, or at least 150-fold less than the binding affinity of the targeting domain for its target.

In some aspects, methods of targeting nanovesicles to a specific organ, tissue or cell are provided, comprising the steps of fusing a targeting domain to the remaining portion of a polypeptide of the disclosure and getting the polypeptide expressed in nanovesicles.

Antigen binding molecules serving as targeting domains, may be monospecific, bispecific or multispecific, i.e., they may target one or more epitopes of the same target or different targets. The more specificities are displayed on the nanovesicle, the more specific its targeting is. In some embodiments, the antigen binding molecule is selected from the group consisting of:
i) a full-length antibody molecule (such as an IgG, an IgM, an IgA, an IgM or an IgE);
ii) an antibody fragment such as a CDR, a Dab, a Fab, a Fab′, a F(ab)′2, a Fd fragment, a Fv fragment, a disulfide linked Fv, a scFab, a nanobody, a minimal recognition unit, a VHH or a V-NAR domain;
iii) a non-antibody scaffold such as an affibody, an affilin molecule, an affitin, an AdNectin, an anticalin, an avimer, a centyrin, a lipocalin mutein, a DARPin, a fynomer, a Knottin, a Kunitz-type domain, a nanofitin, a tetranectin or a trans-body; iv) a fusion polypeptide comprising one or more antibody domains, such as a bi-scFv, aBITE, a diabody, di-scFv, probody, tascFv (tandem scFv), triabody, tribody, tetrabody, IgGACH2, DVD-Ig, MATCH, a minibody, a scFv, a scFv-Fc, bispecific F(ab′)2, F(ab′)3, monovalent IgG;
v) a soluble T-cell receptor (sTCR);
vi) a peptide, such as natural peptide, a recombinant peptide, a synthetic peptide; and/or
vii) a viral protein such as the receptor binding domain of a viral spike protein (such as of coronavirus) or hemagglutinin (HA) of influenza, Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein, or fragments thereof, respectively.

As explained above, a polypeptide described herein can comprise a purification domain that can facilitate purification of nanovesicles comprising the polypeptide. In certain embodiments, a binding partner of the purification domain is attached to a solid phase to enable purification, e.g., chromatography and/or membrane-based purification. In specific embodiments, the purification domain and the binding partner bind to each other with high affinity under a first set of condition(s) and with low affinity under a second set of conditions, thereby allowing nanovesicles comprising a polypeptide that contains the purification domain to be immobilized on the solid phase under the first set of condition(s) and later eluted from the solid phase under the second set of condition(s). In certain embodiments, the purification domain is an affinity tag. In certain embodiments, the purification domain is a modified Fc domain described in Section 5.2.5 and its binding partner comprises the Fc binding site of an Fc receptor (such as a neonatal Fc receptor (FcRn)).

In certain embodiments, a polypeptide described herein comprises a purification domain that allows nanovesicles comprising the polypeptide to be eluted from its immobilized binding partner under a mild condition, for example, at a mild pH (e.g., pH 7-pH 9).

The polypeptide may or may not comprise an affinity tag which is typically a short sequence having affinity to a binding agent. Such an affinity tag can be used for purification or removal of the nanovesicles comprising the polypeptide of the disclosure with a binding agent specific to the affinity tag. Exemplary embodiments of affinity tags include, without being limited to, His tag, GST tag, glutathione-S-transferase, S-peptide, HA, Myc, FLAG™ (Sigma-Aldrich Co.), MBP, intenin, SUMO, Protein A, and Protein G.

### 5.2.5 Modified Fc Domain

In various embodiments, a polypeptide described herein further comprises a modified Fe domain of an immunoglobulin. See FIG. 8 for schematic illustrations of exemplary Eph receptor derived polypeptides, with a modified Fc domain. In certain embodiments, the modified Fc domain can be fused in-frame to the remaining portion of the polypeptide. In certain embodiments, the modified Fc domain is fused to the remaining portion of the polypeptide via a linker (e.g., a linker sequence). In some embodiments, the modified Fc domain is covalently fused to the remaining portion of the polypeptide via a linker (e.g., a linker sequence). In specific embodiments, the linker is a peptide linker. In a specific embodiment, the peptide linker comprises an amino acid sequence of (GGGS)n (SEQ ID NO:231), wherein n is an integer number from 1 to 10. In a specific embodiment, the peptide linker comprises an amino acid sequence of GGGS. In a specific embodiment, the peptide linker comprises an amino acid sequence of (GGGS)2 (SEQ ID NO:232). In a specific embodiment, the peptide linker comprises an amino acid sequence of (GGGS)3 (SEQ ID NO:233).

Such a modified Fc domain can be N- or C-terminal to (e.g., N-terminally and/or C-terminally fused to) the remaining portion of the polypeptide or placed between the different domains of the remaining portion of the polypeptide. In certain embodiments, a modified Fc domain is presented towards the external space of a nanovesicle. In some embodiments, the modified Fc domain is N-terminal to (e.g., N-terminally fused to) the ephrin ligand binding domain of the polypeptide. In some embodiments, the modified Fc domain is N-terminal to (e.g., N-terminally fused to) the ephrin receptor cysteine rich domain of the polypeptide. In some embodiments, the modified Fc domain is N-terminal to (e.g., N-terminally fused to) the ephrin receptor FN1 domain. In some embodiments, the modified Fc domain is N-terminal to (e.g., N-terminally fused to) the ephrin receptor FN2 domain. In some embodiments, the modified Fc domain is N-terminal to (e.g., N-terminally fused to) the TM domain (e.g., the ephrin receptor TM domain). In certain embodiments, a modified Fc domain is presented towards the lumen of a nanovesicle. In some embodiments, the modified Fc domain is C-terminal to (e.g., C-terminally fused to) the TM domain (e.g., the ephrin receptor TM domain). In some embodiments, the modified Fc domain is C-terminal to (e.g., C-terminally fused to) the ephrin receptor JM domain. In some embodiments, the modified Fc domain is C-terminal to (e.g., C-terminally fused to) the ephrin receptor KD. In some embodiments, the modified Fc domain is C-terminal to (e.g., C-terminally fused to) the SAM linker domain (e.g., the ephrin receptor SAM linker domain). In some embodiments, the modified Fc domain is C-terminal to (e.g., C-terminally fused to) the SAM domain (e.g., the ephrin receptor SAM domain). In some embodiments, the modified Fe domain is C-terminal to (e.g., C-terminally fused to) the ephrin receptor PBM domain.

In certain embodiments, the modified Fe domain is N-terminal to (e.g., N-terminally fused to) a targeting domain described in this disclosure. In certain embodiments, the modified Fc domain is C-terminal to (e.g., C-terminally fused to) a targeting domain described in this disclosure. In certain embodiments, the modified Fc domain is N-terminal to (e.g., N-terminally fused to) a purification domain described in this disclosure. In certain embodiments, the modified Fc domain is C-terminal to (e.g., C-terminally fused to) a purification domain described in this disclosure. In certain embodiments, the modified Fc domain is N-terminal to (e.g., N-terminally fused to) a cargo (e.g., a cargo protein) described in this disclosure. In certain embodiments, the modified Fc domain is C-terminal to (e.g., C-terminally fused to) a cargo (e.g., a cargo protein) described in this disclosure. In certain embodiments, the modified Fc domain is N-terminal to (e.g., N-terminally fused to) a cargo binding domain described in this disclosure. In certain embodiments, the modified Fc domain is C-terminal to (e.g., C-terminally fused to) a cargo binding domain described in this disclosure.

In certain embodiments, the modified Fc domain is capable of specifically binding to the Fc binding site of a neonatal Fc receptor (FcRn), and lacks the ability to form homodimers. In certain embodiments, the dissociation constant of the modified Fc domain bound to the FcRn at a pH of 6.5 has a value of at most 10⁻⁴ M. In certain embodiments, the dissociation constant of the modified Fc domain bound to the FcRn at a pH of 7.4 has a value of at least 10⁻⁴ M. In certain embodiments, the modified Fc domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid. In certain embodiments, the modified Fc domain is capable of specifically binding to the amino acid sequence between position 135-158 of human FcRn (SEQ ID NO: 228) and/or mouse FcRn (SEQ ID NO: 227).

In certain embodiments, the polypeptide comprising a modified Fc domain does not substantially bind to C1q, FcγRI, FcγRII or FcγRIII.

In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fe domain, is decreased by at least 10%, 20%, 30%, 40%, or 50% compared to an unmodified Fc domain.

In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 1.5, 2, 3, 4, or 5-fold, compared to an unmodified Fc domain.

In certain embodiments, the modified Fc domain comprises from N-terminus to C-terminus: a modified CH2 domain that is modified to decrease effector function relative to the unmodified CH2 domain, and a modified CH3 domain that is modified to lack the ability to form homodimers.

In certain embodiments, a modified Fc domain (such as a modified Fc domain described herein) is used as a purification domain as described in Section 5.2.4, which can facilitate the purification of nanovesicles comprising a polypeptide that contains the modified Fc domain. When a modified Fc domain is used as a purification domain, its binding partner used for purification, e.g., the binding partner attached to a solid phase, comprises the Fc binding site of an Fc receptor (such as a neonatal Fc receptor (FcRn)). In specific embodiments, the modified Fc domain and its binding partner bind to each other with high affinity under a first set of condition(s) and with low affinity under a second set of conditions, thereby allowing nanovesicles comprising a polypeptide that contains the modified Fc domain to be immobilized on the solid phase under the first set of condition(s) and later eluted from the solid phase under the second set of condition(s). In various embodiments, the modified Fc domain present on a polypeptide described herein enable large scale purification of nanovesicles comprising such polypeptide.

In certain embodiments, a polypeptide comprises a modified Fc domain (such as a modified Fc domain described herein) that improves pharmacokinetic properties of nanovesicles comprising the polypeptide, e.g., by at least 10%, at least 20%, at least 30%, at least 40%, at least 50%, at least 60%, at least 70%, at least 80%, at least 90%, at least 100%, at least 200%, at least 400%, at least 800%, at least 1,000%, or at least 10,000%, relative to what nanovesicles would exhibit without the polypeptide. In specific embodiments, a polypeptide comprises a modified Fc domain (such as a modified Fc domain described herein) that extends the half-life of nanovesicles comprising the polypeptide in the circulation, e.g., by at least 10%, at least 20%, at least 30%, at least 40%, at least 50%, at least 60%, at least 70%, at least 80%, at least 90%, at least 100%, at least 200%, at least 400%, at least 800%, at least 1,000%, or at least 10,000%, relative to what nanovesicles would exhibit without the polypeptide.

In a specific embodiment, a polypeptide described herein comprises in N-terminus to C-terminus direction: a targeting domain (e.g., a targeting monobody), optionally a linker, a modified Fc domain (e.g., a monomeric Fc domain), optionally a linker, an ephrin receptor CR domain (e.g., an EphB2 CR domain), a first ephrin receptor FN III domain and a second ephrin receptor FN III domain (e.g., the first and second EphB2 FN III domains), a TM domain (e.g., an EphB2 TM domain), an ephrin receptor JM domain (e.g., an EphA2 JM domain), and an ephrin receptor KD (e.g., an EphA2 KD).

In a specific embodiment, a polypeptide described herein comprises in N-terminus to C-terminus direction: a targeting monobody, optionally a linker, a monomeric Fc domain, optionally a linker, an EphB2 CR domain, the first and second EphB2 FN III domains, an EphB2 TM domain, an EphA2 JM domain, and an EphA2 KD.

In a specific embodiment, a polypeptide described herein comprises in N-terminus to C-terminus direction: a targeting monobody, a linker, a monomeric Fc domain, a linker, an EphB2 CR domain, the first and second EphB2 FN III domains, an EphB2 TM domain, an EphA2 JM domain, and an EphA2 KD.

### 5.2.6 Linkers

As is normally the case with fusion proteins, the two components that are normally included in the fusion protein may be linked directly in a contiguous fashion in the fusion protein, or they may be linked and/or attached to each other using a variety of linkers.

In various embodiments, any of the two domains present in a polypeptide described herein and any of the two portions of a polypeptide described herein may be fused together via a linker, preferably a peptide linker. For example, a cargo (e.g., a cargo protein), a cargo binding domain, a targeting domain, a purification domain, and/or a modified Fc domain as described herein can be fused to the remaining portion of the polypeptide via one or more linkers, preferably one or more peptide linkers. Any of the peptide linkers may comprise a length of at least 5 residues, at least 10 residues, at least 15 residues, at least 20 residues, at least 25 residues, at least 30 residues or more. In other embodiments, the linkers comprises a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues. In some embodiments, the linker comprises a flexible linker. In some embodiments, the linker comprises a glycine-serine linker, i.e., a linker that consists primarily of, or entirely of, stretches of glycine and serine residues. In some embodiments, the linker comprises a (G4S)n linker (GGGGS)n (SEQ ID NO:234), wherein n is an integer number from 1 to 10. In some embodiments, the linker comprises a G4S (SEQ ID NO:242) linker, a (G4S)2 (SEQ ID NO:235) linker, a (G4S)3 (SEQ ID NO:236) linker, a (G4S)2-G4 (SEQ ID NO:237) linker, or a G3S-(G4S)4-G2 (SEQ ID NO:238) linker. In a specific embodiment, the peptide linker comprises an amino acid sequence of (GGGS)n (SEQ ID NO:231), wherein n is an integer number from 1 to 10. In a specific embodiment, the peptide linker comprises an amino acid sequence of GGGS. In a specific embodiment, the peptide linker comprises an amino acid sequence of (GGGS)2 (SEQ ID NO:232). In a specific embodiment, the peptide linker comprises an amino acid sequence of (GGGS)3 (SEQ ID NO:233). In some embodiments, the linker is a glycine-serine linker comprising one or more modifications.

For example, the ectodomain of a polypeptide described herein may be fused via a linker to a transmembrane domain at its C-terminal end and also be fused to a fusion moiety (e.g., an exogenous biologically active molecule disclosed herein, such as an antigen, targeting moiety, adjuvant, immune modulator, a cargo binding domain, a targeting domain, a purification domain, and/or a modified Fc domain) via a linker at its N-terminal end.

Accordingly, depending on the type of transmembrane domain used, possible linear configurations for such fusion proteins may be illustrated as follows in N-terminal to C-terminal order:
(fusion moiety)-L-(LBD)-(flexible domain)-L-(transmembrane domain)-, or
(fusion moiety)-L-(flexible domain)-L-(transmembrane domain) or
(fusion moiety)-L-(ectodomain)-L-(transmembrane domain)
   where each L in the formulae represents a direct peptide bond linking two domains or a linker as described above. The two Ls in the same formula can be the same peptide bond or linker or can be different peptide bonds or linkers. Such configurations will provide for an extracellular or surface presentation of the fusion moiety.

Alternatively, possible linear configurations for such fusion proteins may be illustrated in the following form, again in N-terminal to C-terminal order:
(ectodomain)-L-(transmembrane domain)-L-(fusion moiety) (if a Type I transmembrane protein-derived transmembrane domain is used),
(fusion moiety)-L-(transmembrane domain)-L-(ectodomain) (if a Type II transmembrane protein-derived transmembrane domain is used),
(flexible domain)-L-(transmembrane domain)-L-(fusion moiety) (if a Type I transmembrane protein-derived transmembrane domain is used), or
(fusion moiety)-L-(transmembrane domain)-L-(flexible domain) (if a Type II transmembrane protein-derived transmembrane domain is used),
   where each L in the formulae represents a direct peptide bond linking two domains or a linker as described above. The two Ls in the same formula can be the same peptide bond or linker or can be different peptide bonds or linkers. These configurations will provide for an intracellular or intraluminar presentation of the fusion moiety. Intracellular and/or intraluminar presentation of heterologous polypeptides may provide increased protection, e.g., from proteases, and potentially elicit fewer off-target interactions, as the therapeutic enzyme would not be able to interact with external molecules.

If a polypeptide described herein comprises two fusion moieties, each fusion moiety may be connected at the C-terminal end to a linker. Accordingly, possible linear configurations for such polypeptides may be illustrated as follows in N-terminal to C-terminal order:
(fusion moiety1)-L-(fusion moiety2)-L-(ectodomain)-L-(transmembrane domain), or
(fusion moiety1)-L-(fusion moiety2)-L-(flexible domain)-L-(transmembrane domain),
   where each L in the formulae represents a direct peptide bond linking two domains or a linker as described above. The two Ls in the same formula can be the same peptide bond or linker or can be different peptide bonds or linkers.

Alternatively, possible linear configurations in N-terminal to C-terminal order for polypeptides comprising two fusion moieties may be illustrated as follows:
(fusion moiety1)-L-(ectodomain)-L-(transmembrane domain)-L-(fusion moiety2), or
(fusion moiety1)-L-(flexible domain)-L-(transmembrane domain)-L-(fusion moiety2),
   where each L in the formulae represents a direct peptide bond linking two domains or a linker as described above. The two Ls in the same formula can be the same peptide bond or linker or can be different peptide bonds or linkers.

In specific embodiments, the polypeptide comprises an ectodomain and a transmembrane domain that are derived from EphA1. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA1, and wherein said polypeptide exhibits decreased or no binding to ephrins as compared to the parental Eph receptor. In some embodiments, the polypeptide comprises an amino acid sequence identical or similar to SEQ ID NO:198 or a fragment thereof and has at least 70%, at least 80%, at least 85%, at least 90%, at least 95%, at least 96%, at least 97%, at least 98%, or at least 99% sequence identity to SEQ ID NO: 198, and wherein said polypeptide exhibits decreased or no binding to ephrins as compared to the parental Eph receptor. In some embodiments, the portion of the polypeptide derived from EphA1 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA1-derived portion. In some embodiments, the one or more heterologous proteins are fused to the C-terminus of said EphA1-derived portion. In some embodiments, the one or more heterologous proteins are fused to both the N-terminus and the C-terminus of said EphA1-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA1-derived portion. In certain embodiments said targeting domain is fused to the C-terminus of a modified Fc domain (e.g., as described in section 5.2.5), and said modified Fc domain is fused to the N-terminus of said EphA1-derived portion.

In specific embodiments, the polypeptide comprises an ectodomain and a transmembrane domain that are derived from EphA2. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA2, and wherein said polypeptide exhibits decreased or no binding to ephrins as compared to the parental Eph receptor. In some embodiments, the polypeptide comprises an amino acid sequence identical or similar to SEQ ID NO: 199 or a fragment thereof and has at least 70%, at least 80%, at least 85%, at least 90%, at least 95%, at least 96%, at least 97%, at least 98%, or at least 99% sequence identity to SEQ ID NO: 199, and wherein said polypeptide exhibits decreased or no binding to ephrins as compared to the parental Eph receptor. In some embodiments, the portion of the polypeptide derived from EphA2 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA2-derived portion. In some embodiments, the one or more heterologous proteins are fused to the C-terminus of said EphA2-derived portion. In some embodiments, the one or more heterologous proteins are fused to both the N-terminus and the C-terminus of said EphA2-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA2-derived portion. In certain embodiments said targeting domain is fused to the C-terminus of a modified Fc domain (e.g., as described in section 5.2.5), and said modified Fc domain is fused to the N-terminus of said EphA2-derived portion.

In specific embodiments, the polypeptide comprises an ectodomain and a transmembrane domain that are derived from EphA3. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA3, and wherein said polypeptide exhibits decreased or no binding to ephrins as compared to the parental Eph receptor. In some embodiments, the polypeptide comprises an amino acid sequence identical or similar to SEQ ID NO: 200 or a fragment thereof and has at least 70%, at least 80%, at least 85%, at least 90%, at least 95%, at least 96%, at least 97%, at least 98%, or at least 99% sequence identity to SEQ ID NO: 200, and wherein said polypeptide exhibits decreased or no binding to ephrins as compared to the parental Eph receptor. In some embodiments, the portion of the polypeptide derived from EphA3 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA3-derived portion. In some embodiments, the one or more heterologous proteins are fused to the C-terminus of said EphA3-derived portion. In some embodiments, the one or more heterologous proteins are fused to both the N-terminus and the C-terminus of said EphA3-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA3-derived portion. In certain embodiments said targeting domain is fused to the C-terminus of a modified Fc domain (e.g., as described in section 5.2.5), and said modified Fc domain is fused to the N-terminus of said EphA3-derived portion.

In specific embodiments, the polypeptide comprises an ectodomain and a transmembrane domain that are derived from EphA4. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA4, and wherein said polypeptide exhibits decreased or no binding to ephrins as compared to the parental Eph receptor. In some embodiments, the polypeptide comprises an amino acid sequence identical or similar to SEQ ID NO: 201 or a fragment thereof and has at least 70%, at least 80%, at least 85%, at least 90%, at least 95%, at least 96%, at least 97%, at least 98%, or at least 99% sequence identity to SEQ ID NO: 201, and wherein said polypeptide exhibits decreased or no binding to ephrins as compared to the parental Eph receptor. In some embodiments, the portion of the polypeptide derived from EphA4 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA4-derived portion. In some embodiments, the one or more heterologous proteins are fused to the C-terminus of said EphA4-derived portion. In some embodiments, the one or more heterologous proteins are fused to both the N-terminus and the C-terminus of said EphA4-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA4-derived portion. In certain embodiments said targeting domain is fused to the C-terminus of a modified Fc domain (e.g., as described in section 5.2.5), and said modified Fc domain is fused to the N-terminus of said EphA4-derived portion.

In specific embodiments, the polypeptide comprises an ectodomain and a transmembrane domain that are derived from EphA5. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA5 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA5, and wherein said polypeptide exhibits decreased or no binding to ephrins as compared to the parental Eph receptor. In some embodiments, the polypeptide comprises an amino acid sequence identical or similar to SEQ ID NO: 202 or a fragment thereof and has at least 70%, at least 80%, at least 85%, at least 90%, at least 95%, at least 96%, at least 97%, at least 98%, or at least 99% sequence identity to SEQ ID NO: 202, and wherein said polypeptide exhibits decreased or no binding to ephrins as compared to the parental Eph receptor. In some embodiments, the portion of the polypeptide derived from EphA5 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA5-derived portion. In some embodiments, the one or more heterologous proteins are fused to the C-terminus of said EphA5-derived portion. In some embodiments, the one or more heterologous proteins are fused to both the N-terminus and the C-terminus of said EphA5-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA5-derived portion. In certain embodiments said targeting domain is fused to the C-terminus of a modified Fc domain (e.g., as described in section 5.2.5), and said modified Fc domain is fused to the N-terminus of said EphA5-derived portion.

In specific embodiments, the polypeptide comprises an ectodomain and a transmembrane domain that are derived from EphA6. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA6 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA6, and wherein said polypeptide exhibits decreased or no binding to ephrins as compared to the parental Eph receptor. In some embodiments, the polypeptide comprises an amino acid sequence identical or similar to SEQ ID NO: 203 or a fragment thereof and has at least 70%, at least 80%, at least 85%, at least 90%, at least 95%, at least 96%, at least 97%, at least 98%, or at least 99% sequence identity to SEQ ID NO: 203, and wherein said polypeptide exhibits decreased or no binding to ephrins as compared to the parental Eph receptor. In some embodiments, the portion of the polypeptide derived from EphA6 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA6-derived portion. In some embodiments, the one or more heterologous proteins are fused to the C-terminus of said EphA6-derived portion. In some embodiments, the one or more heterologous proteins are fused to both the N-terminus and the C-terminus of said EphA6-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA6-derived portion. In certain embodiments said targeting domain is fused to the C-terminus of a modified Fc domain (e.g., as described in section 5.2.5), and said modified Fc domain is fused to the N-terminus of said EphA6-derived portion.

In specific embodiments, the polypeptide comprises an ectodomain and a transmembrane domain that are derived from EphA7. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA7 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA7, and wherein said polypeptide exhibits decreased or no binding to ephrins as compared to the parental Eph receptor. In some embodiments, the polypeptide comprises an amino acid sequence identical or similar to SEQ ID NO: 204 or a fragment thereof and has at least 70%, at least 80%, at least 85%, at least 90%, at least 95%, at least 96%, at least 97%, at least 98%, or at least 99% sequence identity to SEQ ID NO: 204, and wherein said polypeptide exhibits decreased or no binding to ephrins as compared to the parental Eph receptor. In some embodiments, the portion of the polypeptide derived from EphA7 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA7-derived portion. In some embodiments, the one or more heterologous proteins are fused to the C-terminus of said EphA7-derived portion. In some embodiments, the one or more heterologous proteins are fused to both the N-terminus and the C-terminus of said EphA7-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA7-derived portion. In certain embodiments said targeting domain is fused to the C-terminus of a modified Fc domain (e.g., as described in section 5.2.5), and said modified Fc domain is fused to the N-terminus of said EphA7-derived portion.

In specific embodiments, the polypeptide comprises an ectodomain and a transmembrane domain that are derived from EphA8. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA8 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA8, and wherein said polypeptide exhibits decreased or no binding to ephrins as compared to the parental Eph receptor. In some embodiments, the polypeptide comprises an amino acid sequence identical or similar to SEQ ID NO: 205 or a fragment thereof and has at least 70%, at least 80%, at least 85%, at least 90%, at least 95%, at least 96%, at least 97%, at least 98%, or at least 99% sequence identity to SEQ ID NO: 205, and wherein said polypeptide exhibits decreased or no binding to ephrins as compared to the parental Eph receptor. In some embodiments, the portion of the polypeptide derived from EphA8 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA8-derived portion. In some embodiments, the one or more heterologous proteins are fused to the C-terminus of said EphA8-derived portion. In some embodiments, the one or more heterologous proteins are fused to both the N-terminus and the C-terminus of said EphA8-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA8-derived portion. In certain embodiments said targeting domain is fused to the C-terminus of a modified Fc domain (e.g., as described in section 5.2.5), and said modified Fc domain is fused to the N-terminus of said EphA8-derived portion.

In specific embodiments, the polypeptide comprises an ectodomain and a transmembrane domain that are derived from EphA10. In some embodiments, the polypeptide lacks one or more functional or structural domains, such as the LBD. In some embodiments, the polypeptide comprises an amino acid sequence identical or similar to the entire ectodomain and transmembrane domain region of wild-type EphA10 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA10, and wherein said polypeptide exhibits decreased or no binding to ephrins as compared to the parental Eph receptor. In some embodiments, the polypeptide comprises an amino acid sequence identical or similar to SEQ ID NO: 206 or a fragment thereof and has at least 70%, at least 80%, at least 85%, at least 90%, at least 95%, at least 96%, at least 97%, at least 98%, or at least 99% sequence identity to SEQ ID NO: 206, and wherein said polypeptide exhibits decreased or no binding to ephrins as compared to the parental Eph receptor. In some embodiments, the portion of the polypeptide derived from EphA10 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphA10-derived portion. In some embodiments, the one or more heterologous proteins are fused to the C-terminus of said EphA10-derived portion. In some embodiments, the one or more heterologous proteins are fused to both the N-terminus and the C-terminus of said EphA10-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphA10-derived portion. In certain embodiments said targeting domain is fused to the C-terminus of a modified Fc domain (e.g., as described in section 5.2.5), and said modified Fc domain is fused to the N-terminus of said EphA10-derived portion.

In specific embodiments, the polypeptide comprises an ectodomain and a transmembrane domain that are derived from EphB1. In some embodiments, the polypeptide lacks one or more functional or structural domains, such as the LBD. In some embodiments, the polypeptide comprises an amino acid sequence identical or similar to the entire ectodomain and transmembrane domain region of wild-type EphB1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB1, and wherein said polypeptide exhibits decreased or no binding to ephrins as compared to the parental Eph receptor. In some embodiments, the polypeptide comprises an amino acid sequence identical or similar to SEQ ID NO: 207 or a fragment thereof and has at least 70%, at least 80%, at least 85%, at least 90%, at least 95%, at least 96%, at least 97%, at least 98%, or at least 99% sequence identity to SEQ ID NO: 207, and wherein said polypeptide exhibits decreased or no binding to ephrins as compared to the parental Eph receptor. In some embodiments, the portion of the polypeptide derived from EphB1 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphB1-derived portion. In some embodiments, the one or more heterologous proteins are fused to the C-terminus of said EphB1-derived portion. In some embodiments, the one or more heterologous proteins are fused to both the N-terminus and the C-terminus of said EphB1-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphB1-derived portion. In certain embodiments said targeting domain is fused to the C-terminus of a modified Fc domain (e.g., as described in section 5.2.5), and said modified Fc domain is fused to the N-terminus of said EphB1-derived portion.

In specific embodiments, the polypeptide comprises an ectodomain and a transmembrane domain that are derived from EphB2. In some embodiments, the polypeptide lacks one or more functional or structural domains, such as the LBD. In some embodiments, the polypeptide comprises an amino acid sequence identical or similar to the entire ectodomain and transmembrane domain region of wild-type EphB2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB2, and wherein said polypeptide exhibits decreased or no binding to ephrins as compared to the parental Eph receptor. In some embodiments, the polypeptide comprises an amino acid sequence identical or similar to SEQ ID NO: 208 or a fragment thereof and has at least 70%, at least 80%, at least 85%, at least 90%, at least 95%, at least 96%, at least 97%, at least 98%, or at least 99% sequence identity to SEQ ID NO: 208, and wherein said polypeptide exhibits decreased or no binding to ephrins as compared to the parental Eph receptor. In some embodiments, the portion of the polypeptide derived from EphB2 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphB2-derived portion. In some embodiments, the one or more heterologous proteins are fused to the C-terminus of said EphB2-derived portion. In some embodiments, the one or more heterologous proteins are fused to both the N-terminus and the C-terminus of said EphB2-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphB2-derived portion. In certain embodiments said targeting domain is fused to the C-terminus of a modified Fc domain (e.g., as described in section 5.2.5), and said modified Fc domain is fused to the N-terminus of said EphB2-derived portion.

In specific embodiments, the polypeptide comprises an ectodomain and a transmembrane domain that are derived from EphB3. In some embodiments, the polypeptide lacks one or more functional or structural domains, such as the LBD. In some embodiments, the polypeptide comprises an amino acid sequence identical or similar to the entire ectodomain and transmembrane domain region of wild-type EphB3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB3, and wherein said polypeptide exhibits decreased or no binding to ephrins as compared to the parental Eph receptor. In some embodiments, the polypeptide comprises an amino acid sequence identical or similar to SEQ ID NO: 209 or a fragment thereof and has at least 70%, at least 80%, at least 85%, at least 90%, at least 95%, at least 96%, at least 97%, at least 98%, or at least 99% sequence identity to SEQ ID NO: 209, and wherein said polypeptide exhibits decreased or no binding to ephrins as compared to the parental Eph receptor. In some embodiments, the portion of the polypeptide derived from EphB3 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphB3-derived portion. In some embodiments, the one or more heterologous proteins are fused to the C-terminus of said EphB3-derived portion. In some embodiments, the one or more heterologous proteins are fused to both the N-terminus and the C-terminus of said EphB3-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphB3-derived portion. In certain embodiments said targeting domain is fused to the C-terminus of a modified Fc domain (e.g., as described in section 5.2.5), and said modified Fc domain is fused to the N-terminus of said EphB3-derived portion.

In specific embodiments, the polypeptide comprises an ectodomain and a transmembrane domain that are derived from EphB4. In some embodiments, the polypeptide lacks one or more functional or structural domains, such as the LBD. In some embodiments, the polypeptide comprises an amino acid sequence identical or similar to the entire ectodomain and transmembrane domain region of wild-type EphB4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB4, and wherein said polypeptide exhibits decreased or no binding to ephrins as compared to the parental Eph receptor. In some embodiments, the polypeptide comprises an amino acid sequence identical or similar to SEQ ID NO: 210 or a fragment thereof and has at least 70%, at least 80%, at least 85%, at least 90%, at least 95%, at least 96%, at least 97%, at least 98%, or at least 99% sequence identity to SEQ ID NO: 210, and wherein said polypeptide exhibits decreased or no binding to ephrins as compared to the parental Eph receptor. In some embodiments, the portion of the polypeptide derived from EphB4 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphB4-derived portion. In some embodiments, the one or more heterologous proteins are fused to the C-terminus of said EphB4-derived portion. In some embodiments, the one or more heterologous proteins are fused to both the N-terminus and the C-terminus of said EphB4-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphB4-derived portion. In certain embodiments said targeting domain is fused to the C-terminus of a modified Fc domain (e.g., as described in section 5.2.5), and said modified Fc domain is fused to the N-terminus of said EphB4-derived portion.

In specific embodiments, the polypeptide comprises an ectodomain and a transmembrane domain that are derived from EphB6. In some embodiments, the polypeptide lacks one or more functional or structural domains, such as the LBD. In some embodiments, the polypeptide comprises an amino acid sequence identical or similar to the entire ectodomain and transmembrane domain region of wild-type EphB6 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB6, and wherein said polypeptide exhibits decreased or no binding to ephrins as compared to the parental Eph receptor. In some embodiments, the polypeptide comprises an amino acid sequence identical or similar to SEQ ID NO: 211 or a fragment thereof and has at least 70%, at least 80%, at least 85%, at least 90%, at least 95%, at least 96%, at least 97%, at least 98%, or at least 99% sequence identity to SEQ ID NO: 211, and wherein said polypeptide exhibits decreased or no binding to ephrins as compared to the parental Eph receptor. In some embodiments, the portion of the polypeptide derived from EphB6 is fused to one or more heterologous proteins. In certain embodiments, said one or more heterologous proteins comprise one or more polypeptides encoding the full-length of an ephrin receptor endodomain, a structural domain of an ephrin receptor endodomain, or a fragment of an ephrin receptor endodomain (e.g., the KD or a JM domain) as described in Section 5.2.2. In some embodiments, the one or more heterologous proteins are fused to the N-terminus of said EphB6-derived portion. In some embodiments, the one or more heterologous proteins are fused to the C-terminus of said EphB6-derived portion. In some embodiments, the one or more heterologous proteins are fused to both the N-terminus and the C-terminus of said EphB6-derived portion. In some embodiments, the one or more heterologous proteins are human proteins. In some embodiments, the one or more heterologous proteins are targeting domain(s) and/or purification domains (e.g., as described in Section 5.2.4) fused to the N-terminus of said EphB6-derived portion. In certain embodiments said targeting domain is fused to the C-terminus of a modified Fc domain (e.g., as described in section 5.2.5), and said modified Fc domain is fused to the N-terminus of said EphB6-derived portion.

TABLE 20
Exemplary Eph receptor scaffolds
comprising ectodomain and
transmembrane domain).

| SEQ | | |
|---|---|---|
| ID: | Protein | Sequence |
| 198 | EPHA1 | EVTLMDTSKAQGELGWLLDPPKDGWSEQQQ |
| | (27-596) | ILNGTPLYMYQDCPMQGRRDTDHWLRSNWI |
| | | YRGEEASRVHVELQFTVRDCKSFPGGAGPL |
| | | GCKETFNLLYMESDQDVGIQLRRPLFQKVT |
| | | TVAADQSFTIRDLVSGSVKLNVERCSLGRL |
| | | TRRGLYLAFHNPGACVALVSVRVFYQRCPE |
| | | TLNGLAQFPDTLPGPAGLVEVAGTCLPHAR |
| | | ASPRPSGAPRMHCSPDGEWLVPVGRCHCEP |
| | | GYEEGGSGEACVACPSGSYRMDMDTPHCLT |
| | | CPQQSTAESEGATICTCESGHYRAPGEGPQ |
| | | VACTGPPSAPRNLSFSASGTQLSLRWEPPA |
| | | DTGGRQDVRYSVRCSQCQGTAQDGGPCQPC |
| | | GVGVHFSPGARGLTTPAVHVNGLEPYANYT |
| | | FNVEAQNGVSGLGSSGHASTSVSISMGHAE |
| | | SLSGLSLRLVKKEPRQLELTWAGSRPRSPG |
| | | ANLTYELHVLNQDEERYQMVLEPRVLLTEL |
| | | QPDTTYIVRVRMLTPLGPGPFSPDHEFRTS |
| | | PPVSRGLTGGEIVAVIFGLLLGAALLLGIL |
| | | VFRSRRAQRQRQQRQRDRATDVDREDKLWL |
| 199 | EPHA2 | EVVLLDFAAAGGELGWLTHPYGKGWDLMQN |
| | (28-585) | IMNDMPIYMYSVCNVMSGDQDNWLRTNWVY |
| | | RGEAERIFIELKFTVRDCNSFPGGASSCKE |
| | | TFNLYYAESDLDYGTNFQKRLFTKIDTIAP |
| | | DEITVSSDFEARHVKLNVEERSVGPLTRKG |
| | | FYLAFQDIGACVALLSVRVYYKKCPELLQG |
| | | LAHFPETIAGSDAPSLATVAGTCVDHAVVP |
| | | PGGEEPRMHCAVDGEWLVPIGQCLCQAGYE |
| | | KVEDACQACSPGFFKFEASESPCLECPEHT |
| | | LPSPEGATSCECEEGFFRAPQDPASMPCTR |
| | | PPSAPHYLTAVGMGAKVELRWTPPQDSGGR |
| | | EDIVYSVTCEQCWPESGECGPCEASVRYSE |
| | | PPHGLTRTSVTVSDLEPHMNYTFTVEARNG |
| | | VSGLVTSRSFRTASVSINQTEPPKVRLEGR |
| | | STTSLSVSWSIPPPQQSRVWKYEVTYRKKG |
| | | DSNSYNVRRTEGFSVTLDDLAPDTTYLVQV |
| | | QALTQEGQGAGSKVHEFQTLSPEGSGNLAV |
| | | IGGVAVGVVLLLVLAGVGFFIHRRRKNQRA |
| | | RQSPEDVYFSKSEQLKPL |
| 200 | EPHA3 | EVNLLDSKTIQGELGWISYPSHGWEEISGV |
| | (29-590) | DEHYTPIRTYQVCNVMDHSQNNWLRTNWVP |
| | | RNSAQKIYVELKFTLRDCNSIPLVLGTCKE |
| | | TFNLYYMESDDDHGVKFREHQFTKIDTIAA |
| | | DESFTQMDLGDRILKLNTEIREVGPVNKKG |
| | | FYLAFQDVGACVALVSVRVYFKKCPFTVKN |
| | | LAMFPDTVPMDSQSLVEVRGSCVNNSKEED |
| | | PPRMYCSTEGEWLVPIGKCSCNAGYEERGF |
| | | MCQACRPGFYKALDGNMKCAKCPPHSSTQE |
| | | DGSMNCRCENNYFRADKDPPSMACTRPPSS |
| | | PRNVISNINETSVILDWSWPLDTGGRKDVT |
| | | FNIICKKCGWNIKQCEPCSPNVRFLPRQFG |
| | | LTNTTVTVTDLLAHTNYTFEIDAVNGVSEL |
| | | SSPPRQFAAVSITTNQAAPSPVLTIKKDRT |
| | | SRNSISLSWQEPEHPNGIILDYEVKYYEKQ |
| | | EQETSYTILRARGTNVTISSLKPDTIYVFQ |
| | | IRARTAAGYGTNSRKFEFETSPDSFSISGE |
| | | SSQVVMIAISAAVAIILLTVVIYVLIGRFC |
| | | GYKSKHGADEKRLHFGNGHLKL |
| 201 | EPHA4 | EVTLLDSRSVQGELGWIASPLEGGWEEVSI |
| | (30-590) | MDEKNTPIRTYQVCNVMEPSQNNWLRTDWI |
| | | TREGAQRVYIEIKFTLRDCNSLPGVMGTCK |
| | | ETFNLYYYESDNDKERFIRENQFVKIDTIA |
| | | ADESFTQVDIGDRIMKLNTEIRDVGPLSKK |
| | | GFYLAFQDVGACIALVSVRVFYKKCPLTVR |
| | | NLAQFPDTITGADTSSLVEVRGSCVNNSEE |
| | | KDVPKMYCGADGEWLVPIGNCLCNAGHEER |
| | | SGECQACKIGYYKALSTDATCAKCPPHSYS |
| | | VWEGATSCTCDRGFFRADNDAASMPCTRPP |
| | | SAPLNLISNVNETSVNLEWSSPQNTGGRQD |
| | | ISYNVVCKKCGAGDPSKCRPCGSGVHYTPQ |
| | | QNGLKTTKVSITDLLAHTNYTFEIWAVNGV |
| | | SKYNPNPDQSVSVTVTTNQAAPSSIALVQA |
| | | KEVTRYSVALAWLEPDRPNGVILEYEVKYY |
| | | EKDQNERSYRIVRTAARNTDIKGLNPLTSY |
| | | VFHVRARTAAGYGDFSEPLEVTTNTVPSRI |
| | | IGDGANSTVLLVSVSGSVVLVVILIAAFVI |
| | | SRRRSKYSKAKQEADEEKHLN |
| 202 | EPHA5 | EVNLLDSRTVMGDLGWIAFPKNGWEEIGEV |
| | (60-619) | DENYAPIHTYQVCKVMEQNQNNWLLTSWIS |
| | | NEGASRIFIELKFTLRDCNSLPGGLGTCKE |
| | | TFNMYYFESDDQNGRNIKENQYIKIDTIAA |
| | | DESFTELDLGDRVMKLNTEVRDVGPLSKKG |
| | | FYLAFQDVGACIALVSVRVYYKKCPSVVRH |
| | | LAVFPDTITGADSSQLLEVSGSCVNHSVTD |
| | | EPPKMHCSAEGEWLVPIGKCMCKAGYEEKN |
| | | GTCQVCRPGFFKASPHIQSCGKCPPHSYTH |
| | | EEASTSCVCEKDYFRRESDPPTMACTRPPS |
| | | APRNAISNVNETSVFLEWIPPADTGGRKDV |
| | | SYYIACKKCNSHAGVCEECGGHVRYLPRQS |
| | | GLKNTSVMMVDLLAHTNYTFEIEAVNGVSD |
| | | LSPGARQYVSVNVTTNQAAPSPVTNVKKGK |
| | | IAKNSISLSWQEPDRPNGIILEYEIKYFEK |
| | | DQETSYTIIKSKETTITAEGLKPASVYVFQ |
| | | IRARTAAGYGVFSRRFEFETTPVFAASSDQ |
| | | SQIPVIAVSVTVGVILLAVVIGVLLSGSCC |
| | | ECGCGRASSLCAVAHPSLIW |
| 203 | EPHA6 | QVVLLDTTTVLGELGWKTYPLNGWDAITEM |
| | (34-589) | DEHNRPIHTYQVCNVMEPNQNNWLRTNWIS |
| | | RDAAQKIYVEMKFTLRDCNSIPWVLGTCKE |
| | | TFNLFYMESDESHGIKFKPNQYTKIDTIAA |
| | | DESFTQMDLGDRILKLNTEIREVGPIERKG |
| | | FYLAFQDIGACIALVSVRVFYKKCPFTVRN |
| | | LAMFPDTIPRVDSSSLVEVRGSCVKSAEER |
| | | DTPKLYCGADGDWLVPLGRCICSTGYEEIE |
| | | GSCHACRPGFYKAFAGNTKCSKCPPHSLTY |
| | | MEATSVCQCEKGYFRAEKDPPSMACTRPPS |
| | | APRNVVFNINETALILEWSPPSDTGGRKDL |
| | | TYSVICKKCGLDTSQCEDCGGGLRFIPRHT |
| | | GLINNSVIVLDFVSHVNYTFEIEAMNGVSE |
| | | LSFSPKPFTAITVTTDQDAPSLIGVVRKDW |
| | | ASQNSIALSWQAPAFSNGAILDYEIKYYEK |
| | | EHEQLTYSSTRSKAPSVIITGLKPATKYVF |
| | | HIRVRTATGYSGYSQKFEFETGDETSDMAA |
| | | EQGQILVIATAAVGGFTLLVILTLFFLITG |
| | | RCQWYIKAKMKSEEKRRNHLQNGHL |
| 204 | EPHA7 | AKEVLLLDSKAQQTELEWISSPPNGWEEIS |
| | (30-607) | GLDENYTPIRTYQVCQVMEPNQNNWLRTNW |
| | | ISKGNAQRIFVELKFTLRDCNSLPGVLGTC |
| | | KETFNLYYYETDYDTGRNIRENLYVKIDTI |
| | | AADESFTQGDLGERKMKLNTEVREIGPLSK |
| | | KGFYLAFQDVGACIALVSVKVYYKKCWSII |
| | | ENLAIFPDTVTGSEFSSLVEVRGTCVSSAE |
| | | EEAENAPRMHCSAEGEWLVPIGKCICKAGY |
| | | QQKGDTCEPCGRGFYKSSSQDLQCSRCPTH |
| | | SFSDKEGSSRCECEDGYYRAPSDPPYVACT |
| | | RPPSAPQNLIFNINQTTVSLEWSPPADNGG |
| | | RNDVTYRILCKRCSWEQGECVPCGSNIGYM |
| | | PQQTGLEDNYVTVMDLLAHANYTFEVEAVN |
| | | GVSDLSRSQRLFAAVSITTGQAAPSQVSGV |
| | | MKERVLQRSVELSWQEPEHPNGVITEYEIK |
| | | YYEKDQRERTYSTVKTKSTSASINNLKPGT |
| | | VYVFQIRAFTAAGYGNYSPRLDVATLEEAT |
| | | GKMFEATAVSSEQNPVIIIAVVAVAGTIIL |
| | | VFMVFGFIIGRRHCGYSKADQEGDEELYFH |
| | | FKFPGTKT |
| 205 | EPHA8 | EVNLLDTSTIHGDWGWLTYPAHGWDSINEV |
| | (31-589) | DESFQPIHTYQVCNVMSPNQNNWLRTSWVP |
| | | RDGARRVYAEIKFTLRDCNSMPGVLGTCKE |
| | | TFNLYYLESDRDLGASTQESQFLKIDTIAA |
| | | DESFTGADLGVRRLKLNTEVRSVGPLSKRG |
| | | FYLAFQDIGACLAILSLRIYYKKCPAMVRN |
| | | LAAFSEAVTGADSSSLVEVRGQCVRHSEER |
| | | DTPKMYCSAEGEWLVPIGKCVCSAGYEERR |
| | | DACVACELGFYKSAPGDQLCARCPPHSHSA |
| | | APAAQACHCDLSYYRAALDPPSSACTRPPS |
| | | APVNLISSVNGTSVTLEWAPPLDPGGRSDI |
| | | TYNAVCRRCPWALSRCEACGSGTRFVPQQT |
| | | SLVQASLLVANLLAHMNYSFWIEAVNGVSD |
| | | LSPEPRRAAVVNITTNQAAPSQVVVIRQER |
| | | AGQTSVSLLWQEPEQPNGIILEYEIKYYEK |
| | | DKEMQSYSTLKAVTTRATVSGLKPGTRYVF |
| | | QVRARTSAGCGRFSQAMEVETGKPRPRYDT |
| | | RTIVWICLTLITGLVVLLLLLICKKRHCGY |
| | | SKAFQDSDEEKMHYQNGQA |
| 206 | EPHA10 | EVILLDSKASQAELGWTALPSNGWEEISGV |
| | (35-604) | DEHDRPIRTYQVCNVLEPNQDNWLQTGWIS |
| | | RGRGQRIFVELQFTLRDCSSIPGAAGTCKE |
| | | TFNVYYLETEADLGRGRPRLGGSRPRKIDT |
| | | IAADESFTQGDLGERKMKLNTEVREIGPLS |
| | | RRGFHLAFQDVGACVALVSVRVYYKQCRAT |
| | | VRGLATFPATAAESAFSTLVEVAGTCVAHS |
| | | EGEPGSPPRMHCGADGEWLVPVGRCSCSAG |
| | | FQERGDFCEACPPGFYKVSPRRPLCSPCPE |
| | | HSRALENASTFCVCQDSYARSPTDPPSASC |
| | | TRPPSAPRDLQYSLSRSPLVLRLRWLPPAD |
| | | SGGRSDVTYSLLCLRCGREGPAGACEPCGP |
| | | RVAFLPRQAGLRERAATLLHLRPGARYTVR |
| | | VAALNGVSGPAAAAGTTYAQVTVSTGPGAP |
| | | WEEDEIRRDRVEPQSVSLSWREPIPAGAPG |
| | | ANDTEYEIRYYEKGQSEQTYSMVKTGAPTV |
| | | TVTNLKPATRYVFQIRAASPGPSWEAQSFN |
| | | PSIEVQTLGEAASGSRDQSPAIVVTVVTIS |
| | | ALLVLGSVMSVLAIWRRPCSYGKGGGDAHD |
| 207 | EPHB 1 | ETLMDTRTATAELGWTANPASGWEEVSGYD |
| | (21-591) | ENLNTIRTYQVCNVFEPNQNNWLLTTFINR |
| | | RGAHRIYTEMRFTVRDCSSLPNVPGSCKET |
| | | FNLYYYETDSVIATKKSAFWSEAPYLKVDT |
| | | IAADESFSQVDFGGRLMKVNTEVRSFGPLT |
| | | RNGFYLAFQDYGACMSLLSVRVFFKKCPSI |
| | | VQNFAVFPETMTGAESTSLVIARGTCIPNA |
| | | EEVDVPIKLYCNGDGEWMVPIGRCTCKPGY |
| | | EPENSVACKACPAGTFKASQEAEGCSHCPS |
| | | NSRSPAEASPICTCRTGYYRADFDPPEVAC |
| | | TSVPSGPRNVISIVNETSIILEWHPPRETG |
| | | GRDDVTYNIICKKCRADRRSCSRCDDNVEF |
| | | VPRQLGLTECRVSISSLWAHTPYTFDIQAI |
| | | NGVSSKSPFPPQHVSVNITTNQAAPSTVPI |
| | | MHQVSATMRSITLSWPQPEQPNGIILDYEI |
| | | RYYEKEHNEFNSSMARSQTNTARIDGLRPG |
| | | MVYVVQVRARTVAGYGKFSGKMCFQTLTDD |
| | | DYKSELREQLPLIAGSAAAGVVFVVSLVAI |
| | | SIVCSRKRAYSKEAVYSDKLQHYSTGRGSP |
| | | GM |
| 208 | EPHB2 | VEETLMDSTTATAELGWMVHPPSGWEEVSG |
| | (19-589) | YDENMNTIRTYQVCNVFESSQNNWLRTKFI |
| | | RRRGAHRIHVEMKFSVRDCSSIPSVPGSCK |
| | | ETFNLYYYEADFDSATKTFPNWMENPWVKV |
| | | DTIAADESFSQVDLGGRVMKINTEVRSFGP |
| | | VSRSGFYLAFQDYGGCMSLIAVRVFYRKCP |
| | | RIIQNGAIFQETLSGAESTSLVAARGSCIA |
| | | NAEEVDVPIKLYCNGDGEWLVPIGRCMCKA |
| | | GFEAVENGTVCRGCPSGTFKANQGDEACTH |
| | | CPINSRTTSEGATNCVCRNGYYRADLDPLD |
| | | MPCTTIPSAPQAVISSVNETSLMLEWTPPR |
| | | DSGGREDLVYNIICKSCGSGRGACTRCGDN |
| | | VQYAPRQLGLTEPRIYISDLLAHTQYTFEI |
| | | QAVNGVTDQSPFSPQFASVNITTNQAAPSA |
| | | VSIMHQVSRTVDSITLSWSQPDQPNGVILD |
| | | YELQYYEKELSEYNATAIKSPTNTVTVQGL |
| | | KAGAIYVFQVRARTVAGYGRYSGKMYFQTM |
| | | TEAEYQTSIQEKLPLIIGSSAAGLVFLIAV |
| | | VVIAIVCNRRGFERADSEYTDKLQHYTSGH |
| | | M |
| 209 | EPHB3 | EETLMDTKWVTSELAWTSHPESGWEEVSGY |
| | (39-605) | DEAMNPIRTYQVCNVRESSQNNWLRTGFIW |
| | | RRDVQRVYVELKFTVRDCNSIPNIPGSCKE |
| | | TFNLFYYEADSDVASASSPFWMENPYVKVD |
| | | TIAPDESFSRLDAGRVNTKVRSFGPLSKAG |
| | | FYLAFQDQGACMSLISVRAFYKKCASTTAG |
| | | FALFPETLTGAEPTSLVIAPGTCIPNAVEV |
| | | SVPLKLYCNGDGEWMVPVGACTCATGHEPA |
| | | AKESQCRPCPPGSYKAKQGEGPCLPCPPNS |
| | | RTTSPAASICTCHNNFYRADSDSADSACTT |
| | | VPSPPRGVISNVNETSLILEWSEPRDLGGR |
| | | DDLLYNVICKKCHGAGGASACSRCDDNVEF |
| | | VPRQLGLTERRVHISHLLAHTRYTFEVQAV |
| | | NGVSGKSPLPPRYAAVNITTNQAAPSEVPT |
| | | LRLHSSSGSSLTLSWAPPERPNGVILDYEM |
| | | KYFEKSEGIASTVTSQMNSVQLDGLRPDAR |
| | | YVVQVRARTVAGYGQYSRPAEFETTSERGS |
| | | GAQQLQEQLPLIVGSATAGLVFVVAVVVIA |
| | | IVCLRKQRHGSDSEYTEKLQQYIAPGM |
| 210 | EPHB4 | EETLLNTKLETADLKWVTFPQVDGQWEELS |
| | (19-583) | GLDEEQHSVRTYEVCDVQRAPGQAHWLRTG |
| | | WVPRRGAVHVYATLRFTMLECLSLPRAGRS |
| | | CKETFTVFYYESDADTATALTPAWMENPYI |
| | | KVDTVAAEHLTRKRPGAEATGKVNVKTLRL |
| | | GPLSKAGFYLAFQDQGACMALLSLHLFYKK |
| | | CAQLTVNLTRFPETVPRELVVPVAGSCVVD |
| | | AVPAPGPSPSLYCREDGQWAEQPVTGCSCA |
| | | PGFEAAEGNTKCRACAQGTFKPLSGEGSCQ |
| | | PCPANSHSNTIGSAVCQCRVGYFRARTDPR |
| | | GAPCTTPPSAPRSVVSRLNGSSLHLEWSAP |
| | | LESGGREDLTYALRCRECRPGGSCAPCGGD |
| | | LTFDPGPRDLVEPWVVVRGLRPDFTYTFEV |
| | | TALNGVSSLATGPVPFEPVNVTTDREVPPA |
| | | VSDIRVTRSSPSSLSLAWAVPRAPSGAVLD |
| | | YEVKYHEKGAEGPSSVRFLKTSENRAELRG |
| | | LKRGASYLVQVRARSEAGYGPFGQEHHSQT |
| | | QLDESEGWREQLALIAGTAVVGVVLVLVVI |
| | | VVAVLCLRKQSNGREAEYSDKHGQYLI |
| 211 | EPHB6 | EEVLLDTTGETSEIGWLTYPPGGWDEVSVL |
| | (33-649) | DDQRRLTRTFEACHVAGAPPGTGQDNWLQT |
| | | HFVERRGAQRAHIRLHFSVRACSSLGVSGG |
| | | TCRETFTLYYRQAEEPDSPDSVSSWHLKRW |
| | | TKVDTIAADESFPSSSSSSSSSSSAAWAVG |
| | | PHGAGQRAGLQLNVKERSFGPLTQRGFYVA |
| | | FQDTGACLALVAVRLFSYTCPAVLRSFASF |
| | | PETQASGAGGASLVAAVGTCVAHAEPEEDG |
| | | VGGQAGGSPPRLHCNGEGKWMVAVGGCRCQ |
| | | PGYQPARGDKACQACPRGLYKSSAGNAPCS |
| | | PCPARSHAPNPAAPVCPCLEGFYRASSDPP |
| | | EAPCTGPPSAPQELWFEVQGSALMLHWRLP |
| | | RELGGRGDLLFNVVCKECEGRQEPASGGGG |
| | | TCHRCRDEVHFDPRQRGLTESRVLVGGLRA |
| | | HVPYILEVQAVNGVSELSPDPPQAAAINVS |
| | | TSHEVPSAVPVVHQVSRASNSITVSWPQPD |
| | | QTNGNILDYQLRYYDQAEDESHSFTLTSET |
| | | NTATVTQLSPGHIYGFQVRARTAAGHGPYG |
| | | GKVYFQTLPQGELSSQLPERLSLVIGSILG |
| | | ALAFLLLAAITVLAVVFQRKRRGTGYTEQL |
| | | QQYSSPGLGVKYYIDPS |

### 5.3 Nucleic Acids, Expression Vectors, Cells, and Methods of Making a Polypeptide

Also provided herein are nucleic acids encoding a polypeptide described herein (e.g., described in Section 5.2), vectors (e.g., expression vectors) comprising a nucleic acid described herein, and cells (e.g., host cells) comprising a nucleic acid or expression vector described herein.

The polypeptides (in particular, Eph receptor derived polypeptides) of the disclosure can be produced using any number of expression systems, including prokaryotic and eukaryotic expression systems. In some embodiments, the expression system is a mammalian cell expression system, such as HEK293T systems. Many such systems are widely available from commercial suppliers. In some embodiments, the polynucleotides encoding the polypeptides (in particular, the Eph receptor derived polypeptides) may be expressed using a single vector, e.g., in a di-cistronic expression unit, or under the control of different promoters. In other embodiments, the polynucleotides encoding the polypeptides (in particular, the Eph receptor derived polypeptides) may be expressed using separate vectors.

The polynucleotides may be present in various different forms and/or in different vectors. For instance, the polynucleotides may be essentially linear, circular, and/or have any secondary and/or tertiary and/or higher order structure. Furthermore, the present disclosure also relates to vectors comprising the polynucleotides, e.g. vectors such as plasmids, any circular or linear DNA polynucleotide, mini-circles, viruses (such as adenoviruses, adeno-associated viruses, lentiviruses, retroviruses), mRNAs, and/or modified mRNAs.

In some aspects, the disclosure provides isolated nucleic acids comprising a nucleic acid sequence encoding any of the polypeptides (in particular, Eph receptor derived polypeptides) as described herein; vectors comprising such nucleic acids; and host cells into which the nucleic acids are introduced that are used to replicate the nucleic acids and/or to express the polypeptides (in particular, Eph receptor derived polypeptides).

In some embodiments, a polynucleotide (e.g., an isolated polynucleotide) comprises a nucleotide sequence encoding a polypeptide (in particular, an Eph receptor derived polypeptide) as disclosed herein (e.g., as described above). In some embodiments, a polynucleotide as described herein is operably linked to a heterologous nucleic acid, e.g., a heterologous promoter.

Suitable vectors containing polynucleotides encoding polypeptides (in particular, Eph receptor derived polypeptides) of the present disclosure, or fragments thereof, include cloning vectors and expression vectors. While the cloning vector selected may vary according to the cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColEl, pCRl, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the cells either as an episome or as an integral part of the chromosomal DNA. Suitable expression vectors include but are not limited to plasmids, viral vectors, including adenoviruses, adeno-associated viruses, lentiviruses, retroviruses, and any other vector. Typically, the coding sequence of the polypeptide is operably linked to a suitable control sequence capable of affecting expression of the DNA in a suitable host. Such a control sequences may include a promoter to affect transcription, an optional operator sequence to control transcription, a sequence encoding suitable ribosome binding sites on the mRNA, enhancers and/or sequences which control termination of transcription and translation.

Suitable cells for cloning or expressing a polynucleotide or vector as described herein include prokaryotic or eukaryotic cells. In some embodiments, the cell is prokaryotic. In some embodiments, the cell is eukaryotic, e.g., a Chinese Hamster Ovary (CHO) cell or lymphoid cell. In some embodiments, the cell is a human cell, e.g., a Human Embryonic Kidney (HEK) cell.

Transfection is the process of introducing nucleic acids into cells by non-viral methods. Transduction is the process whereby foreign DNA is introduced into another cell via a viral vector. Common transfection methods include calcium phosphate, cationic polymers (such as PEI), magnetic beads, electroporation and commercial lipid-based reagents such as Lipofectamine and Fugene. Transduction is mostly used to describe the introduction of recombinant viral vector particles into target cells, while ‘infection’ refers to natural infections of humans or animals with wild-type viruses.

Further to the above-mentioned standard methods of nucleic acid delivery, the nucleic acids provided herein can be targeted to specific sites within the genome of the cell. Such methods include, but are not limited to, CRISPR-Cas9, TALENs, meganucleases designed against a genomic sequence of interest within the host cell, and other technologies for precise editing of genomes, Cre-lox site-specific recombination; zinc-finger mediated integration; and homologous recombination. The nucleic acid may contain a transposon comprising a nucleic acid encoding the polypeptides of the disclosure. In some embodiments, said nucleic acid may further contain a nucleic acid sequence encoding a transposase enzyme. In other embodiments, a system with two nucleic acids is provided wherein a first plasmid contains a transposon comprising a nucleic acid encoding the polypeptides of the disclosure, and a second plasmid contains a nucleic acid sequence encoding a transposase enzyme. Both the first and the second nucleic acids may be co-delivered into a host cell. Cells expressing a polypeptide (in particular, an Eph receptor derived polypeptide) described herein may also be generated by using a combination of gene insertion (using a transposon) and genetic editing (using a nuclease). Exemplary transposons include, but are not limited to, piggyBac and the Sleeping Beauty transposon system (SBTS); whereas exemplary nucleases include, without being limited to, the CRISPR/Cas system, Transcription Activator-Like Effector Nucleases (TALENs) and Zinc finger nucleases (ZFNs).

The genetically-modified cell can contain the exogenous sequences by transient or stable transformation. The exogenous sequences can be transformed as a plasmid. The exogenous sequences can be stably integrated into a genomic sequence of the cell, at a targeted site or in a random site. In some aspects, a stable cell line is generated for production of nanovesicles (e.g., EVs and hybridosomes) comprising polypeptides (in particular, Eph receptor derived polypeptides) disclosed herein. Preferably, the cells are stably transfected with the construct encoding the polypeptide (in particular, the Eph receptor derived polypeptide) of the disclosure, such that a stable cell line is generated. This advantageously results in consistent production of nanovesicles (e.g., EVs and hybridosomes) of uniform quality and yield.

The exogenous sequences encoding for a fragment of the polypeptide disclosed herein (in particular, an Eph receptor derived polypeptide) can be inserted into a genomic sequence of the producer cell, located within, upstream (5′-end) or downstream (3′-end) of an endogenous sequence encoding an transmembrane domain. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous nucleic acid sequences can comprise a sequence encoding a polypeptide (in particular, an Eph receptor derived polypeptide) disclosed herein or a fragment or variant thereof. An extra copy of the sequence encoding a polypeptide (in particular, an Eph receptor derived polypeptide) can be introduced to produce a nanovesicle described herein (e.g., a nanovesicle having a higher density of a Eph receptor derived polypeptide or expressing multiple different Eph receptor derived polypeptide on the surface of the nanovesicle). Exogenous sequences encoding a polypeptide (in particular, an Eph receptor derived polypeptide), a variant or a fragment thereof, can be introduced to produce a lumen-engineered and/or surface-decorated nanovesicle (EV or hybridosome) and optionally a nanovesicle containing the modification or the fragment of the polypeptide (in particular, Eph receptor derived polypeptide).

In some aspects, a cell can be modified, e.g., transfected, with one or more vectors encoding one or more polypeptides (in particular, one or more Eph receptor derived polypeptides) comprising exogenous fusion moieties described herein (e.g., targeting moiety or purification domain).

In another aspect, methods of making a polypeptide (in particular, an Eph receptor derived polypeptide) as described herein are provided. In some embodiments, the method comprises culturing a host cell as described herein (e.g., a cell comprising a nucleic acid or expression vector as described herein) under conditions suitable for expression of the polypeptide (in particular, Eph receptor derived polypeptide). In some embodiments, the polypeptide (in particular, Eph receptor derived polypeptide) is subsequently recovered from the host cell (or host cell culture medium). In some embodiments, the polypeptide (in particular, Eph receptor derived polypeptide) is purified, e.g., by affinity chromatography.

### 5.4 Nanovesicles (e.g., Extracellular Vesicles and Hybridosomes) and Methods of Producing or Purifying Nanovesicles

Also provided herein are nanovesicles (e.g., extracellular vesicles and hybridosomes) comprising a polypeptide described herein (e.g., described in Section 5.2). Another aspect of the present disclosure relates to generation and use of surface-engineered nanovesicles. Nanovesicles comprising the polypeptides (in particular, Eph receptor derived polypeptides) described herein provide important advancements and lead to novel nanovesicle compositions and methods of making the same. Previously, overexpression of exogenous proteins relied on stochastic or random disposition of the exogenous proteins onto the nanovesicles for producing surface-engineered nanovesicles. This resulted in low-level, unpredictable density of the heterologous polypeptides (e.g. targeting domains or purification domains) on nanovesicles.

Thus, in one aspect, a nanovesicle is provided comprising at least one Eph receptor derived polypeptide wherein said Eph receptor derived polypeptide
(i) comprises an ephrin ligand binding domain exhibiting decreased or no binding to ephrins as compared to the parental Eph receptor; and
(ii) comprises a transmembrane domain.

The nanovesicles of the invention disclosure may be native (i.e., produced from a source cell through secretion from the endosomal, endolysomal and/or lysosomal pathway or from the plasma membrane of the source cell) nanovesicles or synthetic ones. Exemplary nanovesicles include, without being limited to, extracellular vesicles (“EVs”), microvesicles (MVs), exosomes, apoptotic bodies, ARMMs, fusosomes, microparticles and cell derived vesicular structures, membrane particles, membrane vesicles, exosome-like vesicles, ectosome-like vesicles, ectosomes or exovesicles or hybridosomes.

In one aspect, Eph receptor derived polypeptides may be present on hybridosomes, i.e., hybrid biocompatible carriers which comprise structural and bioactive elements originating from EVs comprising the Eph receptor derived polypeptide and lipid nanoparticles comprising a tunable fusogenic moiety as described in WO2015110957. In some embodiments, isolated hybridosomes comprising Eph receptor derived polypeptides of the disclosure further comprise a therapeutic molecule.

The present disclosure further provides methods of producing and/or purifying nanovesicles (e.g., EVs and hybridosomes) comprising at least one polypeptide (in particular, at least one Eph receptor derived polypeptide) as described above. The methods may typically comprise the steps of (i) introducing into an EV-producing cell a nucleic acid which encodes the polypeptide (in particular, the Eph receptor derived polypeptide) as described above; and (ii) allowing for the EV-producing cell to produce EVs comprising the polypeptide (in particular, the Eph receptor derived polypeptide), such as cultivating the cell under suitable conditions. As a result of the of the presence of a transmembrane domain, the polypeptides (in particular, Eph receptor derived polypeptides) are efficiently transported to membranes of the cell and displayed in or on the surface of EVs. Subsequently, in step (iii), the EVs may be purified from the culture medium. Such methods may optionally comprise the step of (iv) chemically modifying the purified EVs, for example, to produce synthetic nanovesicles such as hybridosomes.

In one aspect, a method of producing nanovesicles being surface decorated with one or more heterologous polypeptides (e.g. targeting domain) is provided, comprising the steps of
(i) providing a nucleic acid or expression vector encoding a polypeptide (in particular, an Eph receptor derived polypeptide) as described above, comprising one or more heterologous polypeptides (e.g. targeting domains);
(ii) introducing said nucleic acid or expression vector into an EV-producing cell;
(iii) cultivating said cells under suitable conditions so that EVs (e.g. exosomes) are produced; and
(iv) purifying the so produced EVs (e.g. exosomes) comprising the polypeptide (in particular, the Eph receptor derived polypeptide) from the cell culture.

The method may optionally comprise the step of (v) chemically modifying the EVs, for example, to produce synthetic nanovesicles such as hybrisosomes. Hybridosomes are e.g., generated by contacting the EV with a second vesicle produced in vitro, said second vesicle comprising a membrane, a fusogenic, ionizable, cationic lipid (e.g., at a molar concentration of at least 10%, at least 20%, at least 30%, at least 40%, at least 50%, and preferably at least 30% of total lipid of the second vesicle) and optionally a therapeutic agent, thereby uniting said EV with said second vesicle and producing a hybridosome.

In one aspect, a method of producing an EV comprises: a. transfecting cells with a nucleic acid described herein or an expression vector described herein; b. cultivating the cells under suitable conditions for the production of the EV; and c. collecting the EV secreted by the cells.

In one aspect, a method of producing a hybridosome comprises contacting a first EV with a second EV, thereby uniting the first EV with the second EV and producing the hybridosome, wherein said first EV has been produced in vitro, and the first EV comprises (i) a membrane, and (ii) a fusogenic, ionizable, cationic lipid (e.g., at a molar concentration of at least 10%, at least 20%, at least 30%, at least 40%, at least 50%, and preferably at least 30% of total lipid of the first EV), and wherein said second EV has been produced by a method of producing an EV described herein.

Some embodiments of the present invention relate to isolation, purification and sub-fractionation of nanovesicles using a specific binding interaction (i.e. affinity purification) between a purification domain (e.g. a modified Fc domain) linked to the scaffold protein of the disclosure enriched on the nanovesicle membrane and an immobilized binding agent. These methods generally comprise the steps of (1) applying or loading a sample comprising nanovesicle to the immobilized agent, (2) optionally washing away unbound sample components using appropriate buffers that maintain the binding interaction between the purification domain linked to the scaffold protein of nanovesicles and binding agents, and (3) eluting (dissociating and recovering) the nanovesicles comprising the purification domain (e.g. modified Fc domain) linked to the scaffold protein from the immobilized binding agents by altering the buffer conditions so that the binding interaction no longer occurs.

In some aspects, the affinity purification method to purify nanovesicles comprising at least one polypeptide (in particular, at least one Eph receptor derived polypeptide) described herein demonstrate has superior recovery yields compared to other affinity purification of nanovesicles known in the art. For example, nanovesicles comprising at least one polypeptide (in particular, at least one Eph receptor derived polypeptide) described herein can be eluted from the immobilized binding partner at a mild pH (e.g. pH 7-pH 9) compared to conventional affinity purification methods requiring a pH of less than 5 sometimes less than pH of 3 to elute (e.g. dissociate) the nanovesicles from the immobilized binding partner (e.g. protein A).

In one aspect, a method of purifying an EV or a hybridosome comprises: a. providing the EV or hybridosome, wherein the EV or hybridosome comprises a first binding partner (e.g. modified Fc domain), wherein the first binding partner is capable of binding to the Fc binding site of an FcRn in a pH dependent manner; b. contacting at a first pH the EV or hybridosome comprising the first binding partner with a second binding partner, wherein the second binding partner comprises the Fc binding site of the FcRn and is associated with a solid matrix; and c. eluting the EV or hybridosome comprising the first binding partner from the solid matrix at a second pH. In certain embodiments, the method further comprises a washing step at the first pH. In certain embodiments, the first pH is below 6.5. In certain embodiments, the second pH is above 7.4. In certain embodiments, the Fc binding site of the FcRn comprises the amino acid sequence of SEQ ID NO: 230.

In one aspect, a method of purifying an EV or a hybridosome comprises: a. providing the EV or hybridosome, wherein the EV or hybridosome comprises a first binding partner, wherein the first binding partner is capable of binding to the Fc binding site of an FcRn in a pH dependent manner and comprises or consists of a polypeptide (in particular, an Eph receptor derived polypeptide) described herein; b. contacting at a first pH the EV or hybridosome comprising the first binding partner with a second binding partner, wherein the second binding partner comprises the Fc binding site of the FcRn and is associated with a solid matrix; and c. eluting the EV or hybridosome comprising the first binding partner from the solid matrix at a second pH. In certain embodiments, the method further comprises a washing step at the first pH. In certain embodiments, the first pH is below 6.5. In certain embodiments, the second pH is above 7.4. In certain embodiments, the Fc binding site of the FcRn comprises the amino acid sequence of SEQ ID NO: 230.

Nanovesicles (e.g., EVs and hybridosomes) comprising the polypeptides (in particular, the Eph receptor derived polypeptides) of the present disclosure can be produced from any type of mammalian cell that is capable of producing nanovesicles (e.g., EVs) under suitable conditions, for instance in suspension culture or in adherent culture or any other type of culturing system. Source cells as per the present disclosure may also include cells that are capable of producing nanovesicles (e.g., EVs) in vivo. The source cells may be selected from a wide range of cells and cell lines which may grow in suspension or adherent culture or be adapted to suspension growth. Generally, nanovesicles (e.g., EVs and hybridosomes) may be derived from essentially any cell source, be it a primary cell source or an immortalized cell line. The source cell may be either allogeneic, autologous, or even xenogeneic in nature to a patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a subject suffering from a certain indication. For instance, in the context of treating inflammatory or degenerative diseases, allogeneic MSCs or amnion epithelial (AE)s may be highly beneficial as nanovesicles (e.g., EV or hybridosome)-producing cell sources due to the inherent immuno-modulatory of their EVs. Cell lines of particular interest include, without being limited to, anionic fluid derived cells, induced pluripotent cells, human umbilical cord endothelial cells (HUVECs), human embryonic kidney (HEK) cells such as HEK293 cells, HEK293T cells, serum free HEK293 cells, suspension HEK293 cells, endothelial cell lines such as microvascular or lymphatic endothelial cells, erythrocytes, erythroid progenitors, chondrocytes, MSCs of different origin, amnion cells, AE cells, any cells obtained through amniocentesis or from the placenta, airway or alveolar epithelial cells, fibroblasts, endothelial cells, and epithelial cells, etc.

As described above, a source cell can be genetically modified to comprise one or more exogenous sequences (e.g., encoding one or more fusion proteins) to produce nanovesicles described herein. Preferably, the exogenous sequence encoding a polypeptide (in particular, an Eph receptor derived polypeptide) described herein is stably integrated into a genomic sequence of the producer cell, at a targeted site or in a random site. In some aspects, a stable cell line is generated for production of nanovesicles (e.g., EVs) comprising polypeptides (in particular, Eph receptor derived polypeptides) disclosed herein. This advantageously results in consistent production of nanovesicles (e.g., EVs) of uniform quality and yield.

In some embodiments, during EV production, cargo proteins present in the cytosol of the producing cell in the vicinity of the forming EV are captured by cargo binding domain of the polypeptide while the EV is formed. As a result, a cell that is producing both EV and cargo protein can produce some EVs with at least one cargo protein in the lumen of the EVs. In some embodiments, EVs described herein have more cargo proteins in the lumen of the EVs than the naturally occurring amount, e.g., the amount passively captured by a forming EV. In some embodiments, the number of cargo proteins in the lumen of the EV expressing the polypeptides of the disclosure is higher than the number of cargo proteins in the lumen of a reference EV. In some embodiments, the reference EV comprises cargo proteins associated with the EV through a natural process.

In some aspects, nanovesicles comprising polypeptides (in particular, Eph receptor derived polypeptides) of the present disclosure can be produced from a cell transformed with a sequence encoding a full-length, polypeptide (in particular, Eph receptor derived polypeptide) as disclosed herein that may comprise one or more fusion moieties as described above. Any of the polypeptides (in particular, Eph receptor derived polypeptides) described herein can be expressed from a plasmid, an exogenous sequence inserted into the genome or other exogenous nucleic acid, such as a synthetic messenger RNA (mRNA).

In one aspect, the present disclosure provides an EV comprising two or more interacting polypeptides (e.g., scaffold protein), that is produced from a cell of the present disclosure. In some embodiments, the surface density or concentration of the polypeptide (e.g., scaffold protein) on the EV described herein is increased by dimerization or oligomerization. In certain embodiments, the EV comprises polypeptides (e.g., scaffold proteins) described herein, that each comprises a homo-domain dimerization motif (e.g., a CRD homo-dimer motif) and that are interacting with each other to form homo-pairs. In certain embodiments, the EV comprises polypeptides (e.g., scaffold proteins) described herein, that each comprises domains that can undergo hetero-domain dimerization (e.g., LBD-FN dimerization), wherein said domains are capable of interacting with each other to form hetero-pairs. The ability of ephrin receptors to undergo hetero-domain dimerization (e.g., LBD-FN dimerization) is unique in that it can facilitate the formation of a clustering of more than two ephrin receptors (because hetero-domain dimerization involves two different domains, and one ephrin receptor protein through the two different domains present on the same protein can be linked to two other ephrin receptor proteins, see, e.g., FIG. 2B); whereas scaffold proteins that can only undergo homo-domain dimerization usually can only form dimers.

In a further aspect, the present disclosure provides an EV comprising two or more polypeptides (e.g., scaffold protein) and one or more adaptor proteins, which EV is produced from a cell of the present disclosure. The adaptor protein(s) are as described above in Section 5.2.3(c). In one embodiment, the concentration of adaptor proteins in the cytosol can be varied on quite rapid time scales by processes controlling synthesis (e.g., through inducible promoters). In a further embodiment, the concentration of adaptor proteins in the cytosol can be varied on quite rapid time scales by processes controlling dimerization of two or more adaptor fragments (e.g., by using chemically inducible dimerization agents).

In some embodiments, a source cell disclosed herein is further modified to comprise an additional exogenous sequence. For example, an additional exogenous sequence can be introduced to modulate endogenous gene expression or produce a nanovesicle including a certain polypeptide as a payload. In some aspects, the source cell is modified to comprise two exogenous sequences, one encoding a polypeptide (in particular, an Eph receptor derived polypeptide) described herein, or a variant or a fragment thereof, and the other encoding a payload. In some aspects, the source cell is modified to comprise two exogenous sequences, one encoding a polypeptide (in particular, an Eph receptor derived polypeptide) described herein, or a variant or a fragment thereof, and the other encoding a polypeptide (in particular, an Eph receptor derived polypeptide) described herein that comprises an optional targeting moiety. In certain embodiments, the source cell can be further modified to comprise an additional exogenous sequence conferring additional functionalities to the nanovesicles (e.g., payloads, targeting moieties, or purification domains). In some aspects, the source cell is modified to comprise two exogenous sequences, one encoding a polypeptide (in particular, an Eph receptor derived polypeptide) disclosed herein, or a variant or a fragment thereof, and the other encoding a protein conferring the additional functionalities to nanovesicles. In some aspects, the source cell is further modified to comprise one, two, three, four, five, six, seven, eight, nine, or ten or more additional exogenous sequences.

In some embodiments, nanovesicles can be produced from a cell transformed with one or more nucleotide sequences encoding fragments of EphA1. In some embodiments, the nanovesicle comprises a polypeptide comprising fragments of EphA1 but lacking one or more functional or structural domains, such as the LBD. In some embodiments, the produced nanovesicle comprises a polypeptide that comprises an amino acid sequence identical or similar to the entire ectodomain and transmembrane domain region of wild-type EphA1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA1, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No:198 or a fragment thereof and has at least 70%, at least 80%, at least 85%, at least 90%, at least 95%, at least 96%, at least 97%, at least 98%, or at least 99% sequence identity to SEQ ID No: 198, and wherein said polypeptide exhibits decreased or no binding to ephrins as compared to the parental Eph receptor. In some embodiments, the portion of the polypeptide derived from EphA1 is fused to one or more heterologous proteins. In some embodiments, said one or more heterologous proteins are fused to the N-terminus of said EphA1-derived portion. In some embodiments, said one or more heterologous proteins are fused to the C-terminus of said EphA1-derived portion. In some embodiments, said one or more heterologous proteins are fused to both the N-terminus and the C-terminus of said EphA1-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA1-derived portion. In certain embodiments said targeting domain is fused to the C-terminus of a modified Fc domain (e.g., as described in section 5.2.5), and said modified Fc domain is fused to the N-terminus of said EphA1-derived portion.

In some embodiments, nanovesicles can be produced from a cell transformed with one or more nucleotide sequences encoding fragments of EphA2. In some embodiments, the nanovesicle comprises a polypeptide comprising fragments of EphA2 but lacking one or more functional or structural domains, such as the LBD. In some embodiments, the produced nanovesicle comprises a polypeptide that comprises an amino acid sequence identical or similar to the entire ectodomain and transmembrane domain region of wild-type EphA2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA2, and wherein said polypeptide exhibits decreased or no binding to ephrins as compared to the parental Eph receptor. In some embodiments, the portion of the polypeptide derived from EphA2 is fused to one or more heterologous proteins. In some embodiments, the produced nanovesicle comprises a polypeptide that comprises an amino acid sequence identical or similar to SEQ ID No: 199 or a fragment thereof and has at least 70%, at least 80%, at least 85%, at least 90%, at least 95%, at least 96%, at least 97%, at least 98%, or at least 99% sequence identity to SEQ ID No: 199, and wherein said polypeptide exhibits decreased or no binding to ephrins as compared to the parental Eph receptor. In some embodiments, the portion of the polypeptide derived from EphA2 is fused to one or more heterologous proteins. In some embodiments, said one or more heterologous proteins are fused to the N-terminus of said EphA2-derived portion. In some embodiments, said one or more heterologous proteins are fused to the C-terminus of said EphA2-derived portion. In some embodiments, said one or more heterologous proteins are fused to both the N-terminus and the C-terminus of said EphA2-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA2-derived portion. In certain embodiments said targeting domain is fused to the C-terminus of a modified Fe domain (e.g., as described in section 5.2.5), and said modified Fc domain is fused to the N-terminus of said EphA2-derived portion.

In some embodiments, nanovesicles can be produced from a cell transformed with one or more nucleotide sequences encoding fragments of wild-type EphA3. In some embodiments, the nanovesicle comprises a polypeptide comprising fragments of EphA3 but lacking one or more functional or structural domains, such as the LBD. In some embodiments, the produced nanovesicle comprises a polypeptide that comprises an amino acid sequence identical or similar to the entire ectodomain and transmembrane domain region of EphA3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA3, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 200 or a fragment thereof and has at least 70%, at least 80%, at least 85%, at least 90%, at least 95%, at least 96%, at least 97%, at least 98%, or at least 99% sequence identity to SEQ ID No: 200, and wherein said polypeptide exhibits decreased or no binding to ephrins as compared to the parental Eph receptor. In some embodiments, the portion of the polypeptide derived from EphA3 is fused to one or more heterologous proteins. In some embodiments, said one or more heterologous proteins are fused to the N-terminus of said EphA3-derived portion. In some embodiments, said one or more heterologous proteins are fused to the C-terminus of said EphA3-derived portion. In some embodiments, said one or more heterologous proteins are fused to both the N-terminus and the C-terminus of said EphA3-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA3-derived portion. In certain embodiments said targeting domain is fused to the C-terminus of a modified Fe domain (e.g., as described in section 5.2.5), and said modified Fc domain is fused to the N-terminus of said EphA3-derived portion.

In some embodiments, nanovesicles can be produced from a cell transformed with one or more nucleotide sequences encoding fragments of wild-type EphA4. In some embodiments, the nanovesicle comprises a polypeptide comprising fragments of EphA4 but lacking one or more functional or structural domains, such as the LBD. In some embodiments, the produced nanovesicle comprises a polypeptide that comprises an amino acid sequence identical or similar to the entire ectodomain and transmembrane domain region of EphA4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA4, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 201 or a fragment thereof and has at least 70%, at least 80%, at least 85%, at least 90%, at least 95%, at least 96%, at least 97%, at least 98%, or at least 99% sequence identity to SEQ ID No: 201, and wherein said polypeptide exhibits decreased or no binding to ephrins as compared to the parental Eph receptor. In some embodiments, the portion of the polypeptide derived from EphA4 is fused to one or more heterologous proteins. In some embodiments, said one or more heterologous proteins are fused to the N-terminus of said EphA4-derived portion. In some embodiments, said one or more heterologous proteins are fused to the C-terminus of said EphA4-derived portion. In some embodiments, said one or more heterologous proteins are fused to both the N-terminus and the C-terminus of said EphA4-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA4-derived portion. In certain embodiments said targeting domain is fused to the C-terminus of a modified Fc domain (e.g., as described in section 5.2.5), and said modified Fc domain is fused to the N-terminus of said EphA4-derived portion.

In some embodiments, nanovesicles can be produced from a cell transformed with one or more nucleotide sequences encoding fragments of wild-type EphA5. In some embodiments, the nanovesicle comprises a polypeptide comprising fragments of EphA5 but lacking one or more functional or structural domains, such as the LBD. In some embodiments, the produced nanovesicle comprises a polypeptide that comprises an amino acid sequence identical or similar to the entire ectodomain and transmembrane domain region of EphA5 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA5, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 202 or a fragment thereof and has at least 70%, at least 80%, at least 85%, at least 90%, at least 95%, at least 96%, at least 97%, at least 98%, or at least 99% sequence identity to SEQ ID No: 202, and wherein said polypeptide exhibits decreased or no binding to ephrins as compared to the parental Eph receptor. In some embodiments, the portion of the polypeptide derived from EphA5 is fused to one or more heterologous proteins. In some embodiments, said one or more heterologous proteins are fused to the N-terminus of said EphA5-derived portion. In some embodiments, said one or more heterologous proteins are fused to the C-terminus of said EphA5-derived portion. In some embodiments, said one or more heterologous proteins are fused to both the N-terminus and the C-terminus of said EphA5-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA5-derived portion. In certain embodiments said targeting domain is fused to the C-terminus of a modified Fc domain (e.g., as described in section 5.2.5), and said modified Fc domain is fused to the N-terminus of said EphA5-derived portion.

In some embodiments, nanovesicles can be produced from a cell transformed with one or more nucleotide sequences encoding fragments of wild-type EphA6. In some embodiments, the nanovesicle comprises a polypeptide comprising fragments of EphA6 but lacking one or more functional or structural domains, such as the LBD. In some embodiments, the produced nanovesicle comprises a polypeptide that comprises an amino acid sequence identical or similar to the entire ectodomain and transmembrane domain region of EphA6 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA6, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 203 or a fragment thereof and has at least 70%, at least 80%, at least 85%, at least 90%, at least 95%, at least 96%, at least 97%, at least 98%, or at least 99% sequence identity to SEQ ID No: 203, and wherein said polypeptide exhibits decreased or no binding to ephrins as compared to the parental Eph receptor. In some embodiments, the portion of the polypeptide derived from EphA6 is fused to one or more heterologous proteins. In some embodiments, said one or more heterologous proteins are fused to the N-terminus of said EphA6-derived portion. In some embodiments, said one or more heterologous proteins are fused to the C-terminus of said EphA6-derived portion. In some embodiments, said one or more heterologous proteins are fused to both the N-terminus and the C-terminus of said EphA6-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA6-derived portion. In certain embodiments said targeting domain is fused to the C-terminus of a modified Fc domain (e.g., as described in section 5.2.5), and said modified Fc domain is fused to the N-terminus of said EphA6-derived portion.

In some embodiments, nanovesicles can be produced from a cell transformed with one or more nucleotide sequences encoding fragments of wild-type EphA7. In some embodiments, the nanovesicle comprises a polypeptide comprising fragments of EphA7 but lacking one or more functional or structural domains, such as the LBD. In some embodiments, the produced nanovesicle comprises a polypeptide that comprises an amino acid sequence identical or similar to the entire ectodomain and transmembrane domain region of EphA7 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA7, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 204 or a fragment thereof and has at least 70%, at least 80%, at least 85%, at least 90%, at least 95%, at least 96%, at least 97%, at least 98%, or at least 99% sequence identity to SEQ ID No: 204, and wherein said polypeptide exhibits decreased or no binding to ephrins as compared to the parental Eph receptor. In some embodiments, the portion of the polypeptide derived from EphA7 is fused to one or more heterologous proteins. In some embodiments, said one or more heterologous proteins are fused to the N-terminus of said EphA7-derived portion. In some embodiments, said one or more heterologous proteins are fused to the C-terminus of said EphA7-derived portion. In some embodiments, said one or more heterologous proteins are fused to both the N-terminus and the C-terminus of said EphA7-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA7-derived portion. In certain embodiments said targeting domain is fused to the C-terminus of a modified Fc domain (e.g., as described in section 5.2.5), and said modified Fc domain is fused to the N-terminus of said EphA7-derived portion.

In some embodiments, nanovesicles can be produced from a cell transformed with one or more nucleotide sequences encoding fragments of wild-type EphA8. In some embodiments, the nanovesicle comprises a polypeptide comprising fragments of EphA8 but lacking one or more functional or structural domains, such as the LBD. In some embodiments, the produced nanovesicle comprises a polypeptide that comprises an amino acid sequence identical or similar to the entire ectodomain and transmembrane domain region of EphA8 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA8, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 205 or a fragment thereof and has at least 70%, at least 80%, at least 85%, at least 90%, at least 95%, at least 96%, at least 97%, at least 98%, or at least 99% sequence identity to SEQ ID No: 205, and wherein said polypeptide exhibits decreased or no binding to ephrins as compared to the parental Eph receptor. In some embodiments, the portion of the polypeptide derived from EphA8 is fused to one or more heterologous proteins. In some embodiments, said one or more heterologous proteins are fused to the N-terminus of said EphA8-derived portion. In some embodiments, said one or more heterologous proteins are fused to the C-terminus of said EphA8-derived portion. In some embodiments, said one or more heterologous proteins are fused to both the N-terminus and the C-terminus of said EphA8-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA8-derived portion. In certain embodiments said targeting domain is fused to the C-terminus of a modified Fe domain (e.g., as described in section 5.2.5), and said modified Fc domain is fused to the N-terminus of said EphA8-derived portion.

In some embodiments, nanovesicles can be produced from a cell transformed with one or more nucleotide sequences encoding fragments of wild-type EphA10. In some embodiments, the nanovesicle comprises a polypeptide comprising fragments of EphA10 but lacking one or more functional or structural domains, such as the LBD. In some embodiments, the produced nanovesicle comprises a polypeptide that comprises an amino acid sequence identical or similar to the entire ectodomain and transmembrane domain region of EphA10 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA10, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 206 or a fragment thereof and has at least 70%, at least 80%, at least 85%, at least 90%, at least 95%, at least 96%, at least 97%, at least 98%, or at least 99% sequence identity to SEQ ID No: 206, and wherein said polypeptide exhibits decreased or no binding to ephrins as compared to the parental Eph receptor. In some embodiments, the portion of the polypeptide derived from EphA10 is fused to one or more heterologous proteins. In some embodiments, said one or more heterologous proteins are fused to the N-terminus of said EphA10-derived portion. In some embodiments, said one or more heterologous proteins are fused to the C-terminus of said EphA10-derived portion. In some embodiments, said one or more heterologous proteins are fused to both the N-terminus and the C-terminus of said EphA10-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphA10-derived portion. In certain embodiments said targeting domain is fused to the C-terminus of a modified Fc domain (e.g., as described in section 5.2.5), and said modified Fe domain is fused to the N-terminus of said EphA10-derived portion.

In some embodiments, nanovesicles can be produced from a cell transformed with one or more nucleotide sequences encoding fragments of wild-type EphB1. In some embodiments, the nanovesicle comprises a polypeptide comprising fragments of EphB1 but lacking one or more functional or structural domains, such as the LBD. In some embodiments, the produced nanovesicle comprises a polypeptide that comprises an amino acid sequence identical or similar to the entire ectodomain and transmembrane domain region of EphB1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB1, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 207 or a fragment thereof and has at least 70%, at least 80%, at least 85%, at least 90%, at least 95%, at least 96%, at least 97%, at least 98%, or at least 99% sequence identity to SEQ ID No: 207, and wherein said polypeptide exhibits decreased or no binding to ephrins as compared to the parental Eph receptor. In some embodiments, the portion of the polypeptide derived from EphB1 is fused to one or more heterologous proteins. In some embodiments, said one or more heterologous proteins are fused to the N-terminus of said EphB1-derived portion. In some embodiments, said one or more heterologous proteins are fused to the C-terminus of said EphB1-derived portion. In some embodiments, said one or more heterologous proteins are fused to both the N-terminus and the C-terminus of said EphB1-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphB1-derived portion. In certain embodiments said targeting domain is fused to the C-terminus of a modified Fc domain (e.g., as described in section 5.2.5), and said modified Fc domain is fused to the N-terminus of said EphB1-derived portion.

In some embodiments, nanovesicles can be produced from a cell transformed with one or more nucleotide sequences encoding fragments of wild-type EphB2. In some embodiments, the nanovesicle comprises a polypeptide comprising fragments of EphB2 but lacking one or more functional or structural domains, such as the LBD. In some embodiments, the produced nanovesicle comprises a polypeptide that comprises an amino acid sequence identical or similar to the entire ectodomain and transmembrane domain region of EphB2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB2, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 208 or a fragment thereof and has at least 70%, at least 80%, at least 85%, at least 90%, at least 95%, at least 96%, at least 97%, at least 98%, or at least 99% sequence identity to SEQ ID No: 208, and wherein said polypeptide exhibits decreased or no binding to ephrins as compared to the parental Eph receptor. In some embodiments, the portion of the polypeptide derived from EphB2 is fused to one or more heterologous proteins. In some embodiments, said one or more heterologous proteins are fused to the N-terminus of said EphB2-derived portion. In some embodiments, said one or more heterologous proteins are fused to the C-terminus of said EphB2-derived portion. In some embodiments, said one or more heterologous proteins are fused to both the N-terminus and the C-terminus of said EphB2-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphB2-derived portion. In certain embodiments said targeting domain is fused to the C-terminus of a modified Fc domain (e.g., as described in section 5.2.5), and said modified Fc domain is fused to the N-terminus of said EphB2-derived portion.

In some embodiments, nanovesicles can be produced from a cell transformed with one or more nucleotide sequences encoding fragments of wild-type EphB3. In some embodiments, the nanovesicle comprises a polypeptide comprising fragments of EphB3 but lacking one or more functional or structural domains, such as the LBD. In some embodiments, the produced nanovesicle comprises a polypeptide that comprises an amino acid sequence identical or similar to the entire ectodomain and transmembrane domain region of EphB3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB3, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 209 or a fragment thereof and has at least 70%, at least 80%, at least 85%, at least 90%, at least 95%, at least 96%, at least 97%, at least 98%, or at least 99% sequence identity to SEQ ID No: 209, and wherein said polypeptide exhibits decreased or no binding to ephrins as compared to the parental Eph receptor. In some embodiments, the portion of the polypeptide derived from EphB3 is fused to one or more heterologous proteins. In some embodiments, said one or more heterologous proteins are fused to the N-terminus of said EphB3-derived portion. In some embodiments, said one or more heterologous proteins are fused to the C-terminus of said EphB3-derived portion. In some embodiments, said one or more heterologous proteins are fused to both the N-terminus and the C-terminus of said EphB3-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphB3-derived portion. In certain embodiments said targeting domain is fused to the C-terminus of a modified Fc domain (e.g., as described in section 5.2.5), and said modified Fc domain is fused to the N-terminus of said EphB3-derived portion.

In some embodiments, nanovesicles can be produced from a cell transformed with one or more nucleotide sequences encoding fragments of wild-type EphB4. In some embodiments, the nanovesicle comprises a polypeptide comprising fragments of EphB4 but lacking one or more functional or structural domains, such as the LBD. In some embodiments, the produced nanovesicle comprises a polypeptide that comprises an amino acid sequence identical or similar to the entire ectodomain and transmembrane domain region of EphB4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB4, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 210 or a fragment thereof and has at least 70%, at least 80%, at least 85%, at least 90%, at least 95%, at least 96%, at least 97%, at least 98%, or at least 99% sequence identity to SEQ ID No: 210, and wherein said polypeptide exhibits decreased or no binding to ephrins as compared to the parental Eph receptor. In some embodiments, the portion of the polypeptide derived from EphB4 is fused to one or more heterologous proteins. In some embodiments, said one or more heterologous proteins are fused to the N-terminus of said EphB4-derived portion. In some embodiments, said one or more heterologous proteins are fused to the C-terminus of said EphB4-derived portion. In some embodiments, said one or more heterologous proteins are fused to both the N-terminus and the C-terminus of said EphB4-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphB4-derived portion. In certain embodiments said targeting domain is fused to the C-terminus of a modified Fe domain (e.g., as described in section 5.2.5), and said modified Fc domain is fused to the N-terminus of said EphB4-derived portion.

In some embodiments, nanovesicles can be produced from a cell transformed with one or more nucleotide sequences encoding fragments of wild-type EphB6. In some embodiments, the nanovesicle comprises a polypeptide comprising fragments of EphB6 but lacking one or more functional or structural domains, such as the LBD. In some embodiments, the produced nanovesicle comprises a polypeptide that comprises an amino acid sequence identical or similar to the entire ectodomain and transmembrane domain region of EphB6 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB6, and wherein said polypeptide exhibits decreased or no binding to ephrins as compared to the parental Eph receptor. In some embodiments, the produced nanovesicle comprises a polypeptide that comprises an amino acid sequence identical or similar to SEQ ID No: 211 or a fragment thereof and has at least 70%, at least 80%, at least 85%, at least 90%, at least 95%, at least 96%, at least 97%, at least 98%, or at least 99% sequence identity to SEQ ID No: 211, and wherein said polypeptide exhibits decreased or no binding to ephrins as compared to the parental Eph receptor. In some embodiments, the portion of the polypeptide derived from EphB6 is fused to one or more heterologous proteins. In some embodiments, said one or more heterologous proteins are fused to the N-terminus of said EphB6-derived portion. In some embodiments, said one or more heterologous proteins are fused to the C-terminus of said EphB6-derived portion. In some embodiments, said one or more heterologous proteins are fused to both the N-terminus and the C-terminus of said EphB6-derived portion. In some embodiments, the one or more heterologous proteins are human proteins. In some embodiments, said one or more heterologous proteins comprise targeting domain(s) and/or purification domain(s) (e.g., as described in Section 5.2.4) fused to the N-terminus of said EphB6-derived portion. In certain embodiments said targeting domain is fused to the C-terminus of a modified Fe domain (e.g., as described in section 5.2.5), and said modified Fc domain is fused to the N-terminus of said EphB6-derived portion.

In some embodiments, nanovesicles comprising at least one polypeptide (in particular, at least one Eph receptor derived polypeptide) from the modified source cell, have a higher density of the at least one polypeptide (in particular, at least one Eph receptor derived polypeptide) compared to native nanovesicles isolated from an unmodified cell of the same or similar cell type. In some embodiments, nanovesicles of the disclosure contain a polypeptide (in particular, an Eph receptor derived polypeptide) described herein at a density 2-, 4-, 8-, 16-, 32-, 64-, 100-, 200-, 400-, 800-, 1,000-fold or higher than a native nanovesicle isolated from an unmodified cell of the same or similar cell type. In some embodiments, the polypeptide (in particular, Eph receptor derived polypeptide) is present at 2-, 4-, 8-, 16-, 32-, 64-, 100-, 200-, 400-, 800-, 1,000-fold or a higher density on the nanovesicle surface than fusion proteins on other nanovesicle surfaces similarly modified using a conventional scaffold protein (e.g., a tetraspanin molecule, like CD63). To quantify the amount or level polypeptide expressed on a nanovesicle (e.g., EV), any appropriate method known in the art can be used. In some aspects, the amount of polypeptides (in particular, at least one Eph receptor derived polypeptide) expressed on an nanovesicle (e.g., EV) can be assessed by measuring the number of peptide spectral matches in a given sample comprising a nanovesicle (e.g., EV) using liquid chromatography with tandem mass spectrometry (LC-MS/MS).

The polypeptides (in particular, Eph receptor derived polypeptides) of the present disclosure may provide an advantage when expressed on nanovesicles compared to native EVs or synthetic nanovesicles known in the field.

In some aspects, when the polypeptide (in particular, Eph receptor derived polypeptide) is expressed on nanovesicles, the transmembrane domain provides anchoring to the membrane, to which the ectodomain of the polypeptide (in particular, Eph receptor derived polypeptide) is covalently linked (e.g. fused), as well as any one or more optional heterologous polypeptides (e.g. targeting domains). As outlined above, such anchoring allows for directing one or more polypeptide (in particular, Eph receptor derived polypeptide) described herein reliably either on the surface of nanovesicles or into the lumen of the nanovesicle, depending on which placement is preferred.

In some aspects, the nanovesicles comprising at least one polypeptide (in particular, at least one Eph receptor derived polypeptide) described herein demonstrate superior characteristics compared to nanovesicles known in the art. For example, Eph receptor derived polypeptides comprising different transmembrane domains or fragments thereof can be more highly enriched on the surface of a nanovesicle than naturally occurring ones or the nanovesicles produced using conventional EV proteins. Moreover, the surface of nanovesicles comprising Eph receptor derived polypeptides of the present disclosure can have greater, more specific, or more controlled biological activity (e.g. targeting to specific cells or half-life) compared to naturally occurring nanovesicles or the nanovesicles produced using conventional transmembrane domains (e.g. Lamp2b, PTGFRN, CD63 or CD81). In some embodiments, the Eph receptor derived polypeptide is present on the surface of the nanovesicle at a higher density than a conventional scaffold protein of a different nanovesicle. In some embodiments, the Eph receptor derived polypeptide exhibits increased oligomerization than a conventional scaffold protein of a different nanovesicle. Furthermore, the scaffold proteins of the present disclosure can undergo hetero-domain dimerization or clustering (e.g., head-to tail configuration) as opposed to other protein scaffolds that can be expressed on nanovesicles (e.g., Lamp2b, PTGFRN, CD63 or CD81). Examples of conventional scaffold proteins include, without being limited to, CD9, CD63, CD81, PDGFR, GPI anchor proteins, lactadherin, LAMP2, LAMP2B, and fragments thereof. In some embodiments, the nanovesicle exhibits increased oligomerization when compared to nanovesicles comprising the parental Eph receptor. In some embodiments, the nanovesicle exhibits increased scaffold protein density when compared to nanovesicles comprising the parental Eph receptor.

In some embodiments, the nanovesicle comprises a polypeptide described herein with a domain that can undergo hetero-domain dimerization (e.g., head-to-tail dimerization). In specific embodiments, the nanovesicle comprises a polypeptide described herein that can undergo a hetero-domain dimerization (e.g., head-to-tail dimerization) between an LBD and a FN domain.

In some embodiments, the nanovesicle comprises a polypeptide described herein that can undergo a homo-domain dimerization. In specific embodiments, the nanovesicle comprises a polypeptide described herein that can undergo a homo-domain dimerization via CRD.

In some embodiments, nanovesicles described herein comprise at least one polypeptide (in particular, at least one Eph receptor derived polypeptide) described herein comprising in the lumen (e.g., after the C-terminus of the transmembrane domain): (i) a YX₁DX₂X₃X₄YEDP motif, wherein X₁ is I or V, X₂ is P or L, X₃ is Q, H, F, D, E, or S, X₄ is A or T (SEQ ID NO:240); or (ii) a FX₁DX₂X₃X₄FEDP motif, wherein X₁ is I or V, X₂ is P or L, X₃ is Q, H, F, D, E, or S, X₄ is A or T (SEQ ID NO:241).

In some embodiments, the methods of producing nanovesicles described herein further comprise the step of characterizing nanovesicles comprising polypeptides (in particular, Eph receptor derived polypeptides). In some embodiments, contents of said nanovesicles can be extracted for study and characterization. In some embodiments, nanovesicles are isolated and characterized by metrics including, but not limited to, size, shape, morphology, or molecular compositions such as nucleic acids, proteins, metabolites, and lipids as well as half-life and pharmacodynamics.

In one aspect, the nanovesicles comprising a scaffold protein and a modified Fc domain can bestow several desirable properties upon the nanovesicle including increased serum half-life, shorter blood clearance and improved affinity purification. In some embodiments, the nanovesicles described herein can be modified to increase or decrease their half-life in circulation. In some embodiments, the half-life of the therapeutic cargo in the nanovesicle comprising the polypeptide described herein in circulation can be modified by altering the half-life of the nanovesicle. In some instances, the half-life is increased and the increase can be, for instance from about 1.5-fold to 20-fold for a therapeutic agent payload maintained in the nanovesicle comprising polypeptides described herein when compared to a therapeutic agent not contained in the nanovesicle and the half-life being measured in a serum-containing solution.

In certain embodiments, presence or absence of the nanovesicle and/or the therapeutic molecule payload in the circulatory system, is determined by the presence or absence of certain polypeptides or fragments thereof on the nanovesicle, for example, a modified Fc domain polypeptide or a functional fragment thereof.

In some embodiments, the nanovesicles comprising the polypeptides described herein are capable of being present in the circulatory system or tissue of a subject for an extended period of time, allowing the delivery of a more efficient therapeutic effect than what can be achieved by nanovesicles devoid of said polypeptides. Half-life extension is a particular advantage when compared to current EV-based therapies not involving scaffold proteins comprising modified Fc domains.

Effective amounts of scaffold proteins comprising modified Fc domains include 1, 2, 3, 4, 5, 6, 7, 8, 9, 10, 20, 30, 40, 60, 80, 100 or more polypeptides per nanovesicle. Alternatively, an effective amount is the amount capable of extending the nanovesicle half-life by 10%, 20%, 30%, 40%, 50%, 60%, 70%, 80%, 90%, 100%, 200%, 400%, 800%, 1,000%, or 10,000% relative to the half-life that the nanovesicle would exhibit without the polypeptides.

In some embodiments, the methods described herein comprise measuring the size of nanovesicles and/or populations of nanovesicles included in the purified fractions. In some embodiments, nanovesicle size is measured as the longest measurable dimension. Generally, the longest general dimension of a nanovesicle is also referred to as its diameter.

Nanovesicle size can be measured using various methods known in the art, for example, nanoparticle tracking analysis, multi-angle light scattering, single angle light scattering, size exclusion chromatography, analytical ultracentrifugation, field flow fractionation, laser diffraction, tunable resistive pulse sensing, or dynamic light scattering.

In some embodiments, the methods described herein comprise measuring the density of polypeptides (in particular, Eph receptor derived polypeptides) on the nanovesicle surface. The surface density can be calculated or presented as the mass per unit area, the number of proteins per area, number of molecules or intensity of molecule signal per nanovesicle, molar amount of the protein, etc. The surface density can be experimentally measured by methods known in the art, for example, by using bio-layer interferometry (BLI), FACS, Western blotting, fluorescence (e.g., GFP-fusion protein) detection, nano-flow cytometry, ELISA, alphaLISA, and/or densitometry by measuring bands on a protein gel.

In other embodiments, a composition of isolated nanovesicles has an amount and/or concentration of desired nanovesicles at or above an acceptable amount and/or concentration. In other embodiments, the composition of isolated nanovesicles is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 990.99%, 990.999%, 990.9999%, or greater than 99.99990%) as compared to the starting material. In some embodiments, isolated nanovesicle preparations are substantially free of residual biological products. In some embodiments, the isolated nanovesicle preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Being substantially free of residual biological products can also mean that the nanovesicle composition contains no detectable producer cells and that only nanovesicles are detectable.

Furthermore, the nanovesicles of the present disclosure may also comprise additional payloads, in addition to the polypeptide (in particular, Eph receptor derived polypeptide) which may be incorporated into the nanovesicle membrane. The nanovesicles can comprise various macromolecular cargo either within the internal space, displayed on the external surface of the nanovesicle, and/or spanning the membrane. Said cargo can comprise nucleic acids, proteins, carbohydrates, lipids, small molecules, and/or combinations thereof. In some embodiments, the internal volume of the nanovesicle contains at least one bioactive agent originating from an extracellular vesicle (e.g. endogenous polynucleotides, enzymes or polypeptides) and at least one bioactive agent encapsulated in a nanovesicle manufactured in vitro. In another embodiment, the internal volume of the nanovesicle only comprises natural components originating from the extracellular vesicles and may be further treated.

In some embodiments, hybridosomes are used to produce pharmaceutical compositions that facilitate or enhance the encapsulation and release of encapsulated materials (e.g., active agents) to one or more target extracellular vesicles (e.g., by permeating or fusing with the lipid membranes of extracellular vesicles). For example, when a lipid-based composition comprises or is otherwise enriched with one or more of the ionizable lipids, the phase transition in the lipid bilayer of the one or more extracellular vesicles may facilitate the delivery of the encapsulated materials (e.g., active agents encapsulated in a lipid nanoparticle) into one or more hybridosomes. In one embodiment of this disclosure, hybridosomes can be manufactured to encapsulate enzymatic and bioactive catalytic compounds that upon integration into the hybridosome are capable of interacting with one or more compounds originating from the extracellular vesicles. For example, hybridosomes can be manufactured to contain ribonucleases, capable of degradation of any endogenous polynucleotides transferred into a hybridosome by the extracellular vesicles.

### 5.5 Compositions and Kits

In another aspect, compositions and kits are provided, comprising a polypeptide (in particular, an Eph receptor derived polypeptide), nanovesicle, nucleic acid, expression vector, and/or a cell of the disclosure (e.g., as described in Sections 5.2-5.4). Such compositions can, e.g., be a cosmetic, a diagnostic, or a pharmaceutical composition.

In certain embodiments, a composition as described herein is useful as a medicament. Typically, such a medicament includes a therapeutically effective amount of a composition provided herein. Accordingly, a respective composition can be used for the production of a medicament useful in the treatment of disorders. Thus, in one embodiment, pharmaceutical compositions and kits comprising a polypeptide (in particular, an Eph receptor derived polypeptide), nanovesicle, nucleic acid, expression vector, and/or a cell of the disclosure are provided. In some embodiments, provided are pharmaceutical compositions and kits comprising a nanovesicle of the disclosure (i.e., a nanovesicle comprising a polypeptide (in particular, an Eph receptor derived polypeptide) as described above).

In some embodiments, a pharmaceutical composition comprises a polypeptide (in particular, an Eph receptor derived polypeptide), nanovesicle, nucleic acid, expression vector, and/or a cell described herein and further comprises one or more pharmaceutically acceptable carriers, excipients and/or diluent. Guidance for preparing formulations can be found in any number of handbooks for pharmaceutical preparation and formulation that are known to those of skill in the art.

Pharmaceutically acceptable carriers include any solvents, dispersion media, or coatings that are physiologically compatible and that preferably do not interfere with or otherwise inhibit the activity of the active agent. Various pharmaceutically acceptable excipients are well-known in the art.

In some embodiments, the pharmaceutically acceptable carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acids or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

Pharmaceutical compositions can be manufactured in a manner that is known to those of skill in the art, e.g., by means of conventional mixing, dissolving, granulating, dragee-making, emulsifying, encapsulating, entrapping or lyophilizing processes.

Typically, a pharmaceutical composition for use in in vivo administration is sterile. Sterilization can be accomplished according to methods known in the art, e.g., sterile filtration.

Dosages and desired drug concentration of pharmaceutical compositions of the disclosure may vary depending on the particular use envisioned.

### 5.6 Therapeutic and Diagnostic Uses

The nanovesicles comprising the polypeptide (in particular, Eph receptor derived polypeptide) of the present disclosure (e.g., as described in Section 5.4), as well as nucleic acids and expression vectors encoding such polypeptides (e.g., as described in Section 5.3), cells capable of expressing such polypeptides (e.g., as described in Section 5.3), and compositions and kits comprising the foregoing (e.g., as described in Section 5.5) may be used for treating, monitoring, preventing and/or diagnosing a number of diseases and disorders (e.g. cancer, inflammation, or inflammation associated with cancer).

Thus, in one aspect, provided herein is a method of delivering a therapeutic or diagnostic agent to a target cell or tissue, wherein the method comprises providing an extracellular vesicle or hybridosome described herein to said target cell or tissue.

In one aspect, a method of treating a disease or disorder is provided. The method comprises the steps of administering a pharmaceutically effective amount of a composition as described herein (i.e. a composition comprising or capable of expressing a polypeptide (in particular, an Eph receptor derived polypeptide)) to a subject in need thereof. In one embodiment, the method comprises administering a pharmaceutically effective amount of a pharmaceutical composition described above.

The subject in need of a treatment can be a human or a non-human animal. Typically, the subject is a mammal, e.g., an ape, a dog, a guinea pig, a horse, a monkey, a mouse, a pig, a rabbit or a rat. In case of an animal model, the animal might be genetically engineered to develop a disorder or to show the characteristics of a disease.

In some embodiments, the subject has a cancer, an inflammatory disorder, autoimmune disease, a chronic disease, inflammation, damaged organ function, an infectious disease, metabolic disease, degenerative disorder, genetic disease (e.g., a genetic deficiency, a recessive genetic disorder, or a dominant genetic disorder), or an injury. In some embodiments, the subject has an infectious disease and the nanovesicle comprises an antigen for the infectious disease. In some embodiments, the subject has a genetic deficiency and the nanovesicle comprises a protein for which the subject is deficient, or a nucleic acid (e.g., mRNA) encoding the protein, or a DNA encoding the protein, or a chromosome encoding the protein, or a nucleus comprising a nucleic acid encoding the protein. In some embodiments, the subject has a dominant genetic disorder, and the nanovesicle comprises a nucleic acid inhibitor (e.g., shRNA, siRNA or miRNA) of the dominant mutant allele. In some embodiments, the subject has a dominant genetic disorder, and/or the nanovesicle comprises a nucleic acid inhibitor (e.g., shRNA, siRNA or miRNA) of the dominant mutant allele, and/or the nanovesicle also comprises an mRNA encoding a non-mutated allele of the mutated gene that is not targeted by the nucleic acid inhibitor. In some embodiments, the subject is in need of vaccination. In some embodiments, the subject is in need of regeneration, e.g., of an injured site.

In some embodiments, the composition is administered to the subject at least 1, 2, 3, 4, or 5 times.

In some embodiments, the nanovesicle comprising a polypeptide (in particular, an Eph receptor derived polypeptide) described herein targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse or human. In some embodiments, at least 0.1%, 0.5%, 1%, 1.5%, 2%, 2.5%, 3%, 4%, 5%0, 10%, 20%, 30%, 40%, 50%, 60%, 70%, 80%, or 90% of the nanovesicles comprising a polypeptide (in particular, Eph receptor derived polypeptide) described herein in an administered composition are present in the target tissue after 24, 48, or 72 hours.

In some embodiments, the composition as described above is administered to a subject at a therapeutically effective amount or dose. Illustrative dosages include a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In some embodiments, the compositions as disclosed herein are used for the treatment of cancer. In certain embodiments, the cancer is a primary cancer of the CNS, such as glioma, glioblastoma multiforme, meningioma, astrocytoma, acoustic neuroma, chondroma, oligodendroglioma, medulloblastomas, ganglioglioma, Schwannoma, neurofibroma, neuroblastoma, or an extradural, intramedullary or intradural tumor. In some embodiments, the cancer is a solid tumor, or in other embodiments, the cancer is a non-solid tumor. Solid-tumor cancers include tumors of the central nervous system, breast cancer, prostate cancer, skin cancer (including basal cell carcinoma, cell carcinoma, squamous cell carcinoma and melanoma), cervical cancer, uterine cancer, lung cancer, ovarian cancer, testicular cancer, thyroid cancer, astrocytoma, glioma, pancreatic cancer, mesotheliomas, gastric cancer, liver cancer, colon cancer, rectal cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas. In some embodiments, the use of a nanovesicle as disclosed herein in the manufacture of a medicament for treating cancer is provided.

In some embodiments, the compositions as disclosed herein may be used in the treatment of an autoimmune or inflammatory disease. Examples of such diseases include, but are not limited to, ankylosing spondylitis, arthritis, osteoarthritis, rheumatoid arthritis, psoriatic arthritis, asthma, scleroderma, stroke, atherosclerosis, Crohn's disease, colitis, ulcerative colitis, dermatitis, diverticulitis, fibrosis, idiopathic pulmonary fibrosis, fibromyalgia, hepatitis, irritable bowel syndrome (IBS), lupus, systemic lupus erythematous (SLE), nephritis, multiple sclerosis, and ulcerative colitis. In some embodiments, the use of a nanovesicle as disclosed herein in the manufacture of a medicament for treating an autoimmune or inflammatory disease is provided.

In some embodiments, the compositions as disclosed herein may be used in the treatment of a cardiovascular disease, such as coronary artery disease, heart attack, abnormal heart rhythms or arrhythmias, heart failure, heart valve disease, congenital heart disease, heart muscle disease, cardiomyopathy, pericardial disease, aorta disease, marfan syndrome, vascular disease, or blood vessel disease.

The compositions of the present disclosure may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the nanovesicle and/or the therapeutic molecule.

A nanovesicle as disclosed herein may be used for detection or diagnostic purposes in vivo and/or in vitro which encompasses quantitative and/or qualitative detection. Likewise, a polypeptide (in particular, Eph receptor derived polypeptide), a nucleic acid, an expression vector and/or a cell described in the preceding text can be used accordingly as detailed in this section.

For diagnostic applications or detection purposes, the nanovesicle may include a moiety that is detectable, e.g., detectable through biological imaging, including radiology or magnetic resonance imaging. In some embodiments, the nanovesicle comprises a reporter protein or a detectable label. In some embodiments, the nanovesicle as disclosed herein is coupled to one or more substances that can be recognized by a detector substance. By way of example, the nanovesicle may be covalently linked to biotin, which can be detected by means of its capability to bind to streptavidin.

In certain embodiments, the nanovesicle is useful for detecting its presence in a sample, preferably a sample of biological origin, such as, e.g., from a human subject. Non-limiting examples of biological samples include blood, biopsy, cerebrospinal fluid, lymph, urine, and/or non-blood tissues. In certain embodiments, a biological sample includes a cell or tissue from human patients.

Thus, in some aspects, methods are provided, including the steps of: (i) contacting a subject or a biological sample with a nanovesicle of the disclosure comprising a detectable moiety; (ii) allowing for the nanovesicle to interact with the subject or sample; and (iii) detecting the nanovesicle. Such methods may be in vitro or in vivo methods. In some embodiments, such methods are methods for localizing a nanovesicle.

### 6. ADDITIONAL SEQUENCES

TABLE 21
Additional sequences disclosed herein are:

| SEQ ID | | |
|---|---|---|
| NO: | Description | Sequences |
| 212 | Human | MERRWPLGLGLVLLLCAPLPPGARAKEVTL |
| | EPHA1 | MDTSKAQGELGWLLDPPKDGWSEQQQILNG |
| | | TPLYMYQDCPMQGRRDTDHWLRSNWIYRGE |
| | | EASRVHVELQFTVRDCKSFPGGAGPLGCKE |
| | | TFNLLYMESDQDVGIQLRRPLFQKVTTVAA |
| | | DQSFTIRDLVSGSVKLNVERCSLGRLTRRG |
| | | LYLAFHNPGACVALVSVRVFYQRCPETLNG |
| | | LAQFPDTLPGPAGLVEVAGTCLPHARASPR |
| | | PSGAPRMHCSPDGEWLVPVGRCHCEPGYEE |
| | | GGSGEACVACPSGSYRMDMDTPHCLTCPQQ |
| | | STAESEGATICTCESGHYRAPGEGPQVACT |
| | | GPPSAPRNLSFSASGTQLSLRWEPPADTGG |
| | | RQDVRYSVRCSQCQGTAQDGGPCQPCGVGV |
| | | HFSPGARGLTTPAVHVNGLEPYANYTFNVE |
| | | AQNGVSGLGSSGHASTSVSISMGHAESLSG |
| | | LSLRLVKKEPRQLELTWAGSRPRSPGANLT |
| | | YELHVLNQDEERYQMVLEPRVLLTELQPDT |
| | | TYIVRVRMLTPLGPGPFSPDHEFRTSPPVS |
| | | RGLTGGEIVAVIFGLLLGAALLLGILVFRS |
| | | RRAQRQRQQRQRDRATDVDREDKLWLKPYV |
| | | DLQAYEDPAQGALDFTRELDPAWLMVDTVI |
| | | GEGEFGEVYRGTLRLPSQDCKTVAIKTLKD |
| | | TSPGGQWWNFLREATIMGQFSHPHILHLEG |
| | | VVTKRKPIMIITEFMENGALDAFLREREDQ |
| | | LVPGQLVAMLQGIASGMNYLSNHNYVHRDL |
| | | AARNILVNQNLCCKVSDFGLTRLLDDFDGT |
| | | YETQGGKIPIRWTAPEAIAHRIFTTASDVW |
| | | SFGIVMWEVLSFGDKPYGEMSNQEVMKSIE |
| | | DGYRLPPPVDCPAPLYELMKNCWAYDRARR |
| | | PHFQKLQAHLEQLLANPHSLRTIANFDPRM |
| | | TLRLPSLSGSDGIPYRTVSEWLESIRMKRY |
| | | ILHFHSAGLDTMECVLELTAEDLTQMGITL |
| | | PGHQKRILCSIQGFKD |
| 213 | Human | MELQAARACFALLWGCALAAAAAAQGKEVV |
| | EPHA2 | LLDFAAAGGELGWLTHPYGKGWDLMQNIMN |
| | | DMPIYMYSVCNVMSGDQDNWLRTNWVYRGE |
| | | AERIFIELKFTVRDCNSFPGGASSCKETFN |
| | | LYYAESDLDYGTNFQKRLFTKIDTIAPDEI |
| | | TVSSDFEARHVKLNVEERSVGPLTRKGFYL |
| | | AFQDIGACVALLSVRVYYKKCPELLQGLAH |
| | | FPETIAGSDAPSLATVAGTCVDHAVVPPGG |
| | | EEPRMHCAVDGEWLVPIGQCLCQAGYEKVE |
| | | DACQACSPGFFKFEASESPCLECPEHTLPS |
| | | PEGATSCECEEGFFRAPQDPASMPCTRPPS |
| | | APHYLTAVGMGAKVELRWTPPQDSGGREDI |
| | | VYSVTCEQCWPESGECGPCEASVRYSEPPH |
| | | GLTRTSVTVSDLEPHMNYTFTVEARNGVSG |
| | | LVTSRSFRTASVSINQTEPPKVRLEGRSTT |
| | | SLSVSWSIPPPQQSRVWKYEVTYRKKGDSN |
| | | SYNVRRTEGFSVTLDDLAPDTTYLVQVQAL |
| | | TQEGQGAGSKVHEFQTLSPEGSGNLAVIGG |
| | | VAVGVVLLLVLAGVGFFIHRRRKNQRARQS |
| | | PEDVYFSKSEQLKPLKTYVDPHTYEDPNQA |
| | | VLKFTTEIHPSCVTRQKVIGAGEFGEVYKG |
| | | MLKTSSGKKEVPVAIKTLKAGYTEKQRVDF |
| | | LGEAGIMGQFSHHNIIRLEGVISKYKPMMI |
| | | ITEYMENGALDKFLREKDGEFSVLQLVGML |
| | | RGIAAGMKYLANMNYVHRDLAARNILVNSN |
| | | LVCKVSDFGLSRVLEDDPEATYTTSGGKIP |
| | | IRWTAPEAISYRKFTSASDVWSFGIVMWEV |
| | | MTYGERPYWELSNHEVMKAINDGFRLPTPM |
| | | DCPSAIYQLMMQCWQQERARRPKFADIVSI |
| | | LDKLIRAPDSLKTLADFDPRVSIRLPSTSG |
| | | SEGVPFRTVSEWLESIKMQQYTEHFMAAGY |
| | | TAIEKVVQMINDDIKRIGVRLPGHQKRIAY |
| | | SLLGLKDQVNTVGIPI |
| 214 | Human | MDCQLSILLLLSCSVLDSFGELIPQPSNEV |
| | EPHA3 | NLLDSKTIQGELGWISYPSHGWEEISGVDE |
| | | HYTPIRTYQVCNVMDHSQNNWLRTNWVPRN |
| | | SAQKIYVELKFTLRDCNSIPLVLGTCKETF |
| | | NLYYMESDDDHGVKFREHQFTKIDTIAADE |
| | | SFTQMDLGDRILKLNTEIREVGPVNKKGFY |
| | | LAFQDVGACVALVSVRVYFKKCPFTVKNLA |
| | | MFPDTVPMDSQSLVEVRGSCVNNSKEEDPP |
| | | RMYCSTEGEWLVPIGKCSCNAGYEERGFMC |
| | | QACRPGFYKALDGNMKCAKCPPHSSTQEDG |
| | | SMNCRCENNYFRADKDPPSMACTRPPSSPR |
| | | NVISNINETSVILDWSWPLDTGGRKDVTFN |
| | | IICKKCGWNIKQCEPCSPNVRFLPRQFGLT |
| | | NTTVTVTDLLAHTNYTFEIDAVNGVSELSS |
| | | PPRQFAAVSITTNQAAPSPVLTIKKDRTSR |
| | | NSISLSWQEPEHPNGIILDYEVKYYEKQEQ |
| | | ETSYTILRARGTNVTISSLKPDTIYVFQIR |
| | | ARTAAGYGTNSRKFEFETSPDSFSISGESS |
| | | QVVMIAISAAVAIILLTVVIYVLIGRFCGY |
| | | KSKHGADEKRLHFGNGHLKLPGLRTYVDPH |
| | | TYEDPTQAVHEFAKELDATNISIDKVVGAG |
| | | EFGEVCSGRLKLPSKKEISVAIKTLKVGYT |
| | | EKQRRDFLGEASIMGQFDHPNIIRLEGVVT |
| | | KSKPVMIVTEYMENGSLDSFLRKHDAQFTV |
| | | IQLVGMLRGIASGMKYLSDMGYVHRDLAAR |
| | | NILINSNLVCKVSDFGLSRVLEDDPEAAYT |
| | | TRGGKIPIRWTSPEAIAYRKFTSASDVWSY |
| | | GIVLWEVMSYGERPYWEMSNQDVIKAVDEG |
| | | YRLPPPMDCPAALYQLMLDCWQKDRNNRPK |
| | | FEQIVSILDKLIRNPGSLKIITSAAARPSN |
| | | LLLDQSNVDITTFRTTGDWLNGVWTAHCKE |
| | | IFTGVEYSSCDTIAKISTDDMKKVGVTVVG |
| | | PQKKIISSIKALETQSKNGPVPV |
| 215 | Human | MAGIFYFALFSCLFGICDAVTGSRVYPANE |
| | EPHA4 | VTLLDSRSVQGELGWIASPLEGGWEEVSIM |
| | | DEKNTPIRTYQVCNVMEPSQNNWLRTDWIT |
| | | REGAQRVYIEIKFTLRDCNSLPGVMGTCKE |
| | | TFNLYYYESDNDKERFIRENQFVKIDTIAA |
| | | DESFTQVDIGDRIMKLNTEIRDVGPLSKKG |
| | | FYLAFQDVGACIALVSVRVFYKKCPLTVRN |
| | | LAQFPDTITGADTSSLVEVRGSCVNNSEEK |
| | | DVPKMYCGADGEWLVPIGNCLCNAGHEERS |
| | | GECQACKIGYYKALSTDATCAKCPPHSYSV |
| | | WEGATSCTCDRGFFRADNDAASMPCTRPPS |
| | | APLNLISNVNETSVNLEWSSPQNTGGRQDI |
| | | SYNVVCKKCGAGDPSKCRPCGSGVHYTPQQ |
| | | NGLKTTKVSITDLLAHTNYTFEIWAVNGVS |
| | | KYNPNPDQSVSVTVTTNQAAPSSIALVQAK |
| | | EVTRYSVALAWLEPDRPNGVILEYEVKYYE |
| | | KDQNERSYRIVRTAARNTDIKGLNPLTSYV |
| | | FHVRARTAAGYGDFSEPLEVTTNTVPSRII |
| | | GDGANSTVLLVSVSGSVVLVVILIAAFVIS |
| | | RRRSKYSKAKQEADEEKHLNQGVRTYVDPF |
| | | TYEDPNQAVREFAKEIDASCIKIEKVIGVG |
| | | EFGEVCSGRLKVPGKREICVAIKTLKAGYT |
| | | DKQRRDFLSEASIMGQFDHPNIIHLEGVVT |
| | | KCKPVMIITEYMENGSLDAFLRKNDGRFTV |
| | | IQLVGMLRGIGSGMKYLSDMSYVHRDLAAR |
| | | NILVNSNLVCKVSDFGMSRVLEDDPEAAYT |
| | | TRGGKIPIRWTAPEAIAYRKFTSASDVWSY |
| | | GIVMWEVMSYGERPYWDMSNQDVIKAIEEG |
| | | YRLPPPMDCPIALHQLMLDCWQKERSDRPK |
| | | FGQIVNMLDKLIRNPNSLKRTGTESSRPNT |
| | | ALLDPSSPEFSAVVSVGDWLQAIKMDRYKD |
| | | NFTAAGYTTLEAVVHVNQEDLARIGITAIT |
| | | HQNKILSSVQAMRTQMQQMHGRMVPV |
| 216 | Human | MRGSGPRGAGRRRPPSGGGDTPITPASLAG |
| | EPHA5 | CYSAPRRAPLWTCLLLCAALRTLLASPSNE |
| | | VNLLDSRTVMGDLGWIAFPKNGWEEIGEVD |
| | | ENYAPIHTYQVCKVMEQNQNNWLLTSWISN |
| | | EGASRIFIELKFTLRDCNSLPGGLGTCKET |
| | | FNMYYFESDDQNGRNIKENQYIKIDTIAAD |
| | | ESFTELDLGDRVMKLNTEVRDVGPLSKKGF |
| | | YLAFQDVGACIALVSVRVYYKKCPSVVRHL |
| | | AVFPDTITGADSSQLLEVSGSCVNHSVTDE |
| | | PPKMHCSAEGEWLVPIGKCMCKAGYEEKNG |
| | | TCQVCRPGFFKASPHIQSCGKCPPHSYTHE |
| | | EASTSCVCEKDYFRRESDPPTMACTRPPSA |
| | | PRNAISNVNETSVFLEWIPPADTGGRKDVS |
| | | YYIACKKCNSHAGVCEECGGHVRYLPRQSG |
| | | LKNTSVMMVDLLAHTNYTFEIEAVNGVSDL |
| | | SPGARQYVSVNVTTNQAAPSPVTNVKKGKI |
| | | AKNSISLSWQEPDRPNGIILEYEIKYFEKD |
| | | QETSYTIIKSKETTITAEGLKPASVYVFQI |
| | | RARTAAGYGVFSRRFEFETTPVFAASSDQS |
| | | QIPVIAVSVTVGVILLAVVIGVLLSGSCCE |
| | | CGCGRASSLCAVAHPSLIWRCGYSKAKQDP |
| | | EEEKMHFHNGHIKLPGVRTYIDPHTYEDPN |
| | | QAVHEFAKEIEASCITIERVIGAGEFGEVC |
| | | SGRLKLPGKRELPVAIKTLKVGYTEKQRRD |
| | | FLGEASIMGQFDHPNIIHLEGVVTKSKPVM |
| | | IVTEYMENGSLDTFLKKNDGQFTVIQLVGM |
| | | LRGISAGMKYLSDMGYVHRDLAARNILINS |
| | | NLVCKVSDFGLSRVLEDDPEAAYTTRGGKI |
| | | PIRWTAPEAIAFRKFTSASDVWSYGIVMWE |
| | | VVSYGERPYWEMTNQD |
| 217 | Human | VIKAVEEGYRLPSPMDCPAALYQLMLDCWQ |
| | EPHA6 | KERNSRPKFDEIVNMLDKLIRNPSSLKTLV |
| | | NASCRVSNLLAEHSPLGSGAYRSVGEWLEA |
| | | IKMGRYTEIFMENGYSSMDAVAQVTLEDLR |
| | | RLGVTLVGHQKKIMNSLQEMKVQLVNGMVP |
| | | LMGGCEVREFLLQFGFFLPLLTAWPGDCSH |
| | | VSNNQVVLLDTTTVLGELGWKTYPLNGWDA |
| | | ITEMDEHNRPIHTYQVCNVMEPNQNNWLRT |
| | | NWISRDAAQKIYVEMKFTLRDCNSIPWVLG |
| | | TCKETFNLFYMESDESHGIKFKPNQYTKID |
| | | TIAADESFTQMDLGDRILKLNTEIREVGPI |
| | | ERKGFYLAFQDIGACIALVSVRVFYKKCPF |
| | | TVRNLAMFPDTIPRVDSSSLVEVRGSCVKS |
| | | AEERDTPKLYCGADGDWLVPLGRCICSTGY |
| | | EEIEGSCHACRPGFYKAFAGNTKCSKCPPH |
| | | SLTYMEATSVCQCEKGYFRAEKDPPSMACT |
| | | RPPSAPRNVVFNINETALILEWSPPSDTGG |
| | | RKDLTYSVICKKCGLDTSQCEDCGGGLRFI |
| | | PRHTGLINNSVIVLDFVSHVNYTFEIEAMN |
| | | GVSELSFSPKPFTAITVTTDQDAPSLIGVV |
| | | RKDWASQNSIALSWQAPAFSNGAILDYEIK |
| | | YYEKEHEQLTYSSTRSKAPSVIITGLKPAT |
| | | KYVFHIRVRTATGYSGYSQKFEFETGDETS |
| | | DMAAEQGQILVIATAAVGGFTLLVILTLFF |
| | | LITGRCQWYIKAKMKSEEKRRNHLQNGHLR |
| | | FPGIKTYIDPDTYEDPSLAVHEFAKEIDPS |
| | | RIRIERVIGAGEFGEVCSGRLKTPGKREIP |
| | | VAIKTLKGGHMDRQRRDFLREASIMGQFDH |
| | | PNIIRLEGVVTKRSFPAIGVEAFCPSFLRA |
| | | GFLNSIQAPHPVPGGGSLPPRIPAGRPVMI |
| | | VVEYMENGSLDSFLRKHDGHFTVIQLVGML |
| | | RGIASGMKYLSDMGYVHRDLAARNILVNSN |
| | | LVCKVSDFGLSRVLEDDPEAAYTTTGGKIP |
| | | IRWTAPEAIAYRKESSASDAWSYGIVMWEV |
| | | MSYGERPYWEMSNQDVILSIEEGYRLPAPM |
| | | GCPASLHQLMLHCWQKERNHRPKFTDIVSF |
| | | LDKLIRNPSALHTLVEDILVMPESPGEVPE |
| | | YPLFVTVGDWLDSIKMGQYKNNFVAAGFTT |
| | | FDLISRMSIDDIRRIGVILIGHQRRIVSSI |
| | | QTLRLHMMHIQEKGFHV |
| 218 | Human | MVFQTRYPSWIILCYIWLLRFAHTGEAQAA |
| | EPHA7 | KEVLLLDSKAQQTELEWISSPPNGWEEISG |
| | | LDENYTPIRTYQVCQVMEPNQNNWLRTNWI |
| | | SKGNAQRIFVELKFTLRDCNSLPGVLGTCK |
| | | ETFNLYYYETDYDTGRNIRENLYVKIDTIA |
| | | ADESFTQGDLGERKMKLNTEVREIGPLSKK |
| | | GFYLAFQDVGACIALVSVKVYYKKCWSIIE |
| | | NLAIFPDTVTGSEFSSLVEVRGTCVSSAEE |
| | | EAENAPRMHCSAEGEWLVPIGKCICKAGYQ |
| | | QKGDTCEPCGRGFYKSSSQDLQCSRCPTHS |
| | | FSDKEGSSRCECEDGYYRAPSDPPYVACTR |
| | | PPSAPQNLIFNINQTTVSLEWSPPADNGGR |
| | | NDVTYRILCKRCSWEQGECVPCGSNIGYMP |
| | | QQTGLEDNYVTVMDLLAHANYTFEVEAVNG |
| | | VSDLSRSQRLFAAVSITTGQAAPSQVSGVM |
| | | KERVLQRSVELSWQEPEHPNGVITEYEIKY |
| | | YEKDQRERTYSTVKTKSTSASINNLKPGTV |
| | | YVFQIRAFTAAGYGNYSPRLDVATLEEATG |
| | | KMFEATAVSSEQNPVIIIAVVAVAGTIILV |
| | | FMVFGFIIGRRHCGYSKADQEGDEELYFHF |
| | | KFPGTKTYIDPETYEDPNRAVHQFAKELDA |
| | | SCIKIERVIGAGEFGEVCSGRLKLPGKRDV |
| | | AVAIKTLKVGYTEKQRRDFLCEASIMGQFD |
| | | HPNVVHLEGVVTRGKPVMIVIEFMENGALD |
| | | AFLRKHDGQFTVIQLVGMLRGIAAGMRYLA |
| | | DMGYVHRDLAARNILVNSNLVCKVSDFGLS |
| | | RVIEDDPEAVYTTTGGKIPVRWTAPEAIQY |
| | | RKFTSASDVWSYGIVMWEVMSYGERPYWDM |
| | | SNQDVIKAIEEGYRLPAPMDCPAGLHQLML |
| | | DCWQKERAERPKFEQIVGILDKMIRNPNSL |
| | | KTPLGTCSRPISPLLDQNTPDFTTFCSVGE |
| | | WLQAIKMERYKDNFTAAGYNSLESVARMTI |
| | | EDVMSLGITLVGHQKKIMSSIQTMRAQMLH |
| | | LHGTGIQV |
| 219 | Human | MAPARGRLPPALWVVTAAAAAATCVSAARG |
| | EPHA8 | EVNLLDTSTIHGDWGWLTYPAHGWDSINEV |
| | | DESFQPIHTYQVCNVMSPNQNNWLRTSWVP |
| | | RDGARRVYAEIKFTLRDCNSMPGVLGTCKE |
| | | TFNLYYLESDRDLGASTQESQFLKIDTIAA |
| | | DESFTGADLGVRRLKLNTEVRSVGPLSKRG |
| | | FYLAFQDIGACLAILSLRIYYKKCPAMVRN |
| | | LAAFSEAVTGADSSSLVEVRGQCVRHSEER |
| | | DTPKMYCSAEGEWLVPIGKCVCSAGYEERR |
| | | DACVACELGFYKSAPGDQLCARCPPHSHSA |
| | | APAAQACHCDLSYYRAALDPPSSACTRPPS |
| | | APVNLISSVNGTSVTLEWAPPLDPGGRSDI |
| | | TYNAVCRRCPWALSRCEACGSGTRFVPQQT |
| | | SLVQASLLVANLLAHMNYSFWIEAVNGVSD |
| | | LSPEPRRAAVVNITTNQAAPSQVVVIRQER |
| | | AGQTSVSLLWQEPEQPNGIILEYEIKYYEK |
| | | DKEMQSYSTLKAVTTRATVSGLKPGTRYVF |
| | | QVRARTSAGCGRFSQAMEVETGKPRPRYDT |
| | | RTIVWICLTLITGLVVLLLLLICKKRHCGY |
| | | SKAFQDSDEEKMHYQNGQAPPPVFLPLHHP |
| | | PGKLPEPQFYAEPHTYEEPGRAGRSFTREI |
| | | EASRIHIEKIIGSGDSGEVCYGRLRVPGQR |
| | | DVPVAIKALKAGYTERQRRDFLSEASIMGQ |
| | | FDHPNIIRLEGVVTRGRLAMIVTEYMENGS |
| | | LDTFLRTHDGQFTIMQLVGMLRGVGAGMRY |
| | | LSDLGYVHRDLAARNVLVDSNLVCKVSDFG |
| | | LSRVLEDDPDAAYTTTGGKIPIRWTAPEAI |
| | | AFRTFSSASDVWSFGVVMWEVLAYGERPYW |
| | | NMTNRDVISSVEEGYRLPAPMGCPHALHQL |
| | | MLDCWHKDRAQRPRFSQIVSVLDALIRSPE |
| | | SLRATATVSRCPPPAFVRSCFDLRGGSGGG |
| | | GGLTVGDWLDSIRMGRYRDHFAAGGYSSLG |
| | | MVLRMNAQDVRALGITLMGHQKKILGSIQT |
| | | MRAQLTSTQGPRRHL |
| 220 | Human | METCAGPHPLRLFLCRMQLCLALLLGPWRP |
| | EPHA10 | GTAEEVILLDSKASQAELGWTALPSNGWEE |
| | | ISGVDEHDRPIRTYQVCNVLEPNQDNWLQT |
| | | GWISRGRGQRIFVELQFTLRDCSSIPGAAG |
| | | TCKETFNVYYLETEADLGRGRPRLGGSRPR |
| | | KIDTIAADESFTQGDLGERKMKLNTEVREI |
| | | GPLSRRGFHLAFQDVGACVALVSVRVYYKQ |
| | | CRATVRGLATFPATAAESAFSTLVEVAGTC |
| | | VAHSEGEPGSPPRMHCGADGEWLVPVGRCS |
| | | CSAGFQERGDFCEACPPGFYKVSPRRPLCS |
| | | PCPEHSRALENASTFCVCQDSYARSPTDPP |
| | | SASCTRPPSAPRDLQYSLSRSPLVLRLRWL |
| | | PPADSGGRSDVTYSLLCLRCGREGPAGACE |
| | | PCGPRVAFLPRQAGLRERAATLLHLRPGAR |
| | | YTVRVAALNGVSGPAAAAGTTYAQVTVSTG |
| | | PGAPWEEDEIRRDRVEPQSVSLSWREPIPA |
| | | GAPGANDTEYEIRYYEKGQSEQTYSMVKTG |
| | | APTVTVTNLKPATRYVFQIRAASPGPSWEA |
| | | QSFNPSIEVQTLGEAASGSRDQSPAIVVTV |
| | | VTISALLVLGSVMSVLAIWRRPCSYGKGGG |
| | | DAHDEEELYFHFKVPTRRTFLDPQSCGDLL |
| | | QAVHLFAKELDAKSVTLERSLGGGRFGELC |
| | | CGCLQLPGRQELLVAVHMLRDSASDSQRLG |
| | | FLAEALTLGQFDHSHIVRLEGVVTRGSTLM |
| | | IVTEYMSHGALDGFLRRHEGQLVAGQLMGL |
| | | LPGLASAMKYLSEMGYVHRGLAARHVLVSS |
| | | DLVCKISGFGRGPRDRSEAVYTTMSGRSPA |
| | | LWAAPETLQFGHFSSASDVWSFGIIMWEVM |
| | | AFGERPYWDMSGQDVIKAVEDGFRLPPPRN |
| | | CPNLLHRLMLDCWQKDPGERPRFSQIHSIL |
| | | SKMVQDPEPPKCALTTCPRPPTPLADRAFS |
| | | TFPSFGSVGAWLEALDLCRYKDSFAAAGYG |
| | | SLEAVAEMTAQDLVSLGISLAEHREALLSG |
| | | ISALQARVLQLQGQGVQV |
| 221 | Human | MALDYLLLLLLASAVAAMEETLMDTRTATA |
| | EPHB1 | ELGWTANPASGWEEVSGYDENLNTIRTYQV |
| | | CNVFEPNQNNWLLTTFINRRGAHRIYTEMR |
| | | FTVRDCSSLPNVPGSCKETFNLYYYETDSV |
| | | IATKKSAFWSEAPYLKVDTIAADESFSQVD |
| | | FGGRLMKVNTEVRSFGPLTRNGFYLAFQDY |
| | | GACMSLLSVRVFFKKCPSIVQNFAVFPETM |
| | | TGAESTSLVIARGTCIPNAEEVDVPIKLYC |
| | | NGDGEWMVPIGRCTCKPGYEPENSVACKAC |
| | | PAGTFKASQEAEGCSHCPSNSRSPAEASPI |
| | | CTCRTGYYRADFDPPEVACTSVPSGPRNVI |
| | | SIVNETSIILEWHPPRETGGRDDVTYNIIC |
| | | KKCRADRRSCSRCDDNVEFVPRQLGLTECR |
| | | VSISSLWAHTPYTFDIQAINGVSSKSPFPP |
| | | QHVSVNITTNQAAPSTVPIMHQVSATMRSI |
| | | TLSWPQPEQPNGIILDYEIRYYEKEHNEFN |
| | | SSMARSQTNTARIDGLRPGMVYVVQVRART |
| | | VAGYGKFSGKMCFQTLTDDDYKSELREQLP |
| | | LIAGSAAAGVVFVVSLVAISIVCSRKRAYS |
| | | KEAVYSDKLQHYSTGRGSPGMKIYIDPFTY |
| | | EDPNEAVREFAKEIDVSFVKIEEVIGAGEF |
| | | GEVYKGRLKLPGKREIYVAIKTLKAGYSEK |
| | | QRRDFLSEASIMGQFDHPNIIRLEGVVTKS |
| | | RPVMIITEFMENGALDSFLRQNDGQFTVIQ |
| | | LVGMLRGIAAGMKYLAEMNYVHRDLAARNI |
| | | LVNSNLVCKVSDFGLSRYLQDDTSDPTYTS |
| | | SLGGKIPVRWTAPEAIAYRKFTSASDVWSY |
| | | GIVMWEVMSFGERPYWDMSNQDVINAIEQD |
| | | YRLPPPMDCPAALHQLMLDCWQKDRNSRPR |
| | | FAEIVNTLDKMIRNPASLKTVATITAVPSQ |
| | | PLLDRSIPDFTAFTTVDDWLSAIKMVQYRD |
| | | SFLTAGFTSLQLVTQMTSEDLLRIGITLAG |
| | | HQKKILNSIHSMRVQISQSPTAMA |
| 222 | Human | MALRRLGAALLLLPLLAAVEETLMDSTTAT |
| | EPHB2 | AELGWMVHPPSGWEEVSGYDENMNTIRTYQ |
| | | VCNVFESSQNNWLRTKFIRRRGAHRIHVEM |
| | | KFSVRDCSSIPSVPGSCKETFNLYYYEADF |
| | | DSATKTFPNWMENPWVKVDTIAADESFSQV |
| | | DLGGRVMKINTEVRSFGPVSRSGFYLAFQD |
| | | YGGCMSLIAVRVFYRKCPRIIQNGAIFQET |
| | | LSGAESTSLVAARGSCIANAEEVDVPIKLY |
| | | CNGDGEWLVPIGRCMCKAGFEAVENGTVCR |
| | | GCPSGTFKANQGDEACTHCPINSRTTSEGA |
| | | TNCVCRNGYYRADLDPLDMPCTTIPSAPQA |
| | | VISSVNETSLMLEWTPPRDSGGREDLVYNI |
| | | ICKSCGSGRGACTRCGDNVQYAPRQLGLTE |
| | | PRIYISDLLAHTQYTFEIQAVNGVTDQSPF |
| | | SPQFASVNITTNQAAPSAVSIMHQVSRTVD |
| | | SITLSWSQPDQPNGVILDYELQYYEKELSE |
| | | YNATAIKSPTNTVTVQGLKAGAIYVFQVRA |
| | | RTVAGYGRYSGKMYFQTMTEAEYQTSIQEK |
| | | LPLIIGSSAAGLVFLIAVVVIAIVCNRRGF |
| | | ERADSEYTDKLQHYTSGHMTPGMKIYIDPF |
| | | TYEDPNEAVREFAKEIDISCVKIEQVIGAG |
| | | EFGEVCSGHLKLPGKREIFVAIKTLKSGYT |
| | | EKQRRDFLSEASIMGQFDHPNVIHLEGVVT |
| | | KSTPVMIITEFMENGSLDSFLRQNDGQFTV |
| | | IQLVGMLRGIAAGMKYLADMNYVHRDLAAR |
| | | NILVNSNLVCKVSDFGLSRFLEDDTSDPTY |
| | | TSALGGKIPIRWTAPEAIQYRKFTSASDVW |
| | | SYGIVMWEVMSYGERPYWDMTNQDVINAIE |
| | | QDYRLPPPMDCPSALHQLMLDCWQKDRNHR |
| | | PKFGQIVNTLDKMIRNPNSLKAMAPLSSGI |
| | | NLPLLDRTIPDYTSFNTVDEWLEAIKMGQY |
| | | KESFANAGFTSFDVVSQMMMEDILRVGVTL |
| | | AGHQKKILNSIQVMRAQMNQIQSVEGQPLA |
| | | RRPRATGRTKRCQPRDVTKKTCNSNDGKKK |
| | | GMGKKKTDPGRGREIQGIFFKEDSHKESND |
| | | CSCGG |
| 223 | Human | MARARPPPPPSPPPGLLPLLPPLLLLPLLL |
| | EPHB3 | LPAGCRALEETLMDTKWVTSELAWTSHPES |
| | | GWEEVSGYDEAMNPIRTYQVCNVRESSQNN |
| | | WLRTGFIWRRDVQRVYVELKFTVRDCNSIP |
| | | NIPGSCKETFNLFYYEADSDVASASSPFWM |
| | | ENPYVKVDTIAPDESFSRLDAGRVNTKVRS |
| | | FGPLSKAGFYLAFQDQGACMSLISVRAFYK |
| | | KCASTTAGFALFPETLTGAEPTSLVIAPGT |
| | | CIPNAVEVSVPLKLYCNGDGEWMVPVGACT |
| | | CATGHEPAAKESQCRPCPPGSYKAKQGEGP |
| | | CLPCPPNSRTTSPAASICTCHNNFYRADSD |
| | | SADSACTTVPSPPRGVISNVNETSLILEWS |
| | | EPRDLGGRDDLLYNVICKKCHGAGGASACS |
| | | RCDDNVEFVPRQLGLTERRVHISHLLAHTR |
| | | YTFEVQAVNGVSGKSPLPPRYAAVNITTNQ |
| | | AAPSEVPTLRLHSSSGSSLTLSWAPPERPN |
| | | GVILDYEMKYFEKSEGIASTVTSQMNSVQL |
| | | DGLRPDARYVVQVRARTVAGYGQYSRPAEF |
| | | ETTSERGSGAQQLQEQLPLIVGSATAGLVF |
| | | VVAVVVIAIVCLRKQRHGSDSEYTEKLQQY |
| | | IAPGMKVYIDPFTYEDPNEAVREFAKEIDV |
| | | SCVKIEEVIGAGEFGEVCRGRLKQPGRREV |
| | | FVAIKTLKVGYTERQRRDFLSEASIMGQFD |
| | | HPNIIRLEGVVTKSRPVMILTEFMENCALD |
| | | SFLRLNDGQFTVIQLVGMLRGIAAGMKYLS |
| | | EMNYVHRDLAARNILVNSNLVCKVSDFGLS |
| | | RFLEDDPSDPTYTSSLGGKIPIRWTAPEAI |
| | | AYRKFTSASDVWSYGIVMWEVMSYGERPYW |
| | | DMSNQDVINAVEQDYRLPPPMDCPTALHQL |
| | | MLDCWVRDRNLRPKFSQIVNTLDKLIRNAA |
| | | SLKVIASAQSGMSQPLLDRTVPDYTTFTTV |
| | | GDWLDAIKMGRYKESFVSAGFASFDLVAQM |
| | | TAEDLLRIGVTLAGHQKKILSSIQDMRLQM |
| | | NQTLPVQV |
| 224 | Human | MELRVLLCWASLAAALEETLLNTKLETADL |
| | EPHB4 | KWVTFPQVDGQWEELSGLDEEQHSVRTYEV |
| | | CDVQRAPGQAHWLRTGWVPRRGAVHVYATL |
| | | RFTMLECLSLPRAGRSCKETFTVFYYESDA |
| | | DTATALTPAWMENPYIKVDTVAAEHLTRKR |
| | | PGAEATGKVNVKTLRLGPLSKAGFYLAFQD |
| | | QGACMALLSLHLFYKKCAQLTVNLTRFPET |
| | | VPRELVVPVAGSCVVDAVPAPGPSPSLYCR |
| | | EDGQWAEQPVTGCSCAPGFEAAEGNTKCRA |
| | | CAQGTFKPLSGEGSCQPCPANSHSNTIGSA |
| | | VCQCRVGYFRARTDPRGAPCTTPPSAPRSV |
| | | VSRLNGSSLHLEWSAPLESGGREDLTYALR |
| | | CRECRPGGSCAPCGGDLTFDPGPRDLVEPW |
| | | VVVRGLRPDFTYTFEVTALNGVSSLATGPV |
| | | PFEPVNVTTDREVPPAVSDIRVTRSSPSSL |
| | | SLAWAVPRAPSGAVLDYEVKYHEKGAEGPS |
| | | SVRFLKTSENRAELRGLKRGASYLVQVRAR |
| | | SEAGYGPFGQEHHSQTQLDESEGWREQLAL |
| | | IAGTAVVGVVLVLVVIVVAVLCLRKQSNGR |
| | | EAEYSDKHGQYLIGHGTKVYIDPFTYEDPN |
| | | EAVREFAKEIDVSYVKIEEVIGAGEFGEVC |
| | | RGRLKAPGKKESCVAIKTLKGGYTERQRRE |
| | | FLSEASIMGQFEHPNIIRLEGVVTNSMPVM |
| | | ILTEFMENGALDSFLRLNDGQFTVIQLVGM |
| | | LRGIASGMRYLAEMSYVHRDLAARNILVNS |
| | | NLVCKVSDFGLSRFLEENSSDPTYTSSLGG |
| | | KIPIRWTAPEAIAFRKFTSASDAWSYGIVM |
| | | WEVMSFGERPYWDMSNQDVINAIEQDYRLP |
| | | PPPDCPTSLHQLMLDCWQKDRNARPRFPQV |
| | | VSALDKMIRNPASLKIVARENGGASHPLLD |
| | | QRQPHYSAFGSVGEWLRAIKMGRYEESFAA |
| | | AGFGSFELVSQISAEDLLRIGVTLAGHQKK |
| | | ILASVQHMKSQAKPGTPGGTGGPAPQY |
| 225 | Human | MATEGAAQLGNRVAGMVCSLWVLLLVSSVL |
| | EPHB6 | ALEEVLLDTTGETSEIGWLTYPPGGWDEVS |
| | | VLDDQRRLTRTFEACHVAGAPPGTGQDNWL |
| | | QTHFVERRGAQRAHIRLHFSVRACSSLGVS |
| | | GGTCRETFTLYYRQAEEPDSPDSVSSWHLK |
| | | RWTKVDTIAADESFPSSSSSSSSSSSAAWA |
| | | VGPHGAGQRAGLQLNVKERSFGPLTQRGFY |
| | | VAFQDTGACLALVAVRLFSYTCPAVLRSFA |
| | | SFPETQASGAGGASLVAAVGTCVAHAEPEE |
| | | DGVGGQAGGSPPRLHCNGEGKWMVAVGGCR |
| | | CQPGYQPARGDKACQACPRGLYKSSAGNAP |
| | | CSPCPARSHAPNPAAPVCPCLEGFYRASSD |
| | | PPEAPCTGPPSAPQELWFEVQGSALMLHWR |
| | | LPRELGGRGDLLFNVVCKECEGRQEPASGG |
| | | GGTCHRCRDEVHFDPRQRGLTESRVLVGGL |
| | | RAHVPYILEVQAVNGVSELSPDPPQAAAIN |
| | | VSTSHEVPSAVPVVHQVSRASNSITVSWPQ |
| | | PDQINGNILDYQLRYYDQAEDESHSFTLTS |
| | | ETNTATVTQLSPGHIYGFQVRARTAAGHGP |
| | | YGGKVYFQTLPQGELSSQLPERLSLVIGSI |
| | | LGALAFLLLAAITVLAVVFQRKRRGTGYTE |
| | | QLQQYSSPGLGVKYYIDPSTYEDPCQAIRE |
| | | LAREVDPAYIKIEEVIGTGSFGEVRQGRLQ |
| | | PRGRREQTVAIQALWAGGAESLQMTFLGRA |
| | | AVLGQFQHPNILRLEGVVTKSRPLMVLTEF |
| | | MELGPLDSFLRQREGQFSSLQLVAMQRGVA |
| | | AAMQYLSSFAFVHRSLSAHSVLVNSHLVCK |
| | | VARLGHSPQGPSCLLRWAAPEVIAHGKHTT |
| | | SSDVWSFGILMWEVMSYGERPYWDMSEQEV |
| | | LNAIEQEFRLPPPPGCPPGLHLLMLDTWQK |
| | | DRARRPHFDQLVAAFDKMIRKPDTLQAGGD |
| | | PGERPSQALLTPVALDFPCLDSPQAWLSAI |
| | | GLECYQDNFSKFGLCTFSDVAQLSLEDLPA |
| | | LGITLAGHQKKLLHHIQLLQQHLRQQGSVE |
| | | V |
| 226 | EphA4 | LAQFPDTITGADTSSLVEVRGSCVNNSEEK |
| | fragment | DVPKMYCGADGEWLVPIGNCLCNAGHEERS |
| | | GECQACKIGYYKALSTDATCAKCPPHSYSV |
| | | WEGATSCTCDRGFFRADNDAASMPCTRPPS |
| | | APLNLISNVNETSVNLEWSSPQNTGGRQDI |
| | | SYNVVCKKCGAGDPSKCRPCGSGVHYTPQQ |
| | | NGLKTTKVSITDLLAHTNYTFEIWAVNGVS |
| | | KYNPNPDQSVSVTVTTNQAAPSSIALVQAK |
| | | EVTRYSVALAWLEPDRPNGVILEYEVKYYE |
| | | KDQNERSYRIVRTAARNTDIKGLNPLTSYV |
| | | FHVRARTAAGYGDFSEPLEVTTNTVPSRII |
| | | GDGANSTVLLVSVSGSVVLVVILIAAFVIS |
| | | RRRSKYSKAKQEADEEKHLN |
| 227 | Mouse | MGMPLPWALSLLLVLLPQTWGSETRPPLMY |
| | FcRn | HLTAVSNPSTGLPSFWATGWLGPQQYLTYN |
| | | SLRQEADPCGAWMWENQVSWYWEKETTDLK |
| | | SKEQLFLEALKTLEKILNGTYTLQGLLGCE |
| | | LASDNSSVPTAVFALNGEEFMKFNPRIGNW |
| | | TGEWPETEIVANLWMKQPDAARKESEFLLN |
| | | SCPERLLGHLERGRRNLEWKEPPSMRLKAR |
| | | PGNSGSSVLTCAAFSFYPPELKFRFLRNGL |
| | | ASGSGNCSTGPNGDGSFHAWSLLEVKRGDE |
| | | HHYQCQVEHEGLAQPLTVDLDSSARSSVPV |
| | | VGIVLGLLLVVVAIAGGVLLWGRMRSGLPA |
| | | PWLSLSGDDSGDLLPGGNLPPEAEPQGANA |
| | | FPATS |
| 228 | Human | MGVPRPQPWALGLLLFLLPGSLGAESHLSL |
| | FcRn | LYHLTAVSSPAPGTPAFWVSGWLGPQQYLS |
| | | YNSLRGEAEPCGAWVWENQVSWYWEKETTD |
| | | LRIKEKLFLEAFKALGGKGPYTLQGLLGCE |
| | | LGPDNTSVPTAKFALNGEEFMNFDLKQGTW |
| | | GGDWPEALAISQRWQQQDKAANKELTFLLF |
| | | SCPHRLREHLERGRGNLEWKEPPSMRLKAR |
| | | PSSPGFSVLTCSAFSFYPPELQLRFLRNGL |
| | | AAGTGQGDFGPNSDGSFHASSSLTVKSGDE |
| | | HHYCCIVQHAGLAQPLRVELESPAKSSVLV |
| | | VGIVIGVLLLTAAAVGGALLWRRMRSGLPA |
| | | PWISLRGDDTGVLLPTPGEAQDADLKDVNV |
| | | IPATA |
| 229 | Signal | MALRRLGAALLLLPLLAAVSDVPRDLEVVA |
| | peptide- | ATPTSLLISWYYPFCAFYYRITYGETGGNS |
| | Monobody- | PVQEFTVPRSPDTATISGLKPGVDYTITVY |
| | Linker- | AVTCLGSYSRPISINYRTGGGGSGGGGSGG |
| | modified | APEAAGGPSVFLFPPKPKDTLMISRTPEVT |
| | Fc-Linker- | CVVVDVSHEDPEVKFNWYVDGVEVHNAKTK |
| | EphB2- | PREEQYNSTYRVVSVLTVLHQDWLNGKEYK |
| | Turboluc | CKVSNKALGAPIEKTISKAKGQPREPQVYT |
| | | KPPSRDELTKNQVSLSCLVKGFYPSDIAVE |
| | | WESNGQPENNYKTTVPVLDSDGSFRLASYL |
| | | TVDKSRWQQGNVFSCSVMHEALHNHYTQKS |
| | | LSLSPGGGSGGGSGGGSGGGSRKCPRIIQN |
| | | GAIFQETLSGAESTSLVAARGSCIANAEEV |
| | | DVPIKLYCNGDGEWLVPIGRCMCKAGFEAV |
| | | ENGTVCRGCPSGTFKANQGDEACTHCPINS |
| | | RTTSEGATNCVCRNGYYRADLDPLDMPCTT |
| | | IPSAPQAVISSVNETSLMLEWTPPRDSGGR |
| | | EDAVYNIICKSCGSGRGACTRCGDNVQYAP |
| | | RQLGLTEPRIYASDLLAHTQYTFEIQAVNG |
| | | VTDQSPFSPQFASVNITTNQAAPSAVSIMH |
| | | QVSRTVDSITLSWSQPDQPNGVILDYELQY |
| | | YEKELSEYNATAIKSPTNTVTVQGLKAGAI |
| | | YVFQVRARTVAGYGRYSGKMYFQTMTEAEY |
| | | QTEIQEKLPLIIGSSAAGLVFLIAVVVISI |
| | | VCNRRGFERADSEYTDKLQHYTSGHMTPGM |
| | | KIYIDPFTYEDPNEAVREFAKEIDISCVKI |
| | | EQVIGAGEFGEVCSGHLKLPGKREIFVAIK |
| | | TLKSGYTEKQRRDFLSEASIMGQFDHPNVI |
| | | HLEGVVTKSTPVMIITEFMENGSLDSFLRQ |
| | | NDGQFTVIQLVGMLRGIAAGMKYLADMNYV |
| | | HRDLAARNILVNSNLVCKVSDFGLSRFLED |
| | | DTSDPTYTSALGGKIPIRWTAPEAIQYRKF |
| | | TSASDVWSYGIVMWEVMSFGERPYWDMTNQ |
| | | DVINAIEQDYRLPPPMDCPSALHQLMLDCW |
| | | QKDRNHRPKFGQIVNTLDKMIRNPNSLKAM |
| | | APLSSGINLPLGGGEAEAERGKLPGKKLPL |
| | | EVLIELEANARKAGCTRGCLICLSKIKCTA |
| | | KMKKYIPGRCADYGGDKKTGQAGIVGAIVD |
| | | IPEISGFKEMEPMEQFIAQVDRCADCTTGC |
| | | LKGLANVKCSDLLKKWLPGRCATFADKIQS |
| | | EVDNIKGLAGD |
| 230 | Consesus | LNGEEFMX₁FX₂X₃X₄X₅GX₆WX₇GX₈W |
| | Sequence | (wherein X₁, X₂, X₃, X₄, |
| | for Fc- | X₅, X₆, X₇, and X₈ each |
| | FcRn | is any amino acid) |
| | binding | |
| 231 | Linker | (GGGS)ₙ |
| | | (wherein n is an integer |
| | | number from 1 to 10) |
| 232 | Linker | (GGGS)₂ |
| 233 | Linker | (GGGS)₃ |
| 234 | Linker | (GGGGS)n |
| | | (wherein n is an integer |
| | | number from 1 to 10) |
| 235 | Linker | (GGGGS)₂ |
| 236 | Linker | (GGGGS)₃ |
| 237 | Linker | (G₄S)₂-G₄ |
| 238 | Linker | G₃S-(G₄S)₄-G₂ |

### 7. EXAMPLES

The following are examples, illustrating the methods and compositions disclosed herein. It is understood that various other embodiments may be practiced, given the general description provided above.

### 7.1 Example 1: EV Production

A stable cell line, expressing a polypeptide comprising from N- to C-Terminus: a targeting monobody-linker1-modified monomeric Fc-linker2-EphB2 scaffold-linker3-turboluc (wherein the EphB2 scaffold comprised residues 195-905 of EphB2, lacking a LBD, and containing the following amino acid substitutions L356A I395A S536E A562S, Y822F relative to SEQ TD NO: 222), was generated. See SEQ ID NO: 229 for the sequence of the full fusion protein. Cells from the cell line were grown and EVs were isolated from the supermatant of cultures of stable clones. Specifically, EV-containing media was collected and clarified by differential centrifugation. The supermatant was then filtered with a 0.22 um syringe or bottle-top filter and further processed by different purification steps. For larger scale productions, high density cultures were maintained in a stirred bioreactor in perfusion mode, whereby the harvested perfusion supermatant was pre-clarified and filtered by an alternating tangential flow system fitted with a 0.2 um hollow fiber filter. EVs were isolated and purified from the clarified conditioned media using a variety of methods, typically a combination of dia-/ultrafiltration with tangential flow filtration and flow through based multimodal chromatography and/or bind and elute chromatography steps. Purified EVs were then frozen and stored for downstream analysis.

To confirm the presence of fusion proteins in the EV samples, the fusion proteins were detected by western blot. Briefly, SDS-PAGE was performed according to manufacturer's instruction, whereby samples containing 4 ug protein were loaded per well. Proteins from the SDS-PAGE gel were transferred to PVDF membrane according to manufacturer's instruction (iBlot2, Thermo). Membranes were blocked in 10 ml 5% skimmed milk in PBS-T and probed with the anti-turboluc antibodies and appropriate secondary antibodies according to supplier's instruction. Bands were recorded by chemiluminescence detection. As shown in FIG. 13, the produced EVs contained the full length scaffold protein with intraluminal turboluc.

### 7.2 Example 2: FcRn Binding Immunoassay

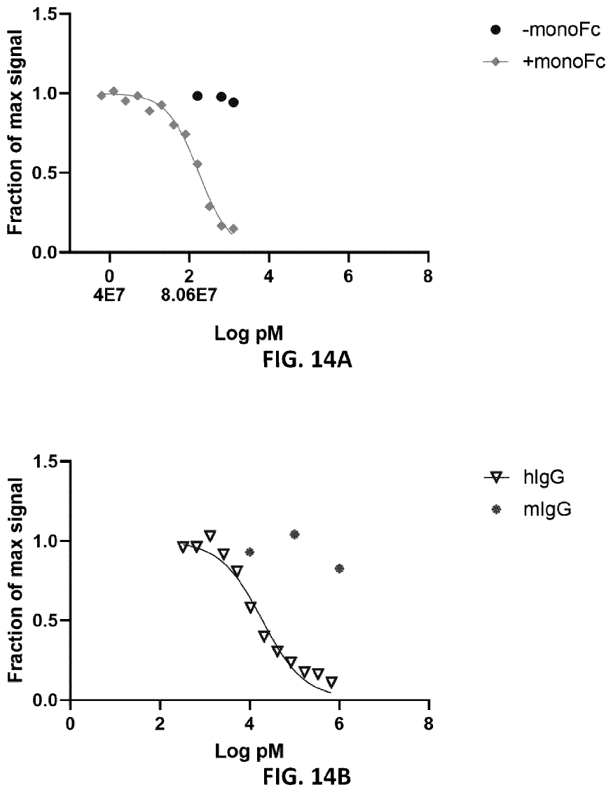

A Lumit™ FcRn Binding Immunoassay (Promega) assay was performed with purified EVs described in Example 1 and native Hek293 EVs. Samples of said EVs and a human IgG1 and a mouse IgG1 as controls were each serially diluted and incubated with a split FcRn/Tracer according to the manufacturer instructions (Tracer and FcRn were 10× diluted). Detection reagent was added and luminescence was detected on a plate reader. As shown in FIG. 14A, purified EVs described in Example 1 were able to bind FcRn while native EVs did not bind to FcRn. As shown in FIG. 14B, human IgG1 was able to bind FcRn while mouse IgG1 did not.

### 7.3 Example 3: FcRn Affinity Purification

A recombinant single chain FcRn (scFcRn) construct containing the mouse IgG kappa chain leader sequence as the secretion signal followed by the mature B2M sequence connected through (GGGGS)3 (SEQ ID NO:236) to the mature sequence of the FCGRT heavy chain and C-tag was designed in silico and synthesized by a commercial DNA synthesis vendor. Recombinant scFcRn protein was produced from the construct and purified. The scFcRn protein was then loaded to a C-tag column (Thermo Scientific) following procedures from the instruction manual. The resin was then washed with 25 mM MES pH 5.8, 150 mM NaCl.

A stable cell line, expressing a polypeptide comprising from N- to C-Terminus: a targeting monobody—linker-modified monomeric Fc—linker-EphA4 fragment-GFP tag (containing residues 29-590 of EphA4 and an amino acid substitution of F154A relative to SEQ ID NO: 215), was generated and cultivated, and the supernatant was collected, clarified and concentrated. The pH of the harvested supernatant was adjusted to pH 5.8 and then loaded on the equilibrated column and further washed with 25 mM MES pH 5.8, 150 mM NaCl. Bound sample was eluted with 50 mM Tris pH 7.4, 150 mM NaCl (reverse flow).

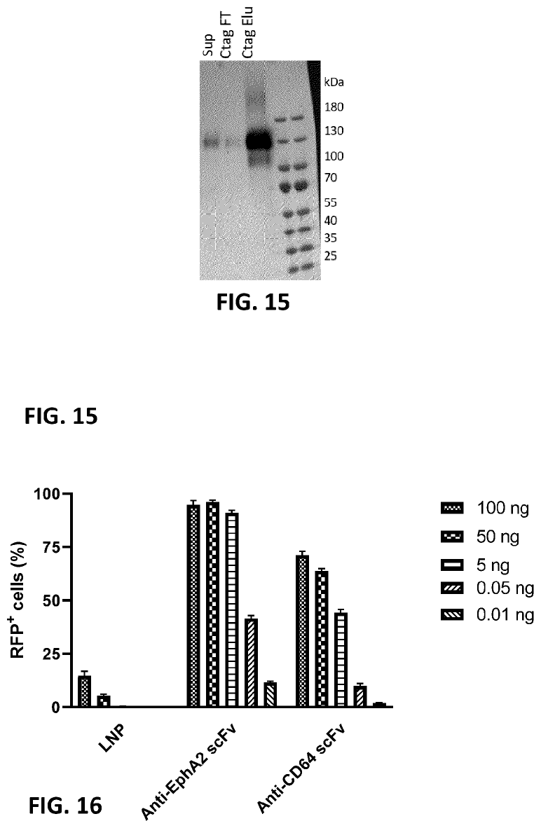

To confirm the presence of fusion proteins in the samples, the proteins were detected by western blot. Briefly, SDS-PAGE was performed according to manufacturer's instruction, whereby samples containing 4 ug protein were loaded per well. Proteins from the SDS-PAGE gel were transferred to PVDF membrane according to manufacturer's instruction (iBlot2, Thermo). Membranes were blocked in 10 ml 5% skimmed milk in PBS-T and probed with anti-EphA4 antibody (ECM Biosciences, Cat. No. EM2801) and appropriate secondary antibodies according to supplier's instruction. Bands were recorded by chemiluminescence detection. As shown in FIG. 15, the elution sample showed an enrichment in EphA4 signal.

### 7.4 Example 4: Targeting and mRNA Delivery

Cre mRNA (Trilink) loaded lipid nanoparticles were prepared on a Nanoassemblr microfluidic system according to the manufacturer's instructions. Depending on the desired formulation, a lipid formulation consisting of an ionizable lipid (e.g., MC3), a zwitterionic lipid (e.g., distearoylphosphatidylcholine (DSPC), dioleoylglycerophosphocholine (DOPC), a component to provide membrane integrity (such as a sterol, e.g., cholesterol) and a conjugated lipid molecule (such as a PEG-lipid, e.g., 1-(monomethoxy-polyethyleneglycol)-2,3-dimyristoylglycerol, with an average PEG molecular weight of 2000 (“PEG-DMG”)) was prepared. Furthermore, an aqueous mRNA solution was prepared in 25 mM acetate buffer at pH 4.0. For each formulation the aqueous mRNA solution was mixed with the ethanol-lipid solution with a flow rate ratio of 3:1 (Aq:Et) at room temperature.

For exosome production, two stable cell lines were generated. The first cell line comprised an anti-EphA2 scFv-linker-EphB4 fragment (stretching residues 195-579 of EphB4, lacking the LBD, and having amino acid substitutions of L355A, V393A, S531E and A595S, with respect to SEQ ID NO: 224) linked to an endodomain of EphA2 (stretching residues 581-976 and having amino acid substitutions Y588F and Y594F with respect to SEQ ID NO: 213). A second cell line comprised the same scaffold protein as the first cell line but the scFv was replaced with an anti-CD64 scFv. Cells were grown in stirred bioreactors in perfusion mode and exosome isolation was performed by tangential flow filtration followed by Captocore 700 liquid chromatography as described in Nordin et al., Methods in Molecular Biology, vol 1953. Humana Press, New York, NY (2019), which is herein incorporated in its entirety by reference.

Commercially available human embryonic kidney cells (HEK-293T) expressing a “LoxP-GFP-stop-LoxP-RFP” cassette under CMV promoter (Gen target Inc.) were confirmed to be expressing EphA2 receptor by flow cytometry. The efficiency of the delivery of targeted nanovesicles displaying an anti-EphA2 scfv on their surface versus non-targeted nanovesicles (displaying an anti-CD64 scFv) was assayed by the delivery of Cre mRNA. If functional Cre mRNA was successfully delivered, Cre mRNA would be translated to Cre protein, which would enter the nucleus, excise the floxed stop codon and turn on RFP expression. For testing the delivery efficiency, the HEK-293T cells (2×10⁴/well) were seeded in a 96-well plate and transfected for 48 h with 100 ng, 50 ng, 5 ng, 0.05 and 0.01 ng of Cre mRNA, respectively. Transfection was mediated using hybridosomes generated by fusing exosomes with lipid nanoparticles as outlined in U.S. Patent Application Publication No. 2016/0354313 A1. As a comparison, additional HEK-293T cells were transfected with lipid nanoparticles (LNPs).

After 48h, cells were detached and GFP/RFP expression was detected by flow cytometry (CytoFlex). The percentage of cells that were RFP+, in which delivery of functional Cre mRNA occurred,) is shown in FIG. 16. The anti-EphA2 scfv improved the delivery of functional Cre mRNA by an order of magnitude.

### 7.5 Example 5: SH2 Based Lumen Loading

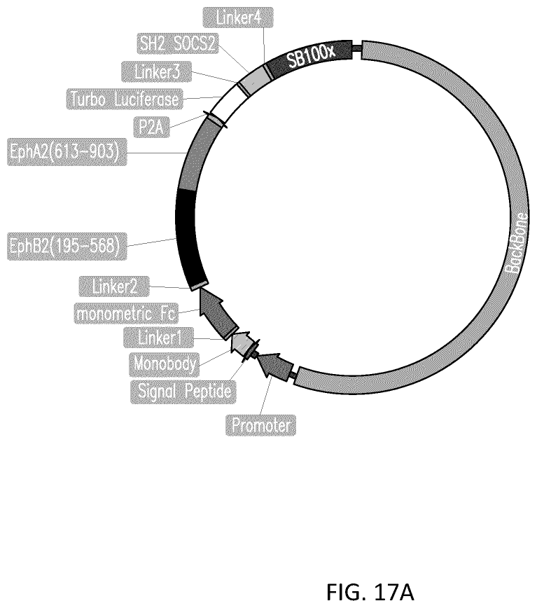

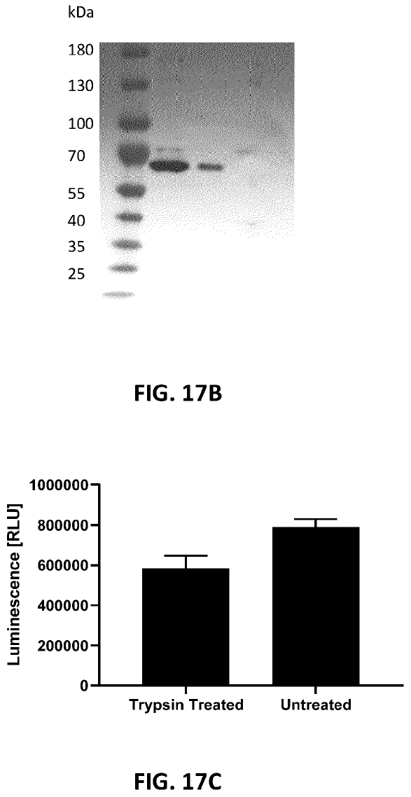

A lentiviral polycistronic construct as illustrated in FIG. 17A was constructed. A stable cell line comprising the construct was generated. The stable cell line expressed two proteins from the construct: (1) a fusion protein comprising a targeting monobody, a monomeric Fc (monoFC), an EphB2 flexible domain lacking the ligand binding domain (LBD), an EphB2 transmembrane domain (TM), an EphA2 juxtamembrane (JM) domain, and an EphA2 kinase domain (KD); and (2) a fusion protein comprising from N- to C-terminus: a luciferase, linker, the SH2 domain of SOCS2, linker, and SBX100 (a sleeping beauty transposase) (referred to as turboluc-SH2-SBX).

The EVs from these producer cells were then purified from the conditioned media of the stable cell line. After EV purification the presence of the fusion protein comprising a luciferase, the SH2 domain of SOCS2, and SBX100 (a sleeping beauty transposase) was confirmed by western blot. The western blot was immunoblotted with antibodies against the luciferase (thermo PA1-178). FIG. 17B. shows that the turboluc-SH2-SBX protein was present in the EV lysate.

To confirm that the turboluc-SH2-SBX protein was in the lumen of the EVs, engineered EVs were either treated with trypsin for 30 minutes at 37° C. or incubated in PBS at 37° C. The samples were left to cool to room temperature and luciferase activity was measured by TurboLuc™ Luciferase One-Step Glow Assay Kit, according to the manufacturer's instructions. FIG. 17C shows that EVs treated with trypsin retained luciferase activity. As trypsin is membrane-impermeable and the turboluc-SH2-SBX protein was protected, the data showed that the turboluc-SH2-SBX protein was present in the lumen of the EVs.

### 7.6 Example 6: Blood Clearance after IV Administration of Modified Fc Hybridosomes

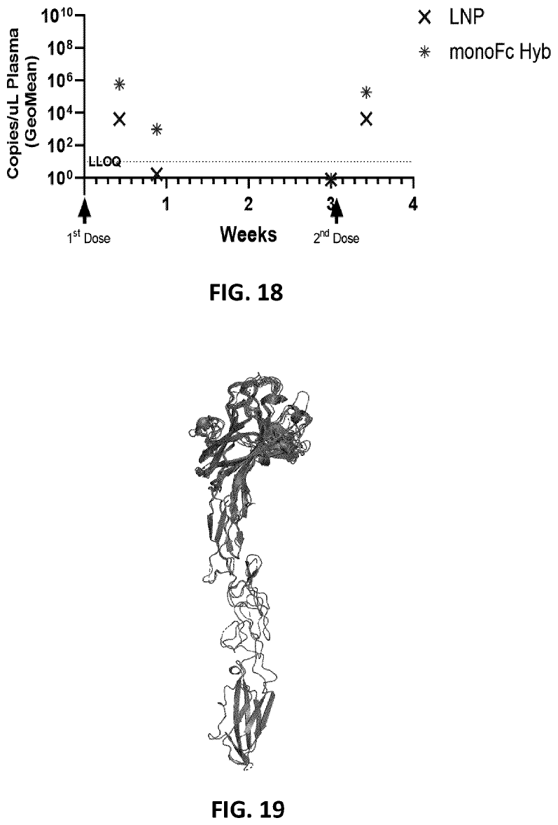

Exosomes are considered to have a very short half-life and circulation time. To test the blood clearance of hybridosomes comprising EphB2 scaffold described in Example 1, nude immunocompetent SKH1 mice (6-8 weeks old, n=6/group) were injected intravenously with DNA loaded lipid nanoparticles or hybridosomes (0.5 mg/kg). The DNA cargo encoded a promoter, a reporter transgene and a BGH poly(A). The lipid nanoparticles were prepared on a Nanoassemblr™ microfluidic system (Precision NanoSystems) according to the manufacturer's instructions. Animals were re-dosed on day 21, post administration. In order to monitor blood clearance, on days 3, 6, 21 (pre-second dose) and 24, respectively, twenty microliters of blood were drawn from the tail vein and processed to plasma. Two microliters of diluted plasma were used in a Taqman qPCR assay to quantify the DNA sequence, specifically the BGH Poly A sequence, by comparing against a standard curve on the same plate. Recovery efficiency of DNA from naïve mouse plasma was determined by spiking the DNA vector into mouse plasma. As shown in FIG. 18, hybridosomes comprising a targeting monobody-modified Fc domain fused to a EphB2 scaffold protein could be detected in the mouse plasma 6 days post administration while on the same day the plasma copy number was below the detection limit for the LNP treated group.

What is claimed is:

1. An extracellular vesicle (EV) comprising a polypeptide, wherein the polypeptide comprises in N-terminus to C-terminus direction:
a. an ephrin receptor cysteine-rich (CR) domain;
b. a first ephrin receptor fibronectin type III (FN III) domain and a second ephrin receptor FN III domain; and
c. a transmembrane (TM) domain;
wherein the polypeptide lacks (i) ephrin binding activity, (ii) ephrin receptor kinase activity, or (iii) both ephrin binding activity and ephrin receptor kinase activity.

2. A hybridosome comprising a polypeptide, wherein the polypeptide comprises in N-terminus to C-terminus direction:
a. an ephrin receptor CR domain;
b. a first ephrin receptor FN III domain and a second ephrin receptor FN III domain; and
c. a TM domain;
wherein the polypeptide lacks (i) ephrin binding activity, (ii) ephrin receptor kinase activity, or (iii) both ephrin binding activity and ephrin receptor kinase activity.

3. The EV of claim 1 or the hybridosome of claim 2, wherein the polypeptide lacks ephrin binding activity.

4. The EV or hybridosome of any one of claims 1-3, wherein the polypeptide further comprises a targeting domain N-terminal to the ephrin receptor CR domain.

5. The EV or hybridosome of claim 4, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

6. The EV or hybridosome of claim 4 or 5, wherein the targeting domain specifically binds to a marker.

7. The EV or hybridosome of claim 6, wherein the marker is a tumor-associated antigen.

8. The EV or hybridosome of claim 7, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

9. The EV or hybridosome of any one of claims 1-8, wherein the polypeptide further comprises a cargo protein or a cargo binding domain C-terminal to the TM domain.

10. The EV or hybridosome of claim 9, wherein the cargo protein or cargo binding domain is fused to the remaining portion of the polypeptide via a linker.

11. The EV or hybridosome of claim 10, wherein the cargo protein or cargo binding domain is covalently fused to the remaining portion of the polypeptide via a linker.

12. The EV or hybridosome of claim 10 or 11, wherein the linker is a peptide linker.

13. The EV or hybridosome of claim 12, wherein the peptide linker comprises an amino acid sequence of (GGGS)n (SEQ ID NO: 226), wherein n is an integer number from 1 to 10.

14. The EV or hybridosome of claim 12, wherein the peptide linker comprises an amino acid sequence of GGGS.

15. The EV or hybridosome of any one of claims 9-14, wherein the polypeptide comprises a cargo binding domain that is capable of binding to a cargo protein directly, or indirectly via a scaffold binding domain (SBD) linked to the cargo protein.

16. The EV or hybridosome of claim 15, wherein the binding between the cargo binding domain and the cargo protein is a non-covalent binding.

17. The EV or hybridosome of claim 15 or 16, wherein the binding between the cargo binding domain and the cargo protein is a reversible binding.

18. The EV or hybridosome of any one of claims 15-17, wherein the binding between the cargo binding domain and the cargo protein is capable of being controlled.

19. The EV or hybridosome of claim 18, wherein the binding between the cargo binding domain and the cargo protein is capable of being controlled by pH.

20. The EV or hybridosome of claim 18, wherein the binding between the cargo binding domain and the cargo protein is capable of being controlled by ionic strength.

21. The EV or hybridosome of any one of claims 15-20, wherein the binding between the cargo binding domain and the cargo protein is capable of being controlled such that the cargo protein is bound to the cargo binding domain in vitro but is released from the cargo binding domain in vivo.

22. The EV or hybridosome of any one of claims 15-20, wherein the binding between the cargo binding domain and the cargo protein is capable of being controlled such that the cargo protein is released from the cargo binding domain in a manner dependent on the subcellular compartment in which they are located.

23. The EV or hybridosome of any one of claims 15-22, wherein the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine.

24. The EV or hybridosome of claim 23, wherein the domain that is capable of binding to phosphotyrosine is a phosphotyrosine binding (PTB) domain.

25. The EV or hybridosome of claim 23, wherein the domain that is capable of binding to phosphotyrosine is a Src homology 2 (SH2) domain.

26. The EV or hybridosome of any one of claims 15-22, wherein the cargo binding domain comprises a first sterile α-motif (SAM) domain and the cargo protein or the SBD comprises a second SAM domain, and the binding between the cargo binding domain and the cargo protein is a binding between the first SAM domain and the second SAM domain.

27. The EV or hybridosome of any one of claims 15-22, wherein the cargo binding domain comprises a PDZ binding motif (PBM) domain and the cargo protein or the SBD comprises a PDZ domain, and the binding between the cargo binding domain and the cargo protein is a binding between the PBM domain and the PDZ domain.

28. The EV or hybridosome of any one of claims 15-22, wherein the cargo binding domain comprises a PDZ domain and the cargo protein or the SBD comprises a PBM domain, and the binding between the cargo binding domain and the cargo protein is a binding between the PDZ domain and the PBM domain.

29. The EV or hybridosome of any one of claims 9-14, wherein the polypeptide comprises a cargo protein.

30. The EV or hybridosome of any one of claims 1-8, wherein the polypeptide further comprises an ephrin receptor JM domain that is capable of binding to a cargo protein directly, or indirectly via a SBD linked to the cargo protein, and is C-terminal to the TM domain.

31. The EV or hybridosome of claim 30, wherein the binding between the ephrin receptor JM domain and the cargo protein is a non-covalent binding.

32. The EV or hybridosome of claim 30 or 31, wherein the binding between the ephrin receptor JM domain and the cargo protein is a reversible binding.

33. The EV or hybridosome of any one of claims 30-32, wherein the binding between the ephrin receptor JM domain and the cargo protein is capable of being controlled.

34. The EV or hybridosome of claim 33, wherein the binding between the ephrin receptor JM domain and the cargo protein is capable of being controlled by pH.

35. The EV or hybridosome of claim 33, wherein the binding between the ephrin receptor JM domain and the cargo protein is capable of being controlled by ionic strength.

36. The EV or hybridosome of any one of claims 30-35, wherein the binding between the ephrin receptor JM domain and the cargo protein is capable of being controlled such that the cargo protein is bound to the ephrin receptor JM domain in vitro but is released from the ephrin receptor JM domain in vivo.

37. The EV or hybridosome of any one of claims 30-35, wherein the binding between the ephrin receptor JM domain and the cargo protein is capable of being controlled such that the cargo protein is released from the ephrin receptor JM domain in a manner dependent on the subcellular compartment in which they are located.

38. The EV or hybridosome of any one of claims 30-37, wherein the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine.

39. The EV or hybridosome of claim 38, wherein the domain that is capable of binding to phosphotyrosine is a PTB domain.

40. The EV or hybridosome of claim 38, wherein the domain that is capable of binding to phosphotyrosine is an SH2 domain.

41. The EV or hybridosome of any one of claims 38-40, wherein the ephrin receptor JM domain comprises:
(i) a (X₁)-Ptyr-(X₂) motif, wherein Ptyr is a phosphotyrosine, X₁ is Y, P, V, I, T, or F, and X₂ is I, V, L, or A;
(ii) a (X₃)-Ptyr-(X₄) motif, wherein Ptyr is a phosphotyrosine, X₃ is T, A, or S, and X₄ is E or G; or
(iii) both (i) and (ii).

42. The EV or hybridosome of any one of claims 1-8, wherein the polypeptide further comprises an ephrin receptor KD that is capable of binding to a cargo protein directly, or indirectly via a SBD linked to the cargo protein, and is C-terminal to the TM domain.

43. The EV or hybridosome of claim 42, wherein the binding between the ephrin receptor KD and the cargo protein is a non-covalent binding.

44. The EV or hybridosome of claim 42 or 43, wherein the binding between the ephrin receptor KD and the cargo protein is a reversible binding.

45. The EV or hybridosome of any one of claims 42-44, wherein the binding between the ephrin receptor KD and the cargo protein is capable of being controlled.

46. The EV or hybridosome of claim 45, wherein the binding between the ephrin receptor KD and the cargo protein is capable of being controlled by pH.

47. The EV or hybridosome of claim 45, wherein the binding between the ephrin receptor KD and the cargo protein is capable of being controlled by ionic strength.

48. The EV or hybridosome of any one of claims 42-47, wherein the binding between the ephrin receptor KD and the cargo protein is capable of being controlled such that the cargo protein is bound to the ephrin receptor KD in vitro but is released from the ephrin receptor KD in vivo.

49. The EV or hybridosome of any one of claims 42-47, wherein the binding between the ephrin receptor KD and the cargo protein is capable of being controlled such that the cargo protein is released from the ephrin receptor KD in a manner dependent on the subcellular compartment in which they are located.

50. The EV or hybridosome of any one of claims 42-49, wherein the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine.

51. The EV or hybridosome of claim 50, wherein the domain that is capable of binding to phosphotyrosine is a PTB domain.

52. The EV or hybridosome of claim 50, wherein the domain that is capable of binding to phosphotyrosine is an SH2 domain.

53. The EV or hybridosome of any one of claims 50-52, wherein the KD comprises an (X₇)-Ptyr-(X₈) motif in the activation loop, wherein Ptyr is a phosphotyrosine, X₇ is T, V, or A, and X₈ is E or T.

54. The EV or hybridosome of any one of claims 1-8, wherein the polypeptide further comprises a SAM linker domain that is capable of binding to a cargo protein directly, or indirectly via a SBD linked to the cargo protein, and is C-terminal to the TM domain.

55. The EV or hybridosome of claim 54, wherein the binding between the SAM linker domain and the cargo protein is a non-covalent binding.

56. The EV or hybridosome of claim 54 or 55, wherein the binding between the SAM linker domain and the cargo protein is a reversible binding.

57. The EV or hybridosome of any one of claims 54-56, wherein the binding between the SAM linker domain and the cargo protein is capable of being controlled.

58. The EV or hybridosome of claim 57, wherein the binding between the SAM linker domain and the cargo protein is capable of being controlled by pH.

59. The EV or hybridosome of claim 57, wherein the binding between the SAM linker domain and the cargo protein is capable of being controlled by ionic strength.

60. The EV or hybridosome of any one of claims 54-59, wherein the binding between the SAM linker domain and the cargo protein is capable of being controlled such that the cargo protein is bound to the SAM linker domain in vitro but is released from the SAM linker domain in vivo.

61. The EV or hybridosome of any one of claims 54-59, wherein the binding between the SAM linker domain and the cargo protein is capable of being controlled such that the cargo protein is released from the SAM linker domain in a manner dependent on the subcellular compartment in which they are located.

62. The EV or hybridosome of any one of claims 54-61, wherein the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid.

63. The EV or hybridosome of any one of claims 54-62, wherein the SAM linker domain is an ephrin receptor SAM linker domain.

64. The EV or hybridosome of any one of claims 1-8, wherein the polypeptide further comprises a SAM domain that is capable of binding to a cargo protein directly, or indirectly via a SBD linked to the cargo protein, and is C-terminal to the TM domain.

65. The EV or hybridosome of claim 64, wherein the binding between the SAM domain and the cargo protein is a non-covalent binding.

66. The EV or hybridosome of claim 64 or 65, wherein the binding between the SAM domain and the cargo protein is a reversible binding.

67. The EV or hybridosome of any one of claims 64-66, wherein the binding between the SAM domain and the cargo protein is capable of being controlled.

68. The EV or hybridosome of claim 67, wherein the binding between the SAM domain and the cargo protein is capable of being controlled by pH.

69. The EV or hybridosome of claim 67, wherein the binding between the SAM domain and the cargo protein is capable of being controlled by ionic strength.

70. The EV or hybridosome of any one of claims 64-69, wherein the binding between the SAM domain and the cargo protein is capable of being controlled such that the cargo protein is bound to the SAM domain in vitro but is released from the SAM domain in vivo.

71. The EV or hybridosome of any one of claims 64-69, wherein the binding between the SAM domain and the cargo protein is capable of being controlled such that the cargo protein is released from the SAM domain in a manner dependent on the subcellular compartment in which they are located.

72. The EV or hybridosome of any one of claims 64-71, wherein the cargo protein or the SBD comprises a second SAM domain, and the binding between the SAM domain and the cargo protein is a binding between the SAM domain and the second SAM domain.

73. The EV or hybridosome of any one of claims 64-71, wherein the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine.

74. The EV or hybridosome of claim 73, wherein the domain that is capable of binding to phosphotyrosine is a PTB domain.

75. The EV or hybridosome of claim 73, wherein the domain that is capable of binding to phosphotyrosine is an SH2 domain.

76. The EV or hybridosome of any one of claims 73-75, wherein the SAM domain comprises a phosphotyrosine in the α2 helix.

77. The EV or hybridosome of claim 76, wherein the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T.

78. The EV or hybridosome of any one of claims 64-77, wherein the SAM domain is an ephrin receptor SAM domain.

79. The EV or hybridosome of any one of claims 1-8, wherein the polypeptide further comprises an ephrin receptor PDZ binding motif (PBM) domain that is capable of binding to a cargo protein directly, or indirectly via a SBD linked to the cargo protein, and is C-terminal to the TM domain.

80. The EV or hybridosome of claim 79, wherein the binding between the ephrin receptor PBM domain and the cargo protein is a non-covalent binding.

81. The EV or hybridosome of claim 79 or 80, wherein the binding between the ephrin receptor PBM domain and the cargo protein is a reversible binding.

82. The EV or hybridosome of any one of claims 79-81, wherein the binding between the ephrin receptor PBM domain and the cargo protein is capable of being controlled.

83. The EV or hybridosome of claim 82, wherein the binding between the ephrin receptor PBM domain and the cargo protein is capable of being controlled by pH.

84. The EV or hybridosome of claim 82, wherein the binding between the ephrin receptor PBM domain and the cargo protein is capable of being controlled by ionic strength.

85. The EV or hybridosome of any one of claims 79-84, wherein the binding between the ephrin receptor PBM domain and the cargo protein is capable of being controlled such that the cargo protein is bound to the ephrin receptor PBM domain in vitro but is released from the ephrin receptor PBM domain in vivo.

86. The EV or hybridosome of any one of claims 79-84, wherein the binding between the ephrin receptor PBM domain and the cargo protein is capable of being controlled such that the cargo protein is released from the ephrin receptor PBM domain in a manner dependent on the subcellular compartment in which they are located.

87. The EV or hybridosome of any one of claims 79-86, wherein the cargo protein or the SBD comprises a PDZ domain, and the binding between the ephrin receptor PBM domain and the cargo protein is a binding between the ephrin receptor PBM domain and the PDZ domain.

88. The EV or hybridosome of any one of claims 9-87, wherein the cargo protein is a therapeutic protein.

89. The EV or hybridosome of claim 88, wherein the therapeutic protein is a therapeutic antibody or an antigen binding fragment thereof.

90. The EV or hybridosome of claim 88, wherein the therapeutic protein is a gene editor or transposase.

91. The EV or hybridosome of any one of claims 9-87, wherein the cargo protein is a diagnostic protein.

92. The EV or hybridosome of claim 91, wherein the diagnostic protein is a fluorescent protein.

93. The EV or hybridosome of any one of claims 1-92, wherein the polypeptide lacks an ephrin receptor ligand binding domain (LBD).

94. The EV or hybridosome of any one of claims 1-92, wherein the polypeptide comprises a mutated ephrin receptor LBD.

95. The EV or hybridosome of any one of claims 1-94, wherein the polypeptide comprises two different domains that allow the polypeptide to undergo hetero-domain dimerization with another polypeptide identical to said polypeptide.

96. The EV or hybridosome of any one of claims 1-94, wherein the polypeptide comprises two different domains that allow the polypeptide to undergo hetero-domain dimerization with another polypeptide identical to said polypeptide, in a head-to-tail configuration.

97. The EV or hybridosome of any one of claims 1-96, wherein the TM domain is an ephrin receptor TM domain.

98. The EV or hybridosome of any one of claims 1-97, wherein any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof.

99. The EV or hybridosome of any one of claims 1-97, wherein any one or more of the ephrin receptor domains of the polypeptide are from or derived from EphA2, EphA4, EphB2, or a combination thereof.

100. The EV or hybridosome of any one of claims 1-99, wherein the polypeptide further comprises a modified Fc domain of an immunoglobulin.

101. The EV or hybridosome of claim 100, wherein the modified Fc domain is N-terminal to the ephrin receptor CR domain.

102. The EV or hybridosome of claim 101, wherein the modified Fc domain is fused to the remaining portion of the polypeptide by a linker sequence.

103. The EV or hybridosome of any one of claims 100-102, wherein the modified Fc domain
a. is capable of specifically binding to the Fc binding site of a neonatal Fc receptor (FcRn); and
b. lacks the ability to form homodimers.

104. The EV or hybridosome of any one of claim 100-103, wherein the dissociation constant of the modified Fc domain bound to the FcRn at a pH of 6.5 has a value of at most 10⁻⁴ M.

105. The EV or hybridosome of any one of claim 100-104, wherein the dissociation constant of the modified Fc domain bound to the FcRn at a pH of 7.4 has a value of at least 10⁻⁴ M.

106. The EV or hybridosome of any one of claim 100-105, wherein the modified Fc domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid.

107. The EV or hybridosome of any one of claim 100-106, wherein the modified Fc domain is capable of specifically binding to the amino acid sequence between position 135-158 of human FcRn (SEQ ID NO: 228) and/or mouse FcRn (SEQ ID NO: 227).

108. The EV or hybridosome of any one of claims 100-107, wherein the polypeptide does not substantially bind to C1q, FcγRI, FcγRII or FcγRIII.

109. The EV or hybridosome of any one of claims 100-108, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain is decreased by at least 10%, 20%, 30%, 40%, or 50% compared to an unmodified Fe domain.

110. The EV or hybridosome of any one of claims 100-109, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 1.5, 2, 3, 4, or 5-fold, compared to an unmodified Fc domain.

111. The EV or hybridosome of any one of claims 100-110, wherein the modified Fc domain comprises from N-terminus to C-terminus:
a. a modified CH2 domain that is modified to decrease effector function relative to the unmodified CH2 domain; and
b. a modified CH3 domain that is modified to lack the ability to form homodimers.

112. The EV or hybridosome of any one of claims 1-111, wherein the first ephrin receptor FN III domain and the second ephrin receptor FN III domain comprise different amino acid sequences.

113. A method of delivering a therapeutic or diagnostic agent to a target cell or tissue, wherein the method comprises providing the extracellular vesicle or hybridosome of any one of claims 1-112 to said target cell or tissue.

114. A polypeptide comprising in N-terminus to C-terminus direction:
a. a targeting domain;
b. an ephrin receptor CR domain;
c. a first ephrin receptor FN III domain and a second ephrin receptor FN III domain; and
d. a TM domain.

115. The polypeptide of claim 114, wherein the polypeptide lacks ephrin binding activity.

116. The polypeptide of claim 114 or 115, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

117. The polypeptide of any one of claims 114-116, wherein the targeting domain specifically binds to a marker.

118. The polypeptide of claim 117, wherein the marker is a tumor-associated antigen.

119. The polypeptide of claim 118, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

120. A polypeptide comprising in N-terminus to C-terminus direction:
a. an ephrin receptor CR domain;
b. a first ephrin receptor FN III domain and a second ephrin receptor FN III domain;
c. a TM domain; and
d. a cargo protein or a cargo binding domain.

121. The polypeptide of claim 120, wherein the polypeptide lacks ephrin binding activity.

122. A polypeptide comprising in N-terminus to C-terminus direction:
a. a targeting domain;
b. an ephrin receptor CR domain;
c. a first ephrin receptor FN III domain and a second ephrin receptor FN III domain;
d. a TM domain; and
e. a cargo protein or a cargo binding domain.

123. The polypeptide of claim 122, wherein the polypeptide lacks ephrin binding activity.

124. The polypeptide of claim 122 or 123, wherein the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

125. The polypeptide of any one of claims 122-124, wherein the targeting domain specifically binds to a marker.

126. The polypeptide of claim 125, wherein the marker is a tumor-associated antigen.

127. The polypeptide of claim 126, wherein the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-l (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

128. The polypeptide of any one of claims 120-127, wherein the cargo protein or cargo binding domain is fused to the remaining portion of the polypeptide via a linker.

129. The polypeptide of claim 128, wherein the cargo protein or cargo binding domain is covalently fused to the remaining portion of the polypeptide via a linker.

130. The polypeptide of claim 128 or 129, wherein the linker is a peptide linker.

131. The polypeptide of claim 130, wherein the peptide linker comprises an amino acid sequence of (GGGS)n (SEQ ID NO: 226), wherein n is an integer number from 1 to 10.

132. The polypeptide of claim 130, wherein the peptide linker comprises an amino acid sequence of GGGS.

133. The polypeptide of any one of claims 120-132, wherein the polypeptide comprises a cargo binding domain that is capable of binding to a cargo protein directly, or indirectly via a SBD linked to the cargo protein.

134. The polypeptide of claim 133, wherein the binding between the cargo binding domain and the cargo protein is a non-covalent binding.

135. The polypeptide of claim 133 or 134, wherein the binding between the cargo binding domain and the cargo protein is a reversible binding.

136. The polypeptide of any one of claims 133-135, wherein the binding between the cargo binding domain and the cargo protein is capable of being controlled.

137. The polypeptide of claim 136, wherein the binding between the cargo binding domain and the cargo protein is capable of being controlled by pH.

138. The polypeptide of claim 136, wherein the binding between the cargo binding domain and the cargo protein is capable of being controlled by ionic strength.

139. The polypeptide of any one of claims 133-138, wherein the binding between the cargo binding domain and the cargo protein is capable of being controlled such that the cargo protein is bound to the cargo binding domain in vitro but is released from the cargo binding domain in vivo.

140. The polypeptide of any one of claims 133-138, wherein the binding between the cargo binding domain and the cargo protein is capable of being controlled such that the cargo protein is released from the cargo binding domain in a manner dependent on the subcellular compartment in which they are located.

141. The polypeptide of any one of claims 133-140, wherein the cargo binding domain comprises a phosphotyrosine and the cargo protein or the SBD comprises a domain that is capable of binding to phosphotyrosine, and the binding between the cargo binding domain and the cargo protein is a binding between the phosphotyrosine and the domain that is capable of binding to phosphotyrosine.

142. The polypeptide of claim 141, wherein the domain that is capable of binding to phosphotyrosine is a PTB domain.

143. The polypeptide of claim 141, wherein the domain that is capable of binding to phosphotyrosine is an SH2 domain.

144. The polypeptide of any one of claims 133-140, wherein the cargo binding domain comprises a first SAM domain and the cargo protein or the SBD comprises a second SAM domain, and the binding between the cargo binding domain and the cargo protein is a binding between the first SAM domain and the second SAM domain.

145. The polypeptide of any one of claims 133-140, wherein the cargo binding domain comprises a PBM domain and the cargo protein or the SBD comprises a PDZ domain, and the binding between the cargo binding domain and the cargo protein is a binding between the PBM domain and the PDZ domain.

146. The polypeptide of any one of claims 133-140, wherein the cargo binding domain comprises a PDZ domain and the cargo protein or the SBD comprises a PBM domain, and the binding between the cargo binding domain and the cargo protein is a binding between the PDZ domain and the PBM domain.

147. The polypeptide of any one of claims 120-132, wherein the polypeptide comprises a cargo protein.

148. The polypeptide of any one of claims 114-119, wherein the polypeptide further comprises an ephrin receptor JM domain that is capable of binding to a cargo protein directly, or indirectly via a SBD, and is C-terminal to the TM domain.

149. The polypeptide of claim 148, wherein the binding between the ephrin receptor JM domain and the cargo protein is a non-covalent binding.

150. The polypeptide of claim 148 or 149, wherein the binding between the ephrin receptor JM domain and the cargo protein is a reversible binding.

151. The polypeptide of any one of claims 148-150, wherein the binding between the ephrin receptor JM domain and the cargo protein is capable of being controlled.

152. The polypeptide of claim 151, wherein the binding between the ephrin receptor JM domain and the cargo protein is capable of being controlled by pH.

153. The polypeptide of claim 151, wherein the binding between the ephrin receptor JM domain and the cargo protein is capable of being controlled by ionic strength.

154. The polypeptide of any one of claims 148-153, wherein the binding between the ephrin receptor JM domain and the cargo protein is capable of being controlled such that the cargo protein is bound to the ephrin receptor JM domain in vitro but is released from the ephrin receptor JM domain in vivo.

155. The polypeptide of any one of claims 148-153, wherein the binding between the ephrin receptor JM domain and the cargo protein is capable of being controlled such that the cargo protein is released from the ephrin receptor JM domain in a manner dependent on the subcellular compartment in which they are located.

156. The polypeptide of any one of claims 148-155, wherein the ephrin receptor JM domain comprises a phosphotyrosine and the cargo protein or the SBD comprises a domain that is capable of binding to phosphotyrosine, and the binding between the ephrin receptor JM domain and the cargo protein is a binding between the phosphotyrosine and the domain that is capable of binding to phosphotyrosine.

157. The polypeptide of claim 156, wherein the domain that is capable of binding to phosphotyrosine is a PTB domain.

158. The polypeptide of claim 156, wherein the domain that is capable of binding to phosphotyrosine is an SH2 domain.

159. The polypeptide of any one of claims 156-158, wherein the ephrin receptor JM domain comprises:
(i) a (X₁)-Ptyr-(X₂) motif, wherein Ptyr is a phosphotyrosine, X₁ is Y, P, V, I, T, or F, and X₂ is I, V, L, or A;
(ii) a (X₃)-Ptyr-(X₄) motif, wherein Ptyr is a phosphotyrosine, X₃ is T, A, or S, and X₄ is E or G; or
(iii) both (i) and (ii).

160. The polypeptide of any one of claims 114-119, wherein the polypeptide further comprises an ephrin receptor KD that is capable of binding to a cargo protein directly, or indirectly via a SBD linked to the cargo protein, and is C-terminal to the TM domain.

161. The polypeptide of claim 160, wherein the binding between the ephrin receptor KD and the cargo protein is a non-covalent binding.

162. The polypeptide of claim 160 or 161, wherein the binding between the ephrin receptor KD and the cargo protein is a reversible binding.

163. The polypeptide of any one of claims 160-162, wherein the binding between the ephrin receptor KD and the cargo protein is capable of being controlled.

164. The polypeptide of claim 163, wherein the binding between the ephrin receptor KD and the cargo protein is capable of being controlled by pH.

165. The polypeptide of claim 163, wherein the binding between the ephrin receptor KD and the cargo protein is capable of being controlled by ionic strength.

166. The polypeptide of any one of claims 160-165, wherein the binding between the ephrin receptor KD and the cargo protein is capable of being controlled such that the cargo protein is bound to the ephrin receptor KD in vitro but is released from the ephrin receptor KD in vivo.

167. The polypeptide of any one of claims 160-165, wherein the binding between the ephrin receptor KD and the cargo protein is capable of being controlled such that the cargo protein is released from the ephrin receptor KD in a manner dependent on the subcellular compartment in which they are located.

168. The polypeptide of any one of claims 160-167, wherein the ephrin receptor KD comprises a phosphotyrosine and the cargo protein or the SBD comprises a domain that is capable of binding to phosphotyrosine, and the binding between the ephrin receptor KD and the cargo protein is a binding between the phosphotyrosine and the domain that is capable of binding to phosphotyrosine.

169. The polypeptide of claim 168, wherein the domain that is capable of binding to phosphotyrosine is a PTB domain.

170. The polypeptide of claim 168, wherein the domain that is capable of binding to phosphotyrosine is an SH2 domain.

171. The polypeptide of any one of claims 168-170, wherein the KD comprises an (X₇)-Ptyr-(X₈) motif in the activation loop, wherein Ptyr is a phosphotyrosine, X₇ is T, V, or A, and X₈ is E or T.

172. The polypeptide of any one of claims 114-119, wherein the polypeptide further comprises a SAM linker domain that is capable of binding to a cargo protein directly, or indirectly via a SBD linked to the cargo protein, and is C-terminal to the TM domain.

173. The polypeptide of claim 172, wherein the binding between the SAM linker domain and the cargo protein is a non-covalent binding.

174. The polypeptide of claim 172 or 173, wherein the binding between the SAM linker domain and the cargo protein is a reversible binding.

175. The polypeptide of any one of claims 172-174, wherein the binding between the SAM linker domain and the cargo protein is capable of being controlled.

176. The polypeptide of claim 175, wherein the binding between the SAM linker domain and the cargo protein is capable of being controlled by pH.

177. The polypeptide of claim 175, wherein the binding between the SAM linker domain and the cargo protein is capable of being controlled by ionic strength.

178. The polypeptide of any one of claims 172-177, wherein the binding between the SAM linker domain and the cargo protein is capable of being controlled such that the cargo protein is bound to the SAM linker domain in vitro but is released from the SAM linker domain in vivo.

179. The polypeptide of any one of claims 172-177, wherein the binding between the SAM linker domain and the cargo protein is capable of being controlled such that the cargo protein is released from the SAM linker domain in a manner dependent on the subcellular compartment in which they are located.

180. The polypeptide of any one of claims 172-179, wherein the SAM linker domain comprises a phosphorylated amino acid or a phosphomimetic amino acid and the cargo protein or the SBD comprises a domain that is capable of binding to the phosphorylated amino acid or phosphomimetic amino acid, and the binding between the SAM linker domain and the cargo protein is a binding between the phosphorylated amino acid or phosphomimetic amino acid and the domain that is capable of binding to the phosphorylated amino acid or phosphomimetic amino acid.

181. The polypeptide of any one of claims 172-180, wherein the SAM linker domain is an ephrin receptor SAM linker domain.

182. The polypeptide of any one of claims 114-119, wherein the polypeptide further comprises a SAM domain that is capable of binding to a cargo protein directly, or indirectly via a SBD linked to the cargo protein, and is C-terminal to the TM domain.

183. The polypeptide of claim 182, wherein the binding between the SAM domain and the cargo protein is a non-covalent binding.

184. The polypeptide of claim 182 or 183, wherein the binding between the SAM domain and the cargo protein is a reversible binding.

185. The polypeptide of any one of claims 182-184, wherein the binding between the SAM domain and the cargo protein is capable of being controlled.

186. The polypeptide of claim 185, wherein the binding between the SAM domain and the cargo protein is capable of being controlled by pH.

187. The polypeptide of claim 185, wherein the binding between the SAM domain and the cargo protein is capable of being controlled by ionic strength.

188. The polypeptide of any one of claims 182-187, wherein the binding between the SAM domain and the cargo protein is capable of being controlled such that the cargo protein is bound to the SAM domain in vitro but is released from the SAM domain in vivo.

189. The polypeptide of any one of claims 182-187, wherein the binding between the SAM domain and the cargo protein is capable of being controlled such that the cargo protein is released from the SAM domain in a manner dependent on the subcellular compartment in which they are located.

190. The polypeptide of any one of claims 182-189, wherein the cargo protein or the SBD comprises a second SAM domain, and the binding between the SAM domain and the cargo protein is a binding between the SAM domain and the second SAM domain.

191. The polypeptide of any one of claims 182-189, wherein the SAM domain comprises a phosphotyrosine and the cargo protein or the SBD comprises a domain that is capable of binding to phosphotyrosine, and the binding between the SAM domain and the cargo protein is a binding between the phosphotyrosine and the domain that is capable of binding to phosphotyrosine.

192. The polypeptide of claim 191, wherein the domain that is capable of binding to phosphotyrosine is a PTB domain.

193. The polypeptide of claim 191, wherein the domain that is capable of binding to phosphotyrosine is an SH2 domain.

194. The polypeptide of any one of claims 191-193, wherein the SAM domain comprises a phosphotyrosine in the α2 helix.

195. The polypeptide of claim 194, wherein the phosphotyrosine in the α2 helix of the SAM domain is in an (X₅)-Ptyr-(X₆) motif, wherein Ptyr is the phosphotyrosine, X₅ is C, R, Q, or H, and X₆ is Q, I, E, K, R, or T.

196. The polypeptide of any one of claims 182-195, wherein the SAM domain is an ephrin receptor SAM domain.

197. The polypeptide of any one of claims 114-119, wherein the polypeptide further comprises an ephrin receptor PDZ binding motif (PBM) domain that is capable of binding to a cargo protein directly, or indirectly via a SBD linked to the cargo protein, and is C-terminal to the TM domain.

198. The polypeptide of claim 197, wherein the binding between the ephrin receptor PBM domain and the cargo protein is a non-covalent binding.

199. The polypeptide of claim 197 or 198, wherein the binding between the ephrin receptor PBM domain and the cargo protein is a reversible binding.

200. The polypeptide of any one of claims 197-199, wherein the binding between the ephrin receptor PBM domain and the cargo protein is capable of being controlled.

201. The polypeptide of claim 200, wherein the binding between the ephrin receptor PBM domain and the cargo protein is capable of being controlled by pH.

202. The polypeptide of claim 200, wherein the binding between the ephrin receptor PBM domain and the cargo protein is capable of being controlled by ionic strength.

203. The polypeptide of any one of claims 197-202, wherein the binding between the ephrin receptor PBM domain and the cargo protein is capable of being controlled such that the cargo protein is bound to the ephrin receptor PBM domain in vitro but is released from the ephrin receptor PBM domain in vivo.

204. The polypeptide of any one of claims 197-202, wherein the binding between the ephrin receptor PBM domain and the cargo protein is capable of being controlled such that the cargo protein is released from the ephrin receptor PBM domain in a manner dependent on the subcellular compartment in which they are located.

205. The polypeptide of any one of claims 197-204, wherein the cargo protein or the SBD comprises a PDZ domain, and the binding between the ephrin receptor PBM domain and the cargo protein is a binding between the ephrin receptor PBM domain and the PDZ domain.

206. The polypeptide of any one of claims 120-205, wherein the cargo protein is a therapeutic protein.

207. The polypeptide of claim 206, wherein the therapeutic protein is a therapeutic antibody or an antigen binding fragment thereof.

208. The EV or hybridosome of claim 206, wherein the therapeutic protein is a gene editor or transposase.

209. The polypeptide of any one of claims 120-205, wherein the cargo protein is a diagnostic protein.

210. The polypeptide of claim 209, wherein the diagnostic protein is a fluorescent protein.

211. The polypeptide of any one of claims 114-210, wherein the polypeptide lacks an ephrin receptor ligand binding domain (LBD).

212. The polypeptide of any one of claims 114-210, wherein the polypeptide comprises a mutated ephrin receptor LBD.

213. The polypeptide of any one of claims 114-210, wherein the polypeptide comprises two different domains that allow the polypeptide to undergo hetero-domain dimerization with another polypeptide identical to said polypeptide.

214. The polypeptide of any one of claims 114-210, wherein the polypeptide comprises two different domains that allow the polypeptide to undergo hetero-domain dimerization with another polypeptide identical to said polypeptide, in a head-to-tail configuration.

215. The polypeptide of any one of claims 114-214, wherein the TM domain is an ephrin receptor TM domain.

216. The polypeptide of any one of claims 114-215, wherein any one or more of the ephrin receptor domains of the polypeptide are from or derived from EphA1, EphA2, EphA3, EphA4, EphA5, EphA6, EphA7, EphA8, EphA10, EphB1, EphB2, EphB3, EphB4, EphB6, or a combination thereof.

217. The polypeptide of any one of claims 114-215, wherein any one or more of the ephrin receptor domains of the polypeptide are from or derived from EphA2, EphA4, EphB2, or a combination thereof.

218. The polypeptide of any one of claims 114-217, wherein the polypeptide further comprises a modified Fc domain of an immunoglobulin.

219. The polypeptide of claim 218, wherein the modified Fc domain is N-terminal to the ephrin receptor CR domain.

220. The polypeptide of claim 219, wherein the modified Fc domain is fused to the remaining portion of the polypeptide by a linker sequence.

221. The polypeptide of any one of claims 218-220, wherein the modified Fc domain
a. is capable of specifically binding to the Fc binding site of a neonatal Fc receptor (FcRn); and
b. lacks the ability to form homodimers.

222. The polypeptide of any one of claim 218-221, wherein the dissociation constant of the modified Fc domain bound to the FcRn at a pH of 6.5 has a value of at most 10⁻⁴ M.

223. The polypeptide of any one of claim 218-222, wherein the dissociation constant of the modified Fc domain bound to the FcRn at a pH of 7.4 has a value of at least 10⁻⁴ M.

224. The polypeptide of any one of claim 218-223, wherein the modified Fc domain is capable of specifically binding to the amino acid sequence LNGEEFMX₁FX₂X₃X₄X₅GX₆WX₇GX₈W (SEQ ID NO: 230), wherein X₁, X₂, X₃, X₄, X₅, X₆, X₇, and X₈ each is any amino acid.

225. The polypeptide of any one of claim 218-224, wherein the modified Fc domain is capable of specifically binding to the amino acid sequence between position 135-158 of human FcRn (SEQ ID NO: 228) and/or mouse FcRn (SEQ ID NO: 227).

226. The polypeptide of any one of claims 218-225, wherein the polypeptide does not substantially bind to C1q, FcγRI, FcγRII or FcγRIII.

227. The polypeptide of any one of claims 218-226, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 10%, 20%, 30%, 40%, or 50% compared to an unmodified Fc domain.

228. The polypeptide of any one of claims 218-227, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 1.5, 2, 3, 4, or 5-fold, compared to an unmodified Fc domain.

229. The polypeptide of any one of claims 218-228, wherein the modified Fc domain comprises from N-terminus to C-terminus:
a. a modified CH2 domain that is modified to decrease effector function relative to the unmodified CH2 domain; and
b. a modified CH3 domain that is modified to lack the ability to form homodimers.

230. The polypeptide of any one of claims 114-229, wherein the first ephrin receptor FN III domain and the second ephrin receptor FN III domain comprise different amino acid sequences.

231. A nucleic acid encoding the polypeptide of any one of claim 114-230.

232. An expression vector comprising the nucleic acid of claim 231.

233. A cell comprising the nucleic acid of claim 231 or the expression vector of claim 232.

234. A method of producing an EV, wherein the method comprises:
a. transfecting cells with the nucleic acid of claim 231 or the expression vector of claim 232;
b. cultivating the cells under suitable conditions for the production of the EV; and
c. collecting the EV secreted by the cells.

235. A method of producing a hybridosome, wherein the method comprises contacting a first EV with a second EV, thereby uniting the first EV with the second EV and producing the hybridosome,
wherein said first EV has been produced in vitro, and the first EV comprises (i) a membrane, and (ii) a fusogenic, ionizable, cationic lipid, and
wherein said second EV has been produced by the method of claim 234.

236. A method of purifying an EV or a hybridosome, wherein the method comprises:
a. providing the EV or hybridosome, wherein the EV or hybridosome comprises a first binding partner, wherein the first binding partner is capable of binding to the Fc binding site of an FcRn in a pH dependent manner; and
b. contacting at a first pH the EV or hybridosome comprising the first binding partner with a second binding partner, wherein the second binding partner comprises the Fc binding site of the FcRn and is associated with a solid matrix; and
c. eluting the EV or hybridosome comprising the first binding partner from the solid matrix at a second pH.

237. The method of claim 236, wherein the method further comprises a washing step at the first pH.

238. The method of claim 236 or 237, wherein the first pH is below 6.5.

239. The method of any one of claims 236-238, wherein the second pH is above 7.4.

240. The method of any one of claims 236-239, wherein the Fc binding site of the FcRn comprises the amino acid sequence of SEQ ID NO: 230.

241. A method of purifying an EV or a hybridosome, wherein the method comprises:
a. providing the EV or hybridosome, wherein the EV or hybridosome comprises a first binding partner, wherein the first binding partner is capable of binding to the Fc binding site of an FcRn in a pH dependent manner and comprises or consists of the polypeptide of any one of claims 111-224; and
b. contacting at a first pH the EV or hybridosome comprising the first binding partner with a second binding partner, wherein the second binding partner comprises the Fc binding site of the FcRn and is associated with a solid matrix; and
c. eluting the EV or hybridosome comprising the first binding partner from the solid matrix at a second pH.

242. The method of claim 241, wherein the method further comprises a washing step at the first pH.

243. The method of claim 241 or 242, wherein the first pH is below 6.5.

244. The method of any one of claims 241-243, wherein the second pH is above 7.4.

245. The method of any one of claims 241-244, wherein the Fc binding site of the FcRn comprises the amino acid sequence of SEQ ID NO: 230.
